SEQUENCE LISTING (234 sequences)

SEQ ID NO: 1 - DNA, 101 bp, Cannabis sativa
  source 1..101: mol_type = genomic DNA
aaaccatttgcagcaagttggccccttgggtcaatgaaggacctctccacgagtagtgctgccatgggcctttcttcaacagttaaagacaaagatctttc

SEQ ID NO: 2 - DNA, 101 bp, Cannabis sativa
  source 1..101: mol_type = genomic DNA
cgatttctgaaatcttgaaaccctagtttttctctcttttggaagagacatagtcttgaggtgtttctcgaatctttagactgagtgatcaatttgttttt

SEQ ID NO: 3 - DNA, 101 bp, Cannabis sativa
  source 1..101: mol_type = genomic DNA
tggaggatgtagtatattgatatagtgctgcaataatattcattctattcttctggataaattatatattctcatcctcatcctctggatcgatccatact

SEQ ID NO: 4 - DNA, 101 bp, Cannabis sativa
  source 1..101: mol_type = genomic DNA
tattaattagaatgtcagaatggcctcagtactagttttacaatgctattacttttgctggacacaattaaaattaagttctcttggagcatttatactgg

SEQ ID NO: 5 - DNA, 101 bp, Cannabis sativa
  source 1..101: mol_type = genomic DNA
gggtcaatctcaaagagcttctccacaatctttgtgtaggcagtttcatggcgtttctcatcggcagcaattgtgccgcatatctgcgccagtttcaggtc

SEQ ID NO: 6 - DNA, 101 bp, Cannabis sativa
  source 1..101: mol_type = genomic DNA
aaactgtgccaagttggcaaacttgccaatctaaggattgtctcactgacgacctcatcttccaaactctgcgaaacaataccattgttaaaaaaactatc

SEQ ID NO: 7 - DNA, 101 bp, Cannabis sativa
  source 1..101: mol_type = genomic DNA
ctcgttaatgtgtgtatctttagttttatttggtttttctttgtaataatggttgtatcctaggttttatgtggtgttggctttggtcgttttgtaaggtt

SEQ ID NO: 8 - DNA, 101 bp, Cannabis sativa
  source 1..101: mol_type = genomic DNA
gtaggatttgatggtgtttatatagagatattagagaatggaattatggggtacttgagttgagttcctatagcatctttctatagcatatgattatgcgg

SEQ ID NO: 9 - DNA, 101 bp, Cannabis sativa
  source 1..101: mol_type = genomic DNA
aaagccgatctagtctactacagcgaatctcaaaataaatttatgctttggagtaattaatcgaccgtgcccatacctaaccataccacccaaaataaaaa

SEQ ID NO: 10 - DNA, 101 bp, Cannabis sativa
  source 1..101: mol_type = genomic DNA
atctttccttcgccgtaagtgaggactcccaatacatttgggaggaggtcactcatgaacatgtgaactttgtctctttttcaacaacaaaaaaaataaaa

SEQ ID NO: 11 - DNA, 101 bp, Cannabis sativa
  source 1..101: mol_type = genomic DNA
ggaggagccagtggaataggccaaagaagtgctgaaatttttgcccaacatggagccaaattagtcatagccgatgttcaagacgaattaggtcactccct

SEQ ID NO: 12 - DNA, 101 bp, Cannabis sativa
  source 1..101: mol_type = genomic DNA
tttaagatcatcatcatttcaattatgttatatcatcacttctctctcccttcatatatactatactatttcttttcttatttaattttgtggtggttatt

SEQ ID NO: 13 - DNA, 101 bp, Cannabis sativa
  source 1..101: mol_type = genomic DNA
atgttgcaccacttatcttagcatctttaacatgtgtactaatactccatggtcgaaggttagcctgaatgtcatgattattgccaacatcaactctcatc

SEQ ID NO: 14 - DNA, 101 bp, Cannabis sativa
  source 1..101: mol_type = genomic DNA
tatttgtttgttcaagactcatgagaatttaattgtttgttatttgattttccatttgtaataaataaagttatagttttttattttttgattttgtatta

SEQ ID NO: 15 - DNA, 101 bp, Cannabis sativa
  source 1..101: mol_type = genomic DNA
tgatctgactgaaatgttcctcaacttgtggctgcaggcttttacgagacacgaattgcatattatgcgtccagaggtgccaatggttatcgttttgcttc

SEQ ID NO: 16 - DNA, 101 bp, Cannabis sativa
  source 1..101: mol_type = genomic DNA
ttggattgaccaccgtcttgttgagagagaagattgtcgttctttggtttgtgggtaattgggtcaattcccattttggctaaacgtttctttagatgtgt

SEQ ID NO: 17 - DNA, 101 bp, Cannabis sativa
  source 1..101: mol_type = genomic DNA
ggcaatcttggtattgctagaaagctttttgatcgaatgcctgaaagagatttggtttcttggaatgtgatgattactgggtatgcaaagcaaggtgaaat

SEQ ID NO: 18 - DNA, 101 bp, Cannabis sativa
  source 1..101: mol_type = genomic DNA
acaaacacagcagcactaagcacagcattattcaacgagggattaagttgcggtgcatgtttccaaataaggtgcgccaacgaccctcaatggtgcctccc

SEQ ID NO: 19 - DNA, 101 bp, Cannabis sativa
  source 1..101: mol_type = genomic DNA
atagtcacgctactaacacgaacaaacaagctttggcccaattgatattctctcaatgtctaaaaaaaaaacaagactaaaatctcctagcatgaatggat

SEQ ID NO: 20 - DNA, 101 bp, Cannabis sativa
  source 1..101: mol_type = genomic DNA
tatatcggattctagtctttctgggcataggtatttgttttctgggtacagtttctctgtctccagaaggaaatggaggaacccttttgttcagattaggt

SEQ ID NO: 21 - DNA, 101 bp, Cannabis sativa
  source 1..101: mol_type = genomic DNA
tctcactttcactaaactttctgctagcaacagaaggcggggtttctctcctcttttctttagaagaaaggacataggtgctgaaagggattctgtcgttt

SEQ ID NO: 22 - DNA, 101 bp, Cannabis sativa
  source 1..101: mol_type = genomic DNA
tatattagcagctaactatacatactcattccacaagttctctcatagtttagtacatcaaaccatctcattctctcagcataaatcttgtagaataatcc

SEQ ID NO: 23 - DNA, 101 bp, Cannabis sativa
  source 1..101: mol_type = genomic DNA
cgtacgtatatatttatataaatatataaattaatttataatattatgatagaaaagtcatccaaatcaactcttgggattggctgttggaagagtgttag

SEQ ID NO: 24 - DNA, 101 bp, Cannabis sativa
  source 1..101: mol_type = genomic DNA
catttatccttctatttgtaatttatttatttaatcaaaacaaaataaatgttacgcaatccaacattcacaagatatataacattaaactcactcactta

SEQ ID NO: 25 - DNA, 101 bp, Cannabis sativa
  source 1..101: mol_type = genomic DNA
gtgggtcatatctctggtgcacacatgaatcctgctgttactctagcttttgctacttttctacattttccatggaaactggtacattatttttttatttc

SEQ ID NO: 26 - DNA, 101 bp, Cannabis sativa
  source 1..101: mol_type = genomic DNA
aaaggggttcttaaagtctggatgcataataacaagaacacacttgggaaaaaagatattaatggcggaggagccagtacaggtggaagaagcaacaataa

SEQ ID NO: 27 - DNA, 101 bp, Cannabis sativa
  source 1..101: mol_type = genomic DNA
gatccttacgtgtggcgccaatacaatgaagttgccaatgaatttcaacaatttcaggttctcttacacaggtttgtagagaatgaaccttttcaaggtcc

SEQ ID NO: 28 - DNA, 101 bp, Cannabis sativa
  source 1..101: mol_type = genomic DNA
aactcaacacccacttttacagctcaagaaatctctccttacaagcctcctattaactcttttccaaatggagagtttcccaatgaatttatgttctctcc

SEQ ID NO: 29 - DNA, 101 bp, Cannabis sativa
  source 1..101: mol_type = genomic DNA
aaaataaaactcaagccgtagatcactacacacaacatattaacaacaatatatcagcatggcttctttcgatgcagttgcctctctgaatttgagattac

SEQ ID NO: 30 - DNA, 101 bp, Cannabis sativa
  source 1..101: mol_type = genomic DNA
tctccctcatcatcatcgtcaccttcggcagattctgccccaacttcctcatagtccttctcaagggcagccaagtcttctctcgcttcagaaaactcacc

SEQ ID NO: 31 - DNA, 101 bp, Cannabis sativa
  source 1..101: mol_type = genomic DNA
aatagtactttgtgttattttttatttatttagtcttttgaaaaaaagaggttaattgtcttttccttattcaaaatttgcggccataacaaacactagct

SEQ ID NO: 32 - DNA, 101 bp, Cannabis sativa
  source 1..101: mol_type = genomic DNA
tttgataaagaaaaatgggaaaagagtttgggataacagaggattaatatgtaatatgatcacatggtgttgcacattgagacatgtacatgaaggcccac

SEQ ID NO: 33 - DNA, 101 bp, Cannabis sativa
  source 1..101: mol_type = genomic DNA
taattagatgggaaatttaatgtaattaaacatacctaattctagaaaatatggctgcaccatatggagtccgtgaatctattgtatgtattttttttacc

SEQ ID NO: 34 - DNA, 101 bp, Cannabis sativa
  source 1..101: mol_type = genomic DNA
gagcgatctggctcggctactagaatgcaggcagggcttgctaggaatggacaaccaacaagaagcctgtcagcatcaaatgctcagcaggcttctccgta

SEQ ID NO: 35 - DNA, 101 bp, Cannabis sativa
  source 1..101: mol_type = genomic DNA
ttacaggaagcaggaccgggaggactgggtggaggaggtatgtggggttcggccactgatgaaaggagtgtctacactaacattgccaacagtcaaaagaa

SEQ ID NO: 36 - DNA, 101 bp, Cannabis sativa
  source 1..101: mol_type = genomic DNA
tttcctttggaagtttggatttagatggtaatttcttcccaaaaagcagactcttgagccactttcctggagattttcccattgcaagaattatgtgttct

SEQ ID NO: 37 - DNA, 101 bp, Cannabis sativa
  source 1..101: mol_type = genomic DNA
ataatacctgttataaacaagattgatcagccaactgctgatcctgaccgtgttatagctcaattaaagtccatgtttgatcttgaccctgctgatgctct

SEQ ID NO: 38 - DNA, 101 bp, Cannabis sativa
  source 1..101: mol_type = genomic DNA
taatcacttactgcatctaaaagaatattagaagtcttgaaatcacgatatattacaggcctttgaacttcttcatgcaaaaaagcaagaccctttgcagc

SEQ ID NO: 39 - DNA, 101 bp, Cannabis sativa
  source 1..101: mol_type = genomic DNA
actgaaactaggattcctgtcataccctaccgtattgaggaacatgacacaatagtaattgctacatagatactcacaactcacgatagactcaaacctta

SEQ ID NO: 40 - DNA, 101 bp, Cannabis sativa
  source 1..101: mol_type = genomic DNA
ttaattataattaaataggcgccatctctcttgcgacagtagtccctttacttctctattcattattcctctcatatcttaattattaaaattaaggatta

SEQ ID NO: 41 - DNA, 101 bp, Cannabis sativa
  source 1..101: mol_type = genomic DNA
ggtatccacagcccctcgacccaaatcaacactcaaatcttcattactgacctcaacgcaaccttctaccatggacttcggacccgcgagattatgcgttg

SEQ ID NO: 42 - DNA, 101 bp, Cannabis sativa
  source 1..101: mol_type = genomic DNA
tattattttatttggttgtcagagtcgctttacgtatcggtgtatttaggcctatattattttgggattcaaaataaaaaataaatgtattattagatttt

SEQ ID NO: 43 - DNA, 101 bp, Cannabis sativa
  source 1..101: mol_type = genomic DNA
atcactaactatacatgtgtgtcatagtgttgattttgtataactacataaaatttcattacaaaatgttttctattttggttgaaacatattcattttat

SEQ ID NO: 44 - DNA, 101 bp, Cannabis sativa
  source 1..101: mol_type = genomic DNA
tttgatagtattattggacactagttagtgaaaatattcatttactatgtcccacaaatttcatcaccatataattatatatagtgtgcggttgttaatgg

SEQ ID NO: 45 - DNA, 101 bp, Cannabis sativa
  source 1..101: mol_type = genomic DNA
atccaactcaaaaggatgaaaatgaccagacagacagtagacacggttacctaaattagtactgtctttgaccattctaagcaaataaataataaaaagag

SEQ ID NO: 46 - DNA, 101 bp, Cannabis sativa
  source 1..101: mol_type = genomic DNA
tatcttcacaaaaacaacaacttgataggttgtgtttattgcaacaacaaaccaaacacaacacaacacaagagcaactttgcgattcgaatggcatttcg

SEQ ID NO: 47 - DNA, 101 bp, Cannabis sativa
  source 1..101: mol_type = genomic DNA
ttgatttctacactaatcgaaattcctgttgtttgtgcataaaccatccaaaattttagatggcgcaggatccaaattccaaggcccctttgataatgttt

SEQ ID NO: 48 - DNA, 101 bp, Cannabis sativa
  source 1..101: mol_type = genomic DNA
atattcaacaaaccatatggcccagagctgctgctgctgcaggtatgtaagcttacattgcaatgcattaatgaatttccatcattgattatgatgagggg

SEQ ID NO: 49 - DNA, 101 bp, Cannabis sativa
  source 1..101: mol_type = genomic DNA
tagtagtggatgtgttgggtggtctggagagtgttggggagcaggattgtgtggtcatcatcatcatccttgctgccctatttgggttattggtagtaggc

SEQ ID NO: 50 - DNA, 101 bp, Cannabis sativa
  source 1..101: mol_type = genomic DNA
attctcaacaaaatcttttcggtttatcattgctgcactgcatccctttgtgatatctgtttttgctttccaagttttatttgtctaagaaggtttgtgtc

SEQ ID NO: 51 - DNA, 101 bp, Cannabis sativa
  source 1..101: mol_type = genomic DNA
ccaaacaaacacacaagagggaatgtgaaaattttcgatgacgtcaccttcaccagccagcgcaccaaataaattaaaaaattaaaaacgacgagtccaaa

SEQ ID NO: 52 - DNA, 101 bp, Cannabis sativa
  source 1..101: mol_type = genomic DNA
agaaagataatggcaatttacatgcctcccttgatgtggcatctgtggaatctgaggggtatgatattaatatgcttcctgggtctggagatcaagctgtg

SEQ ID NO: 53 - DNA, 101 bp, Cannabis sativa
  source 1..101: mol_type = genomic DNA
acttaatttttttataatattattaaatatagtattcttagatagaaattatactgtataactaatgatatgtgtacgtaaacatgacccggaccgacacg

SEQ ID NO: 54 - DNA, 101 bp, Cannabis sativa
  source 1..101: mol_type = genomic DNA
attgtacgaattagaagatgaagaagaagaagtaatggtagaagatgatcatcttcaagaaatgttggtgttcaacaccaaaacgacgtcggttaacaagg

SEQ ID NO: 55 - DNA, 101 bp, Cannabis sativa
  source 1..101: mol_type = genomic DNA
agtcaatgtactagaaaagcatcaacttgaggaattgaatttcatatggagtattaattatgttggttttgaaccaaaacgtggagagggtgttcttgaag

SEQ ID NO: 56 - DNA, 101 bp, Cannabis sativa
  source 1..101: mol_type = genomic DNA
tatattttcttttttagaaaaccacccgccaaatccaaatatttctcaaatttttaaaatccaacataacaagtcaacgatccttgtcataatccaatctc

SEQ ID NO: 57 - DNA, 101 bp, Cannabis sativa
  source 1..101: mol_type = genomic DNA
tcgttaaaatactcacaacatgacattaaaatataataacataataatagattacatacatgggaggcttggtggcattagctctttcccaaccattctca

SEQ ID NO: 58 - DNA, 101 bp, Cannabis sativa
  source 1..101: mol_type = genomic DNA
atattaagcaaaaccataaaaaacaaaaaacaaaagtcatggttttttgtcttggaattggaccccctcctcctaccctagccttatatatacattttctt

SEQ ID NO: 59 - DNA, 101 bp, Cannabis sativa
  source 1..101: mol_type = genomic DNA
cgttaaaacgacatcattttcacaaaacgacattatccagtaaaaggacaataattacaatatgaagccaaactgttcgataaaagtccaagccatggcat

SEQ ID NO: 60 - DNA, 101 bp, Cannabis sativa
  source 1..101: mol_type = genomic DNA
aaatcagttccatttggatcaagagaagaagcattaaagaatggaagtagtttgggttattttaactcagctcaagccttagctgactatgctcacattct

SEQ ID NO: 61 - DNA, 101 bp, Cannabis sativa
  source 1..101: mol_type = genomic DNA
ttatattaagaaataccattgtcaatggtgtcttcactgcagatatatattcctcattgacaaattcggtagaataagatggcaaggctatggattggcaa

SEQ ID NO: 62 - DNA, 101 bp, Cannabis sativa
  source 1..101: mol_type = genomic DNA
tttttcaattttgaaaagttggtcaatttcatccaattaaactaatcataccatgtgtccgtacaatataaaaagtccttacttcatactctagtctagtc

SEQ ID NO: 63 - DNA, 101 bp, Cannabis sativa
  source 1..101: mol_type = genomic DNA
tcatccaatgcaagattttttttttatttcattcttactcctatgtattcgttcattctaaccgagcataccctaagtttcggtaagcagtcattaacact

SEQ ID NO: 64 - DNA, 101 bp, Cannabis sativa
  source 1..101: mol_type = genomic DNA
taaattttgaacttttgaagtgttatacatctcttatctatattttaaaaaatattggcgcgtgcaatacacctcgttagttgaaagaaagagggagagtc

SEQ ID NO: 65 - DNA, 101 bp, Cannabis sativa
  source 1..101: mol_type = genomic DNA
tttgttagtcagagaattttcctcttcctgaggtaagaatacattaggaaaaatcctacaacagctccaacgattatcccagctgttgtgagccagaatgc

SEQ ID NO: 66 - DNA, 101 bp, Cannabis sativa
  source 1..101: mol_type = genomic DNA
ttgctaattaccgggcaacgttcgccatagaaatctaacatctccatagcgcatgatcctaggttgcagttccccgtgtaattttgaggtacattagtgct

SEQ ID NO: 67 - DNA, 101 bp, Cannabis sativa
  source 1..101: mol_type = genomic DNA
tttcttttcggagattcagtactcgatgttggtaacaataataatttagcaacggttgttaaagccaattttcctccttatggacaggacttcaaaaatca

SEQ ID NO: 68 - DNA, 101 bp, Cannabis sativa
  source 1..101: mol_type = genomic DNA
tcttccaaatttttcgttgtggcaatatctaccgatgatacatcgttctcgagagtatcatcctatcacaaaatatacatatatatttcattatttatatt

SEQ ID NO: 69 - DNA, 101 bp, Cannabis sativa
  source 1..101: mol_type = genomic DNA
aaaatatttcgtaaagcattataataaaaattgccaaccttgccaccactcgagggtagtgtattgagagagtgctaattatagcattttattataaaata

SEQ ID NO: 70 - DNA, 101 bp, Cannabis sativa
  source 1..101: mol_type = genomic DNA
gcccactctctcagtttctttagataaataccgacccttcgtttccctttacaaacccaaaaatctctcactgtcgttcctcactaacacacacccccgcg

SEQ ID NO: 71 - DNA, 101 bp, Cannabis sativa
  source 1..101: mol_type = genomic DNA
aaaaatctcctattaatatacattttagggttaataaacttgaacaaattaacaagctatagatctttgcacccaaaattcgaaacaattattctacatgc

SEQ ID NO: 72 - DNA, 101 bp, Cannabis sativa
  source 1..101: mol_type = genomic DNA
aaagttgctaagttgatcgatagctacattgcagaggttgcttctgatgtgaatttaaagcccacaaagattcgttctttagctgaggctataccagggtc

SEQ ID NO: 73 - DNA, 101 bp, Cannabis sativa
  source 1..101: mol_type = genomic DNA
gaggaggaggaaaaggaggagaagccgcgatatctactatcaacataagcggcccttaaggtgggccgctgagatgaagatcttgaatggaggctattatt

SEQ ID NO: 74 - DNA, 101 bp, Cannabis sativa
  source 1..101: mol_type = genomic DNA
ccctttttacaaccgtttcgaagtgattcgccgctcttctcaccttctttcgcctctcctctcttcactctctccacctctttctgctcttatcacagaca

SEQ ID NO: 75 - DNA, 101 bp, Cannabis sativa
  source 1..101: mol_type = genomic DNA
tgcgtgtttctccatgtgagtttcgggttttctcagagagttgagaggttttgatatactgttatgaactgaatcttaagaaattcttgtataatcttagt

SEQ ID NO: 76 - DNA, 101 bp, Cannabis sativa
  source 1..101: mol_type = genomic DNA
tccgagttcttccacatctgtgatagtgtatatgatcccgccaacttgtagaacataagcatactcgtctagcaagtgtggactgatcaccctacggcgat

SEQ ID NO: 77 - DNA, 101 bp, Cannabis sativa
  source 1..101: mol_type = genomic DNA
ccgtcttcctactgatgacaattataatgattatggtattggaggacaacatgaccgtaggtttgattactctccaaaagcatttgacaaaattccatgga

SEQ ID NO: 78 - DNA, 101 bp, Cannabis sativa
  source 1..101: mol_type = genomic DNA
ccatttcgtgtatatcgagtagacgacatagatggatctttgtatgcatctactagcttatcataaaattcgtccctagcaggattattctggtcctcttt

SEQ ID NO: 79 - DNA, 101 bp, Cannabis sativa
  source 1..101: mol_type = genomic DNA
ttaataaatggaagtggtgtggtttggttttggttatgatttgatgaaatcgagtgtggtgtggtgagtcagtgtcgtacttttttgggattggtgtgacg

SEQ ID NO: 80 - DNA, 101 bp, Cannabis sativa
  source 1..101: mol_type = genomic DNA
tcgtccattagctctctacaccatgtcactcgctttggagggcaactacctggtccgaaacacctgcaagttaaccaattcaagctttagttttggctgca

SEQ ID NO: 81 - DNA, 101 bp, Cannabis sativa
  source 1..101: mol_type = genomic DNA
gtgtaagattttttataatcccattaatttataaacccgacccggaaatccacaaacccactttcctttccataatttccatgcacacataaacgacgccg

SEQ ID NO: 82 - DNA, 101 bp, Cannabis sativa
  source 1..101: mol_type = genomic DNA
gtcttcaaatgccagaactgcaaaccagcaaccgataagcaaaccaacagagaaagaagactaaaggtagccatttgtgagttagtctttctattaagttg

SEQ ID NO: 83 - DNA, 101 bp, Cannabis sativa
  source 1..101: mol_type = genomic DNA
aatgcagtcaaagtcatacagagcgggaactctcttgctcagtcttcttctccgttctgagaagtgttgggagtacttgaagcggagttttcagatgttac

SEQ ID NO: 84 - DNA, 101 bp, Cannabis sativa
  source 1..101: mol_type = genomic DNA
caattaagcacgataataattacatgcatacaattatagtaaagtaataactgtaataataacgtgttgatttagataacaaagtgtttatctatttaatt

SEQ ID NO: 85 - DNA, 101 bp, Cannabis sativa
  source 1..101: mol_type = genomic DNA
aattaacctaatacaaatacaaatacattttactcaagcttaagctaaaaggttagctgtaaattttggtcattcattaggtaaagtctgaaaagttatac

SEQ ID NO: 86 - DNA, 101 bp, Cannabis sativa
  source 1..101: mol_type = genomic DNA
cgctttagcccacgtcttaccctgcgaaccacatgaagggtcatatgaggacacatgaccccactcaatttattataaattatattttgtataaaaaattt

SEQ ID NO: 87 - DNA, 101 bp, Cannabis sativa
  source 1..101: mol_type = genomic DNA
gcactaacgatggagtgttcttttcttcttcctatggtaaattgagaatcgagtgttctttttcttatttctagtgaaaaaatgagaatagcacataaagt

SEQ ID NO: 88 - DNA, 101 bp, Cannabis sativa
  source 1..101: mol_type = genomic DNA
aagaaaaactgagtttactataatagcattatgtaataagtggtatgtgtagtgttatggttggcctaccaactaaaactttcaattatgaaatgtccatt

SEQ ID NO: 89 - DNA, 101 bp, Cannabis sativa
  source 1..101: mol_type = genomic DNA
ataataataaaaactgattgatgaataaataaaagttaagtacagtcattagatgtcaatttaagtagttcataattaaccaatccatggatttaaaagtt

SEQ ID NO: 90 - DNA, 101 bp, Cannabis sativa
  source 1..101: mol_type = genomic DNA
acaccatgaaaataactccagcggaccaaggattgaatagggatgtaaaccgggttgggcgggtgcggggattgctctcccacccccatactcgcttctta

SEQ ID NO: 91 - DNA, 101 bp, Cannabis sativa
  source 1..101: mol_type = genomic DNA
aaaaactcagcttcatcatcatcttcttcaatggttaaagatggagtcaagaagggtttatttgaattgcctcgtgatcaccaccgtgaattggaagctga

SEQ ID NO: 92 - DNA, 101 bp, Cannabis sativa
  source 1..101: mol_type = genomic DNA
acccttctctttcgtacccaaatatcacgcccttcttcatggaatttccttatcagagcctacgcttccagccattcacccagagaagccatctgggtgtt

SEQ ID NO: 93 - DNA, 101 bp, Cannabis sativa
  source 1..101: mol_type = genomic DNA
cattgtatgcagcaggtcaagttataggtgcaattttaggagcatttacactacgtgaagtgctagacccaattcaagatattggaaccacaacaccacat

SEQ ID NO: 94 - DNA, 101 bp, Cannabis sativa
  source 1..101: mol_type = genomic DNA
caataatgctttatcaggttcttcttcttcctcttctttcatcccctattaaaatttatatttaatttcaagattatgatgataatttgagatatttttat

SEQ ID NO: 95 - DNA, 101 bp, Cannabis sativa
  source 1..101: mol_type = genomic DNA
catctaatcacaatcaccaacaaatttaagtgctaaagaactatatctttggtaagaaagacacaaacaagtacatatcattatgtcatggttagttttat

SEQ ID NO: 96 - DNA, 101 bp, Cannabis sativa
  source 1..101: mol_type = genomic DNA
ttattggaaattctccaagtttcctaagggcatttagggcctccttccttagtcttccagttgttgtagcaagggcacacagctttattaccataataccc

SEQ ID NO: 97 - DNA, 101 bp, Cannabis sativa
  source 1..101: mol_type = genomic DNA
gaagcactaatccacgaaacccctcaatatgggtctaaagataaagacataaggctacataatccaacaacctttagaaccagttaaaacccaaattaaac

SEQ ID NO: 98 - DNA, 101 bp, Cannabis sativa
  source 1..101: mol_type = genomic DNA
tcagtatgaaatgaatgagtttgatttgaacgaggaagaagctacacactccaagccctagacaagctacatgcacaaacaacattgtattctattattat

SEQ ID NO: 99 - DNA, 101 bp, Cannabis sativa
  source 1..101: mol_type = genomic DNA
gtctttgttgattagtgatgcaaattagtattggttggcaaatattttaggtttaagttaaaagtcaatatataccaaaccaattttatagcaatattaga

SEQ ID NO: 100 - DNA, 101 bp, Cannabis sativa
  source 1..101: mol_type = genomic DNA
atacactatctgtcaacttttctggctggactggtggttggttttgtctcagcatggcggctagctctgctgagtgtggcggtgattcccggcattgcctt

SEQ ID NO: 101 - DNA, 101 bp, Cannabis sativa
  source 1..101: mol_type = genomic DNA
ttgataatgttacagcaattgtccctcaaaggatttgccggagacacagcaaaagctcatgcaaagacaagtatgattgccggggaaggtgtgagcaacat

SEQ ID NO: 102 - DNA, 101 bp, Cannabis sativa
  source 1..101: mol_type = genomic DNA
aaaaactatatataactgttattttgggcctcttgggtctttgctgtatatatagtctaaattttgaccccgggtatccagggacagactacagagtacat

SEQ ID NO: 103 - DNA, 101 bp, Cannabis sativa
  source 1..101: mol_type = genomic DNA
gatattcttcattagcattttcccatgttttgcttatagctacaagatatgtaattatttaagattatagttacaagatatgtaattattaaaaaaaaatt

SEQ ID NO: 104 - DNA, 101 bp, Cannabis sativa
  source 1..101: mol_type = genomic DNA
aaattcaccgcattgactcgaaatggtctgctggcaatgtaatttgactctaagcattctgagtgaacaaggaacagccctctagcaaatgatccaaccta

SEQ ID NO: 105 - DNA, 101 bp, Cannabis sativa
  source 1..101: mol_type = genomic DNA
cttgtcttcttacaagtggggtcacccctagacttatttaaaccccattttggggtagttgggggagattgtattgtatgtctctaaaactctaatagcat

SEQ ID NO: 106 - DNA, 101 bp, Cannabis sativa
  source 1..101: mol_type = genomic DNA
gagttcttctccaccaattctctaaacatcgaagattgaagaaggaggccaagagcagaaggagaagacttgctgcgagtcatactactgccaattagagg

SEQ ID NO: 107 - DNA, 101 bp, Cannabis sativa
  source 1..101: mol_type = genomic DNA
aagaaacaaggtggtggtaaagctgttgaagttgataaagctgacattgtaggggtaacatggatgaaggtcccaagaacaaatcaacttggtgttcggat

SEQ ID NO: 108 - DNA, 101 bp, Cannabis sativa
  source 1..101: mol_type = genomic DNA
taataattattcaaaagaaaagagcctagaaaaggttttagacttaccatatcagcctacatagccttcctttctctaaagaacatagccacaagaaaact

SEQ ID NO: 109 - DNA, 101 bp, Cannabis sativa
  source 1..101: mol_type = genomic DNA
ggtcaccatggtagggttcaccattgaggagcttagcatagtagtccgttcagatttcagactgacttctctttaattcaaattgtggattctgttttttt

SEQ ID NO: 110 - DNA, 101 bp, Cannabis sativa
  source 1..101: mol_type = genomic DNA
tagcactagagagaaacatacatatcatcaaaagacataggcttggatctctgagttagaattggtgagcttgtagaaagcatctgctgctttcccatgaa

SEQ ID NO: 111 - DNA, 101 bp, Cannabis sativa
  source 1..101: mol_type = genomic DNA
cccaaaacaaaaaaaatgctgaggcgagtgctgcaacgagcttccactggccgccatattgccaaatcatactacttggcttctcatctttcaccttcttt

SEQ ID NO: 112 - DNA, 101 bp, Cannabis sativa
  source 1..101: mol_type = genomic DNA
taatcgaaatagagtggaggggagtggcccgttgagaatgttactaaacaaattaagatagagtaagttctctaagtttcctatactgataggaatggatc

SEQ ID NO: 113 - DNA, 101 bp, Cannabis sativa
  source 1..101: mol_type = genomic DNA
ttaaagaatcggaatatttcaaattacgagaatgaaaagatgaagataagttggtctttgaaaattgaaaagggagaagaagccttgttactttccactgg

SEQ ID NO: 114 - DNA, 101 bp, Cannabis sativa
  source 1..101: mol_type = genomic DNA
ggccttacaaccctatacaaaccattgcttgacaatataaagatgtctttcctgttatcttcaccaaaagagaatatgaaaccaagtccagagactgagct

SEQ ID NO: 115 - DNA, 101 bp, Cannabis sativa
  source 1..101: mol_type = genomic DNA
catgtaaagaaactatactaaaagatagaacttactcttattagtacaaaaaggatacgaagaagagtaatcaaaaatcagcagaaagtgacataacatag

SEQ ID NO: 116 - DNA, 101 bp, Cannabis sativa
  source 1..101: mol_type = genomic DNA
cttccctttctgattagagcagggtcaagtttttctacataattagtagtaaaaactatgatcctttcaccaccacaagctgaccaaatcccatcaatgaa

SEQ ID NO: 117 - DNA, 101 bp, Cannabis sativa
  source 1..101: mol_type = genomic DNA
aaactagccggatgctcaaaaaggacatgactccacttagtccgattcccgccataaaagctctcaccagatccattcgtgtaaagctttcgttgccgatt

SEQ ID NO: 118 - DNA, 101 bp, Cannabis sativa
  source 1..101: mol_type = genomic DNA
ttttattacatttgttattatgtcaaatcaaataggggtctattatctctcagaagaaaataaaataggggtctaccacatatagaaatttccaagactcc

SEQ ID NO: 119 - DNA, 101 bp, Cannabis sativa
  source 1..101: mol_type = genomic DNA
aaaatgatagttgttgtagatattcttggactagctttgatggcacgtgctaacacgaaagtgcctctttacgtagactcaagttcgtatgagtcatttgg

SEQ ID NO: 120 - DNA, 101 bp, Cannabis sativa
  source 1..101: mol_type = genomic DNA
ggcactgtctgcatcagaaggcggtggtggcccactcaaactcgtttgcctcgtcaacttaataaaagttcgcctgagcctgcgaaactcaagcaagccaa

SEQ ID NO: 121 - DNA, 101 bp, Cannabis sativa
  source 1..101: mol_type = genomic DNA
tttgcggtgttcttataaagaaagataattatgcaaaatctgatcttgatttatgtgcagattttgtgtccgaaaatgggtcgagattgagtgtttcatcg

SEQ ID NO: 122 - DNA, 101 bp, Cannabis sativa
  source 1..101: mol_type = genomic DNA
ttcaaccacggcagcaacaatacccttcactgtctcctgcataacattgataaagatcacgaacaatgagttatcaaaattatctaactagtctaattaat

SEQ ID NO: 123 - DNA, 101 bp, Cannabis sativa
  source 1..101: mol_type = genomic DNA
aagggaacttagtgaaagagctgttgttgttattacaaaaggagagtaattttacatacctagctttaagtattgttcccatagttcctatttgagttgca

SEQ ID NO: 124 - DNA, 101 bp, Cannabis sativa
  source 1..101: mol_type = genomic DNA
ccatttacatacacagggccttgagaacttacctcaagatgaaaagtctctaaccatttgcatgcttctcccatattctctctcaccccctattaatcatt

SEQ ID NO: 125 - DNA, 101 bp, Cannabis sativa
  source 1..101: mol_type = genomic DNA
taaagagcttattaggagaatactcccttaatgtagatccgattagtctatgtggggcctctagcttgagaatgacccctcaatcgtgataacaattattt

SEQ ID NO: 126 - DNA, 101 bp, Cannabis sativa
  source 1..101: mol_type = genomic DNA
gaaaaaaacaagtaacgagtaacaaatgataggagaatatgaggctaataattcattatggtctctctttcacgaaatgtaagagccaagagggttgagga

SEQ ID NO: 127 - DNA, 101 bp, Cannabis sativa
  source 1..101: mol_type = genomic DNA
actaaaaaattatcactaaaatttaaaaatgattattttttgtagtgataggtgaactttctacattcaaagtagaagttttggctttgttgcatgctatt

SEQ ID NO: 128 - DNA, 101 bp, Cannabis sativa
  source 1..101: mol_type = genomic DNA
ggtgaggtattggagaatgtttcaggggaagttgaattcaaacacgttgagtttgcttacccttctagacccgaaagcataattttcaaggatttttgctt

SEQ ID NO: 129 - DNA, 101 bp, Cannabis sativa
  source 1..101: mol_type = genomic DNA
aatctcccacgaccataattatactgattattagcaggcaatcttgatatatacgaagcatatagtccaatagcaagagctgcacaatcaaacaacatgtg

SEQ ID NO: 130 - DNA, 101 bp, Cannabis sativa
  source 1..101: mol_type = genomic DNA
tattaggagacccagagctaccagacccattctgaccacaatcttttccacttcctgaacccaaagctaccaaaaacttctcattccatctgcgaccacca

SEQ ID NO: 131 - DNA, 101 bp, Cannabis sativa
  source 1..101: mol_type = genomic DNA
tggatgaaaggttacaatgactgtgtccactggtgcatgccaggtcctgtggatcattggagccattttttgatggctgttatgaggaaggaattgggttt

SEQ ID NO: 132 - DNA, 101 bp, Cannabis sativa
  source 1..101: mol_type = genomic DNA
gagaagaagggtgcccaaagaataagaataggccatgaaaacaaagaaggtgaagcctttcaaagtagctgatttatacagaatatgtagaccaaccatac

SEQ ID NO: 133 - DNA, 101 bp, Cannabis sativa
  source 1..101: mol_type = genomic DNA
ttattattattatcatcatcattttacctttaatggcagttctgcaacgaatatgcggtacctaaggacaagctagacaacttcaaaaatgtcacagcaca

SEQ ID NO: 134 - DNA, 101 bp, Cannabis sativa
  source 1..101: mol_type = genomic DNA
ttcattctctaactatttttaattcttctatccttgtacattcattttgtataggtgggagctatctcggatgcttttgaaaatttccaattaaaaacata

SEQ ID NO: 135 - DNA, 101 bp, Cannabis sativa
  source 1..101: mol_type = genomic DNA
ctgaaatcttacctcattaagaatcgaagaaagaagctggctttcaaagtagttgcgcagcttgcactagatcttgcaagagggttggatttgctttcttc

SEQ ID NO: 136 - DNA, 101 bp, Cannabis sativa
  source 1..101: mol_type = genomic DNA
cttacatgtagacacaagaaacgtttgtggtctgtggagtatggagctaaccatataccccgtgtaagagacactacagccctgccaaatgatgagggtaa

SEQ ID NO: 137 - DNA, 101 bp, Cannabis sativa
  source 1..101: mol_type = genomic DNA
ttgaccagagagtcagactaatgtacttggctaacgaaggagatatagatagtattaacgagctgcttgatgaaggtactgatgttaatttcaaggacatc

SEQ ID NO: 138 - DNA, 101 bp, Cannabis sativa
  source 1..101: mol_type = genomic DNA
tggggttggagtcaatgcgtgtagatgttgtgcttctttttgcttggaacgatattttctttgggttattttagcagtaggaaccaaaatgtatcgactct

SEQ ID NO: 139 - DNA, 101 bp, Cannabis sativa
  source 1..101: mol_type = genomic DNA
cacaattgtcgttattggaaaactataggcactcaaaataggggacaaaacgttgttttcaattccctaagaaatgactatttttcttaattttaacataa

SEQ ID NO: 140 - DNA, 101 bp, Cannabis sativa
  source 1..101: mol_type = genomic DNA
attgctgaaaatttcaaatcccctcgattcatctagctagaatcgtttcatggtttctgggttttttcaataattaataactcaatattcctcctcttctt

SEQ ID NO: 141 - DNA, 101 bp, Cannabis sativa
  source 1..101: mol_type = genomic DNA
gatcaatctgcttcccaatcctatgaccatcacaactttcttcccgtaacccttatggactccaatccccactactctcactcccatcgccctgaccacac

SEQ ID NO: 142 - DNA, 101 bp, Cannabis sativa
  source 1..101: mol_type = genomic DNA
aattgttggaatattcaagggctgtgatcattttgatttatttcatgatactgactactcttttcactaacagttcccaaacttaatgcctatatcaatga

SEQ ID NO: 143 - DNA, 101 bp, Cannabis sativa
  source 1..101: mol_type = genomic DNA
acacatacatagaattgtttacgtgcaaatagttactttaatctacctgttgggatcagaaccacttttatgtctcacatgtttatttaatttattcatat

SEQ ID NO: 144 - DNA, 101 bp, Cannabis sativa
  source 1..101: mol_type = genomic DNA
acttccaatgcatttttgacccctgatctcgatgaggcaaaggaatattttgataaggctgcagtgtactttcagcaagcatccgatgaggtttttctcct

SEQ ID NO: 145 - DNA, 20 bp, synthetic construct
  source 1..20: mol_type = other DNA
gacaacaccgaccaacaaca

SEQ ID NO: 146 - DNA, 20 bp, synthetic construct
  source 1..20: mol_type = other DNA
aacccatcatcctcacttgc

SEQ ID NO: 147 - DNA, 20 bp, synthetic construct
  source 1..20: mol_type = other DNA
aggtcttttcgactgccaat

SEQ ID NO: 148 - DNA, 20 bp, synthetic construct
  source 1..20: mol_type = other DNA
ggaggcttattgggttcctt

SEQ ID NO: 149 - DNA, 21 bp, synthetic construct
  source 1..21: mol_type = other DNA
agggactctcttgttgttggt

SEQ ID NO: 150 - DNA, 20 bp, synthetic construct
  source 1..20: mol_type = other DNA
cttccaccwgagccagtttc

SEQ ID NO: 151 - DNA, 20 bp, synthetic construct
  source 1..20: mol_type = other DNA
cccggttatggtattgttgg

SEQ ID NO: 152 - DNA, 20 bp, synthetic construct
  source 1..20: mol_type = other DNA
acggtcgttgacaagtttcc

SEQ ID NO: 153 - DNA, 20 bp, synthetic construct
  source 1..20: mol_type = other DNA
gaagtgcaacaaagccgatt

SEQ ID NO: 154 - DNA, 19 bp, synthetic construct
  source 1..19: mol_type = other DNA
aatccatcgaggcaaccac

SEQ ID NO: 155 - DNA, 20 bp, synthetic construct
  source 1..20: mol_type = other DNA
gttggtgggcgaaatacagt

SEQ ID NO: 156 - DNA, 20 bp, synthetic construct
  source 1..20: mol_type = other DNA
tgtagcaacgagcacgaatc

SEQ ID NO: 157 - DNA, 20 bp, synthetic construct
  source 1..20: mol_type = other DNA
actataccggtggcggaagt

SEQ ID NO: 158 - DNA, 20 bp, synthetic construct
  source 1..20: mol_type = other DNA
tctttggttggaaagccttg

SEQ ID NO: 159 - DNA, 20 bp, synthetic construct
  source 1..20: mol_type = other DNA
acacgcctcatcctttgaat

SEQ ID NO: 160 - DNA, 20 bp, synthetic construct
  source 1..20: mol_type = other DNA
gtgggacgatgaggaatgtt

SEQ ID NO: 161 - DNA, 20 bp, synthetic construct
  source 1..20: mol_type = other DNA
gccttcatgatcttggcttt

SEQ ID NO: 162 - DNA, 20 bp, synthetic construct
  source 1..20: mol_type = other DNA
catttagggcctccttcctt

SEQ ID NO: 163 - DNA, 20 bp, synthetic construct
  source 1..20: mol_type = other DNA
ccaggctcaaatttgctgtt

SEQ ID NO: 164 - DNA, 20 bp, synthetic construct
  source 1..20: mol_type = other DNA
tggagcaaatcctggttgat

SEQ ID NO: 165 - DNA, 20 bp, synthetic construct
  source 1..20: mol_type = other DNA
gccatattggatggtcgaag

SEQ ID NO: 166 - DNA, 20 bp, synthetic construct
  source 1..20: mol_type = other DNA
gcggggagagatcgtttatc

SEQ ID NO: 167 - DNA, 20 bp, synthetic construct
  source 1..20: mol_type = other DNA
atgggaaggggtaagattga

SEQ ID NO: 168 - DNA, 20 bp, synthetic construct
  source 1..20: mol_type = other DNA
tccctcaaattcagcctctg

SEQ ID NO: 169 - DNA, 23 bp, synthetic construct
  source 1..23: mol_type = other DNA
tgctaagagaaaagctggaattt

SEQ ID NO: 170 - DNA, 20 bp, synthetic construct
  source 1..20: mol_type = other DNA
tcttggagatttggctgcat

SEQ ID NO: 171 - DNA, 1431 bp, synthetic construct
  source 1..1431: mol_type = other DNA
atgtcgtctcaggacaacaccgaccaacaacaccaatccactggtgtggaactacacaacgttgttgatgggaactctcaatccagtattgaagtaggatggtatattctcggtgataatcaagagcatgttggtccctatgtttcttctgaattgcttgagcatttcttgaatgggtatctcaccccgagtacactcgtgtggtctgaaggaaggactgaatggcagccattatcctcaatccccgagttgatgacatacatacctcaacaaggagccgatgattctatgccctccaatgatgaagagtttttgaaatggcagaaggagattcaagaggctgaggctgaggcagaagcagaggcaaaaaatggctctactgatttcggcggagcctcgatggataatgatgacagaccttcaaccccaccagagggtgaagaagaattcacagacgatgacgggactacctataaatgggatcgaggtctgagggtttgggttcctcaggaggacatagatagcaaaggtggagaatataagttagaagacatgattttcttaaaagaagaagaggtcttttcgactgccaatatttctaatgttcctgcaagtgaagaagataatacatctaagcaacctgattcctctacaaaacaagttgatgatagcaatgaggcagtggaaggaaaaaatgctaagagaaaattgcctgatgaggaagctgctaagaaggaacccaataagcctcctgatagctggtttgaattaaaagtaaatactcatgtgtatgtgactgggttgcccgaggacgtgacagttgatgaagtggtagaagtattctccaagtgcgggataataaaggaggatacagaaaccaaaaagccccgtataaagctttatgttaacaaagagactggaagaaagaagggagatgcacttgttacatatctgaaggaaccttcagttgctctggctttgcaaattctggatggagcaccttttcgtcctggtgggattccaatgacagtcaccccagccaaatttgagcagaaagggaatacatttatttccaagaaagttgacaacaagaagaaaaagaaacttaagaaggtggaagataagatgctaggatggggtggtcgtgatgatgcgaagttgtgtattccagctactgttgttcttcgttatatgttcatgcctgctgaaatgagggccgatgaaaacttacgttctgagctagaggctgacatacaggaggaatgttcaaagcttggtccactggacaatgttaaggtgtgcgagaatcaccctcaaggtgttgttttagtaagatacaaggacaggaaggatgctcaaaagtgcatagagctgatgaatggaagatgtttggcggtagacagatacatgcaagtgaggatgatgggttag

SEQ ID NO: 172 - DNA, 1348 bp, synthetic construct
  source 1..1348: mol_type = other DNA
caacaccaatccactggtgtggaactacacaacgttgttgatgggaactctcaatccagtattgaagtaggatggtatattctcggtgataatcaagagcatgttggtccctatgtttcttctgaattgcttgagcatttcttgaatgggtatctcaccccgagtacactcgtgtggtctgaaggaaggactgaatggcagccattatcctcaatccccgagttgatgatatacatacctcaacaaggagccgatgattctatgccctccaatgatgaagagtttttgaaatggcagaaggagattcaagaggctgaggctgaggcagaagcagaggcaaaaaatggctctactgatttcggcggagcctcgatggataatgatgacagaccttcaaccccaccagagggtgaagaagaattcacagacgatgacgggactacctataaatgggatcgaggtctgagggtttgggttcctcaggaggacatagatagcaaaggtcgagaatataagttagaagacatgattttcttaaaagaagaagaggtcttttcgactgccaatatttctaatgttcctgcaagtgaagaagataatacatctaagcaacctgattcctctacaaaacaagttgatgatagcaatgaggcagtggaaggaaaaaatgctaagagaaaattgcctgatgaggaagctgctaagaaggaacccaataagcctcctgatagctggtttgaattaaaagtaaatactcatgtgtatgtgactgggttgcccgaggacgtgacagttgatgaagtggtagaagtattctccaagtgcgggataataaaggaggatacagaaaccaaaaagccccgtataaagctttatgttgacaaagagactggaagaaagaagggagatgcacttgttacatatctgaaggaaccttcagttgctctggctttgcaaattctggatggagcaccttttcgtcctggtgggattccaatgacagtcaccccagccaaatttgagcagaaagggaatacatttatttccaagaaagttgacaacaagaagaaaaagaaacttaagaaggtggaagataagatgctaggatggggtggtcgtgatgatgcgaagttgtgtattccagctactgttgttcttcgttatatgttcatgcctgctgaaatgagggccgatgaaaacttacgttctgagctagaggctgacatacaggaggaatgttcaaagcttggtccgctggacaatgttaaggtgtgcgagaatcaccctcaaggtgttgttttagtaagatacaaggacaggaaggatgctcaaaagtgcatagaactga

SEQ ID NO: 173 - protein, 476 aa, Cannabis sativa
MSSQDNTDQQHQSTGVELHNVVDGNSQSSIEVGWYILGDNQEHVGPYVSSELLEHFLNGYLTPSTLVWSEGRTEWQPLSSIPELMTYIPQQGADDSMPSNDEEFLKWQKEIQEAEAEAEAEAKNGSTDFGGASMDNDDRPSTPPEGEEEFTDDDGTTYKWDRGLRVWVPQEDIDSKGGEYKLEDMIFLKEEEVFSTANISNVPASEEDNTSKQPDSSTKQVDDSNEAVEGKNAKRKLPDEEAAKKEPNKPPDSWFELKVNTHVYVTGLPEDVTVDEVVEVFSKCGIIKEDTETKKPRIKLYVNKETGRKKGDALVTYLKEPSVALALQILDGAPFRPGGIPMTVTPAKFEQKGNTFISKKVDNKKKKKLKKVEDKMLGWGGRDDAKLCIPATVVLRYMFMPAEMRADENLRSELEADIQEECSKLGPLDNVKVCENHPQGVVLVRYKDRKDAQKCIELMNGRCLAVDRYMQVRMMG

SEQ ID NO: 174 - protein, 449 aa, Cannabis sativa
QHQSTGVELHNVVDGNSQSSIEVGWYILGDNQEHVGPYVSSELLEHFLNGYLTPSTLVWSEGRTEWQPLSSIPELMIYIPQQGADDSMPSNDEEFLKWQKEIQEAEAEAEAEAKNGSTDFGGASMDNDDRPSTPPEGEEEFTDDDGTTYKWDRGLRVWVPQEDIDSKGREYKLEDMIFLKEEEVFSTANISNVPASEEDNTSKQPDSSTKQVDDSNEAVEGKNAKRKLPDEEAAKKEPNKPPDSWFELKVNTHVYVTGLPEDVTVDEVVEVFSKCGIIKEDTETKKPRIKLYVDKETGRKKGDALVTYLKEPSVALALQILDGAPFRPGGIPMTVTPAKFEQKGNTFISKKVDNKKKKKLKKVEDKMLGWGGRDDAKLCIPATVVLRYMFMPAEMRADENLRSELEADIQEECSKLGPLDNVKVCENHPQGVVLVRYKDRKDAQKCIEL

SEQ ID NO: 175 - DNA, 101 bp, Cannabis sativa
  source 1..101: mol_type = genomic DNA
tgaaggaaggactgaatggcagccattatcctcaatccccgagttgatgacatacatacctcaacaaggagccgatgattctagtaagggtggagtatttt

SEQ ID NO: 176 - DNA, 101 bp, Cannabis sativa
  source 1..101: mol_type = genomic DNA
aaaaaaattaggatacagaaaccaaaaagccccgtataaagctttatgttaacaaagagactggaagaaagaagggagatgcacttgttacatatctgaag

SEQ ID NO: 177 - DNA, 585 bp, synthetic construct
  source 1..585: mol_type = other DNA
atggctagagtactacttaataataataataataataataataataataataataataataatagtagggactctcttgttgttggtagagtaataggagatgttttggatccttttacaagatcagtccctatgagagtgtattatggtaacagagaggtcaacaatggctatgagttcaaaccttctcaacttctcaaccaacctcgagttcatattggtggtcaagatcttaggaccttctacactttgcttatggtggatccggatgctcctaaccctagtgaccccaatctaagggagtatttgcattggttggtgattgatattccaggaagtacaggaccaagtttcggagaagaggtggtgtgctatgagaatccaaggccaagtatgggaattcataggtttgtgtttgtgttgttcaagcaattgggaagacagacagtgtatgcaccgggatggcgtcaaaatttcaacaccagagattttgctgagctttataatcttggtgcccctgttgctgctctctatttcaattgtcagagggaaactggctcaggtggaaggagaccttccaactaa

SEQ ID NO: 178 - DNA, 392 bp, synthetic construct
  source 1..392: mol_type = other DNA
tcagtccctatgagagtgtattatggtaacagagaggtcaacaatggctatgaattcaaaccttctcaacttgttaaccaacctcgtgttcatgttggtggtcaagatcttaggaccttctacactttgcttatggtggatccggatgctcctaaccctagtgaccccaatctaagggagtatttgcattggttggtgatcgatattccaggaactacagggccaagtttcggagaagaggtggtgtgctatgagaatccaaggccaagtatgggaattcataggtttgtgtttgtgttgttcaagcaattgggacggcagacagtgtatgcaccgggatggcgtcaaaatttcaacaccagagattttgctgagctttataatcttggtgc

SEQ ID NO: 179 - DNA, 573 bp, synthetic construct
  source 1..573: mol_type = other DNA
atggctagagtactacttaataataataataataataataataataataatagtagggactctcttgttgttggtagagtaataggagatgttttggatccttttacaagatcagtccctatgagagtgtattatggtaacagagaggtcaacaatggctatgagttcaaaccttctcaacttctcaaccaacctcgtgttcatattggtggtcaagatcttaggaccttctacactttgcttatggtggatccggatgctcctaaccctagtgaccccaatctaagggagtatttgcattggttggtgattgatattccaggaagtacaggaccaagtttcggagaagaggtggtgtgctatgagaatccaaggccaagtatgggaattcatagatttgtgtttgtgttgtttaaacaattgggaaggcagacagtgtatgcaccaggatggcgtcaaaatttcaacaccagagactttgctgagctttacaaccttggtgcccctgttgctgctctctatttcaactgccagagggaaactggctctggtggaaggagaccttccaactaa

SEQ ID NO: 180 - protein, 194 aa, Cannabis sativa
MARVLLNNNNNNNNNNNNNNNSRDSLVVGRVIGDVLDPFTRSVPMRVYYGNREVNNGYEFKPSQLLNQPRVHIGGQDLRTFYTLLMVDPDAPNPSDPNLREYLHWLVIDIPGSTGPSFGEEVVCYENPRPSMGIHRFVFVLFKQLGRQTVYAPGWRQNFNTRDFAELYNLGAPVAALYFNCQRETGSGGRRPSN

SEQ ID NO: 181 - protein, 130 aa, Cannabis sativa
SVPMRVYYGNREVNNGYEFKPSQLVNQPRVHVGGQDLRTFYTLLMVDPDAPNPSDPNLREYLHWLVIDIPGTTGPSFGEEVVCYENPRPSMGIHRFVFVLFKQLGRQTVYAPGWRQNFNTRDFAELYNLG

SEQ ID NO: 182 - protein, 190 aa, Cannabis sativa
MARVLLNNNNNNNNNNNSRDSLVVGRVIGDVLDPFTRSVPMRVYYGNREVNNGYEFKPSQLLNQPRVHIGGQDLRTFYTLLMVDPDAPNPSDPNLREYLHWLVIDIPGSTGPSFGEEVVCYENPRPSMGIHRFVFVLFKQLGRQTVYAPGWRQNFNTRDFAELYNLGAPVAALYFNCQRETGSGGRRPSN

SEQ ID NO: 183 - DNA, 109 bp, Cannabis sativa
  source 1..109: mol_type = genomic DNA
ttattatggctagagtactacttaataataataataataataataataataataataataataataatagtagggactctcttgttgttggtagagtaataggagatgt

SEQ ID NO: 184 - DNA, 100 bp, Cannabis sativa
  source 1..100: mol_type = genomic DNA
tggtaacagagaggtcaacaatggctatgagttcaaaccttctcaacttctcaaccaacctcgagttcatattggtggtcaagatcttaggaccttctac

SEQ ID NO: 185 - DNA, 100 bp, Cannabis sativa
  source 1..100: mol_type = genomic DNA
gtaacagagaggtcaacaatggctatgagttcaaaccttctcaacttctcaaccaacctcgagttcatattggtggtcaagatcttaggaccttctacac

SEQ ID NO: 186 - DNA, 1536 bp, synthetic construct
  source 1..1536: mol_type = other DNA
atgagagcacaaatgtcggagcctaaagacccggcgatcaagctcttcggcaagactataccggtggcggaagttccggttacttccggtgggtctcaaggaccttcagtttcggcttctgcgacttctatggacgataatatggatcaggaacctaaaccaaactcttcttcttctgaggtcattaccagtaaagatgaggatgatagagacacagaaaaggcaacaacaaatgatgatcccacagagactaaagaggaagatggaaaccaacctatgtctcctgaagagccaaggaattcgggtacaacttctggaagttctgagaacccgacaacacctgttgataaggagagtgttacaccgaaagcttcaaagaccgaagaagagcagagcgagggaaataattcacaagaaaagaccttgaaaaaaccagacaagatacttccatgcccccggtgtaatagcatggacaccaagttctgttactacaacaattacaacgttaaccaacccagacacttctgcaagaaatgccaaagatattggactgctggtgggacgatgaggaatgttcctgtgggtgctggtcgtcgtaagaacaagaactctgcttctcactaccgtcacataactgtttctgaagctcttcaaaatggaagaactgatgtttcaagtggaattcactctcctcatccttcaatgaaatctaatggtactgtcctgacatttggctctgatgcacccttatgtgaatcaatggcatctgttctgaatcttgctgataaaaacatgaggaactgcacacagaatggttttcttaaacccgaagaagtaaggactcctgtagcttttaaaagtgtagaaaatggagataacaatgttgatggatcttcaattacaacatcgaattcaaaggatgaggcgtgtaacaatgtttcacaagaacaagtcatacaaaatcctcagggtgttcctccccaaatgccatgctttcctgggcctccttggccgtacccttggaatgctgctcagtggaacccttcggttccaatgcccgctttctgtcctccaggatatcctatgccattttaccctgcacctgcttattggggttgtagtgtaccagggacatggaacatcccttggcttccaatgccaacatctcaaaacccagccgccccgagctctggtcctaactctcctaactccccaacattgggaaaacattcaagggatgaaaacacagttaaatcaaattcctcagaagaggaacaaccaaaagaaagcaattctgagagatgcctttggattccaaaaacattgaggatcgacgatccaggtgaagctgctaggagctctatatgggcaacattggggataaagaatgataaagccgattcaatcagcgggggaggacttttcaaggctttccaaccaaagaacgacgaaaagaaccacagagcagaaaactcagtcttacaagcgaatccagccgcattgtcaagatcgctaagctttcaagagaggtcgtaa

SEQ ID NO: 187 - DNA, 1222 bp, synthetic construct
  source 1..1222: mol_type = other DNA
gaggtcattaccagtaaagatgaggatgatagagacacagaaaaggcaacaacaaatgatgatcccacagagactaaagaggaagatggaaaccaacccatgtctcctgaagagccaagaaattcgggtgcaacttctggaagttctgagaacccaacaacacctgttgataaggagagtgttacaccgaaagcttcaaagaacgaagaagagcagagcgagggaaataattcacaagaaaagaccttgaagaaaccagacaagatacttccatgcccccggtgtaatagcatggacaccaagttctgttactacaacaattacaacgttaaccaaccccgacacttctgcaagaaatgccaaagatattggactgctggtgggacgatgaggaatgttcctgtgggtgctggtcgtcgtaagaacaagaactctgcttctcactaccgtcacataactgtttctgaagctcttcaaaatggaagaactgatgtttcaagtggaattcactctcctcatccttcaatgaaatctaatggtactgtcctgacatttggctctgatgcacccttatgtgaatcaatggcatctgttctgaatcttgctgataaaaacttgaggaactgcacacagaatggttttcttaaacccgaagaagtaaggactcccgtaccttttcaaagtgtagaacatggagataacaatgttgatggatcttcaattacaacatcgaattcaaaggatgaggcgtgtaacaatgtttcacaagaacaagtcatacaaaatcctcagggtgttcctccccaaatgccatgctttcctgggcctccttggccgtacccttggaatgctgctcagtggaacccttcggttccaatgcccgctttctgtcctccaggatatcctatgccattttaccctgcacctgcttattggggttgtagtgtaccagggacatggaacatcccttggcttccaatgccaacatctcaaaacccagccgccccgagctctggtcctaactctcctaactccccaacattgggaaaacattcaagggatgaaaacacagttaaatcaaattcctcagaagaggaacaaccaaaagaaagcaattctgagagatgcctttggattccaaaaacattgaggatcgacgatccaggtgaagctgctaggagctctatatgggcaacattggggataaagaatgataaagccg

SEQ ID NO: 188 - DNA, 1270 bp, synthetic construct
  source 1..1270: mol_type = other DNA
tcggcttctgcgacttctatggacgataatatggatcaggaacctaaaccgaactcttcttcttctgaggtcattaccagtaaagatgaggatgatagagacacagaaaaggcaacaacaaatgatgatcccacagagactaaagaggaagatggaaaccaacctatgtctcctgaagagccaagaaattcgggtgcaacttctggaagttctgagaacccgacaacacctgttgataaggagagtgttacaccgaaagcttcaaagaccgaagaagagcagagcgagggaaataattcacaagaaaagaccttgaagaaaccagacaagatacttccatgcccccggtgtaatagcatggacaccaagttctgttactacaacaattacaacgttaaccaaccccgacacttctgcaagaaatgccaaagatattggactgctggtgggacgatgaggaatgttcctgtgggtgctggtcgtcgtaagaacaagaactctgcttctcactaccgtcacataactgtttctgaagctcttcaaaatggaagaactgatgtttcaagtggaattcactctcctcatccttcaatgaaatctaatggtactgtcctgacatttggctctgatgcacccttatgtgaatcaatggcatctgttctgaatcttgctgataaaaacttgaggaactgcacacagaatggttttcttaaacccgaagaagtaaggactcccgtaccttttcaaagtgtagaacatggagataacaatgttgatggatcttcaattacaacatcgaattcaaaggatgaggcgtgtaacaatgtttcacaagaacaagtcatacaaaatcctcagggtgttcctccccaaatgccatgctttcctgggcctccttggccgtacccttggaatgctgctcagtggaacccttcggttccaatgccagctttctgtcctccaggatatcctatgccattttaccctgcacctgcttattggggttgtagtgtaccagggacatggaacatcccttggcttccaatgccaacatctcaaaacccagccaccccgagctctggtcctaactctcctaactccccaacattgggaaaacattcaagggaggaaaacacagttaaatcaaattcctcagaagaagaacaaccaaaagaaagcaattctgagagatgcctttggattccaaaaacattgaggatcgacgatccaggtgaagctgctaggagctctatatgggcaacattgggga

SEQ ID NO: 189 - DNA, 1213 bp, synthetic construct
  source 1..1213: mol_type = other DNA
cctaaaccaaactcttcttcttctgaggtcattaccagtaaagatgaggatgatagagacacagaaaaggcaacaacaaatgatgatcccacagagactaaagaggaagatggaaaccaacctatgtctcctgaagagccaaggaattcgggtgcaacttctggaagttctgagaacccgacaacacctgttgataaggagagtgttacaccgaaagcttcaaagaccgaagaagagcagagcgagggaaataattcacaagaaaagaccttgaaaaaaccagacaagatacttccatgcccccggtgtaatagcatggacaccaagttctgttactacaacaattacaacgttaaccaaccccgacacttctgcaagaaatgccaaagatattggactgctggtgggacgatgaggaatgttcctgtgggtgctggtcgtcgtaagaacaagaactctgcttctcactaccgtcacataactgtttctgaagctcttcaaaatggaagaactgatgtttcaagtggaattcactctcctcatccttcaatgaaatctaatggtactgtcctgacatttggctctgatgcacccttatgtgaatcaatggcatctgttctgaatcttgctgataaaaacatgaggaactgcacacagaatggttttcttaaacccgaagaagtaaggactcctgtagcttttcaaagtgtagaaaatggagatatcaatgttgatggatcttcaattacaacatcgaattcaaaggatgaggcgtgtaacaatgtttcacaagaacaagtcatacaaaatcctcagggtgttcctccccaaatgccatgctttcctgggcctccttggccgtacccttggaatgctgctcagtggaacccttcggttccaatgcccgctttctgtcctccaggatatcctatgccattttaccctgcacctgcttattggggttgtagtgtaccagggacatggaacatcccttggcttccaatgccaacatctcaaaacccagccaccccgagctctggtcctaactctcctaactccccaacattgggaaaacattcaagggaggaaaacacagttaaatcaaattcctcagaagaagagcaaccaaaagaaagcaattctgagagatgcctttggattccaaaaacattgaggatcgacgatccaggtgaagctgctaggagctctatat

SEQ ID NO: 190 - protein, 511 aa, Cannabis sativa
MRAQMSEPKDPAIKLFGKTIPVAEVPVTSGGSQGPSVSASATSMDDNMDQEPKPNSSSSEVITSKDEDDRDTEKATTNDDPTETKEEDGNQPMSPEEPRNSGTTSGSSENPTTPVDKESVTPKASKTEEEQSEGNNSQEKTLKKPDKILPCPRCNSMDTKFCYYNNYNVNQPRHFCKKCQRYWTAGGTMRNVPVGAGRRKNKNSASHYRHITVSEALQNGRTDVSSGIHSPHPSMKSNGTVLTFGSDAPLCESMASVLNLADKNMRNCTQNGFLKPEEVRTPVAFKSVENGDNNVDGSSITTSNSKDEACNNVSQEQVIQNPQGVPPQMPCFPGPPWPYPWNAAQWNPSVPMPAFCPPGYPMPFYPAPAYWGCSVPGTWNIPWLPMPTSQNPAAPSSGPNSPNSPTLGKHSRDENTVKSNSSEEEQPKESNSERCLWIPKTLRIDDPGEAARSSIWATLGIKNDKADSISGGGLFKAFQPKNDEKNHRAENSVLQANPAALSRSLSFQERS

SEQ ID NO: 191 - protein, 407 aa, Cannabis sativa
EVITSKDEDDRDTEKATTNDDPTETKEEDGNQPMSPEEPRNSGATSGSSENPTTPVDKESVTPKASKNEEEQSEGNNSQEKTLKKPDKILPCPRCNSMDTKFCYYNNYNVNQPRHFCKKCQRYWTAGGTMRNVPVGAGRRKNKNSASHYRHITVSEALQNGRTDVSSGIHSPHPSMKSNGTVLTFGSDAPLCESMASVLNLADKNLRNCTQNGFLKPEEVRTPVPFQSVEHGDNNVDGSSITTSNSKDEACNNVSQEQVIQNPQGVPPQMPCFPGPPWPYPWNAAQWNPSVPMPAFCPPGYPMPFYPAPAYWGCSVPGTWNIPWLPMPTSQNPAAPSSGPNSPNSPTLGKHSRDENTVKSNSSEEEQPKESNSERCLWIPKTLRIDDPGEAARSSIWATLGIKNDKA

SEQ ID NO: 192 - protein, 423 aa, Cannabis sativa
SASATSMDDNMDQEPKPNSSSSEVITSKDEDDRDTEKATTNDDPTETKEEDGNQPMSPEEPRNSGATSGSSENPTTPVDKESVTPKASKTEEEQSEGNNSQEKTLKKPDKILPCPRCNSMDTKFCYYNNYNVNQPRHFCKKCQRYWTAGGTMRNVPVGAGRRKNKNSASHYRHITVSEALQNGRTDVSSGIHSPHPSMKSNGTVLTFGSDAPLCESMASVLNLADKNLRNCTQNGFLKPEEVRTPVPFQSVEHGDNNVDGSSITTSNSKDEACNNVSQEQVIQNPQGVPPQMPCFPGPPWPYPWNAAQWNPSVPMPAFCPPGYPMPFYPAPAYWGCSVPGTWNIPWLPMPTSQNPATPSSGPNSPNSPTLGKHSREENTVKSNSSEEEQPKESNSERCLWIPKTLRIDDPGEAARSSIWATLG

SEQ ID NO: 193 - protein, 404 aa, Cannabis sativa
PKPNSSSSEVITSKDEDDRDTEKATTNDDPTETKEEDGNQPMSPEEPRNSGATSGSSENPTTPVDKESVTPKASKTEEEQSEGNNSQEKTLKKPDKILPCPRCNSMDTKFCYYNNYNVNQPRHFCKKCQRYWTAGGTMRNVPVGAGRRKNKNSASHYRHITVSEALQNGRTDVSSGIHSPHPSMKSNGTVLTFGSDAPLCESMASVLNLADKNMRNCTQNGFLKPEEVRTPVAFQSVENGDINVDGSSITTSNSKDEACNNVSQEQVIQNPQGVPPQMPCFPGPPWPYPWNAAQWNPSVPMPAFCPPGYPMPFYPAPAYWGCSVPGTWNIPWLPMPTSQNPATPSSGPNSPNSPTLGKHSREENTVKSNSSEEEQPKESNSERCLWIPKTLRIDDPGEAARSSI

SEQ ID NO: 194 - DNA, 101 bp, Cannabis sativa
  source 1..101: mol_type = genomic DNA
aggaagatggaaaccaacctatgtctcctgaagagccaaggaattcgggtacaacttctggaagttctgagaacccgacaacacctgttgataaggagagt

SEQ ID NO: 195 - DNA, 101 bp, Cannabis sativa
  source 1..101: mol_type = genomic DNA
cccgacaacacctgttgataaggagagtgttacaccgaaagcttcaaagaccgaagaagagcagagcgagggaaataattcacaagaaaagaccttgaaaa

SEQ ID NO: 196 - DNA, 101 bp, Cannabis sativa
  source 1..101: mol_type = other DNA
cacccttatgtgaatcaatggcatctgttctgaatcttgctgataaaaacatgaggaactgcacacagaatggttttcttaaacccgaagaagtaaggact

SEQ ID NO: 197 - DNA, 101 bp, Cannabis sativa
  source 1..101: mol_type = genomic DNA
actgcacacagaatggttttcttaaacccgaagaagtaaggactcctgtagcttttaaaagtgtagaaaatggagataacaatgttgatggatcttcaatt

SEQ ID NO: 198 - DNA, 101 bp, Cannabis sativa
  source 1..101: mol_type = genomic DNA
cacagaatggttttcttaaacccgaagaagtaaggactcctgtagcttttaaaagtgtagaaaatggagataacaatgttgatggatcttcaattacaaca

SEQ ID NO: 199 - DNA, 101 bp, Cannabis sativa
  source 1..101: mol_type = genomic DNA
tttcttaaacccgaagaagtaaggactcctgtagcttttaaaagtgtagaaaatggagataacaatgttgatggatcttcaattacaacatcgaattcaaa

SEQ ID NO: 200 - DNA, 101 bp, Cannabis sativa
  source 1..101: mol_type = genomic DNA
cgaagaagtaaggactcctgtagcttttaaaagtgtagaaaatggagataacaatgttgatggatcttcaattacaacatcgaattcaaaggatgaggcgt

SEQ ID NO: 201 - DNA, 101 bp, Cannabis sativa
  source 1..101: mol_type = genomic DNA
ggacatggaacatcccttggcttccaatgccaacatctcaaaacccagccgccccgagctctggtcctaactctcctaactccccaacattgggaaaacat

SEQ ID NO: 202 - DNA, 327 bp, synthetic construct
  source 1..327: mol_type = other DNA
atggccatgactaagtttctggccttcatgatcttggctttcatcgccatttccatgatccaaacaactgttatggcttctaatggcaatggaggtcatcacaacaacaagaaaggatatggaccagggagtctgaagagttaccaatgcccatctgaatgcgtgaggaggtgtggaagaactcagtaccacaagccgtgcatgttcttctgccagaaatgttgtaagacctgcctgtgtgtgccaccggggtattatggtaataaagctgtgtgcccttgctacaacaactggaagactaaggaaggaggccctaaatgcccttag

SEQ ID NO: 203 - DNA, 276 bp, synthetic construct
  source 1..276: mol_type = other DNA
gctttcatcgccatttccatgatccaaacaactgttatggcttctaatggcaatggaggtcatcacaacaacaaggaaggatatggaccagggagtctcaagagttaccaatgcccatctgaatgcgtgaggaggtgtggaagaacccagtaccacaagccgtgcatgttcttctgccagaaatgttgtaagacctgcctgtgtgtgccaccggggtattatggtaataaagctgtgtgcccttgctacaacaactggaagaccaaggaaggaggc

SEQ ID NO: 204 - protein, 108 aa, Cannabis sativa
MAMTKFLAFMILAFIAISMIQTTVMASNGNGGHHNNKKGYGPGSLKSYQCPSECVRRCGRTQYHKPCMFFCQKCCKTCLCVPPGYYGNKAVCPCYNNWKTKEGGPKCP

SEQ ID NO: 205 - protein, 92 aa, Cannabis sativa
AFIAISMIQTTVMASNGNGGHHNNKEGYGPGSLKSYQCPSECVRRCGRTQYHKPCMFFCQKCCKTCLCVPPGYYGNKAVCPCYNNWKTKEGG

SEQ ID NO: 206 - DNA, 101 bp, Cannabis sativa
  source 1..101: mol_type = genomic DNA
ttgcattgtaattaattaattaattattttttttaaaatttttattttagaaaggatatggaccagggagtctgaagagttaccgtaagtattttgttatt

SEQ ID NO: 207 - DNA, 1338 bp, synthetic construct
  source 1..1338: mol_type = other DNA
atggcttatggaggtacaccactgggtcccccaccaatggctgctgctgctgcttcatcatcagcctccacagttcgccaggtcaagttggaccgagagagcgagcttcgaatcgaagttggatacgactcctccctcaggctccgacttctaaacggcacggctgagatattcggcaccgaactcccacctgaaatctggctcactttccctcccaggctcaaatttgctgttttcacttggtatggagccacaattgaaatggatggtagtactgaaactgactacactgcagatgagacccctatgattagctacgtgaatgtccatgccatattggatggtcgaagaaaccgtgctaaagcttcgtcctctgatggttccagtacggttcaggggcctcgggtcattgttgtgggacctacagattctgggaagagtacattgtcaagaatgcttcttagttgggcagctaaacagggttggaaacctacttttgtggacttggatattggtcaaggatccataactctaccgggatgtattgctgctactcctattgaaatgcctattgacccagttgaagggattcctcttgagatgcctttggtttacttctatggacacaccactcccagtaacaatgtagaactatacaaaatccttgtgagggagctttctcaactgcttgagagacaatttgcgggtaatgttgaatctcgagctgctggcatggtgatcaataccatgggttggatagaaggaataggatatgagttgcttcttcatgcaatcgacacatttaatatcaacgttgtattggttctgggtcaggaaaaactttgcagtatgctcagagatgttctgaaaaacaagcctgatgtggatgttgtaaaacttcaaagatccggaggagttgtatcgaggaatgtcaaatatcgtcaaaaggctaggagtttgaggataagggaatatttttatggacggataaacgatctctccccgcattctaatattgcgagttttagtgatttattggtctatcgaattggtgggggacctcaggcaccccggtcagccctgccaattggtgcagagcctgcagcagatcctacaagattagtccctgtgaatatcaaccaggatttgctccatctagttcttgctgtttcatttgcgaaagagcctgaggaaattctttcaagtaacgttgccgggtttatatacattactgacattgacattcagaggaagaagatcacatatcttgcaccagctgctggtgatctcccgagcaaatatctgattgtgggaaccttgacatggatcgaaacatag

SEQ ID NO: 208 - DNA, 1338 bp, synthetic construct
  source 1..1338: mol_type = other DNA
atggcttatggaggtacaccactgggtcccccaccaatggcagctgctgctgcttcatcatcagcctccacagttcgccaggtcaagttggaccgagagagcgagcttcgaatcgaagttggatacgactcctccctcaggctccgacttctaaacggcacagctgagatattcggcaccgaactcccacctgaaatctggctcactttccctcccaggctcaaatttgctgttttcacttggtatggagccacaattgaactggatggtagtactgaaactgactacactgcagatgagacccctatgattagctacgtgaatgtccatgccatattggatggtcgaagaaaccgtgctaaagcttcgtcctctgatggttccagtacggttcaggggcctcgggtcattgttgtgggacctacagattctgggaagagtacattgtcaagaatgcttcttagttgggcagctaaacagggttggaaacctacttttgtggacttggatattggtcaaggatccataactctaccgggatgtattgctgctactcctattgaaatgcctattgacccagttgaagggattcctcttgagatgcctttggtttacttctatggacacaccactcccagtaacaatgtagaactatacaaaatccttgtgagggagctttctcaactgcttgagagacaatttgcgggtaatattgaatctcgagctgctggcatggtgatcaataccatgggttggatagaaggaataggatatgagttgcttcttcatgcaatcgacacatttaatatcaacgttgtattggttctgggtcaggaaaaactttgcagtatgctcagagatgttctgaaaaacaagcctgatgtggatgttgtaaaacttcaaagatctggaggagttgtatcgaggaatgtcaaatatcgtcagaaggctaggagtttgaggataagggaatatttttatggaaggataaacgatctctccccgcattctaatattgcgagttttagtgatttattggtctatcgaattggtgggggacctcaggcaccccggtcagccctgccaattggtgcagagcctgcagcagatcctacaagattagtccctgtgaatatcaaccaggatttgctccatctagttcttgctgtttcatttgcgaaagagcctgaggaaattctttcaagcaacgttgccgggtttatatacattactgacattgacattcagaggaagaagattacatatcttgcaccagctgctggtgatctcccgagcaaatatctgattgtgggaaccttgacatggatcgaaacatag

SEQ ID NO: 209 - protein, 445 aa, Cannabis sativa
MAYGGTPLGPPPMAAAAASSSASTVRQVKLDRESELRIEVGYDSSLRLRLLNGTAEIFGTELPPEIWLTFPPRLKFAVFTWYGATIEMDGSTETDYTADETPMISYVNVHAILDGRRNRAKASSSDGSSTVQGPRVIVVGPTDSGKSTLSRMLLSWAAKQGWKPTFVDLDIGQGSITLPGCIAATPIEMPIDPVEGIPLEMPLVYFYGHTTPSNNVELYKILVRELSQLLERQFAGNVESRAAGMVINTMGWIEGIGYELLLHAIDTFNINVVLVLGQEKLCSMLRDVLKNKPDVDVVKLQRSGGVVSRNVKYRQKARSLRIREYFYGRINDLSPHSNIASFSDLLVYRIGGGPQAPRSALPIGAEPAADPTRLVPVNINQDLLHLVLAVSFAKEPEEILSSNVAGFIYITDIDIQRKKITYLAPAAGDLPSKYLIVGTLTWIET

SEQ ID NO: 210 - protein, 445 aa, Cannabis sativa
MAYGGTPLGPPPMAAAAASSSASTVRQVKLDRESELRIEVGYDSSLRLRLLNGTAEIFGTELPPEIWLTFPPRLKFAVFTWYGATIELDGSTETDYTADETPMISYVNVHAILDGRRNRAKASSSDGSSTVQGPRVIVVGPTDSGKSTLSRMLLSWAAKQGWKPTFVDLDIGQGSITLPGCIAATPIEMPIDPVEGIPLEMPLVYFYGHTTPSNNVELYKILVRELSQLLERQFAGNIESRAAGMVINTMGWIEGIGYELLLHAIDTFNINVVLVLGQEKLCSMLRDVLKNKPDVDVVKLQRSGGVVSRNVKYRQKARSLRIREYFYGRINDLSPHSNIASFSDLLVYRIGGGPQAPRSALPIGAEPAADPTRLVPVNINQDLLHLVLAVSFAKEPEEILSSNVAGFIYITDIDIQRKKITYLAPAAGDLPSKYLIVGTLTWIET

SEQ ID NO: 211 - DNA, 101 bp, Cannabis sativa
  source 1..101: mol_type = genomic DNA
ggatgtttattctgtgaaaggttttcacttggtatggagccacaattgaaatggatggtagtactgaaactgactacactgcagatgaggtattatttatc

SEQ ID NO: 212 - DNA, 101 bp, Cannabis sativa
  source 1..101: mol_type = genomic DNA
tccttgtgagggagctttctcaactgcttgagagacaatttgcgggtaatgttgaatctcgagctgctggcatggtgatcaataccatgggttggatagaa

SEQ ID NO: 213 - DNA, 642 bp, synthetic construct
  source 1..642: mol_type = other DNA
atgggaaggggtaagattgagattaagaaaatagagaatacaagtaatagagcagtgacttatgctaagagaaaagctggaattttcaagaaggctaaggagattgctattctttgtgatgttaaagtctcactcatcatcgtttctggatctggcaaaatggatgccatgtgtagtcctgataattcgttgagtaccatatgtgaggaatatcacaaacactccaaagagaagttatggcctgctaagcatgagaacctgcacaatgaaattgaaatggtcaaaaaagaaaatgagaacatggtgatagagctgaggcacctgaatggacaggatatcaatctgaaccactatgaactgatacctattgaggaggcacttgagaatggtcttataagtgttcgtgccagaaagtcggagttagttaacattcttaggcaaaatctggaagctaaagaggaagagcataagcgcctcatttgcgagctgcaccaaaagatggaagtagtaggggatcatgggtatcataatcatcatcatcatcatgatcagaggctgaatttgagggactacaataatgcccatatgcctttagccttccgtgtccagcctatgcagccaaatctccaagagagaatttag

SEQ ID NO: 214 - DNA, 546 bp, synthetic construct
  source 1..546: mol_type = other DNA
aaagctggaattttcaagaaggctaaggagattgctattctttgtgatgttaaagtctcactcatcatcgtttctggatctggcaaaatggatgccatgtgtagtcctgataattcgttgagtaccatatgtgaggaatatcacaaacactccaaagagaagttatggcctgctaagcatgagaacctgcacaatgaaattgaaatggtcaaaaaagaaaatgagaacatggtgatagagctgaggcacctgaatggacaggatatcaatctgaaccactatgaactgatacctattgaggaggcacttgagaatggccttataagtgttcgtgccagaaagtcggagttagttaacattcttaggcaaaatctggaagctaaagaggaagagcataagcgcctcatttgcgagctgcaccaaaagatggaagtagtaggggatcatgggtatcatcatcatcatcatcatgatcagaggctgaatttgagggactacaataatgcccatatgcctttagccttccgtgtccagcctatgcagcca

SEQ ID NO: 215 - protein, 213 aa, Cannabis sativa
MGRGKIEIKKIENTSNRAVTYAKRKAGIFKKAKEIAILCDVKVSLIIVSGSGKMDAMCSPDNSLSTICEEYHKHSKEKLWPAKHENLHNEIEMVKKENENMVIELRHLNGQDINLNHYELIPIEEALENGLISVRARKSELVNILRQNLEAKEEEHKRLICELHQKMEVVGDHGYHNHHHHHDQRLNLRDYNNAHMPLAFRVQPMQPNLQERI

SEQ ID NO: 216 - protein, 182 aa, Cannabis sativa
KAGIFKKAKEIAILCDVKVSLIIVSGSGKMDAMCSPDNSLSTICEEYHKHSKEKLWPAKHENLHNEIEMVKKENENMVIELRHLNGQDINLNHYELIPIEEALENGLISVRARKSELVNILRQNLEAKEEEHKRLICELHQKMEVVGDHGYHHHHHHDQRLNLRDYNNAHMPLAFRVQPMQP

SEQ ID NO: 217 - DNA, 103 bp, Cannabis sativa
  source 1..103: mol_type = genomic DNA
tatatatgcagcaccaaaagatggaagtagtaggggatcatgggtatcataatcatcatcatcatcatgatcagaggctgaatttgagggactacaataatgc

SEQ ID NO: 218 - DNA, 2730 bp, synthetic construct
  source 1..2730: mol_type = other DNA
atgtcttcttctccggtggattattcatcgaaatgtgtgatgtttccggcaagacccggttatggtattgttggtagaaagtgtttgatcaaagctaatcacttcacactccaacttgttaccaccatccataactataagggtctctatcattaccatgtatggataagtcctcaagtaacatccatgaaattaaacagagatattattaaagagttgggaactttgaactatttaggtaatgtgagagttgcctatgatggtagaaatactatctactctgttggggagttaccattttcttcaaatgaatttattcttaagtttcccacaacaaaaacaacaacaaccaccagtgaatgtgagtttaaggttagcatcaggttcgtttcaaggatagacttatgtaatccacaaagctttttacacaccattcaaccacttaacgttgctctcagatcagctccatcagaaatgtatagtgttgtggggaggtctttttatcatgacacaatgagtagagctggtgagcttggagatggtgtaatgtattggaaaggtttctatcaaagtttgaggccaactcaattggggctttctcttaacattgatgtgtcagcaaaggctttttatgaacctattgaggtaactgattttatcttcaaaaactttaatgtgagatacgattcgacgccattggatgatcatgttcgggttcagctaaggaaaaatttaagaggggttaaagtaacatgtagacatttggatgatacaatgttgttcaaagtttttggggtatcgacagagccattgaacaatttaatgtttgatcttgatggtaacacgatttcggttgttacttattttcaagacaagtacgaaattcagctcgaatatgtgacatggcctgctctgcaagtaggaagtgcaacaaagccgatttacttgcccatagaggtttgtagaattgttgaggggcaaagatgctacaagaaactcaaccaaaggcaagtaaccaatctattaagggaaacttgtcaacgaccgtacgaaagggaggaaaccattaggcaaatttttcgacaaaacaattacaatgatcaaaatcttgttcgcgattttggaattaatgttgcaaacgacatgacattgattaatgctcgagttttgcctccaccattgcttttataccatgatactgggaatgaagctactgccaatcctcaactcgggcaatggaatatgatcaataagaaaatgatcaatggtggccatgtaaaatcttggacttgtgtgaacttctcacatgaaaatccaaatattcacaatcgcttttgtgatgaactagtcaatatgtgcatcagtagaggaatggtagggttactcaatctttctgtctcgttatgttttttttcgcgaaattgcataactgacgtttttatcattttggatactcggaataaggctttccaaaccgcgccagtaattccttcgcgcgcttggccagctaacgaaattgaggaggccttgaaatatgttcatgaccaatgcaacatgatactcggaaacaatcaacttcagttgctaatagttatcttgcctgatttttcgggttcttatgggacaattaaacgagtttgtgaaactgagcttggaattgtttcacaatgctgtcggcctaaccatgcagcgaagctcaacaacaagcaatacttggagaatctatcattgaaaatcaataccaaggttggtgggcgaaatacagtgttaagtgatgctcttaataagagaattccttttgtgtctgatgtcccgacgataatctttggcgcggcttttacacatccaaagcccgaagattactcaagcccttcaatagcagctgtggttgcctcgatggattggcccgaggtgaccaaatatagagcgttggtatcagcgcaacagcacaccgaggagatcatccaagatctttataaagtaactacagatgataaagggagcatagtccatggaggaatgataaggcgagttaatcaccattgtgtacatgccgatcaaccggagccaaacctcatcggattatattctaccggttatttgtgtagcacacttggtaaaatctttagtggtttttcattactctgcctcttaatttactctgttttgtgcagagatggcgttagtgatggacagttcaatcgagtgttgctttacgaaatggatgcgataagaaaggcttgttcatcgctcgaggaaggatatcttcctcgagtgacatttgttgctattcagaaaaggtgtcatactcgccttttccctgctgattacaacgatcgcgattcaatggacaagagtggcaacattctaccaggcactgttgttgatactaatatctgccacccaactcacttcgatttttacctcaaaagtcatgctggaattcagggaacaagtcgtcccgctcactaccatgtcttgttcgacgagaacaacttcacagctgatgccttacaagttctcacaaacaatctatgctacacgtatgcaaggtgcacgaaatcagtgtctatcgttcctcctgcatattatgcacatttggctgcgattcgtgctcgttgctacattggagtagcagttgatgaagaagaaactgatgttggcccaagaacttatcaaccccttcaggtgatcaatgggaaccttaaagatgtgatgttctatgtctaa

SEQ ID NO: 219 - DNA, 2016 bp, synthetic construct
  source 1..2016: mol_type = other DNA
ctccaacttgttaccaccatccataactataagggtctctatcattaccatgtatggataagtcctcaagtaacatccatgaaattaaacagagatatcgttaaagagttgggaactttgaactatttaggtaatgtgagagttgcctatgatggtagaaacactatctacactgttggggagttaccattttcttcaaatgaatttattcttaagtttcccacaacaaaaacaacaacaaccaccagtgaatgtgagtttaaggttagcatcaggttcgtttcaaggatagacttatgtaatccacaaagctttttacacaccattcaaccacttaatgttgctctcagatcagctccatcagaaatgtatagtgttgtggggaggtctttttatcatgacacaatgagtagagctggtgagcttggagatggtgtaatgtattggaaaggtttctatcaaagtttgaggccaactcaattggggctttctcttaacattgatgtgtcagcaaaggctttttatgaacctattgaggtaactgattttatcttcaaaaactttaatgtgagatacgattcgacgccattggatgatcatgttcgggttcagctaaggaaaaatttaagaggggttaaagtaacatgtagacatttggatgatacaatgttgttcaaaatttttggggtatcgacagagccattgaacaatttaatgtttgatcttgatggtaacacgatttcggttgttacttactttcaagataagtacgaaattcagctcgaatatgtgacatggcctgctctgcaagtaggaagtgcaacaaagccgatttacttgcccatagaggtttgtagaattgttgaggggcaaagatgctacaagaaactcaaccaaaggcaagtaaccaatctattaagggaaacttgtcaacgaccgtacgaaagggaggaaaccattaggcaaatttttcgacaaaacaattacaatgatcaraatcttgttcgcgattttggaattaatgttgcaaacgacatgacattgattaatgctcgagttttgcctccaccattgcttttataccatgatactgggaatgaagctactgcsaatcctcaactcggrcaatggaatatgatcaataaraaaatgatcaatggtggccatgtaaaatcttggacttgtgtgaacttctcacatgaaaatccaratattcacaatcgcttttgtgatgaactagtcaatatgtgcatcagtagaggaatggctttccaraccaygccagtaattccttcgcgcgcttggccagctaatgaaatcaaggaggccttgaaatatgttcatgaccaatgcaacatgatacttggaaacaaacaacttcagttgctaatagttatcttgcctgatttttcgggttcttatgggacrattaaacgagtttgtgaaactgagctwggaatkgtttcacaatgctgycggcctaaacatgcagcgaagctcaacaacaagcmatacttggagaatctatcattgaaaatcaataccaaggttggtgggcgaaatacagtgttaagtgatgctcttaataagagaattccttttgtgtctgatgtcccgacgataatctttggcgcggcttttacacatccaaagcccgaagattactcaagcccttcaatagcagctgtggttgcctcgatggattggcccgaggtgaccaaatatagagcgttggtatcagcgcaacagcacaccgaggagatcatccaagatctttataaagtaactacagatgataaagggagcatagtccatggaggaatgataagggatcatttaattgctttcagccgatcaaccggagccaaacctcaccggattatattctaccgagatggtgttagtgatggacagttcaatcgagtgttgctttacgaaatggatgcgataagaaaggcttgttcatcgctcgatgaaggatatcttcctcgagtgacatttgttgctattcagaaa

SEQ ID NO: 220 - DNA, 663 bp, synthetic construct
  source 1..663: mol_type = other DNA
ccttttgtgtctgatgtcccgacgataatctttggtgcggcttttacacatccaaagccggaagattactcaagcccttcaatagcagctgtggttgcctcgatggattggcccgaggtgaccaaatatagagcgttggtatcagcgcaacagcacaccgaggagatcatccaagatctttataaagtaactacagatgataaagggagcatagtccatggaggaatgataagggatcatttaattgctttcagccgatcaaccggagccaaacctcaccggattatattctaccgagatggcgttagtgatggacagttcaatcgagtgttgctttacgaaatggatgcgataagaaaggcttgttcatcgctcgaggaagaatatcttcctcgagtgacatttgttgctattcagaaaaggtgtcatactcgccttttccctgctgattacaacgatcgcaattcaatggacaagagtggcaacattctaccaggcactgttgttgatactcatatctgccacccaactcaattcgatttttacctcaaaagtcatgctggaattcagggaacaagtcgtcccgctcactaccatgtcttgttcgacgagaacaacttcacagctgatgccttacaagttctcacaaacaatctatgctac

SEQ ID NO: 221 - protein, 909 aa, Cannabis sativa
MSSSPVDYSSKCVMFPARPGYGIVGRKCLIKANHFTLQLVTTIHNYKGLYHYHVWISPQVTSMKLNRDIIKELGTLNYLGNVRVAYDGRNTIYSVGELPFSSNEFILKFPTTKTTTTTSECEFKVSIRFVSRIDLCNPQSFLHTIQPLNVALRSAPSEMYSVVGRSFYHDTMSRAGELGDGVMYWKGFYQSLRPTQLGLSLNIDVSAKAFYEPIEVTDFIFKNFNVRYDSTPLDDHVRVQLRKNLRGVKVTCRHLDDTMLFKVFGVSTEPLNNLMFDLDGNTISVVTYFQDKYEIQLEYVTWPALQVGSATKPIYLPIEVCRIVEGQRCYKKLNQRQVTNLLRETCQRPYEREETIRQIFRQNNYNDQNLVRDFGINVANDMTLINARVLPPPLLLYHDTGNEATANPQLGQWNMINKKMINGGHVKSWTCVNFSHENPNIHNRFCDELVNMCISRGMVGLLNLSVSLCFFSRNCITDVFIILDTRNKAFQTAPVIPSRAWPANEIEEALKYVHDQCNMILGNNQLQLLIVILPDFSGSYGTIKRVCETELGIVSQCCRPNHAAKLNNKQYLENLSLKINTKVGGRNTVLSDALNKRIPFVSDVPTIIFGAAFTHPKPEDYSSPSIAAVVASMDWPEVTKYRALVSAQQHTEEIIQDLYKVTTDDKGSIVHGGMIRRVNHHCVHADQPEPNLIGLYSTGYLCSTLGKIFSGFSLLCLLIYSVLCRDGVSDGQFNRVLLYEMDAIRKACSSLEEGYLPRVTFVAIQKRCHTRLFPADYNDRDSMDKSGNILPGTVVDTNICHPTHFDFYLKSHAGIQGTSRPAHYHVLFDENNFTADALQVLTNNLCYTYARCTKSVSIVPPAYYAHLAAIRARCYIGVAVDEEETDVGPRTYQPLQVINGNLKDVMFYV

SEQ ID NO: 222 - protein, 672 aa, Cannabis sativa
LQLVTTIHNYKGLYHYHVWISPQVTSMKLNRDIVKELGTLNYLGNVRVAYDGRNTIYTVGELPFSSNEFILKFPTTKTTTTTSECEFKVSIRFVSRIDLCNPQSFLHTIQPLNVALRSAPSEMYSVVGRSFYHDTMSRAGELGDGVMYWKGFYQSLRPTQLGLSLNIDVSAKAFYEPIEVTDFIFKNFNVRYDSTPLDDHVRVQLRKNLRGVKVTCRHLDDTMLFKIFGVSTEPLNNLMFDLDGNTISVVTYFQDKYEIQLEYVTWPALQVGSATKPIYLPIEVCRIVEGQRCYKKLNQRQVTNLLRETCQRPYEREETIRQIFRQNNYNDQNLVRDFGINVANDMTLINARVLPPPLLLYHDTGNEATANPQLGQWNMINKKMINGGHVKSWTCVNFSHENPXIHNRFCDELVNMCISRGMAFQTXPVIPSRAWPANEIKEALKYVHDQCNMILGNKQLQLLIVILPDFSGSYGTIKRVCETELGXVSQCCRPKHAAKLNNKXYLENLSLKINTKVGGRNTVLSDALNKRIPFVSDVPTIIFGAAFTHPKPEDYSSPSIAAVVASMDWPEVTKYRALVSAQQHTEEIIQDLYKVTTDDKGSIVHGGMIRDHLIAFSRSTGAKPHRIIFYRDGVSDGQFNRVLLYEMDAIRKACSSLDEGYLPRVTFVAIQK

SEQ ID NO: 223 - protein, 221 aa, Cannabis sativa
PFVSDVPTIIFGAAFTHPKPEDYSSPSIAAVVASMDWPEVTKYRALVSAQQHTEEIIQDLYKVTTDDKGSIVHGGMIRDHLIAFSRSTGAKPHRIIFYRDGVSDGQFNRVLLYEMDAIRKACSSLEEEYLPRVTFVAIQKRCHTRLFPADYNDRNSMDKSGNILPGTVVDTHICHPTQFDFYLKSHAGIQGTSRPAHYHVLFDENNFTADALQVLTNNLCY

SEQ ID NO: 224 - DNA, 101 bp, Cannabis sativa
  source 1..101: mol_type = genomic DNA
gaggggttaaagtaacatgtagacatttggatgatacaatgttgttcaaagtttttggggtatcgacagagccattgaacaatttaatgtaagtaacataa

SEQ ID NO: 225 - DNA, 101 bp, Cannabis sativa
  source 1..101: mol_type = genomic DNA
aactgacgtttttatcattttggatactcggaataaggctttccaaaccgcgccagtaattccttcgcgcgcttggccagctaacgaaattgaggaggcct

SEQ ID NO: 226 - DNA, 101 bp, Cannabis sativa
  source 1..101: mol_type = genomic DNA
tccaaaccgcgccagtaattccttcgcgcgcttggccagctaacgaaattgaggaggccttgaaatatgttcatgaccaatgcaacatgatactcggaaac

SEQ ID NO: 227 - DNA, 101 bp, Cannabis sativa
  source 1..101: mol_type = genomic DNA
gaggccttgaaatatgttcatgaccaatgcaacatgatactcggaaacaatcaacttcagttgctaatagttatcttgcctgatttttcgggttcttatgg

SEQ ID NO: 228 - DNA, 101 bp, Cannabis sativa
  source 1..101: mol_type = genomic DNA
ttgcctcttgtagggacaattaaacgagtttgtgaaactgagcttggaattgtttcacaatgctgtcggcctaaccatgcagcgaagctcaacaacaagca

SEQ ID NO: 229 - DNA, 101 bp, Cannabis sativa
  source 1..101: mol_type = genomic DNA
cgagtttgtgaaactgagcttggaattgtttcacaatgctgtcggcctaaccatgcagcgaagctcaacaacaagcaatacttggagaatctatcattgaa

SEQ ID NO: 230 - DNA, 101 bp, Cannabis sativa
  source 1..101: mol_type = genomic DNA
ccatggcaatatctaatctgtttataattacaggcttgttcatcgctcgaggaaggatatcttcctcgagtgacatttgttgctattcagaaaaggtgtca

SEQ ID NO: 231 - DNA, 101 bp, Cannabis sativa
  source 1..101: mol_type = genomic DNA
gcaatatctaatctgtttataattacaggcttgttcatcgctcgaggaaggatatcttcctcgagtgacatttgttgctattcagaaaaggtgtcatactc

SEQ ID NO: 232 - DNA, 101 bp, Cannabis sativa
  source 1..101: mol_type = genomic DNA
ttcagaaaaggtgtcatactcgccttttccctgctgattacaacgatcgcgattcaatggacaagagtggcaacattctaccaggttttagtgtgtgaatc

SEQ ID NO: 233 - DNA, 101 bp, Cannabis sativa
  source 1..101: mol_type = genomic DNA
atttgattggttttactattttgcattttgaaggcactgttgttgatactaatatctgccacccaactcacttcgatttttacctcaaaagtcatgctgga

SEQ ID NO: 234 - DNA, 101 bp, Cannabis sativa
  source 1..101: mol_type = genomic DNA
ttttgcattttgaaggcactgttgttgatactaatatctgccacccaactcacttcgatttttacctcaaaagtcatgctggaattcaggtccaaaatgtc

(19) United States
(12) Patent Application Publication
(10) Pub. No.: US 2026/0150797 A1
(43) Pub. Date: Jun. 4, 2026

(54) FLOWER INITATION MARKERS

(71) Applicant: Phylos Bioscience, Inc., Portland, OR (US)

(72) Inventors: Erica Bakker, Portland, OR (US); Alisha Holloway, Portland, OR (US)

(73) Assignee: Phylos Bioscience, Inc., Portland, OR (US)

(21) Appl. No.: 19/123,560

(22) PCT Filed: Oct. 27, 2023
(86) PCT No.: PCT/US2023/078108

§ 371 Date: Apr. 23, 2025

Related U.S. Application Data
(60) Provisional application No. 63/381,558, filed on Oct. 29, 2022.

(51) Int. Cl. A01H 1/04; C12N 15/82; C12Q 1/6895

(52) U.S. Cl. CPC A01H 1/045; C12N 15/827; C12Q 1/6895; C12Q 2600/13; C12Q 2600/156

(57) ABSTRACT

Provided herein is the identification of markers and genes associated with flower initiation in Cannabis. The markers are useful for breeding Cannabis plants that initiate flowering at longer daylengths, which allow for earlier maturation and harvesting and ability to grow outdoors at different latitudes or in greenhouses without artificial light deprivation. Also provided are methods for selecting plants by obtaining nucleic acids and detecting one or more markers that indicate flower initiation at increased daylight hours in order to establish plant lines having shorter harvest period as a result of flowering at increased daylight hours.

### CROSS REFERENCE TO RELATED APPLICATIONS

This claims the benefit of U.S. Provisional Application No. 63/381,558 filed Oct. 29, 2022, which is incorporated herein by reference.

### FIELD

This disclosure relates to flower initiation in Cannabis. In particular, identifying genes and markers involved in the ability to initiate flowering at increased daylight hours and/or in a fewer number of days.

### INCORPORATION OF ELECTRONIC SEQUENCE LISTING

The Sequence Listing is submitted as an XML file, “Sequence.xml,” created on Oct. 27, 2023, 249,856 bytes, which is incorporated by reference herein.

### BACKGROUND

Flower initiation is the process where a plant starts to form flowers in response to environmental cues such as day length (photoperiod) and temperature, as well as internal cues such as age, phytohormones, and carbohydrate status. Floral induction by the photoperiod pathway in photosensitive Cannabis occurs when the night exceeds a critical length, where different genetic backgrounds are known to start flowering at different times under different day lengths. Most Cannabis varieties do not initiate flowers at day lengths greater than 12 hours, for this reason common cultivation practices involve growing plants for at least three weeks under long day length followed by a switch to 12/12 (12 hours light, 12 hours dark) after which plants initiate flowers after around 12 days. Because Cannabis plants initiate flowering during days with lessening amounts of sunlight, selecting plants that initiate flowering with longer daylight hours allows breeders to harvest plants at earlier intervals during the growing season and allows for growing plants outdoors or in greenhouses without artificial light deprivation at different latitudes.

It is advantageous for breeders to be able to control flowering initiation and further to utilize genetic markers to assist with controlling the desired flowering initiation conditions. The present disclosure describes SNP markers associated with the ability to initiate flowers at increased daylight hours (e.g., 18 hours of daylight) as well as initiate flowering in a fewer number of days, which solves the laborious and time-consuming issues associated with traditional breeding methods used to breeding for early flower initiation under longer daylengths.

### SUMMARY

Disclosed are methods of selecting and/or producing Cannabis plants that initiate flowering at increased daylight hours. In some examples, the methods comprise (i) obtaining a nucleic acid sample from a plant or its germplasm; (ii) detecting one or more markers that indicate flower initiation at increased daylight hours, (iii) indicating the flower initiation at increased daylight hours, and (iv) selecting the one or more plants that initiate flowering at increased daylight hours. In some examples, the methods include (i) obtaining a nucleic acid sample from a plant or its germplasm; (ii) detecting one or more markers that indicate flower initiation at increased daylight hours, (iii) crossing the plant comprising the one or more markers, and (iv) obtaining one or more progeny plants comprising the one or more markers and that initiate flowering at increased daylight hours. The markers that indicate flower initiation at increased daylight hours include one or more markers disclosed herein, for example, one or more markers described in any one of Tables 2, 4, 5, and 10. Flowering at increased daylight hours refers to a daylight period greater than 12 hours, for example, 13 to 18 hours of daylight. Crossing includes, for example, outcrossing, backcrossing, sibling crossing, or selfing. In some examples, a Cannabis plant including the one or more markers that indicate flower initiation at increased daylight hours is crossed with a second plant that does not initiate flowering at increased daylight hours.

Detecting one or more markers that indicate flower initiation at increased daylight hours can include analyzing two or more nucleotide positions associated with flower initiation at increased daylight hours as described herein, for example, analyzing at least 3, 5, 10, 15, 20, 25, 30, or 35 nucleotide positions described herein (e.g., one or more markers disclosed in Table 2, 4, 5, or 10). In some examples, detecting one or more markers that indicate flower initiation at increased daylight hours includes analyzing nucleotide positions: 159,096; 241,017; 321,930; 334,676; 351,874; 479,822; 498,442; 531,560; 537,879; 606,518; 639,258; 673,171; 703,062; 709,849; 714,581; 784,607; 1,319,381; and 1,376,341; and detecting at least one marker that indicates flower initiation at increased daylight hours, wherein the reference genome is the Abacus Cannabis reference genome version Csat_AbacusV2, NCBI assembly accession GCA_025232715.1. In some examples, detecting one or more markers that indicate flower initiation at increased daylight hours includes analyzing nucleotide positions: 19,681; 44,981; 87,904; 109,674; 159,096; 378,368; 479,822; 491,066; 568,544; 720,538; 968,829; 1,191,275; 1,200,057; 1,211,168; 1,273,758; 1,277,500; 1,305,959; 1,659,209; 1,690,294; 1,735,848; 1,816,071; 1,872,522; 1,993,648; 2,002,233; 2,020,279; 2,079,928; and 2,267,196, and detecting at least one marker that indicates flower initiation at increased daylight hours, wherein the reference genome is the Abacus Cannabis reference genome version Csat_AbacusV2. NCBI assembly accession GCA_025232715.1. In some examples, detecting one or more markers that indicate flower initiation at increased daylight hours includes analyzing nucleotide positions: 109,674; 159,096; 1,154,438, and detecting at least one marker that indicates flower initiation at increased daylight hours, wherein the reference genome is the Abacus Cannabis reference genome version Csat_AbacusV2, NCBI assembly accession GCA_025232715.1.

In some examples, the methods include selecting a marker that indicates flower initiation in a fewer number of days relative to a control. The control can be, for example, a Cannabis plant with the same genetic background except does not include the marker indicating flower initiation in a fewer number of days. In some examples, a marker that indicates flower initiation in a fewer number of days also indicates flowering at increased daylight hours. In some examples, the one or more markers that indicate flower initiation in a fewer number of days comprise a polymorphism on chromosome 8 relative to a reference genome at nucleotide positions 159,096; 241,017; 321,930; 334,676; 351,874; 479,822; 498,442; 531,560; 537,879; 606,518; 639,258; 673,171; 703,062; 709,849; 714,581; 784,607; 1,319,381; or 1,376,341, wherein the reference genome is the Abacus Cannabis reference genome version Csat_AbacusV2, NCBI assembly accession GCA_025232715.1.

Detecting one or more markers that indicates flower initiation in a fewer number of days can include analyzing two or more nucleotide positions associated with flower initiation in a fewer number of days as disclosed herein, for example, analyzing at least 3, 5, 10, 12, 15, or 18 nucleotide positions. In some examples, detecting one or more markers that indicate flower initiation in a fewer number of days includes analyzing nucleotide positions: 159,096; 241,017; 321,930; 334,676; 351,874; 479,822; 498,442; 531,560; 537,879; 606,518; 639,258; 673,171; 703,062; 709,849; 714,581; 784,607; 1,319,381; and 1,376,341; and detecting at least one marker that indicates flower initiation in a fewer number of days.

In some examples, the methods include crossing a progeny plant (e.g., F1) to produce one or more additional progeny plants, wherein the additional progeny plants (e.g., F2) initiate flowering at increased daylight hours. In some examples, the at least one additional progeny plant comprising the indicated flowering at increased daylight hours phenotype is an F2-F7 progeny plant. Also provided are methods of selecting one or more plants that initiate flowering at increased daylight hours, the method comprising replacing a nucleic acid sequence of a parent plant with a nucleic acid sequence conferring the flower initiation at increased daylight hours phenotype.

Further provided are methods of producing one or more Cannabis plants having modified flowering (e.g., flowering at increased daylight hours and/or initiating flowering in a fewer number of days), comprising: (i) obtaining a nucleic acid sample from a Cannabis plant or its germplasm; (ii) detecting one or more nucleic acid polymorphisms associated with flowering at increased daylight hours or flowering in a fewer number of days in one or more of: (a) EARLY FLOWERING 9 (ELF9); (b) FLOWERING LOCUS T (FT); (c) CYCLIC DOF FACTOR 2 (CDF2); (d) ARGONAUTE 5 (AGO5); (e) GAST1 PROTEIN HOMOLOG 4 (GASA4); (f) CLP-SIMILAR PROTEIN 3 (CLPS3); and (g) PISTILLATA (PI); (iii) crossing the Cannabis plant comprising the one or more nucleic acid polymorphisms, and (iv) obtaining progeny plants comprising of the one or more nucleic acid polymorphisms, thereby producing one or more Cannabis plants having modified flowering. Also provided are methods of identifying one or more Cannabis plants having modified flowering, including: (i) obtaining a nucleic acid sample from a Cannabis plant or its germplasm; (ii) detecting one or more nucleic acid polymorphisms associated with flowering at increased daylight hours or flowering in a fewer number of days in one or more of: (a) EARLY FLOWERING 9 (ELF9); (b) FLOWERING LOCUS T (FT); (c) CYCLIC DOF FACTOR 2 (CDF2); (d) ARGONAUTE 5 (AGO5); (e) GAST1 PROTEIN HOMOLOG 4 (GASA4); (f) CLP-SIMILAR PROTEIN 3 (CLPS3); and (g) PISTILLATA (PI); thereby identifying the one or more Cannabis plants having modified flowering. In some examples, the one or more Cannabis plants having modified flowering is selected for crossing and/or making a product. In some examples, one or more nucleic acid polymorphisms are detected in at least two of ELF9, FT, and CDF2. In some examples, one or more nucleic acid polymorphisms are detected in ELF9, FT, and CDF2.

Also provided are methods of producing a genetically engineered Cannabis plant that initiates flowering at increased daylight hours, including: introducing a genetic modification into ELF9 and/or FT, or introducing a beneficial allele of ELF9 and/or FT. The genetic modification can be a nucleic acid substitution, insertion, or deletion. In some examples, the genetic modification is introduced by mutagenesis or gene editing. The genetic modification or beneficial allele initiates flowering at increased daylight hours relative to a control, such as the Cannabis plant in an unmodified state. In some examples, the methods further include introducing a genetic modification in CDF2 or introducing a beneficial allele of CDF2.

The disclosure includes a plant produced or identified by any of the methods disclosed herein, as well as seed, tissue culture, or protoplast of a plant produced or identified by any of the methods disclosed herein. Also disclosed are methods of using a plant produced or identified by any of the methods disclosed herein, including crossing (breeding) or producing a Cannabis product from a plant produced or identified by any of the methods disclosed herein. In some examples, the Cannabis product is a kief, hashish, bubble hash, an edible product, solvent reduced oil, sludge, e-juice, or tincture.

The foregoing and other features of this disclosure will become more apparent from the following detailed description.

### DETAILED DESCRIPTION

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a plant” includes singular or plural plants and can be considered equivalent to the phrase “at least one plant.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

### Definitions

The term “Abacus” as used herein refers to the Cannabis sativa reference genome known as the Abacus reference genome version Csat_AbacusV2 (NCBI assembly accession GCA_025232715.1, incorporated by reference herein), which is also sometimes referred to as CsaAba2.

The term “about” refers to a range of 5% of the referenced value unless otherwise indicated. For example about 100 refers to a range of 95 to 105.

The term “alternative nucleotide call” is a nucleotide polymorphism relative to a reference nucleotide for a SNP marker that is significantly associated with the causative SNP(s) that confer(s) a desired phenotype (e.g., flowering at increased daylight hours or initiation of flowering in a fewer number of days). Unless otherwise indicated, the reference is the Abacus genome version Csat_AbacusV2 (NCBI assembly accession GCA_025232715.1).

The term “backcrossing” or “to backcross” refers to a process in which a breeder repeatedly crosses hybrid progeny, for example a first generation hybrid (F1), back to one of the parents of the hybrid progeny. Backcrossing can be used to introduce one or more single locus conversions from one genetic background into another.

The term “beneficial” as used herein refers to a genetic element (e.g., gene, allele, or polymorphism) conferring or associated with a desired phenotypic trait, for example, flowering at increased daylight hours or initiation of flowering in a fewer number of days. In some examples, a “beneficial polymorphism” or “beneficial allele” refers to a polymorphism or allele associated with flowering at increased daylight hours. In some examples, a “beneficial polymorphism” or “beneficial allele” refers to a polymorphism or allele associated with flowering in a fewer number of days.

The term “Cannabis” refers to plants of the genus Cannabis, including Cannabis sativa, Cannabis indica, and Cannabis ruderalis.

The term “cell” refers to a prokaryotic or eukaryotic cell, including plant cells, capable of replicating DNA, transcribing RNA, translating polypeptides, and secreting proteins.

The term “coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

The term “control” refers to a reference standard. A control can be a negative or positive control. In some examples, the control is a historical control or a known reference value (or range of values). A practitioner can select a suitable control based on the teachings provided herein. Non-limiting examples of suitable controls include an unmodified plant or a plant not including a marker disclosed herein.

The term “cross”, “crossing”, “cross pollination” or “cross-breeding” refer to the process by which the pollen of one flower on one plant is applied (artificially or naturally) to the ovule (stigma) of a flower on another plant, or “selfing” where pollen from a plant is applied (artificially or naturally) to the ovule (stigma) of the same plant. Backcrossing is a process in which a breeder repeatedly crosses hybrid progeny, for example a first generation hybrid (F1), back to one of the parents of the hybrid progeny. Backcrossing can be used to introduce one or more single locus conversions from one genetic background into another.

The term “daylight hours” as used herein refers to the amount of light per day a plant is exposed to. The light can be natural light, artificial light, or a combination of both. “Increased daylight hours” refers to a light period that is longer than the standard 12 hours at which Cannabis typically initiates flowering. In some examples, increased daylight hours refers to a light period between 12 to 24 hours, for example, 12 to 20, 12 to 18, 12 to 16, 13 to 20, 13 to 18, 13 to 16, 13 to 14, 14 to 20, 14 to 18, 14 to 16, 16 to 20, or 16 to 18 hours of light in a 24 hour cycle. In some examples, increased daylight hours refers to a light period of 13 to 18 hours.

In some examples, increased daylight hours refers to a light period of about 18 hours.

The phrase “days to flower” as used herein refers to the number of days it takes a plant to flower, measured as the days after sow until flower initiation. “Flower initiation” as used herein refers to the physiological process in a plant wherein the shoot apical meristem begins to develop flowers. “Flower initiation at a fewer number of days” refers to flower initiation in a fewer number of days relative to a suitable control. In some examples, flower initiation at a fewer number of days includes flower initiation within 33 to 38 days (on average) after sowing. In another example, flower initiation at a fewer number of days includes flower initiation within 53 to 67 days (on average) after sowing.

The term “detect” or “detecting” refers to any method for determining the presence of a nucleic acid. Methods of detecting nucleic acid polymorphisms, for example, have been described and can include amplification of a target polynucleotide (e.g., by PCR) and/or detection by a probe (e.g., hybridization assays). PCR uses a particular amplification primer pair that specifically hybridize to a target polynucleotide and produce an amplification product (the amplicon). Primers can be designed such that the amplicon can contain a nucleic acid polymorphism of interest. Methods for designing PCR primers and PCR conditions have been described, for example, in Sambrook et al. (2014) Molecular Cloning: A Laboratory Manual (Fourth Edition, Cold Spring Harbor Laboratory Press, Plainview, N.Y.).

The term “expression” or “gene expression” relates to the process by which the coded information of a nucleic acid transcriptional unit (including, e.g., genomic DNA) is converted into product, often including the synthesis of a protein or RNA. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any suitable, known method, including, without limitation, Northern blot, RT-PCR, Western blot, or in vitro, in situ, or in vivo protein activity assay(s). Elevated levels refer to higher than average levels of gene expression in comparison to a reference, e.g., Abacus.

The term “expression cassette” refers to a discrete nucleic acid fragment into which a nucleic acid sequence or fragment can be moved, typically for expression in a host cell. In some examples, an expression cassette is included on a vector, such as an expression vector.

The term “functional” as used herein refers to DNA or amino acid sequences which are of sufficient size and sequence to have the desired function (i.e. the ability to cause expression of a gene resulting in gene activity expected of the gene found in a reference genome, e.g., the Abacus reference genome.)

The term “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “recombinant expression construct”, which are used interchangeably, refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes.

The term “genetic modification” or “genetic alteration” as used herein refers to a change from the wild-type or reference sequence of one or more nucleic acid molecules. Genetic modifications or alterations include without limitation, base pair substitutions, additions and deletions of at least one nucleotide from a nucleic acid molecule of known sequence.

The term “genome” as it applies to plant cells encompasses not only chromosomal DNA found within the nucleus, but organelle DNA found within subcellular components (e.g., mitochondrial, plastid) of the cell.

The term “genotype” refers to the genetic makeup of an individual cell, cell culture, tissue, organism (e.g., a plant), or group of organisms at a particular locus. A “beneficial genotype” refers to a genotype that confers a desired trait, for example, a genotype that confers or is associated with flowering under increased daylight hours.

The term “germplasm” refers to genetic material of or from an individual (e.g., a plant), a group of individuals (e.g., a plant line, variety, or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants can be grown, as well as plant parts, such as leafs, stems, pollen, or cells that can be cultured into a whole plant.

The term “haplotype” refers to the genotype of a plant at a plurality of genetic loci, e.g., a combination of alleles or markers. Haplotype can refer to sequence of polymorphisms at a particular locus, such as a single marker locus, or sequence polymorphisms at multiple loci along a chromosomal segment in a given genome. As used herein, a haplotype can be a nucleic acid region spanning two markers.

A plant is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “hybrid” refers to a variety or cultivar that is the result of a cross of plants of two different varieties. A hybrid, as described here, can refer to plants that are genetically different at any particular number of loci. A hybrid can further include a plant that is a variety that has been bred to have at least one different characteristic from the parent. “F1 hybrid” refers to the first generation hybrid, “F2 hybrid” the second generation hybrid, “F3 hybrid” the third generation, and so on. A hybrid refers to any progeny that is either produced, or developed using research and development to create a new line having at least one distinct characteristic.

The terms “hybridizing specifically to”, “specific hybridization”, or “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. A “stringent hybridization” and “stringent hybridization wash conditions” in the context of nucleic acid hybridization (e.g., as in array, Southern or Northern hybridizations) are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, Ch. 2, “Overview of principles of hybridization and the strategy of nucleic acid probe assays,” Elsevier, N.Y. (“Tijssen”). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook et al. (2014) Molecular Cloning: A Laboratory Manual (Fourth Edition, Cold Spring Harbor Laboratory Press, Plainview, N.Y.)).

As used herein, the term “inbreeding” refers to the production of offspring via the mating between relatives. The plants resulting from the inbreeding process are referred to herein as “inbred plants” or “inbreds.”

The term “introduced” refers to incorporation of a nucleic acid (e.g., expression construct) or protein into a cell. Thus, “introduced” includes “transfection,” “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA). In some examples, a genetic modification (e.g., a substitution, insertion, or deletion) is introduced through a gene editing technique, such as an RNAi, CRISPR/Cas9, ZFN, or TALEN based technique. In some examples, a gene (or vector carrying a gene) is introduced into a cell by transformation, transfection, or transduction. The term “isolated” as used herein means having been removed from its natural environment, or removed from other compounds present when the compound is first formed. The term “isolated” embraces materials isolated from natural sources as well as materials (e.g., nucleic acids and proteins) recovered after preparation by recombinant expression in a host cell, or chemically-synthesized compounds such as nucleic acid molecules, proteins, and peptides.

The terms “initiate transcription,” “initiate expression,” “drive transcription,” and “drive expression” are used interchangeably herein and all refer to the primary function of a promoter. As detailed throughout this disclosure, a promoter is a non-coding genomic DNA sequence, usually upstream (5′) to the relevant coding sequence, and its primary function is to act as a binding site for RNA polymerase and initiate transcription by the RNA polymerase.

The term “locus” refers to a position on a genome that corresponds to a measurable property, e.g., a trait. Thus, an “increased daylight hours flowering trait locus” as used herein is a position on the genome of a subject plant having genetic differences, in comparison to a reference genome (e.g., Abacus), that results in the flowering at increased daylight hours in comparison to a reference plant.

The term “marker,” “genetic marker,” “molecular marker,” “marker nucleic acid,” are used interchangeably and refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Other examples of such markers are restriction fragment length polymorphism (RFLP) markers, amplified fragment length polymorphism (AFLP) markers, single nucleotide polymorphisms (SNPs), microsatellite markers (e.g. SSRs), sequence-characterized amplified region (SCAR) markers, cleaved amplified polymorphic sequence (CAPS) markers or isozyme markers or combinations of the markers described herein which defines a specific genetic and chromosomal location.

The term “marker assisted selection” refers to the diagnostic process of identifying, optionally followed by selecting a plant from a group of plants using the presence of a molecular marker as the diagnostic characteristic or selection criterion. The process usually involves detecting the presence of a certain nucleic acid sequence or polymorphism in the genome of a plant.

The term “modified flowering” includes initiating flowering at increased daylight hours (e.g., about 18 hours daylight) and/or initiating flowering in a fewer number of days (e.g., fewer than 70 days on average, for example, 33 to 38 days or 53 to 67 days on average) relative to a control (e.g., a Cannabis plant without a marker indicating flower initiation in a fewer number of days).

The term “nucleotide” refers to an organic molecule that serves as a monomeric unit of DNA and RNA. The nucleotide position is the position along a chromosome wherein any particular monomeric unit of DNA or RNA is positioned relative to the other monomeric units of DNA or RNA.

The term “offspring” or “progeny” refer to a plant resulting as from a vegetative or sexual reproduction from one or more parent plants. For instance, an offspring/progeny plant may be obtained by cloning or selfing of a parent plant or by crossing two parent plants. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination.

The terms “PCR” or “Polymerase Chain Reaction” refers to a technique for the synthesis of large quantities of specific DNA segments, consisting of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps comprises a cycle.

The term “plant” refers to a whole plant, cell, tissue, or other plant parts. Plant parts include any part(s) of a plant, including, for example and without limitation: seed (including mature seed and immature seed); a plant cutting; a plant cell; a plant cell culture; a plant organ (e.g., pollen, embryos, flowers, trichomes, fruits, shoots, leaves, roots, stems, and explants). Plant tissue refers to any tissue of a plant, including but not limited to, tissue from an embryo, shoot, root, stem, seed, stipule, leaf, trichome, petal, flower bud, flower, ovule, bract, branch, petiole, internode, bark, pubescence, tiller, rhizome, frond, blade, ovule, pollen, stamen. A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks. Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root.

A plant cell is the structural and physiological unit of the plant. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell.” Described herein are plants in the genus of Cannabis and plants derived therefrom, which can be produced by asexual or sexual reproduction.

The term “polymorphism” refers to a difference in the nucleotide or amino acid sequence of a given region as compared to a nucleotide or amino acid sequence in a homologous-region of another individual, in particular, a difference in the nucleotide of amino acid sequence of a given region which differs between individuals of the same species. A polymorphism is generally defined in relation to a reference sequence. Unless indicated otherwise, the reference sequence is the Cannabis Abacus reference genome (version Csat_AbacusV2, NCBI assembly accession GCA_025232715.1) or CDS produced from the Cannabis Abacus reference genome. Polymorphisms include single nucleotide differences, differences in sequence of more than one nucleotide, and single or multiple nucleotide insertions, inversions and deletions; as well as single amino acid differences, differences in sequence of more than one amino acid, and single or multiple amino acid insertions, inversions, and deletions.

The terms “polynucleotide,” “polynucleotide sequence,” “nucleotide sequence,” “nucleic acid sequence,” and “nucleic acid fragment,” are used interchangeably. These terms encompass polymers composed of nucleotide units (ribonucleotides, deoxyribonucleotides, related naturally occurring structural variants, and synthetic non-naturally occurring analogs thereof). The term “oligonucleotide” typically refers to short polynucleotides, generally no greater than 150 nucleotides, for example, no greater than 125 nucleotides, no greater than 100 nucleotides, no greater than 75 nucleotides, no greater than 50 nucleotides, or no greater than 25 nucleotides. It will be understood that when a nucleic acid sequence is represented as a DNA sequence (i.e., A, T, G, C), this also includes an RNA sequence (i.e., A, U, G, C) in which “U” replaces “T.” Nucleic acids can be single- or double-stranded. Exemplary nucleic acids include cDNA, genomic DNA, synthetic DNA, RNA, or mixtures thereof.

The term “polypeptide” or “protein” refers to a polymer of amino acid residues. The terms apply to amino acid polymers in which one or more amino acid residue is an artificial chemical analogue of a corresponding naturally occurring amino acid, as well as to naturally occurring amino acid polymers. The term “amino acid residue” or “amino acid” includes reference to an amino acid that is incorporated into a protein, polypeptide, or peptide. The amino acid can be a naturally occurring amino acid and, unless otherwise limited, can encompass known analogs of natural amino acids that can function in a similar manner as naturally occurring amino acids. As used herein, “recombinant” includes reference to a protein produced using cells that do not have, in their native state, an endogenous copy of the DNA able to express the protein. The cells produce the recombinant protein because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified.

The term “probe,” “nucleic acid probe,” or “oligonucleotide probe” as used herein, is one or more synthetic nucleic acid molecules that are complementary to a nucleic acid sequence of interest (target sequence), and hybridize to a sequence of interest when under hybridization conditions. Probes can be used to detect, analyze, and/or visualize the nucleic acid sequence of interest on a molecular level. Specific hybridization of a probe to a nucleic acid sequence of interest can be detected, for example, through a label on the probe. Probes have a length suitable to achieve a desired specificity to the target sequence, however, are generally at least 10 nucleotides long, for example, at least 15 nucleotides, at least 20 nucleotides, or at least 50 nucleotides long. Probes can be immobilized on a solid surface (e.g., nitrocellulose, glass, quartz, fused silica slides), as in an array. The precise sequence of the particular probes described herein can be modified to a certain degree to produce probes that are “substantially identical” to the disclosed probes, but retain the ability to specifically bind to (i.e., hybridize specifically to) the same targets as the probe from which they were derived. Such modifications are specifically covered by reference to the individual probes described herein.

The term “product” as used in reference to a Cannabis product, is a composition including Cannabis (or an extract thereof). Products include, but are not limited to: a kief, hashish, bubble hash, an edible product, solvent reduced oil, sludge, e-juice, tincture, or other compositions including Cannabis (e.g., a Cannabis plant disclosed herein, or an extract thereof).

The term “quantitative trait loci” or “QTL” refers to the genetic elements controlling a quantitative trait.

The term “reference plant” or “reference genome” refers to a wild-type or reference sequence that SNPs or other markers in a test sample can be compared to in order to detect a modification of the sequence in the test sample. The phrase “Abacus Cannabis reference genome” thus refers to the Abacus reference genome (version Csat_AbacusV2; NCBI assembly accession GCA_025232715.1).

The terms “sequence identity” or “percent identity” are used interchangeably to refer to a sequence comparison based on identical matches between correspondingly identical positions in two or more amino acid or nucleotide sequences that are being compared. The percent identity refers to the extent to which two optimally aligned polynucleotide or peptide sequences are invariant throughout a window of alignment of components, e.g., nucleotides or amino acids. Hybridization experiments and mathematical algorithms known in the art may be used to determine percent identity. Many mathematical algorithms exist as sequence alignment computer programs known in the art that calculate percent identity. These programs may be categorized as either global sequence alignment programs or local sequence alignment programs.

The NCBI Basic Local Alignment Search Tool (BLAST) tool is often used and is available from several sources, including the National Center for Biotechnology Information (blast.ncbi.nlm.nih.gov/Blast.cgi). Various types of BLAST are available, for example, blastp, blastn, blastx, tblastn and tblastx. A description of how to determine sequence identity using this program is available on the NCBI website and other resources. In some examples, percent sequence identity is determined by using BLAST with default parameters.

The terms “similar,” “substantially similar” and “corresponding substantially” as used herein refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood that the disclosure encompasses more than the specific exemplary sequences. A “substantially homologous sequence” refers to variants of the disclosed sequences such as those that result from site-directed mutagenesis, as well as synthetically derived sequences. A substantially homologous sequence of the present disclosure also refers to those fragments of a particular promoter nucleotide sequence disclosed herein that operate to promote the constitutive expression of an operably linked heterologous nucleic acid fragment. These promoter fragments will comprise at least about 20 contiguous nucleotides, preferably at least about 50 contiguous nucleotides, more preferably at least about 75 contiguous nucleotides, even more preferably at least about 100 contiguous nucleotides of the particular promoter nucleotide sequence disclosed herein. The nucleotides of such fragments will usually comprise the TATA recognition sequence of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. Variants of these promoter fragments, such as those resulting from site-directed mutagenesis, are encompassed by the present disclosure.

The term “single nucleotide polymorphism (SNP)” refers to a change in which a single base in the DNA differs from the usual base at that position. These single base changes are called SNPs.

The term “target region” or “nucleic acid target” refers to a nucleotide sequence that resides at a specific chromosomal location. The “target region” or “nucleic acid target” can be specifically recognized by a probe.

The term “transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. The term “transgenic plant” refers to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct. A “transgene” is a gene that has been introduced into the genome by a transformation procedure.

### Cannabis

Cannabis has long been used for drug and industrial purposes, fiber (hemp), for seed and seed oils, for medicinal purposes, and for recreational purposes. Industrial hemp products are made from Cannabis plants selected to produce an abundance of fiber. Some Cannabis varieties have been bred to produce minimal levels of THC, the principal psychoactive constituent responsible for the psychoactivity associated with marijuana. Marijuana has historically consisted of the dried flowers of Cannabis plants selectively bred to produce high levels of THC and other psychoactive cannabinoids. Various extracts including hashish and hash oil are also produced from the plant.

Cannabis is an annual, dioecious, flowering herb. The leaves are palmately compound or digitate, with serrate leaflets. Cannabis normally has imperfect flowers, with staminate “male” and pistillate “female” flowers occurring on separate plants. It is not unusual, however, for individual plants to separately bear both male and female flowers (i.e., have monoecious plants). Although monoecious plants are often referred to as “hermaphrodites,” true hermaphrodites (which are less common in Cannabis) bear staminate and pistillate structures on individual flowers, whereas monoecious plants bear male and female flowers at different locations on the same plant.

The life cycle of Cannabis varies with each variety but can be generally summarized into germination, vegetative growth, and reproductive stages. Because of heavy breeding and selection by humans, most Cannabis seeds have lost dormancy mechanisms and do not require any pre-treatments or winterization to induce germination (See Clarke, R C et al. “Cannabis: Evolution and Ethnobotany” University of California Press 2013). Seeds placed in viable growth conditions are expected to germinate in about 3 to 7 days. The first true leaves of a Cannabis plant contain a single leaflet, with subsequent leaves developing in opposite formation with increasing number of leaflets. Leaflets can be narrow or broad depending on the morphology of the plant grown. Cannabis plants are normally allowed to grow vegetatively for the first 4 to 8 weeks. During this period, the plant responds to increasing light with faster and faster growth. Under ideal conditions, Cannabis plants can grow up to 2.5 inches a day, and are capable of reaching heights of up to 20 feet. Indoor growth pruning techniques tend to limit Cannabis size through careful pruning of apical or side shoots.

Cannabis is diploid, having a chromosome complement of 2n=20, although polyploid individuals have been artificially produced. The first genome sequence of Cannabis, which is estimated to be 820 Mb in size, was published in 2011 by a team of Canadian scientists (Bakel et al, “The draft genome and transcriptome of Cannabis sativa” Genome Biology 12:R102).

Most Cannabis varieties do not initiate flowers at day lengths greater than 12 hours, for this reason common cultivation practices involve growing plants for at least three weeks under long day length followed by a switch to 12/12 (12 hours light, 12 hours dark) after which plants initiate flowers after around 12 days. Thus, markers associated with modified flowering are desirable to allow breeders to harvest plants at earlier intervals during the growing season and grow Cannabis outdoors or in greenhouses without artificial light deprivation at different latitudes.

### Flower Initiation Markers, Haplotypes, and Trait Loci

Disclosed are methods of selecting or producing Cannabis plants that initiate flowering at increased daylight hours. In some examples, the methods include obtaining a nucleic acid sample from a plant or its germplasm; (ii) detecting one or more markers that indicate flower initiation at increased daylight hours, (iii) indicating the flower initiation at increased daylight hours, and (iv) selecting the one or more plants that initiate flowering at increased daylight hours. In some examples, the methods include (i) obtaining a nucleic acid sample from a plant or its germplasm; (ii) detecting one or more markers that indicate flower initiation at increased daylight hours, (iii) crossing the plant comprising the one or more markers, and (iv) obtaining one or more progeny plants comprising the one or more markers and that initiate flowering at increased daylight hours. Crossing includes, for example, outcrossing, backcrossing, sibling crossing, or selfing. In some examples, a Cannabis plant comprising the one or more markers that indicate flower initiation at increased daylight hours is crossed with a second plant that does not initiate flowering at increased daylight hours. Progeny plants that initiate flowering at increased daylight hours can be identified and/or selected. In an example, flowering at increased daylight hours includes flowering when the daylight is greater than 12 hours (e.g., 13 to 18 daylight hours or 16 to 18 daylight hours). Genetic markers associated with flowering at increased daylight hours are described herein, for example, see Table 2, 4, 5, or 10.

The markers of the present disclosure were discovered as described herein, which comprise polymorphisms relative to the Abacus Cannabis reference genome (version Csat_AbacusV2; NCBI assembly accession GCA_025232715.1). In an example, as described in Tables 2, 4, 5, and 10, the markers identify polymorphisms that indicate flower initiation at increased daylight hours. In another example, as described in Tables 3, 6, and 7, the markers identify polymorphisms that indicate flower initiation at a fewer number of days following sow. In some examples, markers that indicate flower initiation at increased daylight hours also indicate flower initiation at a fewer number of days following sow.

The markers of the present disclosure may be described in numerous fashions. To illustrate, for non-limiting exemplary purposes, marker 171_6189, described in Table 2, is described as being positioned at base pair (bp) position 19,681 on chromosome 8 of the Abacus reference genome. Likewise, marker 171_6189 is described as being positioned at nucleotide 51 of SEQ ID NO:1.

In a specific example, the one or more markers that indicate flower initiation at increased daylight hours comprise a polymorphism on chromosome 8 relative to a reference genome at nucleotide positions: 19.681; 44,981; 51,589; 87,904; 159,096; 199,126; 371,700; 378,368; 385,390; 447,673; 555,854; 564,634; 568,544; 720,538; 936,746; 1,042,634; 1,088,206; 1,147,746; 1,191,275; 1,200,057; 1,211,168; 1,216,900; 1,218,786; 1,277,500; 1,305,959; 1,397,056; 1,496,730; 1,515,643; 1,526,023; 1,577,268; 1,582,128; 1,617,208; 1,659,209; 1,690,294; 1,725,273; 1,729,625; 1,735,848; 1,816,071; 1,877,889; 1,933,751; 1,993,648; 2,002,233; 2,020,279; 2,253,641; 2,279,386; 2,364,298; 2,404,096; 63,750; 95,593; 100,604; 109,674; 146,257; 241,017; 321,930; 334,676; 351,874; 395,213; 413,345; 479,822; 491,066; 498,442; 512,584; 531,560; 537,879; 606,518; 639,258; 673,171; 690,591; 703,062; 709,849; 714,581; 784,607; 805,642; 827,002; 859,843; 935,803; 957,083; 963,275; 968,829; 972,965; 978,102; 1,008,257; 1,048,013; 1,076,208; 1,078,159; 1,108,104; 1,134,931; 1,154,438; 1,162,631; 1,193,261; 1,228,323; 1,273,758; 1,281,517; 1,319,381; 1,355,612; 1,376,341; 1,511,014; 1,557,156; 1,595,751; 1,600,294; 1,618,178; 1,622,540; 1,707,047; 1,753,859; 1,783,885; 1,790,563; 1,802,247; 1,810,522; 1,813,955; 1,835,318; 1,844,801; 1,872,522; 1,909,161; 1,911,974; 1,944,792; 1,956,392; 1,971,081; 1,974,016; 1,997,626; 2,061,626; 2,079,392; 2,079,928; 2,087,331; 2,099,627; 2,101,607; 2,127,039; 2,160,732; 2,211,816; 2,267,196; 2,369,020; 2,383,741; 2,503,244; 2,504,080; 2,510,824; 2,816,933; 2,828,279; 2,833,001; 2,846,467; 2,862,613; 2,912,209; 3,098,435; or 3,794,786 wherein the reference genome is the Abacus Cannabis reference genome Csat_AbacusV2, NCBI assembly accession GCA_025232715.1. In some examples, the nucleotide position comprises on chromosome 8: (a) a A/A or G/A genotype at position 19681; (b) a A/A or T/A genotype at position 44981; (c) a C/C or T/C genotype at position 51589; (d) a G/G or A/G genotype at position 87904; (e) a G/G or G/A genotype at position 159096; (f) a G/G or G/C genotype at position 199126; (g) a A/A or G/A genotype at position 371700; (h) a G/G or G/T genotype at position 378368; (i) a G/G or G/A genotype at position 385390; (j) a G/G or A/G genotype at position 447673; (k) a T/T or T/C genotype at position 555854; (l) a T/T or T/C genotype at position 564634; (m) a G/G or G/A genotype at position 568544; (n) a T/T or T/G genotype at position 720538; (o) a A/A or A/T genotype at position 936746; (p) a G/G or G/A genotype at position 1042634; (q) a C/C or T/C genotype at position 1088206; (r) a C/C or C/T genotype at position 1147746; (s) a C/C or T/C genotype at position 1191275; (t) a G/G or G/A genotype at position 1200057; (u) a T/T or C/T genotype at position 1211168; (v) a T/T or T/C genotype at position 1216900; (w) a G/G or A/G genotype at position 1218786; (x) a T/T or G/T genotype at position 1277500; (y) a T/C genotype at position 1305959; (z) a A/A or A/G genotype at position 1397056; (aa) a A/A or A/G genotype at position 1496730; (ab) a T/T or T/C genotype at position 1515643; (ac) a A/A or A/T genotype at position 1526023; (ad) a G/G or A/G genotype at position 1577268; (ae) a T/T or G/T genotype at position 1582128; (af) a C/C or G/C genotype at position 1617208; (ag) a C/C or A/C genotype at position 1659209; (ah) a A/A or A/G genotype at position 1690294; (ai) a G/G or G/A genotype at position 1725273; (aj) a C/C or C/G genotype at position 1729625; (ak) a G/G or T/G genotype at position 1735848; (al) a A/A or T/A genotype at position 1816071; (am) a C/C or A/C genotype at position 1877889; (an) a C/C or C/A genotype at position 1933751; (ao) a T/T or C/T genotype at position 1993648; (ap) a A/A or C/A genotype at position 2002233; (aq) a A/A or A/G genotype at position 2020279; (ar) a C/C or C/A genotype at position 2253641; (as) a C/C or C/A genotype at position 2279386; (at) a G/G or A/G genotype at position 2364298; or (au) a G/G or A/G genotype at position 2404096; (av) a C/C or G/C genotype at position 63750; (aw) a C/C or T/C genotype at position 95593; (ax) a T/T or C/T genotype at position 100604; (ay) a A/A or T/A genotype at position 109674; (az) a A/A or A/C genotype at position 146257; (ba) a A/A or A/G genotype at position 241017; (bb) a G/G or G/A genotype at position 321930; (bc) a T/T or T/A genotype at position 334676; (bd) a A/A or A/T genotype at position 351874; (be) a C/C or C/A genotype at position 395213; (bf) a G/G or A/G genotype at position 413345; (bg) a T/T or T/C genotype at position 479822; (bh) a T/T or T/A genotype at position 491066; (bi) a T/T or C/T genotype at position 498442; (bj) a A/A or G/A genotype at position 512584; (bk) a A/A or A/C genotype at position 531560; (bl) a A/A or A/G genotype at position 537879; (bm) a A/A or G/A genotype at position 606518; (bn) a A/A or A/G genotype at position 639258; (bo) a A/A or G/A genotype at position 673171; (bp) a C/C or C/T genotype at position 690591; (bq) a A/A or A/C genotype at position 703062; (br) a C/C or A/C genotype at position 709849; (bs) a A/A or G/A genotype at position 714581; (bt) a G/G or G/A genotype at position 784607; (bu) a C/C or C/T genotype at position 805642; (bv) a T/T or T/C genotype at position 827002; (bw) a GIG or G/A genotype at position 859843; (bx) a A/A or A/G genotype at position 935803; (by) a A/A or T/A genotype at position 957083; (bz) a C/C or C/T genotype at position 963275; (ca) a T/T or T/A genotype at position 968829; (cb) a C/C or C/A genotype at position 972965; (cc) a A/A or A/G genotype at position 978102; (cd) a T/T or T/A genotype at position 1008257; (ce) a C/C or C/T genotype at position 1048013; (cf) a G/G or G/T genotype at position 1076208; (eg) a A/A or A/G genotype at position 1078159; (ch) a A/A or G/A genotype at position 1108104; (ci) a A/A or A/T genotype at position 1134931; (cj) a G/G or A/G genotype at position 1154438; (ck) a G/G or C/G genotype at position 1162631; (cl) a G/G or G/A genotype at position 1193261; (cm) a C/C or T/C genotype at position 1228323; (cn) a T/T or C/T genotype at position 1273758; (co) a A/A or A/C genotype at position 1281517; (cp) a A/A or G/A genotype at position 1319381; (cq) a G/G or A/G genotype at position 1355612; (cr) a G/G or A/G genotype at position 1376341; (cs) a C/C or C/T genotype at position 1511014; (ct) a G/G or G/T genotype at position 1557156; (cu) a A/A or A/G genotype at position 1595751; (cv) a A/A or A/G genotype at position 1600294; (cw) a T/T or T/C genotype at position 1618178; (cx) a G/G or G/A genotype at position 1622540; (cy) a T/T or T/C genotype at position 1707047; (cz) a G/G or T/G genotype at position 1753859; (da) a G/G or A/G genotype at position 1783885; (db) a G/G or A/G genotype at position 1790563; (dc) a A/A or A/G genotype at position 1802247; (dd) a A/A or C/A genotype at position 1810522; (de) a C/C or C/A genotype at position 1813955; (df) a C/C or C/G genotype at position 1835318; (dg) a G/G or A/G genotype at position 1844801; (dh) a G/G or T/G genotype at position 1872522; (di) a T/T or C/T genotype at position 1909161; (dj) a G/G or A/G genotype at position 1911974; (dk) a A/A or A/G genotype at position 1944792; (dl) a G/G or G/A genotype at position 1956392; (dm) a C/C or C/T genotype at position 1971081; (dn) a T/T or T/C genotype at position 1974016; (do) a T/T or T/C genotype at position 1997626; (dp) a A/A or T/A genotype at position 2061626; (dq) a C/C or T/C genotype at position 2079392; (dr) a G/G or T/G genotype at position 2079928; (ds) a T/T or T/G genotype at position 2087331; (dt) a C/C or T/C genotype at position 2099627; (du) a T/T or A/T genotype at position 2101607; (dv) a G/G or G/A genotype at position 2127039; (dw) a A/A or G/A genotype at position 2160732; (dx) a A/A or A/G genotype at position 2211816; (dy) a T/T or C/T genotype at position 2267196; (dz) a A/A or G/A genotype at position 2369020; (ea) a C/C or T/C genotype at position 2383741; (eb) a G/G or A/G genotype at position 2503244; (ec) a A/A or A/G genotype at position 2504080; (ed) a T/T or A/T genotype at position 2510824; (ee) a T/T or C/T genotype at position 2816933; (ef) a G/G or A/G genotype at position 2828279; (eg) a A/A or G/A genotype at position 2833001; (eh) a C/C or C/G genotype at position 2846467; (ei) a T/T or T/A genotype at position 2862613; (ej) a T/T or C/T genotype at position 2912209; (ek) a T/T or C/T genotype at position 3098435; or (el) a G/G or T/G genotype at position 3794786; wherein the reference genome is the Abacus Cannabis reference genome version Csat_AbacusV2, NCBI assembly accession GCA_025232715.1.

In a non-limiting example, the one or more markers that indicate flower initiation at increased daylight hours comprise a polymorphism on chromosome 8 at any one of nucleotide positions 19,681; 44,981; 87,904; 109,674; 159,096; 378,368; 479,822; 491,066; 568,544; 720,538; 968,829; 1,191,275; 1,200,057; 1,211,168; 1,273,758; 1,277,500; 1,305,959; 1,659,209; 1,690,294; 1,735,848; 1,816,071; 1,872,522; 1,993,648; 2,002,233; 2,020,279; 2,079,928; 2,267,196, wherein the reference genome is the Abacus Cannabis reference genome version Csat_AbacusV2, NCBI assembly accession GCA_025232715.1. In some examples, the nucleotide position comprises on chromosome 8: (a) a A/A or G/A genotype at position 19681; (b) a A/A or T/A genotype at position 44981; (c) a G/G or A/G genotype at position 87904; (d) a A/A or T/A genotype at position 109674; (e) a G/G or G/A genotype at position 159096; (f) a G/G or G/T genotype at position 378368; (g) a T/T or T/C genotype at position 479822; (h) a T/T or T/A genotype at position 491066; (i) a G/G or G/A genotype at position 568544; (j) a T/T or T/G genotype at position 720538; (k) a T/T or T/A genotype at position 968829; (1) a C/C or T/C genotype at position 1191275; (in) a G/G or G/A genotype at position 1200057; (n) a T/T or C/T genotype at position 1211168; (o) a T/T or C/T genotype at position 1273758; (p) a T/T or G/T genotype at position 1277500; (q) a T/C or genotype at position 1305959; (r) a C/C or A/C genotype at position 1659209; (s) a A/A or A/G genotype at position 1690294; (t) a G/G or T/G genotype at position 1735848; (u) a A/A or T/A genotype at position 1816071; (v) a G/G or T/G genotype at position 1872522; (w) a T/T or C/T genotype at position 1993648; (x) a A/A or C/A genotype at position 2002233; (y) a A/A or A/G genotype at position 2020279; (z) a G/G or T/G genotype at position 2079928; or (aa) a T/T or C/T genotype at position 2267196, wherein the reference genome is the Abacus Cannabis reference genome version Csat_AbacusV2, NCBI assembly accession GCA_025232715.1.

In a non-limiting example, the one or more markers that indicate flower initiation at increased daylight hours comprise a polymorphism on chromosome 8 at any one of nucleotide positions 109,674; 159,096; or 1,154,438, wherein the reference genome is the Abacus Cannabis reference genome version Csat_AbacusV2, NCBI assembly accession GCA_025232715.1. In some examples, the nucleotide position comprises on chromosome 8: (a) a A/A or T/A genotype at position 109674; (b) a G/G or G/A genotype at position 159096; or (c) a G/G or A/G genotype at position 1154438.

In another example, the one or more markers that indicate flower initiation at increased daylight hours comprises a polymorphism at position 51 of any one or more of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; or SEQ ID NO: 143. In some examples, the nucleotide position comprises: (a) a A/A or G/A genotype at position 51 of SEQ ID NO: 1; (b) a A/A or T/A genotype at position 51 of SEQ ID NO: 2; (c) a C/C or T/C genotype at position 51 of SEQ ID NO: 3; (d) a G/G or A/G genotype at position 51 of SEQ ID NO: 4; (e) a G/G or G/A genotype at position 51 of SEQ ID NO: 5; (f) a G/G or G/C genotype at position 51 of SEQ ID NO: 6; (g) a A/A or G/A genotype at position 51 of SEQ ID NO: 7; (h) a G/G or G/T genotype at position 51 of SEQ ID NO: 8; (i) a G/G or G/A genotype at position 51 of SEQ ID NO: 9; (j) a G/G or A/G genotype at position 51 of SEQ ID NO: 10; (k) a T/T or T/C genotype at position 51 of SEQ ID NO: 11; (1) a T/T or T/C genotype at position 51 of SEQ ID NO: 12; (m) a G/G or G/A genotype at position 51 of SEQ ID NO: 13; (n) a T/T or T/G genotype at position 51 of SEQ ID NO: 14; (o) a A/A or A/T genotype at position 51 of SEQ ID NO: 15; (p) a G/G or G/A genotype at position 51 of SEQ ID NO: 16; (q) a C/C or T/C genotype at position 51 of SEQ ID NO: 17; (r) a C/C or C/T genotype at position 51 of SEQ ID NO: 18; (s) a C/C or T/C genotype at position 51 of SEQ ID NO: 19; (t) a G/G or G/A genotype at position 51 of SEQ ID NO: 20; (u) a T/T or C/T genotype at position 51 of SEQ ID NO: 21; (v) a T/T or T/C genotype at position 51 of SEQ ID NO: 22; (w) a G/G or A/G genotype at position 51 of SEQ ID NO: 23; (x) a T/T or G/T genotype at position 51 of SEQ ID NO: 24; (y) a T/C genotype at position 51 of SEQ ID NO: 25; (z) a A/A or A/G genotype at position 51 of SEQ ID NO: 26; (aa) a A/A or A/G genotype at position 51 of SEQ ID NO: 27; (ab) a T/T or T/C genotype at position 51 of SEQ ID NO: 28; (ac) a A/A or A/T genotype at position 51 of SEQ ID NO: 29; (ad) a G/G or A/G genotype at position 51 of SEQ ID NO: 30; (ae) a T/T or G/T genotype at position 51 of SEQ ID NO: 31; (af) a C/C or G/C genotype at position 51 of SEQ ID NO: 32; (ag) a C/C or A/C genotype at position 51 of SEQ ID NO: 33; (ah) a A/A or A/G genotype at position 51 of SEQ ID NO: 34; (ai) a G/G or G/A genotype at position 51 of SEQ ID NO: 35; (aj) a C/C or C/G genotype at position 51 of SEQ ID NO: 36; (ak) a G/G or T/G genotype at position 51 of SEQ ID NO: 37; (al) a A/A or T/A genotype at position 51 of SEQ ID NO: 38; (am) a C/C or A/C genotype at position 51 of SEQ ID NO: 39; (an) a C/C or C/A genotype at position 51 of SEQ ID NO: 40; (ao) a T/T or C/T genotype at position 51 of SEQ ID NO: 41; (ap) a A/A or C/A genotype at position 51 of SEQ ID NO: 42; (aq) a A/A or A/G genotype at position 51 of SEQ ID NO: 43; (ar) a C/C or C/A genotype at position 51 of SEQ ID NO: 44; (as) a C/C or C/A genotype at position 51 of SEQ ID NO: 45; (at) a G/G or A/G genotype at position 51 of SEQ ID NO: 46; (au) a G/G or A/G genotype at position 51 of SEQ ID NO: 47; (av) a C/C or G/C genotype at position 51 of SEQ ID NO: 49; (aw) a C/C or T/C genotype at position 51 of SEQ ID NO: 50; (ax) a T/T or C/T genotype at position 51 of SEQ ID NO: 51; (ay) a A/A or T/A genotype at position 51 of SEQ ID NO: 52; (az) a A/A or A/C genotype at position 51 of SEQ ID NO: 53; (ba) a A/A or A/G genotype at position 51 of SEQ ID NO: 54; (bb) a G/G or G/A genotype at position 51 of SEQ ID NO: 55; (bc) a T/T or T/A genotype at position 51 of SEQ ID NO: 56; (bd) a A/A or A/T genotype at position 51 of SEQ ID NO: 57; (be) a C/C or C/A genotype at position 51 of SEQ ID NO: 58; (bf) a G/G or A/G genotype at position 51 of SEQ ID NO: 59; (bg) a T/T or T/C genotype at position 51 of SEQ ID NO: 60; (bh) a T/T or T/A genotype at position 51 of SEQ ID NO: 61; (bi) a T/T or C/T genotype at position 51 of SEQ ID NO: 62; (bj) a A/A or G/A genotype at position 51 of SEQ ID NO: 63; (bk) a A/A or A/C genotype at position 51 of SEQ ID NO: 64; (bl) a A/A or A/G genotype at position 51 of SEQ ID NO: 65; (bm) a A/A or G/A genotype at position 51 of SEQ ID NO: 66; (bn) a A/A or A/G genotype at position 51 of SEQ ID NO: 67; (bo) a A/A or G/A genotype at position 51 of SEQ ID NO: 68; (bp) a C/C or C/T genotype at position 51 of SEQ ID NO: 69; (bq) a A/A or A/C genotype at position 51 of SEQ ID NO: 70; (br) a C/C or A/C genotype at position 51 of SEQ ID NO: 71; (bs) a A/A or G/A genotype at position 51 of SEQ ID NO: 72; (bt) a G/G or G/A genotype at position 51 of SEQ ID NO: 73; (bu) a C/C or C/T genotype at position 51 of SEQ ID NO: 74; (by) a T/T or T/C genotype at position 51 of SEQ ID NO: 75; (bw) a G/G or G/A genotype at position 51 of SEQ ID NO: 76; (bx) a A/A or A/G genotype at position 51 of SEQ ID NO: 77; (by) a A/A or T/A genotype at position 51 of SEQ ID NO: 78; (bz) a C/C or C/T genotype at position 51 of SEQ ID NO: 79; (ca) a T/T or T/A genotype at position 51 of SEQ ID NO: 80; (cb) a C/C or C/A genotype at position 51 of SEQ ID NO: 81; (cc) a A/A or A/G genotype at position 51 of SEQ ID NO: 82; (cd) a T/T or T/A genotype at position 51 of SEQ ID NO: 83; (ce) a C/C or C/T genotype at position 51 of SEQ ID NO: 84; (cf) a G/G or G/T genotype at position 51 of SEQ ID NO: 85; (cg) a A/A or A/G genotype at position 51 of SEQ ID NO: 86; (ch) a A/A or G/A genotype at position 51 of SEQ ID NO: 87; (ci) a A/A or A/T genotype at position 51 of SEQ ID NO: 88; (cj) a G/G or A/G genotype at position 51 of SEQ ID NO: 89; (ck) a G/G or C/G genotype at position 51 of SEQ ID NO: 90; (cl) a G/G or G/A genotype at position 51 of SEQ ID NO: 91; (cm) a C/C or T/C genotype at position 51 of SEQ ID NO: 92; (en) a T/T or C/T genotype at position 51 of SEQ ID NO: 93; (co) a A/A or A/C genotype at position 51 of SEQ ID NO: 94; (cp) a A/A or G/A genotype at position 51 of SEQ ID NO: 95; (cq) a G/G or A/G genotype at position 51 of SEQ ID NO: 96; (cr) a G/G or A/G genotype at position 51 of SEQ ID NO: 97; (cs) a C/C or C/T genotype at position 51 of SEQ ID NO: 98; (ct) a G/G or G/T genotype at position 51 of SEQ ID NO: 99; (cu) a A/A or A/G genotype at position 51 of SEQ ID NO: 100; (cv) a A/A or A/G genotype at position 51 of SEQ ID NO: 101; (cw) a T/T or T/C genotype at position 51 of SEQ ID NO: 102; (cx) a G/G or G/A genotype at position 51 of SEQ ID NO: 103; (cy) a T/T or T/C genotype at position 51 of SEQ ID NO: 104; (cz) a G/G or T/G genotype at position 51 of SEQ ID NO: 105; (da) a G/G or A/G genotype at position 51 of SEQ ID NO: 106; (db) a G/G or A/G genotype at position 51 of SEQ ID NO: 107; (dc) a A/A or A/G genotype at position 51 of SEQ ID NO: 108; (dd) a A/A or C/A genotype at position 51 of SEQ ID NO: 109; (de) a C/C or C/A genotype at position 51 of SEQ ID NO: 110; (df) a C/C or C/G genotype at position 51 of SEQ ID NO: 111; (dg) a G/G or A/G genotype at position 51 of SEQ ID NO: 112; (dh) a G/G or T/G genotype at position 51 of SEQ ID NO: 113; (di) a T/T or C/T genotype at position 51 of SEQ ID NO: 114; (dj) a G/G or A/G genotype at position 51 of SEQ ID NO: 115; (dk) a A/A or A/G genotype at position 51 of SEQ ID NO: 116; (dl) a G/G or G/A genotype at position 51 of SEQ ID NO: 117; (dm) a C/C or C/T genotype at position 51 of SEQ ID NO: 118; (dn) a T/T or T/C genotype at position 51 of SEQ ID NO: 119; (do) a T/T or T/C genotype at position 51 of SEQ ID NO: 120; (dp) a A/A or T/A genotype at position 51 of SEQ ID NO: 121; (dq) a C/C or T/C genotype at position 51 of SEQ ID NO: 122; (dr) a G/G or T/G genotype at position 51 of SEQ ID NO: 123; (ds) a T/T or T/G genotype at position 51 of SEQ ID NO: 124; (dt) a C/C or T/C genotype at position 51 of SEQ ID NO: 125; (du) a T/T or A/T genotype at position 51 of SEQ ID NO: 126; (dv) a G/G or G/A genotype at position 51 of SEQ ID NO: 127; (dw) a A/A or G/A genotype at position 51 of SEQ ID NO: 128; (dx) a A/A or A/G genotype at position 51 of SEQ ID NO: 129; (dy) a T/T or C/T genotype at position 51 of SEQ ID NO: 130; (dz) a A/A or G/A genotype at position 51 of SEQ ID NO: 131; (ea) a C/C or T/C genotype at position 51 of SEQ ID NO: 132; (eb) a G/G or A/G genotype at position 51 of SEQ ID NO: 133; (ec) a A/A or A/G genotype at position 51 of SEQ ID NO: 134; (ed) a T/T or A/T genotype at position 51 of SEQ ID NO: 135; (ee) a T/T or C/T genotype at position 51 of SEQ ID NO: 136; (ef) a G/G or A/G genotype at position 51 of SEQ ID NO: 137; (eg) a A/A or G/A genotype at position 51 of SEQ ID NO: 138; (eh) a C/C or C/G genotype at position 51 of SEQ ID NO: 139; (ei) a T/T or T/A genotype at position 51 of SEQ ID NO: 140; (ej) a T/T or C/T genotype at position 51 of SEQ ID NO: 141; (ek) a T/T or C/T genotype at position 51 of SEQ ID NO: 142; or (el) a G/G or T/G genotype at position 51 of SEQ ID NO: 143.

In some examples, detecting one or more markers that indicate flower initiation at increased daylight hours includes detecting at least two markers, such as at least 3, 5, 10, 15, 20, 25, 30, 35, 40, 45, 50, or more markers. In some examples, at least two markers are detected. In further examples, at least three markers are detected. In another example, at least five markers are detected. In some examples, detecting one or more markers that indicate flower initiation at increased daylight hours includes detecting 1 to 10 markers, such as 1 to 8, 1 to 5, 1 to 3, 2 to 10, 2 to 8, 2 to 5, 2 to 3, 5 to 10, or 5 to 8 markers. In one example, detecting one or more markers that indicate flower initiation at increased daylight hours includes detecting 1 to 3 markers. In another example, detecting one or more markers that indicate flower initiation at increased daylight hours includes detecting 2 to 3 markers. In a further example, detecting one or more markers that indicate flower initiation at increased daylight hours includes detecting 2 to 5 markers.

The present disclosure further describes the discovery of novel haplotype markers for plants, including Cannabis. Haplotypes refer to the genotype of a plant at a plurality of genetic loci, e.g., a combination of alleles or markers. Haplotypes can refer to sequence polymorphisms at a particular locus, such as a single marker locus, or sequence polymorphisms at multiple loci along a chromosomal segment in a given genome. Markers of the present disclosure and within the haplotypes described are significantly correlated to plants having earlier flowering initiation occurring at greater daylengths, which thus can be used to screen plants exhibiting flower initiation at greater daylengths.

In an example, Tables 2-8 describe markers within a haplotype that identify polymorphisms that confer modified flowering. In particular, the tables describe the haplotype both with respect to the left and right flanking markers, and with respect to the left and right flanking positioning on respective chromosomes. To illustrate, for non-limiting exemplary purposes, marker 171_6189 is within a haplotype defined as being between left flanking marker 171_6189 at position 0 on chromosome 8 and right flanking marker 171_11609 at position 25,101 on chromosome 8 of the Abacus reference genome.

In some examples, the one or more markers that indicate flower initiation at increased daylight hours comprise a polymorphism relative to a reference genome within any one or more haplotypes wherein the haplotypes comprise the region on chromosome 8: (a) between positions 0 and 25101; (b) between positions 25101 and 63750; (c) between positions 25101 and 63750; (d) between positions 82833 and 95593; (e) between positions 146257 and 209857; (f) between positions 146257 and 209857; (g) between positions 361045 and 401990; (h) between positions 361045 and 401990; (i) between positions 361045 and 401990; (j) between positions 430259 and 479822; (k) between positions 537879 and 558636; (1) between positions 558636 and 581624; (m) between positions 558636 and 581624; (n) between positions 714581 and 776053; (o) between positions 935803 and 938775; (p) between positions 1033166 and 1060034; (q) between positions 1076208 and 1108104; (r) between positions 1134931 and 1154438; (s) between positions 1184825 and 1193261; (t) between positions 1193261 and 1201662; (u) between positions 1205216 and 1225668; (v) between positions 1205216 and 1225668; (w) between positions 1205216 and 1225668; (x) between positions 1273758 and 1281517; (y) between positions 1295157 and 1319381; (z) between positions 1387657 and 1403412; (aa) between positions 1474748 and 1502974; (ab) between positions 1513180 and 1539957; (ac) between positions 1513180 and 1539957; (ad) between positions 1557156 and 1595751; (ae) between positions 1557156 and 1595751; (af) between positions 1600294 and 1618178; (ag) between positions 1656443 and 1686562; (ah) between positions 1686562 and 1693118; (ai) between positions 1714084 and 1757477; (aj) between positions 1714084 and 1757477; (ak) between positions 1714084 and 1757477; (al) between positions 1813955 and 1822552; (am) between positions 1866553 and 1881061; (an) between positions 1927664 and 1944792; (ao) between positions 1983548 and 1997626; (ap) between positions 1997626 and 2037226; (aq) between positions 1997626 and 2037226; (ar) between positions 2245812 and 2257882; (as) between positions 2272681 and 2285144; (at) between positions 2360594 and 2369020; (au) between positions 2401504 and 2409146; (av) between positions 51589 and 77659; (aw) between positions 84701 and 113796; (ax) between positions 84701 and 113796; (ay) between positions 102277 and 159096; (az) between positions 113796 and 152728; (ba) between positions 236814 and 330022; (bb) between positions 236814 and 330022; (bc) between positions 330022 and 371700; (bd) between positions 330022 and 371700; (be) between positions 385390 and 401990; (bf) between positions 401990 and 430259; (bg) between positions 351874 and 517316; (bh) between positions 351874 and 517316; (bi) between positions 430259 and 503453; (bj) between positions 503453 and 555854; (bk) between positions 503453 and 555854; (bl) between positions 503453 and 555854; (bm) between positions 598071 and 631105; (bn) between positions 631105 and 666249; (bo) between positions 666249 and 686125; (bp) between positions 686125 and 759657; (bq) between positions 686125 and 759657; (br) between positions 686125 and 759657; (bs) between positions 686125 and 759657; (bt) between positions 759657 and 796514; (bu) between positions 801778 and 813205; (by) between positions 813205 and 840127; (bw) between positions 855948 and 869351; (bx) between positions 917565 and 936746; (by) between positions 949136 and 978102; (bz) between positions 949136 and 978102; (ca) between positions 963275 and 972965; (cb) between positions 949136 and 978102; (cc) between positions 972965 and 1008257; (cd) between positions 999905 and 1012614; (ce) between positions 1042634 and 1052535; (cf) between positions 1060034 and 1088206; (cg) between positions 1060034 and 1088206; (ch) between positions 1088206 and 1134931; (ci) between positions 1078159 and 1191275; (cj) between positions 1078159 and 1191275; (ck) between positions 1154438 and 1184825; (cl) between positions 1184825 and 1201662; (cm) between positions 1225668 and 1257300; (cn) between positions 1228323 and 1277500; (co) between positions 1273758 and 1286264; (cp) between positions 1312722 and 1342980; (cq) between positions 1354996 and 1366469; (cr) between positions 1366469 and 1387657; (cs) between positions 1502974 and 1513180; (ct) between positions 1551663 and 1577268; (cu) between positions 1593348 and 1617208; (cv) between positions 1593348 and 1617208; (cw) between positions 1617208 and 1628490; (cx) between positions 1617208 and 1628490; (cy) between positions 1693118 and 1710154; (cz) between positions 1729625 and 1772967; (da) between positions 1772967 and 1796406; (db) between positions 1772967 and 1796406; (dc) between positions 1801046 and 1806699; (dd) between positions 1806699 and 1813955; (de) between positions 1810522 and 1816071; (df) between positions 1829943 and 1844801; (dg) between positions 1824771 and 1849570; (dh) between positions 1849570 and 1896395; (di) between positions 1905430 and 1911974; (dj) between positions 1909161 and 1916102; (dk) between positions 1916102 and 1956392; (dl) between positions 1948922 and 1963045; (dm) between positions 1963045 and 1974016; (dn) between positions 1971081 and 1983548; (do) between positions 1983548 and 2037226; (dp) between positions 2047839 and 2067693; (dq) between positions 2067693 and 2106657; (dr) between positions 2079392 and 2087331; (ds) between positions 2067693 and 2106657; (dt) between positions 2067693 and 2106657; (du) between positions 2067693 and 2106657; (dv) between positions 2114122 and 2129927; (dw) between positions 2145070 and 2167991; (dx) between positions 2198182 and 2289439; (dy) between positions 2198182 and 2289439; (dz) between positions 2339395 and 2373428; (ea) between positions 2377932 and 2391542; (eb) between positions 2457250 and 2540901; (ec) between positions 2503244 and 2510824; (ed) between positions 2457250 and 2540901; (ee) between positions 2812821 and 2825530; (ef) between positions 2825530 and 2881461; (eg) between positions 2825530 and 2881461; (ch) between positions 2825530 and 2881461; (ci) between positions 2825530 and 2881461; (ej) between positions 2908180 and 2934321; (ek) between positions 3090350 and 3118845; or (el) between positions 3776664 and 3797614, wherein the reference genome is the Abacus Cannabis reference genome version Csat_AbacusV2, NCBI assembly accession GCA_025232715.1.

The present disclosure further describes the discovery of novel trait loci for plants, including Cannabis. A chromosomal interval designating a contiguous linear span of genomic DNA that resides usually on a single chromosome is a genetic locus. Thus, an increased daylight hours flowering trait locus is a chromosome interval linked to the increased daylight hours flowering phenotype. A chromosome interval may comprise a quantitative trait locus (“QTL”) linked with a genetic trait and the QTL may comprise a single gene or multiple genes associated with the genetic trait. The boundaries of a chromosome interval comprising a QTL are drawn such that a marker that lies within the chromosome interval can be used as a marker for the genetic trait, as well as markers genetically linked thereto. Each interval comprising a QTL comprises at least one gene conferring a given trait, however knowledge of how many genes are in a particular interval is not necessary to make a composition or practice a method disclosed herein, as such an interval will segregate at meiosis as a linkage block. Accordingly, a chromosomal interval comprising a QTL may therefore be readily introgressed and tracked in a given genetic background using the methods and compositions provided herein.

Identification of chromosomal intervals and QTL is therefore beneficial for detecting and tracking a genetic trait, such as flower initiation in plant populations. In some examples, this is accomplished by identification of markers linked to a particular QTL. The principles of QTL analysis and statistical methods for calculating association between markers and useful QTL include regression analysis, single point marker analysis, complex pedigree analysis, Bayesian MCMC, identity-by-descent analysis, interval mapping, composite interval mapping (CIM), and Haseman-Elston regression. QTL analyzes may be performed with the help of a computer and specialized software available from a variety of known public and commercial sources.

Markers of the present disclosure and within the flower initiation trait loci described are significantly correlated and genetically linked to plants having the ability to flower at increased daylight hours and/or initiate flowering in a fewer number of days, which can be used to screen plants for a desired phenotype.

Also disclosed are markers that indicate flower initiation at a fewer number of days relative to a control. In some examples, the markers that indicate flower initiation at a fewer number of days under increased daylight hours (e.g., 18 hours) relative to a control. The control can be, for example, a Cannabis plant with the same genetic background except does not include the marker indicating flower initiation at a fewer number of days. In some examples, a marker that indicates flower initiation at a fewer number of days also indicates flowering at increased daylight hours. Any of the methods disclosed herein can include selecting a plant including one or more markers indicating flower initiation at a fewer number of days. In some examples, the one or more markers that indicates flower initiation at a fewer number of days comprise a polymorphism on chromosome 8 relative to a reference genome at nucleotide positions 159,096; 241,017; 321,930; 334,676; 351,874; 479,822; 498,442; 531,560; 537,879; 606,518; 639,258; 673,171; 703,062; 709,849; 714,581; 784,607; 1,319,381; or 1,376,341, wherein the reference genome is the Abacus Cannabis reference genome version Csat_AbacusV2, NCBI assembly accession GCA_025232715.1. In some examples, the nucleotide position comprises on chromosome 8: (a) a G/G or G/A genotype at position 159096; (b) a A/A or A/G genotype at position 241017; (c) a G/G or G/A genotype at position 321930; (d) a T/T or T/A genotype at position 334676; (e) a A/A or A/T genotype at position 351874; (f) a T/T or T/C genotype at position 479822; (g) a T/T or C/T genotype at position 498442; (h) a A/A or A/C genotype at position 531560; (i) a A/A or A/G genotype at position 537879; (j) a A/A or G/A genotype at position 606518; (k) a A/A or A/G genotype at position 639258; (l) a A/A or G/A genotype at position 673171; (m) a A/A or A/C genotype at position 703062; (n) a C/C or A/C genotype at position 709849; (o) a A/A or G/A genotype at position 714581; (p) a G/G or G/A genotype at position 784607; (q) a A/A or G/A genotype at position 1319381; or (r) a G/G or A/G genotype at position 1376341. In some examples, the one or more markers that indicate flower initiation at a fewer number of days comprise a polymorphism on chromosome 8 relative to a reference genome at nucleotide positions 159,096, 241,017, and/or 555,854, wherein the reference genome is the Abacus Cannabis reference genome version Csat_AbacusV2, NCBI assembly accession GCA_025232715.1. In some examples, the nucleotide position comprises on chromosome 8: (a) a G/G or G/A genotype at position 159096; (b) a A/A or A/G genotype at position 241017; and/or (c) a T/T or T/C genotype at position 555854. In some examples, the one or more markers that indicate flower initiation at a fewer number of days comprise SNPs: 171_143517, 171_219947, and 171_483960 as described herein.

In some examples, flower initiation at a fewer number of days includes flowering in less than 70 days (on average) after sowing, for example, less than 67, 65, 60, 55, 54, 50, 45, 40, 38, 35, 34, 30, 25, or fewer days (on average) after sowing. In specific, non-limiting examples flower initiation at a fewer number of days includes flowering in less than 34 days, 38 days, 54 days, or 67 days (on average) after sowing. In some examples, flower initiation at a fewer number of days includes flowering within 20 to 70 days (on average) after sowing, for example, 20 to 70 days, 20 to 55 days, 20 to 40 days, 20 to 35 days, 25 to 70 days, 25 to 55 days, 25 to 40 days, 25 to 35 days, 30 to 70 days, 30 to 55 days, 30 to 40 days, 30 to 35 days, 32 to 70 days, 32 to 55 days, 32 to 40 days, 35 to 70 days, 35 to 55 days, or 50 to 70 days on average after sowing. In a specific, non-limiting example, flower initiation at a fewer number of days includes flowering within 33 to 38 days (on average) after sowing. In another non-limiting example, flower initiation at a fewer number of days includes flowering within 53 to 67 days (on average) after sowing. In some examples, flower initiation at a fewer number of days is flower initiation at a fewer number of days under increased daylight hours (e.g., 18 hours).

### Detection of Markers

The present disclosure describes detecting markers (e.g., SNPs) associated with flower initiation at increased daylight hours and/or fewer days to flower initiation. Many suitable methods for analyzing or detecting markers are known, for example, amplification-based methods (e.g., by PCR), sequencing-based techniques, or hybridization methods (e.g., labeled probes).

PCR uses a particular amplification primer pair that specifically hybridize to a target polynucleotide and produce an amplification product (the amplicon). Primers can be designed such that the amplicon can contain a nucleic acid polymorphism of interest. rimers can generate an amplicon of any suitable length that is longer or shorter than those disclosed herein. In some examples, marker amplification produces an amplicon at least 20 nucleotides in length, or alternatively, at least 50 nucleotides in length, or alternatively, at least 100 nucleotides in length, or alternatively, at least 200 nucleotides in length. Methods for designing PCR primers and PCR conditions have been described, for example, in Sambrook et al. (2014) Molecular Cloning: A Laboratory Manual (Fourth Edition, Cold Spring Harbor Laboratory Press, Plainview, N.Y.). A number of parameters in a specific PCR protocol may need to be adjusted to specific laboratory conditions and may be slightly modified, and yet allow for the collection of similar results. The primers can be radiolabeled, or labeled by any other suitable means (e.g., using a non-radioactive fluorescent tag), to allow for rapid visualization of the different size amplicons following an amplification reaction without any additional labeling step or visualization step.

Other examples of nucleic acid amplification methods include, but are not limited to, reverse-transcription PCR (RT-PCR), quantitative real-time PCR (qPCR), quantitative real-time reverse transcriptase PCR (qRT-PCR) (see, e.g., Adams, A beginner's guide to RT-PCR, qPCR and RT-qPCR, Biochemist (Lond) (2020) 42(3): 48-53), isothermal amplification methods (see, e.g., Zanoli et al., Biosensors (2013) 3(1): 18-43), nucleic acid sequence-based amplification (NASBA) (see, e.g., Deiman and Sillekens, Mol Biotechnol (2002) 20(2):163-79), loop-mediated isothermal amplification (LAMP) (see, e.g., Notomi et al., (2000) Nucleic Acids Res. 28(12): e63), helicase-dependent amplification (HDA) (see, e.g., Cao et al., Helicase-dependent amplification of nucleic acids, Curr Protoc Mol Biol, 104:15.11.1-15.11.12, 2013), rolling circle amplification (RCA) (see, e.g, Yao et al. Nature Protocols (2021) 16, 5460-5483), multiple displacement amplification (MDA) (see, e.g, Spits et al. Nature Protocols (2006) 1: 1965-1970), recombinase polymerase amplification (RPA) (see, e.g., Lobato et al., Trends Analyt Chem (2018) 98: 19-35), ligase chain reaction (LCR) (see e.g., Gibriel and Adel, Mutat Res Rev Mutat Res. (2017) 773: 66-90), transcription amplification (see e.g., Kwoh et al. (1989) Proc. Natl. Acad. Sci. USA 86: 1173), self-sustained sequence replication (see e.g., Guatelli et al. (1990) Proc. Natl. Acad. Sci. USA 87: 1874), dot PCR, and linker adapter PCR. Additional information and amplification methods can be found, for example, in Sambrook et al. (2014) Molecular Cloning: A Laboratory Manual (Fourth Edition, Cold Spring Harbor Laboratory Press, Plainview, N.Y.).

The presence of a nucleic acid polymorphism in an amplicon can be determined, for example, by directly sequencing the amplicon, performing a restriction enzyme digest (e.g, restriction fragment length polymorphism (RFLP)), or by using a detection probe. In some implementations, detection includes PCR, quantitative PCR (qPCR), reverse-transcription PCR (RT-PCR), quantitative real-time reverse transcriptase PCR (qRT-PCR), and/or sequencing. In some examples, detection includes PCR, quantitative PCR (qPCR), and/or sequencing.

PCR detection and quantification using dual-labeled fluorogenic oligonucleotide probes, commonly referred to as “TaqMan™” probes, can also be performed according to the present disclosure. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. TaqMan™ probes are oligonucleotides that have a label and a quencher, where the label is released during amplification by the exonuclease action of the polymerase used in amplification, providing a real time measure of amplification during synthesis. A variety of TaqMan™ reagents are commercially available, e.g., from Applied Biosystems as well as from a variety of specialty vendors such as Biosearch Technologies.

In some implementations, detection of nucleic acid polymorphisms includes use of an oligonucleotide primer or probe. In general, synthetic methods for making oligonucleotides, including probes or primers are known. For example, oligonucleotides can be synthesized chemically according to the solid phase phosphoramidite triester method described. Oligonucleotides, including modified oligonucleotides, can also be ordered from a variety of commercial sources. Nucleic acid probes to the marker loci can be cloned and/or synthesized. Any suitable label can be used with a probe. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radio labels, enzymes, and colorimetric labels. Other labels include ligands which bind to antibodies labeled with fluorophores, chemiluminescent agents, and enzymes. A probe can also constitute radio labeled PCR primers that are used to generate a radio labeled amplicon. It is not intended that the nucleic acid probes be limited to any particular size, however, nucleic acid probes are typically 20-100 base pairs.

Amplification is not always required for detection of a nucleic acid polymorphism (e.g., Southern blotting or RFLP detection). Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

In some implementations, a genetic marker (e.g., nucleic acid polymorphism) is detected by sequencing a nucleic acid fragment comprising a target sequence of interest (e.g., a particular haplotype) or by whole genome sequencing (or whole transcriptome sequencing). Non-limiting examples of suitable sequencing methods include capillary electrophoresis (e.g., Sanger sequencing) and high-throughput sequencing (e.g., Illumina® or 454 Sequencing®). High-throughput sequencing includes short read or long read techniques. In some implementations, sequencing includes whole genome sequencing (e.g., sequencing the genome of a Cannabis plant). In some examples, sequencing includes targeted sequencing (sequencing of a particular nucleic acid or amplicon of interest). In some examples, sequencing includes sequencing a transcriptome (RNA-Seq) (e.g., sequencing the transcriptome of a Cannabis plant of interest). In some implementations, sequencing does not include sequencing of RNA. In some implementations, the genome is sequenced.

In general, synthetic methods for making oligonucleotides, including probes, primers, molecular beacons, PNAs, LNAs (locked nucleic acids), etc., are known. For example, oligonucleotides can be synthesized chemically according to the solid phase phosphoramidite triester method described. Oligonucleotides, including modified oligonucleotides, can also be ordered from a variety of commercial sources.

In some examples, detecting one or more markers that indicate flower initiation at increased daylight hours in a method disclosed herein includes analyzing two or more nucleotide positions associated with flower initiation at increased daylight hours as disclosed herein (e.g., in Tables 2, 4, 5, and 10), for example, analyzing at least 3, 5, 10, 15, 20, 25, 30, or 35 nucleotide positions disclosed herein. In some examples, at least 3 nucleotide positions disclosed herein are analyzed. In some examples, at least 10 nucleotide positions disclosed herein are analyzed. In some examples, at least 15 nucleotide positions disclosed herein are analyzed.

In some examples, detecting one or more markers that indicate flower initiation at increased daylight hours includes analyzing nucleotide positions: 159,096; 241,017; 321,930; 334,676; 351,874; 479,822; 498,442; 531,560; 537,879; 606,518; 639,258; 673,171; 703,062; 709,849; 714,581; 784,607; 1,319,381; and 1,376,341; and detecting at least one marker that indicates flower initiation at increased daylight hours, wherein the reference genome is the Abacus Cannabis reference genome version Csat_AbacusV2, NCBI assembly accession GCA_025232715.1. In some examples, detecting one or more markers that indicate flower initiation at increased daylight hours includes analyzing nucleotide positions: 19,681; 44.981; 87,904; 109,674; 159,096; 378,368; 479,822; 491,066; 568,544; 720,538; 968,829; 1,191,275; 1,200,057; 1,211,168; 1,273,758; 1,277,500; 1,305,959; 1,659,209; 1,690,294; 1,735,848; 1,816,071; 1,872,522; 1,993,648; 2,002,233; 2,020,279; 2,079,928; and 2,267,196, and detecting at least one marker that indicates flower initiation at increased daylight hours, wherein the reference genome is the Abacus Cannabis reference genome version Csat_AbacusV2, NCBI assembly accession GCA_025232715.1. In some examples, detecting one or more markers that indicate flower initiation at increased daylight hours includes analyzing nucleotide positions: 109,674; 159,096; 1,154.438, and detecting at least one marker that indicates flower initiation at increased daylight hours, wherein the reference genome is the Abacus Cannabis reference genome version Csat_AbacusV2, NCBI assembly accession GCA_025232715.1. In some examples, the methods include detecting at least two markers that indicates flower initiation at increased daylight hours. In some examples, the methods include detecting at least three markers that indicates flower initiation at increased daylight hours.

In some examples, detecting one or more markers that indicate flower initiation at a fewer number of days in a method disclosed herein includes analyzing two or more nucleotide positions disclosed herein (e.g., in Tables 3, 6, or 7), for example, analyzing at least 3, 5, 10, 12, 15, or 18 nucleotide positions disclosed herein. In some examples, at least 3 nucleotide positions disclosed herein are analyzed. In some examples, at least 10 nucleotide positions disclosed herein are analyzed. In some examples, at least 15 nucleotide positions disclosed herein are analyzed. In some examples, detecting one or more markers that indicate flower initiation at a fewer number of days includes analyzing nucleotide positions: 159,096; 241,017; 321,930; 334,676; 351,874; 479,822; 498,442; 531,560; 537,879; 606,518; 639,258; 673,171; 703,062; 709,849; 714,581; 784,607; 1,319,381; and 1,376,341; and detecting at least one marker that indicates flower initiation at a fewer number of days, wherein the reference genome is the Abacus Cannabis reference genome version Csat_AbacusV2, NCBI assembly accession GCA_025232715.1.

### Flower Initiation Genes

Disclosed are genes conferring modified flowering (e.g., flower initiation at increased daylight hours and/or flower initiation at a fewer number of days). Candidate genes include, but are not limited to, EARLY FLOWERING ELF9; Arabidopsis: AT5G16260; CBDRx version cs10: LOC115699158; T-DNA knockout mutants are flowering under daylength in which wild type Arabidopsis does not initiate flowering (Song, Hae-Ryong, et al. “The RNA binding protein ELF9 directly reduces SUPPRESSOR OF OVEREXPRESSION OF COI transcript levels in Arabidopsis, possibly via nonsense-mediated mRNA decay.” The Plant Cell 21.4 (2009): 1195-1211), a transmembrane protein (Arabidopsis: AT5G16250, CBDRx version cs10: LOC115699160), Ubiquitin-like protein ATG12A (synonym: APG12A; Arabidopsis: AT1G54210; CBDRx version cs10: LOC115698724), Stearoyl-[acyl-carrier-protein]9-desaturase 3 S-ACP-DES3 (synonyms: AAD3, SAD3; Arabidopsis: AT5G16230; CBDRx version cs10: LOC115698723), Chromatin assembly factor 1 subunit FAS1 (synonym: NFB2; Arabidopsis: AT1G65470; LOC115700508), FLOWERING LOCUS T FT (Arabidopsis: AT1 G65480; CBDRx version cs10: LOC115700781; FT has a central position in mediating the onset of flowering (Kobayashi, Yasushi, et al. “A pair of related genes with antagonistic roles in mediating flowering signals.” Science 286.5446 (1999): 1960-1962.; Pin, P. A., and Ove Nilsson. “The multifaceted roles of FLOWERING LOCUS T in plant development.” Plant, cell & environment 35.10 (2012): 1742-1755.)), and an RNA helicase (Arabidopsis: AT2G38770; CBDRx version cs10: LOC115700975). Soluble inorganic pyrophosphatase 6 PPA6 (synonym: PPA; Arabidopsis: AT5G09650; CBDRx version cs10: LOC115701466). Acyl-CoA N-acyltransferase with RING/FYVE/PHD-type zinc finger domain-containing protein (AT2G27980; CBDRx version cs10: LOC115698493), a transcription factor (Arabidopsis: AT3G04930; CBDRx version cs10: LOC115700433) and Cyclin-dependent kinases regulatory subunit 1 CKS1 (Arabidopsis: AT2G27960; CBDRx version cs10: LOC115701496). Magnesium transporter MRS2-11 (synonyms: GMN10, MGT10; Arabidopsis: AT5G22830; CBDRx version cs10: LOC115701116), an F-box protein (Arabidopsis: AT1G47730; CBDRx version cs10: LOC115700410). rRNA 2′-O-methyltransferase fibrillarin 2 FIB2 (synonyms: FLP, MED36_1, MED36A; Arabidopsis: AT4G25630; CBDRx version cs10: LOC115700643; FIB2 is part of the MEDIATOR complex (Guo, Jing, et al. “The CBP/p300 histone acetyltransferases function as plant-specific MEDIATOR subunits in Arabidopsis.” Journal of Integrative Plant Biology 63.4 (2021): 755-771.), other MEDIATOR complex subunits have been described to promote flowering (Iñigo, Sabrina, et al. “PFT1, the MED25 subunit of the plant Mediator complex, promotes flowering through CONSTANS dependent and independent mechanisms in Arabidopsis.” The Plant Journal 69.4 (2012): 601-612.)), a nucleic acid-binding, OB-fold-like protein (Arabidopsis: AT2G40780; CBDRx version cs10: LOC115698837) and Xanthoxin dehydrogenase ABA2 (synonyms: GINI, ISI4, SAN3, SDR1, SIS4, SRE1; Arabidopsis: AT1G52340; CBDRx version cs10: LOC115698918).

In some examples, genes conferring modified flowering (e.g., flower initiation at increased daylight hours and/or flower initiation at a fewer number of days) include (a) EARLY FLOWERING 9 (ELF9); (b) FLOWERING LOCUS T (FT); (c) CYCLIC DOF FACTOR 2 (CDF2); (d) ARGONAUTE 5 (AGO5); (e) GAST1 PROTEIN HOMOLOG 4 (GASA4); (f) CLP-SIMILAR PROTEIN 3 (CLPS3); and (g) PISTILLATA (PI).

Disclosed are methods of producing one or more Cannabis plants having modified flowering (e.g., flower initiation at increased daylight hours and/or flower initiation at a fewer number of days), comprising: (i) obtaining a nucleic acid sample from a Cannabis plant or its germplasm; (ii) detecting one or more nucleic acid polymorphisms associated with flowering at increased daylight hours or flowering in a fewer number of days in one or more of: (a) EARLY FLOWERING 9 (ELF9); (b) FLOWERING LOCUS T (FT); (c) CYCLIC DOF FACTOR 2 (CDF2); (d) ARGONAUTE 5 (AGO5); (e) GAST1 PROTEIN HOMOLOG 4 (GASA4); (f) CLP-SIMILAR PROTEIN 3 (CLPS3); and (g) PISTILLATA (PI); (iii) crossing the Cannabis plant comprising the one or more nucleic acid polymorphisms, and (iv) obtaining progeny plants comprising of the one or more nucleic acid polymorphisms, thereby producing one or more Cannabis plants having modified flowering. Also provided are methods of identifying one or more Cannabis plants having modified flowering (e.g., flower initiation at increased daylight hours and/or flower initiation at a fewer number of days), comprising: (i) obtaining a nucleic acid sample from a Cannabis plant or its germplasm; (ii) detecting one or more nucleic acid polymorphisms associated with flowering at increased daylight hours or flowering in a fewer number of days in one or more of: (a) EARLY FLOWERING 9 (ELF9); (b) FLOWERING LOCUS T (FT); (c) CYCLIC DOF FACTOR 2 (CDF2); (d) ARGONAUTE 5 (AGO5); (e) GAST1 PROTEIN HOMOLOG 4 (GASA4); (f) CLP-SIMILAR PROTEIN 3 (CLPS3); and (g) PISTILLATA (PI); thereby identifying the one or more Cannabis plants having modified flowering. In some examples, the one or more Cannabis plants having modified flowering are selected for crossing and/or making a product. In some examples, the modified flowering is flower initiation at increased daylight hours. In some examples, the modified flowering is flower initiation at a fewer number of days relative to a control. In some examples, the modified flowering is flower initiation at increased daylight hours and flower initiation at a fewer number of days relative to a control.

In some examples, the one or more nucleic acid polymorphisms are detected in ELF9 and comprise: (a) a substitution at position 257 bp from the start codon of the coding sequence of Abacus ELF9 (SEQ ID NO: 171), or (b) a substitution at position 907 bp from the start codon of in the coding sequence of Abacus ELF9 (SEQ ID NO: 171). In some examples, the one or more nucleic acid polymorphisms are detected in FT and comprise: (a) a substitution at position 196 bp from the start codon of Abacus FT (SEQ ID NO: 177), (b) a substitution at position 198 bp from the start codon of Abacus FT (SEQ ID NO: 177), or (c) a deletion of 12 nucleotides at positions 46-57 bp of Abacus FT (SEQ ID NO: 177). In some examples, the one or more nucleic acid polymorphisms are detected in CDF2 and comprise: (a) a substitution at position 793 bp from the start codon of the coding sequence of Abacus CDF2 (SEQ ID NO: 186). (b) a substitution at position 850 bp from the start codon of the coding sequence of Abacus CDF2 (SEQ ID NO: 186), (c) a substitution at position 307 bp from the start codon of the coding sequence of Abacus CDF2 (SEQ ID NO: 186), (d) a substitution at position 380 bp from the start codon of the coding sequence of Abacus CDF2 (SEQ ID NO: 186), (e) a substitution at position 856 bp from the start codon of the coding sequence of Abacus CDF2 (SEQ ID NO: 186), (f) a substitution at position 868 bp from the start codon of the coding sequence of Abacus CDF2 (SEQ ID NO: 186), (g) a substitution at position 878 bp from the start codon of the coding sequence of Abacus CDF2 (SEQ ID NO: 186), or (h) a substitution at position 1180 bp from the start codon of the coding sequence of Abacus CDF2 (SEQ ID NO: 186). In some examples, the one or more nucleic acid polymorphisms are detected in AGO5 and comprise: (a) a substitution at position 787 bp from the start codon of the coding sequence of Abacus AGO5 (SEQ ID NO: 218), (b) a substitution at position 1388 bp from the start codon of the coding sequence of Abacus AGO5 (SEQ ID NO: 218), (c) a substitution at position 1429 bp from the start codon of the coding sequence of Abacus AGO5 (SEQ ID NO: 218), (d) a substitution at position 1482 bp from the start codon of the coding sequence of Abacus AGO5 (SEQ ID NO: 218). (e) a substitution at position 1569 bp from the start codon of the coding sequence of Abacus AGO5 (SEQ ID NO: 218), (f) a substitution at position 1593 bp from the start codon of the coding sequence of Abacus AGO5 (SEQ ID NO: 218), (g) a substitution at position 2082 bp from the start codon of the coding sequence of Abacus AGO5 (SEQ ID NO: 218), (h) a substitution at position 2087 bp from the start codon of the coding sequence of Abacus AGO5 (SEQ ID NO: 218), (i) a substitution at position 2167 bp from the start codon of the coding sequence of Abacus AGO5 (SEQ ID NO: 218), (j) a substitution at position 2218 bp from the start codon of the coding sequence of Abacus AGO5 (SEQ ID NO: 218), or (k) a substitution at position 2238 bp from the start codon of the coding sequence of Abacus AGO5 (SEQ ID NO: 218). In some examples, the one or more nucleic acid polymorphisms detected in GASA4 and comprise: (a) a substitution at position 112 bp from the start codon of the coding sequence of Abacus GASA4 (SEQ ID NO: 202). In some examples, the one or more nucleic acid polymorphisms are detected in CLPS3 and comprise: (a) a substitution at position 262 bp from the start codon of the coding sequence of Abacus CLPS3 (SEQ ID NO: 207), or (b) a substitution at position 712 bp from the start codon of the coding sequence of Abacus CLPS3 (SEQ ID NO: 207). In some examples, the one or more nucleic acid polymorphisms are detected in PI and comprise: (a) a deletion of three nucleotides at positions 529-531 bp of Abacus PI (SEQ ID NO: 213).

In some examples, the one or more nucleic acid polymorphisms are detected in ELF9 and cause: (a) a T86I substitution of Abacus ELF9 (SEQ ID NO: 173), or (b) a N303D substitution of Abacus ELF9 (SEQ ID NO: 173). In some examples, the one or more nucleic acid polymorphisms are detected in FT and cause: (a) a L66V substitution of Abacus FT (SEQ ID NO: 180), or (b) a deletion of amino acid positions 16 to 19 of Abacus FT (SEQ ID NO: 180). In some examples, the one or more nucleic acid polymorphisms are detected in CDF2 and cause: (a) a M265L substitution of Abacus CDF2 (SEQ ID NO: 190), (b) a A284P substitution of Abacus CDF2 (SEQ ID NO: 190), (c) a T103A substitution of Abacus CDF2 (SEQ ID NO: 190), (d) a T127N substitution of Abacus CDF2 (SEQ ID NO: 190), (e) a K286Q substitution of Abacus CDF2 (SEQ ID NO: 190), (f) a N290H substitution of Abacus CDF2 (SEQ ID NO: 190), (g) a N293I substitution of Abacus CDF2 (SEQ ID NO: 190), or (h) a A394T substitution of Abacus CDF2 (SEQ ID NO: 190). In some examples, the one or more nucleic acid polymorphisms are detected in AGO5 and cause: (a) a V263I substitution of Abacus AGO5 (SEQ ID NO: 221), (b) a A463M substitution of Abacus AGO5 (SEQ ID NO: 221), (c) a E477K substitution of Abacus AGO5 (SEQ ID NO: 221), (d) a N494K substitution of Abacus AGO5 (SEQ ID NO: 221), (e) a I523M substitution of Abacus AGO5 (SEQ ID NO: 221), (f) a N531K substitution of Abacus AGO5 (SEQ ID NO: 221), (g) a E594D substitution of Abacus AGO5 (SEQ ID NO: 221). (h) a G696E substitution of Abacus AGO5 (SEQ ID NO: 221), (i) a D723N substitution of Abacus AGO5 (SEQ ID NO: 221), (j) a N740H substitution of Abacus AGO5 (SEQ ID NO: 221), or (k) a H746Q substitution of Abacus AGO5 (SEQ ID NO: 221). In some examples, the one or more nucleic acid polymorphisms are detected in GASA4 and cause: (a) a K38E substitution of Abacus GASA4 (SEQ ID NO: 204). In some examples, the one or more nucleic acid polymorphisms are detected in CLPS3 and cause: (a) a M88L substitution of Abacus CLPS3 (SEQ ID NO: 209), or (b) a V238I substitution of Abacus CLPS3 (SEQ ID NO: 209). In some examples, the one or more nucleic acid polymorphisms are detected in PI and cause: (a) a deletion of amino acid 177 of Abacus PI (SEQ ID NO: 215).

In some examples, the one or more nucleic acid polymorphisms are detected in at least two of: (a) EARLY FLOWERING 9 (ELF9); (b) FLOWERING LOCUS T (FT); (c) CYCLIC DOF FACTOR 2 (CDF2); (d) ARGONAUTE 5 (AGO5); (e) GAST1 PROTEIN HOMOLOG 4 (GASA4); (f) CLP-SIMILAR PROTEIN 3 (CLPS3); and (g) PISTILLATA (PI). In some examples, the one or more nucleic acid polymorphisms are detected in at least two of ELF9, FT, and CDF2. In some examples, the one or more nucleic acid polymorphisms are detected in ELF9, FT, and CDF2.

In some examples, the one or more nucleic acid polymorphisms are detected in ELF9 and comprise a substitution at positions 257 bp and 907 bp from the start codon, wherein the reference is Abacus ELF9 (SEQ ID NO: 171), and are detected in FT and comprise a substitution at positions 196 bp and 198 bp from the start codon, wherein the reference is Abacus FT (SEQ ID NO: 177). In some examples, the one or more nucleic acid polymorphisms are detected in ELF9 and cause a T86I substitution and a N303D substitution, wherein the reference is Abacus ELF9 (SEQ ID NO: 173), and are detected in FT and cause a L66V substitution, wherein the reference is Abacus FT (SEQ ID NO: 180). In some examples, one or more nucleic acid polymorphisms are further detected in CDF2, for example, a substitution at position 793 bp from the start codon, wherein the reference is Abacus CDF2 (SEQ ID NO: 186); and/or a substitution at position 850 bp from the start codon, wherein the reference is Abacus CDF2 (SEQ ID NO: 186). In some examples, the polymorphisms detected in CDF2 cause a M265L substitution and/or a A284P substitution, wherein the reference is Abacus CDF2 (SEQ ID NO: 190).

Also provided are methods of producing a genetically engineered Cannabis plant that initiates flowering at increased daylight hours, comprising: introducing a genetic modification in ELF9, FT, CDF2, AGO5, GASA4, CLPS3, and/or PI or introducing a beneficial allele of ELF9, FT, CDF2, AGO5, GASA4, CLPS3, and/or PI in a Cannabis plant. In some examples, the methods include introducing a genetic modification in ELF9 and FT, or introducing a beneficial allele of ELF9 and FT in a Cannabis plant. A beneficial allele is an allele that confers initiation of flowering at increased daylight hours. The genetic modification can be, for example, a nucleic acid substitution, insertion, or deletion. In some examples, the genetic modification is introduced by mutagenesis or gene editing. Exemplary suitable gene editing techniques include RNA interference (RNAi), clustered regularly interspaced short palindromic repeats/CRISPR associated protein (CRISPR/Cas), zinc-finger nucleases (ZFN), or transcription activator-like effector nucleases (TALEN)-based editing system. The genetic modification or beneficial allele initiates flowering at increased daylight hours relative to a control, such as the Cannabis plant in an unmodified state. In some examples, the methods further include introducing a genetic modification in CDF2 or introducing a beneficial allele of CDF2. In some examples, introducing the genetic modification or beneficial allele initiates flowering at increased daylight hours relative to the Cannabis plant in an unmodified state. Preferred substantially similar nucleic acid sequences encompassed by this disclosure are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. It is well understood that many levels of sequence identity are useful in identifying related polynucleotide sequences. Useful examples of percent identities are those listed above, or also preferred is any integer percentage from 72% to 100%, such as 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In an example, an isolated polynucleotide is provided comprising a nucleotide sequence having at least 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity compared to the claimed sequence, based on the Clustal V method of alignment with pairwise alignment default parameters (KTUPLE=2, GAP PENALTY=5, WINDOW=4 and DIAGONALS SAVED=4).

Local sequence alignment programs are similar in their calculation, but only compare aligned fragments of the sequences rather than utilizing an end-to-end analysis. Local sequence alignment programs such as BLAST® can be used to compare specific regions of two sequences. A BLAST® comparison of two sequences results in an E-value, or expectation value, that represents the number of different alignments with scores equivalent to or better than the raw alignment score, S, that are expected to occur in a database search by chance. The lower the E value, the more significant the match. Because database size is an element in E-value calculations, E-values obtained by BLASTing against public databases, such as GENBANK, have generally increased over time for any given query/entry match. In setting criteria for confidence of polypeptide function prediction, a “high” BLAST® match is considered herein as having an E-value for the top BLAST® hit of less than 1E-30; a medium BLASTX E-value is 1E-30 to 1E-8; and a low BLASTX E-value is greater than 1E-8. The protein function assignment in the present disclosure is determined using combinations of E-values, percent identity, query coverage and hit coverage. Query coverage refers to the percent of the query sequence that is represented in the BLAST® alignment. Hit coverage refers to the percent of the database entry that is represented in the BLAST® alignment. In one example of the disclosure, function of a query polypeptide is inferred from function of a protein homolog where either (1) hit_p<1e-30 or % identity>35% AND query_coverage>50% AND hit_coverage>50%, or (2) hit_p<1e-8 AND query_coverage>70% AND hit_coverage>70%. The following abbreviations are produced during a BLAST® analysis of a sequence. SEQ_NUM provides the SEQ ID NO for the listed recombinant polynucleotide sequences. CONTIG_ID provides an arbitrary sequence name taken from the name of the clone from which the cDNA sequence was obtained. PROTEIN_NUM provides the SEQ ID NO for the recombinant polypeptide sequence NCBI_GI provides the GenBank ID number for the top BLAST® hit for the sequence. The top BLAST® hit is indicated by the National Center for Biotechnology Information GenBank Identifier number. NCBI_GI_DESCRIPTION refers to the description of the GenBank top BLAST® hit for sequence. E_VALUE provides the expectation value for the top BLAST® match. MATCH_LENGTH provides the length of the sequence which is aligned in the top BLAST® match TOP_HIT_PCT_IDENT refers to the percentage of identically matched nucleotides (or residues) that exist along the length of that portion of the sequences which is aligned in the top BLAST® match. CAT_TYPE indicates the classification scheme used to classify the sequence. GO_BP=Gene Ontology Consortium—biological process; GO_CC=Gene Ontology Consortium—cellular component; GO_MF=Gene Ontology Consortium molecular function; KEGG=KEGG functional hierarchy (KEGG=Kyoto Encyclopedia of Genes and Genomes); EC=Enzyme Classification from ENZYME data bank release 25.0; POI=Pathways of Interest. CAT_DESC provides the classification scheme subcategory to which the query sequence was assigned. PRODUCT_CAT_DESC provides the FunCAT annotation category to which the query sequence was assigned. PRODUCT_HIT_DESC provides the description of the BLAST® hit which resulted in assignment of the sequence to the function category provided in the cat_desc column. HIT_E provides the E value for the BLAST® hit in the hit_desc column. PCT_IDENT refers to the percentage of identically matched nucleotides (or residues) that exist along the length of that portion of the sequences which is aligned in the BLAST® match provided in hit_desc. QRY_RANGE lists the range of the query sequence aligned with the hit. HIT_RANGE lists the range of the hit sequence aligned with the query. provides the percent of query sequence length that matches QRY_CVRG provides the percent of query sequence length that matches to the hit (NCBI) sequence in the BLAST® match (% qry cvrg=(match length/query total length)×100). HIT_CVRG provides the percent of hit sequence length that matches to the query sequence in the match generated using BLAST® (% hit cvrg=(match lengthy hit total length)×100).

Methods for aligning sequences for comparison are well-known in the art. Various programs and alignment algorithms are described. In an example, the subject disclosure relates to calculating percent identity between two polynucleotides or amino acid sequences using an AlignX alignment program of the Vector NTI suite (Invitrogen, Carlsbad, Calif.). The AlignX alignment program is a global sequence alignment program for polynucleotides or proteins. In an example, the subject disclosure relates to calculating percent identity between two polynucleotides or amino acid sequences using the MegAlign program of the LASERGENE bioinformatics computing suite (MegAlign™ (.COPYRGT.1993-2016). DNASTAR. Madison, Wis.). The MegAlign program is a global sequence alignment program for polynucleotides or proteins.

### Cannabis Breeding

Cannabis is an important and valuable crop. Thus, a continuing goal of Cannabis plant breeders is to develop stable, high yielding Cannabis cultivars that are agronomically sound. To accomplish this goal, the Cannabis breeder preferably selects and develops Cannabis plants with traits that result in superior cultivars. The plants described herein can be used to produce new plant varieties. Any of the methods disclosed herein can include crossing a first generation progeny plant (e.g., F1) to produce one or more additional progeny plants, wherein the additional progeny plants initiate flowering at increased daylight hours. In an example, the at least one additional progeny plant comprising the indicated flowering at increased daylight hours phenotype is an F2-F7 progeny plant. In some examples, the at least one additional progeny plant comprising the indicated flowering at increased daylight hours phenotype comprises a second generation progeny plant (e.g., F2). In some examples, the plants are used to develop new, unique, and superior varieties or hybrids with desired phenotypes.

The development of commercial Cannabis cultivars requires the development of Cannabis varieties, the crossing of these varieties, and the evaluation of the crosses. Pedigree breeding and recurrent selection breeding methods may be used to develop cultivars from breeding populations. Breeding programs may combine desirable traits from two or more varieties or various broad-based sources into breeding pools from which cultivars are developed by selfing and selection of desired phenotypes. The new cultivars may be crossed with other varieties and the hybrids from these crosses are evaluated to determine which have commercial potential.

Details of existing Cannabis plants varieties and breeding methods are described in Potter et al. (2011, World Wide Weed: Global Trends in Cannabis Cultivation and Its Control), Holland (2010, The Pot Book: A Complete Guide to Cannabis, Inner Traditions/Bear & Co, ISBN1594778981, 9781594778988), Green I (2009, The Cannabis Grow Bible: The Definitive Guide to Growing Marijuana for Recreational and Medical Use, Green Candy Press, 2009, ISBN 1931160589, 9781931160582), Green II (2005, The Cannabis Breeder's Bible: The Definitive Guide to Marijuana Genetics, Cannabis Botany and Creating Strains for the Seed Market, Green Candy Press, 1931160279, 9781931160278), Starks (1990, Marijuana Chemistry: Genetics, Processing & Potency, ISBN 0914171399, 9780914171393), Clarke (1981, Marijuana Botany, an Advanced Study: The Propagation and Breeding of Distinctive Cannabis, Ronin Publishing, ISBN 091417178X, 9780914171782), Short (2004, Cultivating Exceptional Cannabis: An Expert Breeder Shares His Secrets, ISBN 1936807122, 9781936807123), Cervantes (2004, Marijuana Horticulture: The Indoor/Outdoor Medical Grower's Bible, Van Patten Publishing, ISBN 187882323X, 9781878823236), Franck et al. (1990, Marijuana Grower's Guide, Red Eye Press, ISBN 0929349016, 9780929349015), Grotenhermen and Russo (2002, Cannabis and Cannabinoids: Pharmacology, Toxicology, and Therapeutic Potential, Psychology Press, ISBN 0789015080, 9780789015082), Rosenthal (2007, The Big Book of Buds: More Marijuana Varieties from the World's Great Seed Breeders, ISBN 1936807068, 9781936807062), Clarke, RC (Cannabis: Evolution and Ethnobotany 2013 (In press)), King, J (Cannabible Vols 1-3, 2001-2006), and four volumes of Rosenthal's Big Book of Buds series (2001, 2004, 2007, and 2011).

Pedigree selection, where both single plant selection and mass selection practices are employed, may be used for the generating varieties as described herein. Pedigree selection, also known as the “Vilmorin system of selection,” is described in Fehr, Walter; Principles of Cultivar Development, Volume T, Macmillan Publishing Co. Pedigree breeding is used commonly for the improvement of self-pollinating crops or inbred lines of cross-pollinating crops. Two parents which possess favorable, complementary traits are crossed to produce an F1. An F2 population is produced by selfing one or several F1's or by intercrossing two F1's (sib mating). Selection of the best individuals usually begins in the F2 population; then, beginning in the F3, the best individuals in the best families are usually selected. Replicated testing of families, or hybrid combinations involving individuals of these families, often follows in the F4 generation to improve the effectiveness of selection for traits with low heritability. At an advanced stage of inbreeding (e.g., F6 and F7), the best lines or mixtures of phenotypically similar lines are tested for potential release as new cultivars.

Choice of breeding or selection methods depends on the mode of plant reproduction, the heritability of the trait(s) being improved, and the type of cultivar used commercially (e.g., F1 hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

Mass and recurrent selections can be used to improve populations of either self- or cross-pollinating crops. A genetically variable population of heterozygous individuals may be identified or created by intercrossing several different parents. The best plants may be selected based on individual superiority, outstanding progeny, or excellent combining ability. Preferably, the selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent may be selected and repeatedly crossed (backcrossed) to the recurrent parent. The resulting plant is expected to have the attributes of the recurrent parent (e.g., cultivar) and the desirable trait transferred from the donor parent.

A single-seed descent procedure refers to planting a segregating population, harvesting a sample of one seed per plant, and using the one-seed sample to plant the next generation. When the population has advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

Mutation breeding is another method of introducing new traits into Cannabis varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The complexity of inheritance also influences the choice of the breeding method. Backcross breeding may be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Additional breeding methods are known e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066). Cannabis genome has been sequenced (Bakel et al., The draft genome and transcriptome of Cannabis sativa, Genome Biology, 12(10):R102, 2011). Molecular markers for Cannabis plants are described in Datwyler et al. (Genetic variation in hemp and marijuana (Cannabis sativa L.) according to amplified fragment length polymorphisms, J Forensic Sci. 2006 March; 51(2):371-5), Pinarkara et al., (RAPD analysis of seized marijuana (Cannabis sativa L.) in Turkey, Electronic Journal of Biotechnology, 12(1), 2009), Hakki et al., (Inter simple sequence repeats separate efficiently hemp from marijuana (Cannabis sativa L.), Electronic Journal of Biotechnology, 10(4), 2007), Datwyler et al., (Genetic Variation in Hemp and Marijuana (Cannabis sativa L.) According to Amplified Fragment Length Polymorphisms, J Forensic Sci, March 2006, 51(2):371-375), Gilmore et al. (Isolation of microsatellite markers in Cannabis sativa L. (marijuana), Molecular Ecology Notes, 3(1):105-107, March 2003), Pacifico et al., (Genetics and marker-assisted selection of chemotype in Cannabis sativa L.), Molecular Breeding (2006) 17:257-268), and Mendoza et al., (Genetic individualization of Cannabis sativa by a short tandem repeat multiplex system, Anal Bioanal Chem (2009) 393:719-726).

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

### Marker Assisted Selection Breeding

In an example, marker assisted selection (MAS) is used to produce plants with desired traits. MAS is a powerful shortcut to selecting for desired phenotypes and for introgressing desired traits into cultivars (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

Introgression refers to the transmission of a desired allele of a genetic locus from one genetic background to another, which is significantly assisted through MAS. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like.

The introgression of one or more desired loci from a donor line into another is achieved via repeated backcrossing to a recurrent parent accompanied by selection to retain one or more loci from the donor parent. Markers associated with flower initiation at increased daylight hours may be assayed in progeny and those progeny with one or more desired markers are selected for advancement. In another aspect, one or more markers can be assayed in the progeny to select for plants with the genotype of the agronomically elite parent. This disclosure anticipates that trait introgressed phenotype relating to flower initiation at increased daylight hours will require more than one generation, wherein progeny are crossed to the recurrent (agronomically elite) parent or selfed. Selections are made based on the presence of one or more flower initiation markers and can also be made based on the recurrent parent genotype, wherein screening is performed on a genetic marker and/or phenotype basis. In another example, markers of this disclosure can be used in conjunction with other markers, ideally at least one on each chromosome of the Cannabis genome, to track the flower initiation phenotypes.

Genetic markers are used to identify plants that contain a desired genotype at one or more loci, and that are expected to transfer the desired genotype, along with a desired phenotype to their progeny. Genetic markers can be used to identify plants containing a desired genotype at one locus, or at several unlinked or linked loci (e.g., a haplotype), and that would be expected to transfer the desired genotype, along with a desired phenotype to their progeny. The present disclosure provides the means to identify plants that flower at increased daylight hours by identifying plants having flower initiation specific markers.

In general, MAS uses polymorphic markers that have been identified as having a significant likelihood of co-segregation with a desired trait. Such markers are presumed to map near a gene or genes that give the plant its desired phenotype, and are considered indicators for the desired trait, and are termed QTL markers. Plants are tested for the presence or absence of a desired allele in the QTL marker.

Genomic selection is another form of marker-assisted selection in which a very large number of genetic markers covering the whole genome are used. With genomic selection, all SNPs are included, each with a different level of effect, in a model to explain the variation of the trait. Genomic selection is based on the analysis of many SNPs, for example tens of thousands or even millions of SNPs. This high number of SNP markers is used as input in a genomic prediction formula that predicts the desired phenotype for MAS.

Identification of plants or germplasm that include a marker locus or marker loci linked to a desired trait or traits provides a basis for performing MAS. Plants that comprise favorable markers or favorable alleles are selected for, while plants that comprise markers or alleles that are negatively correlated with the desired trait can be selected against. Desired markers and/or alleles can be introgressed into plants having a desired (e.g., elite or exotic) genetic background to produce an introgressed plant or germplasm having the desired trait. In some aspects, it is contemplated that a plurality of markers for desired traits are sequentially or simultaneously selected and/or introgressed. The combinations of markers that are selected for in a single plant are not limited, and can include any combination of markers disclosed herein or any marker linked to the markers disclosed herein, or any markers located within the QTL intervals defined herein.

In some examples, a first Cannabis plant or germplasm exhibiting a desired trait (the donor) can be crossed with a second Cannabis plant or germplasm (the recipient, e.g., an elite or exotic Cannabis, depending on characteristics that are desired in the progeny) to create an introgressed Cannabis plant or germplasm as part of a breeding program. In some aspects, the recipient plant can also contain one or more loci associated with one or more desired traits, which can be qualitative or quantitative trait loci. In another aspect, the recipient plant can contain a transgene. Following the cross, nucleic acids from sample plants of the progeny plants or their germplasm can be obtained, which allows screening of the nucleic acids to detect one or more markers that are genetically linked to the desired trait. Marker-assisted selection can then be used to select progeny Cannabis plants comprising the one or more markers genetically linked to the desired trait, thereby producing Cannabis plants comprising the desired activity.

MAS, as described herein, using additional markers flanking either side of the DNA locus provide further efficiency because an unlikely double recombination event would be needed to simultaneously break linkage between the locus and both markers. Moreover, using markers tightly flanking a locus, one can reduce linkage drag by more accurately selecting individuals that have less of the potentially deleterious donor parent DNA. Any marker linked to or among the chromosome intervals described herein can thus find use within the scope of this disclosure.

Similarly, by identifying plants lacking a desired marker locus, plants having unfavorable flowering initiation daylengths can be identified and eliminated from subsequent crosses. These marker loci can be introgressed into any desired genomic background, germplasm, plant, line, variety, etc., as part of an overall MAS breeding program designed to flowering initiation timing. The disclosure also provides chromosome QTL intervals and identified trait loci that can be used in MAS to select plants that demonstrate different flower initiation traits. The QTL intervals and trait loci can also be used to counter-select plants that have less favorable flower initiation traits.

Thus, the disclosure permits detection of the presence or absence of desired flower initiation genotypes in the genomes of Cannabis plants as part of a MAS program, as described herein. In one example, a breeder ascertains the genotype at one or more markers for a parent having favorable flowering initiation, and the genotype at one or more markers for a parent with unfavorable flowering initiation timing. A breeder can then reliably track the inheritance of the favorable flowering initiation alleles through subsequent populations derived from crosses between the two parents by genotyping offspring with the markers used on the parents and comparing the genotypes at those markers with those of the parents. Depending on how tightly linked the marker alleles are with the trait, progeny that share genotypes with the parent having favorable flowering initiation can be reliably predicted to express the desirable phenotype and progeny that share genotypes with the parent having unfavorable flower initiation, which can be reliably predicted to express the undesirable phenotype. Thus, the laborious, inefficient, and potentially inaccurate process of manually phenotyping the progeny for advantageous flowering initiation traits is avoided.

Closely linked markers flanking the locus of interest that have alleles in linkage disequilibrium with flowering initiation alleles at that locus may be effectively used to select for progeny plants with desirable advantageous flowering initiation traits. Thus, the markers described herein, such as those listed in Tables 4 and 5, as well as other markers genetically linked to the same chromosome interval, may be used to select for Cannabis plants with different traits relating to flowering initiation. Often, a set of these markers will be used, (e.g., 2 or more, 3 or more, 4 or more, 5 or more) in the flanking regions of the locus. Optionally, as described above, a marker flanking or within the actual locus may also be used. The parents and their progeny may be screened for these sets of markers, and the markers that are polymorphic between the two parents used for selection. In an introgression program, this allows for selection of the gene or locus genotype at the more proximal polymorphic markers and selection for the recurrent parent genotype at the more distal polymorphic markers.

In an example, MAS is used to select one or more Cannabis plants comprising flower initiation at increased daylight hours, the method comprising: obtaining nucleic acids from a sample plant or its germplasm; (ii) detecting one or more markers that indicate flower initiation at increased daylight hours, (iii) indicating the flower initiation at increased daylight hours, and (iv) selecting the one or more plants that initiate flowering at increased daylight hours. In an example, the sample plant(s) is a progeny plant obtained from a cross between a first plant that initiates flowering at increased daylight hours, and a second plant that does not initiate flowering at increased daylight hours.

In another example, MAS is used to select one or more Cannabis plants comprising flower initiation at a fewer number of days, the method comprising: i) obtaining nucleic acids from a sample plant or its germplasm; (ii) detecting one or more markers that indicate flower initiation at a fewer number of days. (iii) indicating the initiation of flowering at a fewer number of days, and (iv) selecting the one or more plants that initiate flowering at a fewer number of days. In an example, the sample plant(s) is a progeny plant obtained from a cross between a first plant that initiates flowering at a fewer number of days, and a second plant that does not initiate flowering at a fewer number of days. In some examples, a plant is selected for further breeding (crossing) or for making a Cannabis product (e.g., a kief, hashish, bubble hash, an edible product, solvent reduced oil, sludge, e-juice, or tincture).

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes may in some circumstances be more informative than single SNPs and can be more descriptive of any particular genotype. Haplotypes of the present disclosure are described herein, for example in Tables 2-8, and can be used for marker assisted selection.

The choice of markers actually used to practice the disclosure is not limited and can be any marker that is genetically linked to the intervals as described herein, which includes markers mapping within the intervals. In certain examples, the disclosure further provides markers closely genetically linked to, or within approximately 0.5 cM of, the markers provided herein and chromosome intervals whose borders fall between or include such markers, and including markers within approximately 0.4 cM, 0.3 cM, 0.2 cM, and about 0.1 cM of the markers provided herein.

In some examples the markers, haplotypes, and trait loci described above can be used for marker assisted selection to produce additional progeny plants comprising the indicated preferential flowering initiation periods. In some examples, backcrossing may be used in conjunction with marker-assisted selection.

### Gene Editing

In some methods disclosed herein, gene editing is used to develop Cannabis plants with modified flowering (e.g., plants that flower at increased daylight hours and/or initiate flowering in a fewer number of days). In some examples, the methods include introducing a genetic modification or beneficial allele disclosed herein (e.g., a genetic modification or beneficial allele of ELF9, FT, CDF2, AGO5, GASA4, CLPS3, or PI) that cause modified flowering. Introducing a beneficial allele into a Cannabis plant includes introducing a beneficial allele into a plant cell, plant tissue, or plant part. Introducing a beneficial allele includes introducing a full-length gene (e.g., an expression cassette including a beneficial allele) as well as introducing a mutation in an endogenous gene (modifying an endogenous gene), such that the sequence is changed to a beneficial allele (e.g., mutagenesis or gene editing). Specific, exemplary beneficial alleles of ELF9, FT, CDF2, AGO5, GASA4, CLPS3, and PI are described herein (e.g., see, Example 2). In some examples, a genetic modification or beneficial allele of ELF9 and/or FT is introduced into the Cannabis plant. In some examples, the method further includes introducing a genetic modification or beneficial allele of CDF2 into the Cannabis plant. In some examples, a beneficial allele is introduced into a Cannabis plant cell or tissue, and a Cannabis plant is regenerated from the transformed cell or tissue.

In some examples, modifying an endogenous Cannabis gene includes introducing a nucleic acid substitution, insertion, or deletion into the gene. In some examples the modification is homozygous or heterozygous in the modified plant. In some examples, the modification is heterozygous in the modified plant. In some examples, the modification is homozygous in the modified plant. In some examples, the genetic modification is introduced by mutagenesis or a gene editing technique (e.g., RNAi, CRISPR/Cas9, ZFN, or TALEN based systems).

In other examples, the methods include (i) replacing a nucleic acid sequence of a parent plant with a nucleic acid sequence conferring flowering initiation at increased daylight hours or at a fewer number of days, (ii) crossing or selfing the parent plant, thereby producing a plurality of progeny seed, and (iii), selecting one or more progeny plants grown from the progeny seed that comprise the nucleic acid sequence conferring flowering initiation at increased daylight hours and/or at a fewer number of days thereby selecting plants having flowering initiation at increased daylight hours or at a fewer number of days.

Methods of gene editing have been described, and many methods can be used with the present disclosure. For example, the action of genome editing proteins and various endogenous DNA repair pathways can be used to engineer a trait in a plant, such as a Cannabis plant. These pathways may be normally present in a cell or may be induced by the action of the genome editing protein. Using genetic and chemical tools to over-express or suppress one or more genes or elements of these pathways can improve the efficiency and/or outcome of the methods of the disclosure. For example, it can be useful to over-express certain homologous recombination pathway genes or suppression of non-homologous pathway genes, depending upon the desired modification.

For example, gene function can be modified using antisense modulation using at least one antisense compound, including antisense DNA, antisense RNA, a ribozyme, DNAzyme, a locked nucleic acid (LNA) and an aptamer. In some examples the molecules are chemically modified. In other examples the antisense molecule is antisense DNA or an antisense DNA analog.

RNA interference (RNAi) is another method known in the art to reduce gene function in plants, which is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs. The dsRNA used to initiate RNAi, may be isolated from native source or produced by known means, e.g., transcribed from DNA. Plasmids and vectors for generating RNAi molecules against target sequence are now readily available from commercial sources.

DNAzyme molecules, enzymatic oligonucleotides, and mutagenesis are other commonly known methods for reducing gene function. Any available mutagenesis procedure can be used, including but not limited to, site-directed point mutagenesis, random point mutagenesis, in vitro or in vivo homologous recombination (DNA shuffling), uracil-containing templates, oligonucleotide-directed mutagenesis, phosphorothioate-modified DNA mutagenesis, mutagenesis using gapped duplex DNA, point mismatch repair, repair-deficient host strains, restriction-selection and restriction-purification, deletion mutagenesis, total gene synthesis, double-strand break repair, zinc-finger nucleases (ZFN), transcription activator-like effector nucleases (TALEN), any other known mutagenesis procedure.

Clustered regularly interspaced short palindromic repeats (CRISPR)/CRISPR associated protein (Cas) system comprises genome engineering tools based on the bacterial CRISPR/Cas prokaryotic adaptive imnnune system. This RNA-based technology is very specific and allows targeted cleavage of genomic DNA guided by a customizable small noncoding RNA, resulting in gene modifications by both non-homologous end joining (NHEJ) and homology-directed repair (HDR) mechanisms (Belhaj K. et al., 2013. Plant Methods 2013, 9:39). In some examples, a CRISPR/Cas system comprises a CRISPR/Cas9 system. CRISPR-based gene editing systems need not be limited to Cas9 systems, as other analogous editing enzymes are known, e.g., MAD7.

In some examples, modifying a Cannabis gene to a beneficial allele includes a step of introducing a nucleic acid into a Cannabis host cell. Methods for transformation of plant cells required for gene editing are known, and the selection of the most appropriate transformation technique for a particular example of the disclosure may be determined by the practitioner. Suitable methods may include electroporation of plant protoplasts, liposome-mediated transformation, polyethylene glycol (PEG) mediated transformation, transformation using viruses, micro-injection of plant cells, micro-projectile bombardment of plant cells, and Agrobacterium tumefaciens mediated transformation. Transformation means introducing a nucleotide sequence in a plant in a manner to cause stable or transient expression of the sequence.

In plant transformation techniques (e.g., vacuum-infiltration, floral spraying or floral dip procedures) are known and may be used to introduce expression cassettes of the disclosure (typically in an Agrobacterium vector) into meristematic or germline cells of a whole plant. Such methods provide a simple and reliable method of obtaining transformants at high efficiency while avoiding the use of tissue culture. (see, e.g., Chung et at. 2000 Transgenic Res. 9:471-476; and Desfeux et at. 2000 Plant Physiol 123:895-904). In these examples, seed produced by the plant comprise the expression cassettes encoding the genome editing proteins of the disclosure. The seed can be selected based on the ability to germinate under conditions that inhibit germination of the untransformed seed.

If transformation techniques require use of tissue culture, transformed cells may be regenerated into plants in accordance with known techniques. The regenerated plants may then be grown, and crossed with the same or different plant varieties using traditional breeding techniques to produce seed, which are then selected under the appropriate conditions.

The expression cassette can be integrated into the genome of the plant cells, in which case subsequent generations will express the genome editing proteins of the disclosure. Alternatively, the expression cassette is not integrated into the genome of the plant's cell, in which case the genome editing protein is transiently expressed in the transformed cells and is not expressed in subsequent generations.

A genome editing protein itself may be introduced into the plant cell. In these examples, the introduced genome editing protein is provided in sufficient quantity to modify the cell but does not persist after a contemplated period of time has passed or after one or more cell divisions. In such examples, no further steps are needed to remove or segregate away the genome editing protein and the modified cell. In these examples, the genome editing protein is prepared in vitro prior to introduction to a plant cell using known recombinant expression systems (bacterial expression, in vitro translation, yeast cells, insect cells and the like). After expression, the protein is isolated, refolded if needed, purified and optionally treated to remove any purification tags, such as a His-tag. Once crude, partially purified, or more completely purified genome editing proteins are obtained, they may be introduced to a plant cell via electroporation, by bombardment with protein coated particles, by chemical transfection or by some other means of transport across a cell membrane.

The genome editing protein can also be expressed in Agrobacterium as a fusion protein, fused to an appropriate domain of a virulence protein that is translocated into plants (e.g., VirD2, VirE2, VirE2 and VirF). The Vir protein fused with the genome editing protein travels to the plant cell's nucleus, where the genome editing protein would produce the desired double stranded break in the genome of the cell. (see, Vergunst et al. 2000 Science 290:979-82).

In some examples, gene editing is used with plant breeding to develop Cannabis plants having modified flowering (e.g., flower at increased daylight hours and/or initiate flowering at a fewer number of days). In a non-limiting example, a nucleic acid sequence of a plant is replaced with a nucleic acid sequence conferring a modified flowering phenotype. The plant is crossed or selfed to produce progeny. One or more progeny plants comprising the nucleic acid sequence conferring modified flowering are selected (or one or more progeny plants having modified flowering are selected), thereby selecting plants having modified flowering. The Cannabis plant can be Cannabis sativa, Cannabis indica, or Cannabis ruderalis. In a non-limiting example, the Cannabis plant is Cannabis sativa.

### Plants and Products

Also disclosed are Cannabis plants that have modified flowering (e.g., flower at increased daylight hours and/or initiate flowering at a fewer number of days). In some examples, the Cannabis plants having modified flowering include a non-naturally occurring beneficial allele of one or more of ELF9, FT, CDF2, AGO5, GASA4, CLPS3, and PI. Exemplary beneficial alleles are disclosed herein. The beneficial alleles can be introduced into Cannabis plants using genetic engineering or breeding techniques (e.g., MAS). Also included in the disclosure are Cannabis plants made by any of the methods disclosed herein. Material derived from the Cannabis plants, including seed, tissue, or cells (including protoplasts); or progeny of the plant, such as F1 or F2 progeny, are also encompassed by this disclosure. The Cannabis plant can be Cannabis sativa, Cannabis indica, or Cannabis ruderalis. In a non-limiting example, the Cannabis plant is Cannabis sativa.

Further disclosed are Cannabis products. The product may be any product known in the Cannabis arts, and can include, but is not limited to, extracts, a kief, hashish, bubble hash, an edible product, a flower, a seed, solvent reduced oil, sludge, e-juice, or tincture. As used herein, Cannabis sludges are solvent-free Cannabis extracts made via multigas extraction including the refrigerant 134A, butane, iso-butane and propane in a ratio that delivers a very complete and balanced extraction of cannabinoids and essential oils. Products can also be prepared by any other method known in the art, including but not limited to lyophilization.

Also disclosed are compositions including (or derived from) any of the Cannabis plants disclosed herein. In some examples, the composition is for pulmonary administration. The compositions include, but are not limited to, dry powder compositions consisting of the powder of a Cannabis oil described herein, and the powder of a suitable carrier and/or lubricant. The compositions for pulmonary administration can be inhaled from any suitable dry powder inhaler device. In certain instances, the compositions may be conveniently delivered in the form of an aerosol spray from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

For oral administration, a pharmaceutical composition or a medicament can take the form of, e.g., a tablet or a capsule prepared by conventional means with a pharmaceutically acceptable excipient. Preferred are tablets and gelatin capsules comprising the active ingredient(s), together with (a) diluents or fillers, e.g., lactose, dextrose, sucrose, mannitol, maltodextrin, lecithin, agarose, xanthan gum, guar gum, sorbitol, cellulose (e.g., ethyl cellulose, microcrystalline cellulose), glycine, pectin, polyacrylates and/or calcium hydrogen phosphate, calcium sulfate, (b) lubricants; e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium or potassium salt, or effervescent mixtures; (e) wetting agents, e.g., sodium lauryl sulfate, and/or (f) absorbents, colorants, flavors and sweeteners. Tablets can be either uncoated or coated according to methods known in the art. The excipients described herein can also be used for preparation of buccal dosage forms and sublingual dosage forms (e.g., films and lozenges) as described, for example, in U.S. Pat. Nos. 5,981,552 and 8,475,832. Formulation in chewing gums as described, for example, in U.S. Pat. No. 8,722,022, is also contemplated.

Further preparations for oral administration can take the form of, for example, solutions, syrups, suspensions, and toothpastes. Liquid preparations for oral administration can be prepared by conventional means with pharmaceutically acceptable additives, for example, suspending agents, for example, sorbitol syrup, cellulose derivatives, or hydrogenated edible fats; emulsifying agents, for example, lecithin, xanthan gum, or acacia; non-aqueous vehicles, for example, almond oil, sesame oil, hemp seed oil, fish oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate.

Typical formulations for topical administration include creams, ointments, sprays, lotions, hydrocolloid dressings, and patches, as well as eye drops, ear drops, and deodorants. Cannabis oils can be administered via transdermal patches as described, for example, in U.S. Pat. Appl. Pub. No. 2015/0126595 and U.S. Pat. No. 8,449,908. Formulation for rectal or vaginal administration is also contemplated. The Cannabis oils can be formulated, for example, as suppositories containing conventional suppository bases such as cocoa butter and other glycerides as described in U.S. Pat. Nos. 5,508,037 and 4,933,363. Compositions can contain other solidifying agents such as shea butter, beeswax, kokum butter, mango butter, illipe butter, tamanu butter, carnauba wax, emulsifying wax, soy wax, castor wax, rice bran wax, and candelilla wax. Compositions can further include clays (e.g., Bentonite, French green clays, Fuller's earth, Rhassoul clay, white kaolin clay) and salts (e.g., sea salt, Himalayan pink salt, and magnesium salts such as Epsom salt).

The compositions disclosed herein can be formulated for parenteral administration by injection, for example, by bolus injection or continuous infusion. Formulations for injection can be presented in unit dosage form, for example, in ampoules or in multi-dose containers, optionally with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure, buffers, and/or other ingredients. Alternatively, the compositions can be in powder form for reconstitution with a suitable vehicle, for example, a carrier oil, before use. In addition, the compositions may also contain other therapeutic agents or substances.

The compositions can be prepared according to conventional mixing, granulating, and/or coating methods, and contain from about 0.1 to about 75%, preferably from about 1 to about 50%, of the Cannabis oil extract. In general, subjects receiving a Cannabis oil composition orally are administered doses ranging from about 1 to about 2000 mg of Cannabis oil. A small dose ranging from about 1 to about 20 mg can typically be administered orally when treatment is initiated, and the dose can be increased (e.g., doubled) over a period of days or weeks until the maximum dose is reached.

### Kits for Use in Diagnostic Applications

Kits for use in diagnostic, research, and prognostic applications are also provided by the disclosure. Such kits may include any or all of the following: assay reagents, buffers, nucleic acids for detecting the target sequences and other hybridization probes and/or primers (e.g., probes or primers disclosed herein). The kits may include instructional materials containing directions (i.e., protocols) for the practice of the methods of this disclosure. While the instructional materials typically comprise written or printed materials, they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD ROM), cloud-based media, and the like. Such media may include addresses to internet sites that provide such instructional materials.

### Additional Aspects

Clause 1. A method for selecting one or more plants that flower at increased daylight hours, the method comprising i) obtaining nucleic acids from a sample plant or its germplasm; (ii) detecting one or more markers that indicate flower initiation at increased daylight hours, (iii) indicating the flower initiation at increased daylight hours, and (iv) selecting the one or more plants that initiate flowering at increased daylight hours.

Clause 2. The method of claim 1 wherein the sample plant(s) is a progeny plant obtained from a cross between a first plant that initiates flowering at increased daylight hours, and a second plant that does not initiate flowering at increased daylight hours.

Clause 3. The method of claim 1 wherein the one or more markers comprises a polymorphism on chromosome 8 relative to a reference genome at nucleotide positions: 19,681; 44,981; 51,589; 87,904; 159,096; 199,126; 371,700; 378,368; 385,390; 447,673; 555,854; 564,634; 568,544; 720,538; 936,746; 1,042,634; 1,088,206; 1,147,746; 1,191,275; 1,200,057; 1,211,168; 1,216,900; 1,218,786; 1,277,500; 1,305,959; 1,397,056; 1,496,730; 1,515,643; 1,526,023; 1,577,268; 1,582,128; 1,617,208; 1,659,209; 1,690,294; 1,725,273; 1,729,625; 1,735,848; 1,816,071; 1,877,889; 1,933,751; 1,993,648; 2,002,233; 2,020,279; 2,253,641; 2,279,386; 2,364,298; or 2,404,096; wherein the reference genome is the Abacus Cannabis reference genome.

Clause 4. The method of claim 3 wherein the nucleotide position comprises on chromosome 8: (a) a A/A or G/A genotype at position 19681; (b) a A/A or T/A genotype at position 44981; (c) a C/C or T/C genotype at position 51589; (d) a G/G or A/G genotype at position 87904; (e) a G/G or G/A genotype at position 159096; (f) a GIG or G/C genotype at position 199126; (g) a A/A or G/A genotype at position 371700; (h) a G/G or G/T genotype at position 378368; (i) a G/G or G/A genotype at position 385390; (j) a G/G or A/G genotype at position 447673; (k) a T/T or T/C genotype at position 555854; (1) a T/T or T/C genotype at position 564634; (m) a G/G or G/A genotype at position 568544; (n) a T/T or T/G genotype at position 720538; (o) a A/A or A/T genotype at position 936746; (p) a G/G or G/A genotype at position 1042634; (q) a C/C or T/C genotype at position 1088206; (r) a C/C or C/T genotype at position 1147746; (s) a C/C or T/C genotype at position 1191275; (t) a G/G or G/A genotype at position 1200057; (u) a T/T or C/T genotype at position 1211168; (v) a T/T or T/C genotype at position 1216900; (w) a G/G or A/G genotype at position 1218786; (x) a T/T or G/T genotype at position 1277500; (y) a T/C genotype at position 1305959; (z) a A/A or A/G genotype at position 1397056; (aa) a A/A or A/G genotype at position 1496730; (ab) a T/T or T/C genotype at position 1515643; (ac) a A/A or A/T genotype at position 1526023; (ad) a G/G or A/G genotype at position 1577268; (ae) a T/T or G/T genotype at position 1582128; (af) a C/C or G/C genotype at position 1617208; (ag) a C/C or A/C genotype at position 1659209; (ah) a A/A or A/G genotype at position 1690294; (ai) a G/G or G/A genotype at position 1725273; (aj) a C/C or C/G genotype at position 1729625; (ak) a G/G or T/G genotype at position 1735848; (al) a A/A or T/A genotype at position 1816071; (am) a C/C or A/C genotype at position 1877889; (an) a C/C or C/A genotype at position 1933751; (ao) a T/T or C/T genotype at position 1993648; (ap) a A/A or C/A genotype at position 2002233; (aq) a A/A or A/G genotype at position 2020279; (ar) a C/C or C/A genotype at position 2253641; (as) a C/C or C/A genotype at position 2279386; (at) a GIG or A/G genotype at position 2364298; or(au) a GIG or A/G genotype at position 2404096; wherein the reference genome is the Abacus Cannabis reference genome.

Clause 5. The method of claim 1 wherein the one or more markers comprises a polymorphism at position 51 of any one or more of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; or SEQ ID NO: 47.

Clause 6. The method of claim 5 wherein the nucleotide position comprises:

(a) a A/A or G/A genotype at position 51 of SEQ ID NO: 1; (b) a A/A or T/A genotype at position 51 of SEQ ID NO: 2; (c) a C/C or T/C genotype at position 51 of SEQ ID NO: 3; (d) a GIG or A/G genotype at position 51 of SEQ ID NO: 4; (e) a GIG or G/A genotype at position 51 of SEQ ID NO: 5; (f) a GIG or G/C genotype at position 51 of SEQ ID NO: 6; (g) a A/A or G/A genotype at position 51 of SEQ ID NO: 7; (h) a GIG or G/T genotype at position 51 of SEQ ID NO: 8; (i) a GIG or G/A genotype at position 51 of SEQ ID NO: 9; (j) a GIG or A/G genotype at position 51 of SEQ ID NO: 10; (k) a TIT or T/C genotype at position 51 of SEQ ID NO: 11; (1) a T/T or T/C genotype at position 51 of SEQ ID NO: 12; (m) a GIG or G/A genotype at position 51 of SEQ ID NO: 13; (n) a T/T or T/G genotype at position 51 of SEQ ID NO: 14; (o) a A/A or A/T genotype at position 51 of SEQ ID NO: 15; (p) a GIG or G/A genotype at position 51 of SEQ ID NO: 16; (q) a C/C or T/C genotype at position 51 of SEQ ID NO: 17; (r) a C/C or C/T genotype at position 51 of SEQ ID NO: 18; (s) a C/C or T/C genotype at position 51 of SEQ ID NO: 19; (t) a GIG or G/A genotype at position 51 of SEQ ID NO: 20; (u) a T/T or C/T genotype at position 51 of SEQ ID NO: 21; (v) a T/T or T/C genotype at position 51 of SEQ ID NO: 22; (w) a GIG or A/G genotype at position 51 of SEQ ID NO: 23; (x) a TIT or G/T genotype at position 51 of SEQ ID NO: 24; (y) a T/C genotype at position 51 of SEQ ID NO: 25; (z) a A/A or A/G genotype at position 51 of SEQ ID NO: 26; (aa) a A/A or A/G genotype at position 51 of SEQ ID NO: 27; (ab) a T/T or T/C genotype at position 51 of SEQ ID NO: 28; (ac) a A/A or A/T genotype at position 51 of SEQ ID NO: 29; (ad) a GIG or A/G genotype at position 51 of SEQ ID NO: 30; (ae) a T/T or G/T genotype at position 51 of SEQ ID NO: 31; (af) a C/C or G/C genotype at position 51 of SEQ ID NO: 32; (ag) a C/C or A/C genotype at position 51 of SEQ ID NO: 33; (ah) a A/A or A/G genotype at position 51 of SEQ ID NO: 34; (ai) a GIG or G/A genotype at position 51 of SEQ ID NO: 35; (aj) a C/C or C/G genotype at position 51 of SEQ ID NO: 36; (ak) a GIG or T/G genotype at position 51 of SEQ ID NO: 37; (al) a A/A or T/A genotype at position 51 of SEQ ID NO: 38; (am) a C/C or A/C genotype at position 51 of SEQ ID NO: 39; (an) a C/C or C/A genotype at position 51 of SEQ ID NO: 40; (ao) a T/T or C/T genotype at position 51 of SEQ ID NO: 41; (ap) a A/A or C/A genotype at position 51 of SEQ ID NO: 42; (aq) a A/A or A/G genotype at position 51 of SEQ ID NO: 43; (ar) a C/C or C/A genotype at position 51 of SEQ ID NO: 44; (as) a C/C or C/A genotype at position 51 of SEQ ID NO: 45; (at) a G/G or A/G genotype at position 51 of SEQ ID NO: 46; or (au) a G/G or A/G genotype at position 51 of SEQ ID NO: 47.

Clause 7. The method of claim 1 wherein the one or more markers comprises a polymorphism relative to a reference genome within any one or more haplotypes wherein the haplotypes comprise the region on chromosome 8: (a) between positions 0 and 25101; (b) between positions 25101 and 63750; (c) between positions 25101 and 63750; (d) between positions 82833 and 95593; (e) between positions 146257 and 209857; (f) between positions 146257 and 209857; (g) between positions 361045 and 401990; (h) between positions 361045 and 401990; (i) between positions 361045 and 401990; (j) between positions 430259 and 479822; (k) between positions 537879 and 558636; (1) between positions 558636 and 581624; (m) between positions 558636 and 581624; (n) between positions 714581 and 776053; (o) between positions 935803 and 938775; (p) between positions 1033166 and 1060034; (q) between positions 1076208 and 1108104; (r) between positions 1134931 and 1154438; (s) between positions 1184825 and 1193261; (t) between positions 1193261 and 1201662; (u) between positions 1205216 and 1225668; (v) between positions 1205216 and 1225668; (w) between positions 1205216 and 1225668; (x) between positions 1273758 and 1281517; (y) between positions 1295157 and 1319381; (z) between positions 1387657 and 1403412; (aa) between positions 1474748 and 1502974; (ab) between positions 1513180 and 1539957; (ac) between positions 1513180 and 1539957; (ad) between positions 1557156 and 1595751; (ae) between positions 1557156 and 1595751; (af) between positions 1600294 and 1618178; (ag) between positions 1656443 and 1686562; (ah) between positions 1686562 and 1693118; (ai) between positions 1714084 and 1757477; (aj) between positions 1714084 and 1757477; (ak) between positions 1714084 and 1757477; (al) between positions 1813955 and 1822552; (am) between positions 1866553 and 1881061; (an) between positions 1927664 and 1944792; (ao) between positions 1983548 and 1997626; (ap) between positions 1997626 and 2037226; (aq) between positions 1997626 and 2037226; (ar) between positions 2245812 and 2257882; (as) between positions 2272681 and 2285144; (at) between positions 2360594 and 2369020; or (au) between positions 2401504 and 2409146; wherein the reference genome is the Abacus Cannabis reference genome.

Clause 8. The method of claim 1 wherein the flowering at increased daylight hours occurs when the daylight is greater 12 hours.

Clause 9. The method of claim 3 wherein the method further includes a method for selecting one or more plants that initiate flowering at a fewer number of days, the method comprising i) detecting two or more markers that indicate flower initiation at a fewer number of days, (ii) indicating the initiation of flowering at a fewer number of days, and (iii) selecting the one or more plants that initiate flowering at a fewer number of days.

Clause 10. The method of claim 9 wherein the sample plant(s) is a progeny plant obtained from a cross between a first plant that initiates flowering at a fewer number of days, and a second plant that does not initiate flowering at a fewer number of days.

Clause 11. The method of claim 9 wherein at least a first of the two or more markers comprises a marker derived from group (1) and at least a second of the two or more markers comprises a marker derived from group (2) wherein:
(a) the group (1) markers (i) comprise one or more polymorphisms on chromosome 8 relative to the reference genome at nucleotide positions: 19,681; 44,981; 51,589; 87,904; 199,126; 378,368; 385,390; 447,673; 564,634; 568,544; 720,538; 936,746; 1,042,634; 1,088,206; 1,147,746; 1,191,275; 1,200,057; 1,211,168; 1,216,900; 1,218,786; 1,277,500; 1,305,959; 1,397,056; 1,496,730; 1,515,643; 1,526,023; 1,577,268; 1,582,128; 1,617,208; 1,659,209; 1,690,294; 1,725,273; 1,729,625; 1,735,848; 1,816,071; 1,877,889; 1,933,751; 1,993,648; 2,002,233; 2,020,279; 2,253,641; 2,279,386; 2,364,298; or 2,404,096; and (ii) do not include any of the polymorphisms on chromosome 8 relative to the reference genome at nucleotide positions 159,096; 371,700, and 555,854; and
(b) the group (2) markers comprise one or more polymorphisms on chromosome 8 relative to the reference genome at nucleotide positions: 159,096; 371,700; or 555,854.

Clause 12. The method of claim 11 wherein the nucleotide position comprises on chromosome 8:

(a) a A/A or G/A genotype at position 19681; (b) a A/A or T/A genotype at position 44981; (c) a C/C or T/C genotype at position 51589; (d) a G/G or A/G genotype at position 87904; (e) a GIG or G/A genotype at position 159096; (f) a G/G or G/C genotype at position 199126; (g) a A/A or G/A genotype at position 371700; (h) a G/G or G/T genotype at position 378368; (i) a G/G or G/A genotype at position 385390; (j) a G/G or A/G genotype at position 447673; (k) a T/T or T/C genotype at position 555854; (1) a T/T or T/C genotype at position 564634; (m) a G/G or G/A genotype at position 568544; (n) a T/T or T/G genotype at position 720538; (o) a A/A or A/T genotype at position 936746; (p) a G/G or G/A genotype at position 1042634; (q) a C/C or T/C genotype at position 1088206; (r) a C/C or C/T genotype at position 1147746; (s) a C/C or T/C genotype at position 1191275; (t) a G/G or G/A genotype at position 1200057; (u) a T/T or C/T genotype at position 1211168; (v) a T/T or T/C genotype at position 1216900; (w) a G/G or A/G genotype at position 1218786; (x) a T/T or G/T genotype at position 1277500; (y) a T/C genotype at position 1305959; (z) a A/A or A/G genotype at position 1397056; (aa) a A/A or A/G genotype at position 1496730; (ab) a T/T or T/C genotype at position 1515643; (ac) a A/A or A/T genotype at position 1526023; (ad) a G/G or A/G genotype at position 1577268; (ae) a T/T or G/T genotype at position 1582128; (af) a C/C or G/C genotype at position 1617208; (ag) a C/C or A/C genotype at position 1659209; (ah) a A/A or A/G genotype at position 1690294; (ai) a G/G or G/A genotype at position 1725273; (aj) a C/C or C/G genotype at position 1729625; (ak) a G/G or T/G genotype at position 1735848; (al) a A/A or T/A genotype at position 1816071; (am) a C/C or A/C genotype at position 1877889; (an) a C/C or C/A genotype at position 1933751; (ao) a T/T or C/T genotype at position 1993648; (ap) a A/A or C/A genotype at position 2002233; (aq) a A/A or A/G genotype at position 2020279; (ar) a C/C or C/A genotype at position 2253641; (as) a C/C or C/A genotype at position 2279386; (at) a G/G or A/G genotype at position 2364298; or (au) a G/G or A/G genotype at position 2404096; wherein the reference genome is the Abacus Cannabis reference genome.

Clause 13. The method of claim 9 wherein at least a first of the two or more markers comprises a marker derived from group (1) and at least a second of the two or more markers comprises a marker derived from group (2) wherein:
(a) the group (1) markers (i) comprise a polymorphism at position 51 of one or more of SEQ ID NO:1; SEQ ID NO:2; SEQ ID NO:3; SEQ ID NO:4; SEQ ID NO:6; SEQ ID NO:8; SEQ ID NO:9; SEQ ID NO:10; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; or SEQ ID NO: 47; and do not include a polymorphism at position 51 of any of SEQ ID NO:5; SEQ ID NO:7; or SEQ ID NO:11; and
(b) the group (2) markers comprise a polymorphism at position 51 of one or more of SEQ ID NO:5; SEQ ID NO:7; or SEQ ID NO:11.

Clause 14. The method of claim 13 wherein the nucleotide position comprises: (a) a A/A or G/A genotype at position 51 of SEQ ID NO: 1;

(b) a A/A or T/A genotype at position 51 of SEQ ID NO: 2; (c) a C/C or T/C genotype at position 51 of SEQ ID NO: 3; (d) a G/G or A/G genotype at position 51 of SEQ ID NO: 4; (e) a G/G or G/A genotype at position 51 of SEQ ID NO: 5; (f) a G/G or G/C genotype at position 51 of SEQ ID NO: 6; (g) a A/A or G/A genotype at position 51 of SEQ ID NO: 7; (h) a GIG or G/T genotype at position 51 of SEQ ID NO: 8; (i) a G/G or G/A genotype at position 51 of SEQ ID NO: 9; (j) a G/G or A/G genotype at position 51 of SEQ ID NO: 10; (k) a T/T or T/C genotype at position 51 of SEQ ID NO: 11; (1) a T/T or T/C genotype at position 51 of SEQ ID NO: 12; (m) a G/G or G/A genotype at position 51 of SEQ ID NO: 13; (n) a T/T or T/G genotype at position 51 of SEQ ID NO: 14; (o) a A/A or A/T genotype at position 51 of SEQ ID NO: 15; (p) a G/G or G/A genotype at position 51 of SEQ ID NO: 16; (q) a C/C or T/C genotype at position 51 of SEQ ID NO: 17; (r) a C/C or C/T genotype at position 51 of SEQ ID NO: 18; (s) a C/C or T/C genotype at position 51 of SEQ ID NO: 19; (t) a G/G or G/A genotype at position 51 of SEQ ID NO: 20; (u) a T/T or C/T genotype at position 51 of SEQ ID NO: 21; (v) a T/T or T/C genotype at position 51 of SEQ ID NO: 22; (w) a G/G or A/G genotype at position 51 of SEQ ID NO: 23; (x) a T/T or G/T genotype at position 51 of SEQ ID NO: 24; (y) a T/C genotype at position 51 of SEQ ID NO: 25; (z) a A/A or A/G genotype at position 51 of SEQ ID NO: 26; (aa) a A/A or A/G genotype at position 51 of SEQ ID NO: 27; (ab) a T/T or T/C genotype at position 51 of SEQ ID NO: 28; (ac) a A/A or A/T genotype at position 51 of SEQ ID NO: 29; (ad) a G/G or A/G genotype at position 51 of SEQ ID NO: 30; (ae) a T/T or G/T genotype at position 51 of SEQ ID NO: 31; (af) a C/C or G/C genotype at position 51 of SEQ ID NO: 32; (ag) a C/C or A/C genotype at position 51 of SEQ ID NO: 33; (ah) a A/A or A/G genotype at position 51 of SEQ ID NO: 34; (ai) a G/G or G/A genotype at position 51 of SEQ ID NO: 35; (aj) a C/C or C/G genotype at position 51 of SEQ ID NO: 36; (ak) a G/G or T/G genotype at position 51 of SEQ ID NO: 37; (al) a A/A or T/A genotype at position 51 of SEQ ID NO: 38; (am) a C/C or A/C genotype at position 51 of SEQ ID NO: 39; (an) a C/C or C/A genotype at position 51 of SEQ ID NO: 40; (ao) a T/T or C/T genotype at position 51 of SEQ ID NO: 41; (ap) a A/A or C/A genotype at position 51 of SEQ ID NO: 42; (aq) a A/A or A/G genotype at position 51 of SEQ ID NO: 43; (ar) a C/C or C/A genotype at position 51 of SEQ ID NO: 44; (as) a C/C or C/A genotype at position 51 of SEQ ID NO: 45; (at) a G/G or A/G genotype at position 51 of SEQ ID NO: 46; or (au) a G/G or A/G genotype at position 51 of SEQ ID NO: 47.

Clause 15. The method of claim 9 wherein the number of days averages 33.8 days.

Clause 16. The method of claim 1 wherein the selecting comprises marker assisted selection.

Clause 17. The method of claim 1 wherein the detecting comprises an oligonucleotide probe.

Clause 18. The method of claim 1 further comprising crossing the one or more plants comprising the indicated flower initiation at increased daylight hours phenotype to produce one or more F1 or additional progeny plants, wherein at least one of the F1 or additional progeny plants comprises the indicated flower initiation at increased daylight hours phenotype.

Clause 19. The method of claim 18 wherein the crossing comprises selfing, sibling crossing, or backcrossing.

Clause 20. The method of claim 19 wherein the at least one additional progeny plant comprising the indicated flowering at increased daylight hours phenotype comprises an F2-F7 progeny plant.

Clause 21. The method of claim 19 wherein the selfing, sibling crossing, or backcrossing comprises marker-assisted selection for at least two generations.

Clause 22. The method of claim 1 wherein the plant comprises a Cannabis plant.

Clause 23. A method for selecting one or more plants that initiate flowering at increased daylight hours, the method comprising replacing a nucleic acid sequence of a parent plant with a nucleic acid sequence conferring the flower initiation at increased daylight hours phenotype.

Clause 24. A plant selected by the method of claim 1.

Clause 25. A seed of the plant of claim 24.

Clause 26. A tissue culture of cells produced from the plant of claim 24.

Clause 27. A plant generated from the tissue culture of claim 26.

Clause 28. A protoplast produced from the plant of claim 24.

Clause 29. A method of generating a processed Cannabis product comprising the use of the plant of claim 26.

Clause 30. A Cannabis product produced from the plant of claim 24.

Clause 31. The product of claim 46 wherein the product is a kief, hashish, bubble hash, an edible product, solvent reduced oil, sludge, e-juice, or tincture.

### EXAMPLES

Aspects of the present teachings can be further understood in light of the following examples, which should not be construed as limiting the scope of the present teachings in any way.

### Example 1

Discovery of Markers Associated with Flower Initiation

### Plant Material and Phenotyping

F2 cannabis populations were created by crossing accessions differing for their ability to initiate flowers at day lengths greater than 12 hours. In total, three cannabis accessions that were able to form flower clusters with at least six pistils at 18/6 (=18 hours light, followed by 6 hours darkness during a 24 hour cycle) were crossed with photosensitive cannabis accessions that were unable to form flower clusters with at least six pistils at 18/6. For each parental cross 3-4 F1 plants were subsequently selfed to create F2 populations (Table 1).

TABLE 1
Projects and Cannabis mapping populations
used to map flower initiation traits.

| | | | | #Plants 6+ | |
|---|---|---|---|---|---|
| | | #F2 | Plant | pistils | Day |
| Project | Parent | populations | count | under 18/6 | length |
| 22ALV1 | 20GAQ-1229 | 3 | 147 | 106 | 18/6 |
| 22ALV2 | PTVOG-21-18 | 3 | 115 | 41 | 18/6 |
| 23ALV1 | 20GAQ-1072 | 4 | 139 | 89 | 18/6 |
| 22TRP2 | 20GAQ-1072 | 1 | 77 | NA | 12/12 |
| 23TRP1 | 20GAQ-1072 | 3 | 90 | NA | 12/12 |

First column: project code where plants were evaluated; Second column: name of parent which is capable of forming flower clusters with at least six pistils at 18/6; Third column: number of F2 populations evaluated involving parent listed in second column; Fourth column: number of plants evaluated from seed; Fifth column: number of plants forming flower clusters with at least six pistils under 18/6; Sixth column: day length under which plants were evaluated: 18/6 = 18 hours light, followed by 6 hours dark during a 24 hour cycle, 12/12 = 12 hours light, followed by 12 hours dark during a 24 hour cycle.

For 22ALV1 three F2 populations were grown from seed in a greenhouse at 18/6 during the summer of 2022 (n=147; Table 1). Plants were checked for flower initiation three times a week between 7-43 days after sow. Two traits were subsequently recorded: Flower Initiation (=the ability of a plant to initiate flowers, which is measured by recording the first date that a plant forms flower clusters with at least six pistils) and Days to Flower Initiation (=number of days it takes a plant to initiate flowers, which is measured as the number of days from sowing seeds until the first date a plant forms flower clusters with at least six pistils). Flower Initiation was recorded as a “1” if an accession was able to form clusters of flowers with at least six pistils during the course of the evaluation which lasted till day 43 after sow, otherwise the accession was scored a “0.” Days to Flower Initiation was recorded as the first day after the sow date that clusters of flowers with at least six pistils were observed. In total, 106 of the 147 evaluated accessions (72% of all evaluated 22ALV1 F2 accessions) initiated flowers between 28-43 days after sow with the vast majority of all evaluated flowering 22ALV1 F2 accessions initiating flowers at 18/6 between 32-37 days after sow. The parental variety which was able to initiate flowering under 18/6 was grown alongside the F2 plants as two accessions grown from seed. Flower initiation of those two accessions occurred at days 28 and 30 days after sow. The other parental variety was grown as three accessions from seed alongside the F2 plants, none of those initiated flowers under 18/6. After four weeks of vegetative growth under 18/6 lighting, both parents formed flowers after switching to 12/12 lighting.

For 22ALV2 three F2 populations were grown from seed in a greenhouse at 18/6 during winter of 2022/2023 (n=115; Table 1). Plants were checked for flower initiation three times a week between 7-74 days after sow. Flower Initiation and Days to Flower Initiation were recorded similarly as for 22ALV1. In total, 41 of the 115 evaluated accessions (36% of all evaluated 22ALV2 F2 accessions) initiated flowers between 51-74 days after sow.

For 23ALV1 four F2 populations were grown from seed in a greenhouse at 18/6 during summer of 2023 (n=139; Table 1). Plants were checked for flower initiation three times a week between 7-66 days after sow. Ten plants that were close to forming clusters of flowers with at least six pistils were recorded as having reached that stage at day 69 after sow. Flower Initiation and Days to Flower Initiation were recorded similarly as for 22ALV1. In total, 99 of the 142 evaluated accessions (70% of all evaluated 23ALV1 F2 accessions) initiated flowers between 50-69 days after sow.

For 22TRP2 and 23TRP1, one and three F2 populations, respectively, were grown from seed in a greenhouse during winter and spring of 2023 (n=77 and 90, respectively; Table 1). 22TRP2 was grown under 18/6 for 44 days after sowing, whereas 23TRP1 was grown under 18/6 for 33 days after sowing. Plants were checked for the presence of flower clusters with at least six pistils three times a week between 3-19 days after the lights were changed from 18 hours light (18/6) to 12 hours light (12/12), which from here onwards will be referred to as Days to Flower Initiation After 12/12 Flip. Days to Flower Initiation After 12/12 Flip ranged between 6-19 days and 11-18 days for 22TRP2 and 23TRP1, respectively. Subsequently, accessions that formed flower clusters on the first day of the observed range in each project were scored a “1”, whereas all other accessions were scored a “0”. In total, 38 of the 77 evaluated 22TRP2 accessions formed flower clusters with at least six pistils at 6 Days to Flower Initiation After 12/12 Flip (49% of all evaluated 22TRP2 accessions). For 23TRP1, in total 27 of the 90 evaluated 23TRP1 accessions formed flower clusters with at least pistils at 11 Days to Flower Initiation After 12/12 Flip (30% of all evaluated 23TRP1 accessions).

### Association Mapping

All F2 populations (Table 1) and the parents were genotyped with an Illumina bead array. After initial SNP QC of the mapping population accessions, further filtering steps were performed to filter out known low quality SNPs, followed by filtering for missing data (<10-25%;) and minor allele frequency (>1%) using vcftools (Danecek, Petr, et al. “The variant call format and VCFtools.” Bioinformatics 27.15 (2011): 2156-2158). For nested association mapping (NAM), missing data (<10-15%) were subsequently imputed (R package NAM “snpQC” option; Xavier, Alencar, et al. “NAM: association studies in multiple populations.” Bioinformatics 31.23 (2015): 3862-3864).

For ALV1 Fisher Exact test of Flower Initiation was performed per SNP (n=29,912; SNPs were filtered for <15% missing data) with the statistical package R. For this purpose two sets of 4×4 tables were created per SNP with the first set of 4×4 tables comparing counts of homozygous reference allele and ability to flower under 18/6, homozygous reference allele and the inability to flower under 18/6, homozygous alternate allele combined with heterozygous and the ability to flower under 18/6, homozygous alternate allele combined with heterozygous and the inability to flower under 18/6. The second set of 4×4 tables compared counts of homozygous alternate allele and the ability to flower under 18/6, homozygous alternate allele and the inability to flower under 18/6, homozygous reference allele combined with heterozygous and the ability to flower under 18/6, homozygous reference allele combined with heterozygous and the inability to flower under 18/6. Subsequently, the most significant p-value of the two Fisher Exact tests per SNP was recorded. In total, 47 SNP markers located between 19,681-2,404,096 hp on chromosome 8 of the Abacus reference genome (version Csat_AbacusV2; NCBI assembly accession GCA_025232715.1) were found to be significantly associated with the ability to initiate flowers under 18/6 (Bonferroni multi-test threshold: p=1.67E-06; Table 2). These 47 SNP markers are part of a locus which constitutes an association peak with SNP marker 171_988338 at position 1,088,206 bp on chromosome 8 being most significantly associated with the ability to initiate flowers under 18/6 (p=3.72E-18).

Subsequently, nested association mapping (NAM) was performed for Days to Flower Initiation in the set of 106 22ALV1 F2 accessions that were able to initiate flowers under 18/6. After application of QC filters (see for details the previous paragraph) 28,893 SNPs (missing data<15%) were available for mapping. NAM was performed using the R package NAM after missing data imputation as described above using seed lots as family structure and a kinship matrix to control for relatedness (GWAS2 function). In total, three SNP markers located between 159,096-555,854 bp on chromosome 8 of the Abacus reference genome were found to be significantly associated with Days to Flower Initiation (Bonferroni multi-test threshold: p=1.73E-06; Table 3). Since these three SNP markers were associated with similar p-values and because of their close proximity on the Abacus reference genome no distinction could be made regarding a singular main SNP marker associated with this trait, all three SNP markers are part of the locus for flower initiation.

For 22ALV2 Fisher Exact test of Flower Initiation was performed per SNP (n=299,12; SNPs were filtered for <10% missing data) similarly as for 22ALV1. In total, 21 SNP markers located between 109,674-2,816,933 bp on chromosome 8 of the Abacus reference genome (version Csat_AbacusV2; NCBI assembly accession GCA_025232715.1) were found to be significantly associated with the ability to initiate flowers under 18/6 (Bonferroni multi-test threshold: p=1.67E-06; Table 4). These 21 SNP markers are part of the same locus identified through 22ALV1.

For 23ALV1 Fisher Exact test of Flower Initiation was performed per SNP (n=38,039; SNPs were filtered for <25% missing data) similarly as for 22ALV1. In total, 101 SNP markers located between 19,681-3,794,786 bp on chromosome 8 of the Abacus reference genome (version Csat_AbacusV2; NCBI assembly accession GCA_025232715.1) were found to be significantly associated with the ability to initiate flowers under 18/6 (Flower Initiation trait; Bonferroni multi-test threshold: p=1.31E-06; Table 5). These 101 SNP markers are part of the same locus identified through 22ALV1 and 22ALV2. NAM of Days to Flower Initiation of 23ALV1 based on 99 accessions that were able to form flower clusters with at least six pistils (n=24,307 SNPs after filtering for <15% missing data) resulted in the identification of 17 significant SNP markers (Bonferroni multi-test threshold: p=2.06E-06; Table 6) between positions 241,017-1,376,341 bp on chromosome 8 of the Abacus reference genome (version Csat_AbacusV2; NCBI assembly accession GCA_025232715.1). Significant SNPs identified after Fisher Exact Test of Flower Initiation and NAM of Days to Flower Initiation were located at the same locus as identified in 22ALV1 and 22ALV2.

NAM of Days to Flower Initiation of the combined set of 246 accessions from 22ALV1, 22ALV2, and 23ALV1 that were able to form flower clusters with at least 6 pistils under 18/6 (n=31,289 SNPs after filtering for <15% missing data) resulted in the identification of 15 significant SNP markers (Bonferroni multi-test threshold: p=1.60E-06; Table 7) between positions 25,101-4,801,680 bp on chromosome 8 of the Abacus reference genome (version Csat_AbacusV2; NCBI assembly accession GCA_025232715.1). Significant SNPs were located at the same locus as identified in 22ALV1, 22ALV2, 23ALV1, 22TRP2, and 23TRP1.

For 22TRP2 Fisher Exact test was performed per SNP (n=10,672; SNPs were filtered for <10% missing data) based on accessions that were able to form flower clusters with at least six pistils at six Days to Flower Initiation After 12/12 Flip versus more than six Days to Flower Initiation After 12/12 Flip, similarly as for 22ALV1, 22ALV2, and 23ALV1. For 23TRP1 Fisher Exact test was performed per SNP (n=16,783; SNPs were filtered for <10% missing data) based on accessions that were able to form flower clusters with at least six pistils at 11 Days to Flower Initiation After 12/12 Flip versus more than 11 Days to Flower Initiation After 12/12 Flip, similarly as for 22ALV1, 22ALV2, 23ALV1, and 22TRP2. Combining p-values through multiplication across the two experiments which evaluated F2 populations derived from the same two mapping population parents (22TRP2 and 23TRP1) resulted in 327 significant SNP markers (Bonferroni multi-test threshold: p=1.82E-06) with a peak at the beginning of chromosome 8. Of those 327 SNP markers 31 (171_429974,171_219947,171_289630,171_319576,171_448590,171_477640,171_483960,133361_6612, 123467_5978,171_573381,171_606839,171_624259,171_636730,171_643517,171_648249,171_702968, 171_724002,171_745360,171_857167,171_908334,171_1008238,171_1082805,171_1190666, 171_1219675,171_1240501,171_1357163,171_1436811,171_1441354,171_1459473,171_1463835, and 171_11609) were also significantly associated with Flower Initiation and/or Days to Flower Initiation under 18/6 (marker with an \hat{ } in tables 2, 4, 5, 6, and 7). There were 18 SNP markers which were associated with Flower Initiation with a p-value<1.0E-07 (Table 8). These 18 SNP markers were located between 321,930-957,083 bp on chromosome 8 of the Abacus reference genome (version Csat_AbacusV2; NCBI assembly accession GCA_025232715.1) and are part of the same locus identified through 22ALV1, 22ALV2, and 23ALV1.

Table 9 provides a listing of the sequences of the present disclosure, which are located at position 51 of each respective sequence.

TABLE 2
Significant Fisher Exact test associations for Flower Initiation (=the ability
to initiate flowers) under 18/6 for the set of 147 22ALV1 F2 accessions.

| | | | | | | | | | Position | Position |
|---|---|---|---|---|---|---|---|---|---|---|
| | | | | | | | | | left | right |
| | | | Genotype | | | | Left | Right | flanking | flanking |
| | | | to | | | Abacus | flanking | flanking | SNP of | SNP of |
| | | | initiate | | | reference | SNP of | SNP of | marker | marker |
| | SNP marker | | flowers | Ref | Alt | genome | marker | marker | haplotype | haplotype |
| SEQ ID | name | p-value | under 18/6 | call | call | (bp) | haplotype | haplotype | (bp) | (bp) |
| SEQ ID | 171_6189 | 7.76E−12 | B, X | G | A | 19,681 | 171_6189* | 171_11609 | 0 | 25,101 |
| NO: 1 | | | | | | | | | | |
| SEQ ID | 171_31491 | 2.57E−10 | B, X | T | A | 44,981 | 171_11609 | 171_48451 | 25,101 | 63,750 |
| NO: 2 | | | | | | | | | | |
| SEQ ID | 171_38099 | 8.20E−11 | B, X | T | C | 51,589 | 171_11609 | 171_48451 | 25,101 | 63,750 |
| NO: 3 | | | | | | | | | | |
| SEQ ID | Cannabis.v1_scf1780- | 8.20E−11 | B, X | A | G | 87,904 | 142713_1935743 | 171_80245 | 82,833 | 95,593 |
| NO: 4 | 26461_101 | | | | | | | | | |
| SEQ ID | 171_143517 | 1.54E−14 | A, X | G | A | 159,096 | 171_130678 | 171_188787 | 146,257 | 209,857 |
| NO: 5 | | | | | | | | | | |
| SEQ ID | 171_178548 | 1.66E−11 | A, X | G | C | 199,126 | 171_130678 | 171_188787 | 146,257 | 209,857 |
| NO: 6 | | | | | | | | | | |
| SEQ ID | 171_339402 | 4.14E−16 | B, X | G | A | 371,700 | 171_328747 | 171_370217 | 361,045 | 401,990 |
| NO: 7 | | | | | | | | | | |
| SEQ ID | 171_346595 | 2.92E−09 | A, X | G | T | 378,368 | 171_328747 | 171_370217 | 361,045 | 401,990 |
| NO: 8 | | | | | | | | | | |
| SEQ ID | 171_353618 | 2.95E−07 | A, X | G | A | 385,390 | 171_328747 | 171_370217 | 361,045 | 401,990 |
| NO: 9 | | | | | | | | | | |
| SEQ ID | 171_399703 | 1.28E−07 | B, X | A | G | 447,673 | 149040_3510 | 171_429974 | 430,259 | 479,822 |
| NO: 10 | | | | | | | | | | |
| SEQ ID | 171_501931 | 1.82E−14 | A, X | T | C | 555,854 | 171_483960 | 171_504713 | 537,879 | 558,636 |
| NO: 11 | | | | | | | | | | |
| SEQ ID | 133361_2702 | 3.86E−14 | A, X | T | C | 564,634 | 171_504713 | 132292_12607 | 558,636 | 581,624 |
| NO: 12 | | | | | | | | | | |
| SEQ ID | 133361_6612 | 1.80E−08 | A, X | G | A | 568,544 | 171_504713 | 132292_12607 | 558,636 | 581,624 |
| NO: 13 | | | | | | | | | | |
| SEQ ID | 171_654206 | 3.28E−11 | A, X | T | G | 720,538 | 171_648249 | 171_694414 | 714,581 | 776,053 |
| NO: 14 | | | | | | | | | | |
| SEQ ID | Cannabis.v1_scf4041- | 9.97E−07 | A, X | A | T | 936,746 | 171_835887 | 171_838859 | 935,803 | 938,775 |
| NO: 15 | 1538_100 | | | | | | | | | |
| SEQ ID | 171_942768 | 2.98E−15 | A, X | G | A | 1,042,634 | 171_933239 | 171_960166 | 1,033,166 | 1,060,034 |
| NO: 16 | | | | | | | | | | |
| SEQ ID | 171_988338 | 3.72E−18 | B, X | T | C | 1,088,206 | 171_976341 | 171_1008238 | 1,076,208 | 1,108,104 |
| NO: 17 | | | | | | | | | | |
| SEQ ID | 171_1037290 | 5.92E−07 | A, X | C | T | 1,147,746 | 171_1024475 | 171_1043983 | 1,134,931 | 1,154,438 |
| NO: 18 | | | | | | | | | | |
| SEQ ID | 171_1080819 | 1.62E−16 | B, X | T | C | 1,191,275 | 171_1074370 | 171_1082805 | 1,184,825 | 1,193,261 |
| NO: 19 | | | | | | | | | | |
| SEQ ID | 171_1089602 | 7.27E−10 | A, X | G | A | 1,200,057 | 171_1082805 | 171_1091205 | 1,193,261 | 1,201,662 |
| NO: 20 | | | | | | | | | | |
| SEQ ID | 171_1100713 | 1.00E−11 | B, X | C | T | 1,211,168 | 171_1094760 | 171_1115181 | 1,205,216 | 1,225,668 |
| NO: 21 | | | | | | | | | | |
| SEQ ID | 171_1106446 | 2.32E−10 | A, X | T | C | 1,216,900 | 171_1094760 | 171_1115181 | 1,205,216 | 1,225,668 |
| NO: 22 | | | | | | | | | | |
| SEQ ID | 171_1108332 | 2.15E−10 | B, X | A | G | 1,218,786 | 171_1094760 | 171_1115181 | 1,205,216 | 1,225,668 |
| NO: 23 | | | | | | | | | | |
| SEQ ID | 171_1147276 | 7.14E−07 | B, X | G | T | 1,277,500 | un29717_60_61 | 171_1151293 | 1,273,758 | 1,281,517 |
| NO: 24 | | | | | | | | | | |
| SEQ ID | 171_1177247 | 1.08E−14 | X | T | C | 1,305,959 | 171_1164893 | 171_1190666 | 1,295,157 | 1,319,381 |
| NO: 25 | | | | | | | | | | |
| SEQ ID | 171_1261218 | 1.56E−11 | A, X | A | G | 1,397,056 | 171_1251819 | 171_1267576 | 1,387,657 | 1,403,412 |
| NO: 26 | | | | | | | | | | |
| SEQ ID | 171_1342861 | 1.99E−14 | A, X | A | G | 1,496,730 | 171_1320977 | 171_1349123 | 1,474,748 | 1,502,974 |
| NO: 27 | | | | | | | | | | |
| SEQ ID | 171_1361793 | 4.85E−08 | A, X | T | C | 1,515,643 | 171_1359330 | 171_1386128 | 1,513,180 | 1,539,957 |
| NO: 28 | | | | | | | | | | |
| SEQ ID | 171_1372205 | 3.37E−15 | A, X | A | T | 1,526,023 | 171_1359330 | 171_1386128 | 1,513,180 | 1,539,957 |
| NO: 29 | | | | | | | | | | |
| SEQ ID | 171_1418329 | 3.39E−13 | B, X | A | G | 1,577,268 | 129515_2372 | 171_1436811 | 1,557,156 | 1,595,751 |
| NO: 30 | | | | | | | | | | |
| SEQ ID | 171_1423189 | 7.14E−10 | B, X | G | T | 1,582,128 | 129515_2372 | 171_1436811 | 1,557,156 | 1,595,751 |
| NO: 31 | | | | | | | | | | |
| SEQ ID | 171_1458503 | 2.55E−13 | B, X | G | C | 1,617,208 | 171_1441354 | 171_1459473 | 1,600,294 | 1,618,178 |
| NO: 32 | | | | | | | | | | |
| SEQ ID | 171_1500507 | 4.19E−07 | B, X | A | C | 1,659,209 | 171_1497741 | 171_1518577 | 1,656,443 | 1,686,562 |
| NO: 33 | | | | | | | | | | |
| SEQ ID | 171_1522309 | 2.10E−08 | A, X | A | G | 1,690,294 | 171_1518577 | 171_1525133 | 1,686,562 | 1,693,118 |
| NO: 34 | | | | | | | | | | |
| SEQ ID | 171_1557279 | 1.66E−12 | A, X | G | A | 1,725,273 | 171_1546095 | 171_1589328 | 1,714,084 | 1,757,477 |
| NO: 35 | | | | | | | | | | |
| SEQ ID | 171_1561630 | 8.93E−10 | A, X | C | G | 1,729,625 | 171_1546095 | 171_1589328 | 1,714,084 | 1,757,477 |
| NO: 36 | | | | | | | | | | |
| SEQ ID | 171_1567853 | 3.79E−08 | B, X | T | G | 1,735,848 | 171_1546095 | 171_1589328 | 1,714,084 | 1,757,477 |
| NO: 37 | | | | | | | | | | |
| SEQ ID | 171_1647947 | 2.39E−07 | B, X | T | A | 1,816,071 | 171_1645831 | 171_1654428 | 1,813,955 | 1,822,552 |
| NO: 38 | | | | | | | | | | |
| SEQ ID | 171_1709761 | 1.20E−10 | B, X | A | C | 1,877,889 | 171_1698426 | Cannabis.v1_scf3841- | 1,866,553 | 1,881,061 |
| NO: 39 | | | | | | | | 21929_100 | | |
| SEQ ID | 171_1765646 | 5.81E−08 | A, X | C | A | 1,933,751 | 171_1759594 | 171_1776686 | 1,927,664 | 1,944,792 |
| NO: 40 | | | | | | | | | | |
| SEQ ID | 171_1821517 | 2.43E−09 | B, X | C | T | 1,993,648 | 171_1811417 | 171_1825490 | 1,983,548 | 1,997,626 |
| NO: 41 | | | | | | | | | | |
| SEQ ID | 171_1830089 | 2.79E−09 | B, X | C | A | 2,002,233 | 171_1825490 | 171_1865085 | 1,997,626 | 2,037,226 |
| NO: 42 | | | | | | | | | | |
| SEQ ID | 171_1848136 | 2.19E−07 | A, X | A | G | 2,020,279 | 171_1825490 | 171_1865085 | 1,997,626 | 2,037,226 |
| NO: 43 | | | | | | | | | | |
| SEQ ID | 171_2067142 | 1.51E−06 | A, X | C | A | 2,253,641 | 171_2059313 | 171_2071383 | 2,245,812 | 2,257,882 |
| NO: 44 | | | | | | | | | | |
| SEQ ID | 171_2092878 | 2.75E−09 | A, X | C | A | 2,279,386 | 171_2086176 | 171_2098637 | 2,272,681 | 2,285,144 |
| NO: 45 | | | | | | | | | | |
| SEQ ID | 171_2175812 | 4.16E−08 | B.X | A | G | 2,364,298 | 171_2172108 | 171_2180533 | 2,360,594 | 2,369,020 |
| NO: 46 | | | | | | | | | | |
| SEQ ID | 125705_984 | 6.63E−07 | B, X | A | G | 2,404,096 | 171_2213016 | 171_2217246 | 2,401,504 | 2,409,146 |
| NO: 47 | | | | | | | | | | |

First column, SNP marker number; Second column, SNP marker name,
\hat{ }= SNP marker is also significantly associated with Days to Flower Initiation after Flip under 12/12; Third column, Fisher Exact test p-value; Fourth column, beneficial genotype for ability to initiate flowers under 18/6 (A = homozygous for reference allele, B = homozygous for alternative allele, X = heterozygous); Fifth column, reference allele call; Sixth column, alternative allele call; Seventh column, Abacus reference genome position in bp on chromosome 8; Eighth column, left flanking SNP of haplotype surrounding SNP marker,,
*= SNP marker is the first on chromosome 8, therefore no left flanking marker other than the marker itself could be identified, the start of chromosome 8 is listed as the left flanking marker; Ninth column, right flanking SNP of haplotype surrounding SNP marker; Tenth column, Abacus reference genome position in bp for left flanking SNP of haplotype surrounding SNP marker; Eleventh column, Abacus reference genome position in bp for right flanking SNP of haplotype surrounding SNP marker.

TABLE 3
Significant NAM results for Days to Flower Initiation (=days after sow until flower initiation)
under 18/6 for the set of 106 22ALV1 F2 accessions which were able to initiate flowers under 18/6.

| | | | | | | Abacus | Left | Right | Position left | Position right |
|---|---|---|---|---|---|---|---|---|---|---|
| | | | | | | reference | flanking | flanking | flanking SNP | flanking SNP |
| | SNP | | | | | genome | SNP of | SNP of | of marker | of marker |
| | marker | | | Ref | Alt | position | marker | marker | haplotype | haplotype |
| SEQ ID | name | p-value | Genotype | call | call | (bp) | haplotype | haplotype | (bp) | (bp) |
| SEQ ID | 171_143517 | 3.16E−07 | A, X | G | A | 159,096 | 171_130678 | 171_178548 | 146,257 | 199,126 |
| NO: 5 | | | | | | | | | | |
| SEQ ID | 171_339402 | 3.16E−07 | B, X | G | A | 371,700 | 171_328747 | 171_346595 | 361,045 | 378,368 |
| NO: 7 | | | | | | | | | | |
| SEQ ID | 171_501931 | 3.98E−07 | A, X | T | C | 555,854 | 171_483960 | 171_504713 | 537,879 | 558,636 |
| NO: 11 | | | | | | | | | | |

First column, SNP marker number; Second column, SNP marker name; Third column, NAM p-value; Fourth column, beneficial genotype for early flowering under 18/6 (A = homozygous for reference allele, B = homozygous for alternative allele, X = heterozygous); Fifth column, reference allele call; Sixth column, alternative allele call; Seventh column, Abacus reference genome position in bp on chromosome 8; Eighth column, left flanking SNP of haplotype surrounding SNP marker; Ninth column, right flanking SNP of haplotype surrounding SNP marker; Tenth column, Abacus reference genome position in bp for left flanking SNP of haplotype surrounding SNP marker; Eleventh column, Abacus reference genome position in bp for right flanking SNP of haplotype surrounding SNP marker.

TABLE 4
Significant Fisher Exact test associations for Flower Initiation (=the ability to initiate flowers) under 18/6 for the set of 22ALV2 F2 accessions.

| | | | | | | | | | Position | Position |
|---|---|---|---|---|---|---|---|---|---|---|
| | | | | | | | | | left | right |
| | | | | | | Abacus | Left | Right | flanking | flanking |
| | | | | | | reference | flanking | flanking | SNP of | SNP of |
| | SNP | | | | | genome | SNP of | SNP of | marker | marker |
| | marker | | Geno- | Ref | Alt | position | marker | marker | haplotype | haplotype |
| SEQ ID | name | p-value | type | call | call | (bp) | haplotype | haplotype | (bp) | (bp) |
| SEQ ID | 171_94326 | 2.25E−09 | B, X | T | A | 109,674 | 171_86929 | 171_143517 | 102,277 | 159,096 |
| NO: 52 | | | | | | | | | | |
| SEQ ID | 171_346595 | 1.98E−08 | A, X | G | T | 378,368 | 171_319576 | 171_467459 | 351,874 | 517,316 |
| NO: 8 | | | | | | | | | | |
| SEQ ID | 171_429974\hat{ } | 1.43E−09 | A, X | T | C | 479,822 | 171_319576 | 171_467459 | 351,874 | 517,316 |
| NO: 60 | | | | | | | | | | |
| SEQ ID | 171_441217 | 1.14E−06 | A, X | T | A | 491,066 | 171_319576 | 171_467459 | 351,874 | 517,316 |
| NO: 61 | | | | | | | | | | |
| SEQ ID | 171_868913 | 2.2E−07 | A, X | T | A | 968,829 | 171_863359 | 171_873049 | 963,275 | 972,965 |
| NO: 80 | | | | | | | | | | |
| SEQ ID | 171_878186 | 2.2E−07 | A, X | A | G | 978,102 | 171_873049 | 171_908334 | 972,965 | 1,008,257 |
| NO: 82 | | | | | | | | | | |
| SEQ ID | 171_1024475 | 7E−09 | A, X | A | T | 1,134,931 | 171_978291 | 171_1080819 | 1,078,159 | 1,191,275 |
| NO: 88 | | | | | | | | | | |
| SEQ ID | 171_1043983 | 6.5E−09 | B, X | A | G | 1,154,438 | 171_978291 | 171_1080819 | 1,078,159 | 1,191,275 |
| NO: 89 | | | | | | | | | | |
| SEQ ID | un29717_60_61 | 2.58E−07 | B, X | C | T | 1,273,758 | 171_1117836 | 171_1147276 | 1,228,323 | 1,277,500 |
| NO: 93 | | | | | | | | | | |
| SEQ ID | 171_1567853 | 5.4E−07 | B, X | T | G | 1,735,848 | 171_1561630 | 171_1607775 | 1,729,625 | 1,772,967 |
| NO: 37 | | | | | | | | | | |
| SEQ ID | 171_1585716 | 1.68E−09 | B, X | T | G | 1,753,859 | 171_1561630 | 171_1607775 | 1,729,625 | 1,772,967 |
| NO: 105 | | | | | | | | | | |
| SEQ ID | 171_1676674 | 2.26E−08 | B, X | A | G | 1,844,801 | 171_1656647 | Cannabis.v1_{—} | 1,824,771 | 1,849,570 |
| NO: 112 | | | | | | | | scf2574- | | |
| | | | | | | | | 4059_101 | | |
| SEQ ID | 171_1704395 | 2.26E−08 | B, X | T | G | 1,872,522 | Cannabis.v1_{—} | 171_1728324 | 1,849,570 | 1,896,395 |
| NO: 113 | | | | | | | scf2574- | | | |
| | | | | | | | 4059_101 | | | |
| SEQ ID | 171_1776686 | 5.4E−07 | A, X | A | G | 1,944,792 | 171_1748030 | 171_1784261 | 1,916,102 | 1,956,392 |
| NO: 116 | | | | | | | | | | |
| SEQ ID | 171_1907774 | 1.18E−06 | B, X | T | G | 2,079,928 | 171_1907238 | 171_1915168 | 2,079,392 | 2,087,331 |
| NO: 123 | | | | | | | | | | |
| SEQ ID | Cannabis.v1_scf2769- | 7.84E−07 | A, X | A | G | 2,211,816 | 171_2012138 | 171_2102714 | 2,198,182 | 2,289,439 |
| NO: 129 | 57335_101 | | | | | | | | | |
| SEQ ID | 171_2080692 | 1.18E−06 | B, X | C | T | 2,267,196 | 171_2012138 | 171_2102714 | 2,198,182 | 2,289,439 |
| NO: 130 | | | | | | | | | | |
| SEQ ID | 171_2180533 | 9.91E−07 | B, X | G | A | 2,369,020 | 171_2151023 | Cannabis.v1_{—} | 2,339,395 | 2,373,428 |
| NO: 131 | | | | | | | | scf1512- | | |
| | | | | | | | | 73706_101 | | |
| SEQ ID | 171_2307670 | 3.53E−07 | B, X | A | G | 2,503,244 | 171_2265326 | 149699_7334 | 2,457,250 | 2,540,901 |
| NO: 133 | | | | | | | | | | |
| SEQ ID | 171_2315250 | 5.85E−08 | B, X | A | T | 2,510,824 | 171_2265326 | 149699_7334 | 2,457,250 | 2,540,901 |
| NO: 135 | | | | | | | | | | |
| SEQ ID | 171_2588227 | 8.05E−07 | B, X | C | T | 2,816,933 | 171_2584115 | 171_2596893 | 2,812,821 | 2,825,530 |
| NO: 136 | | | | | | | | | | |

First column, SNP marker number; Second column, SNP marker name,
\hat{ }= SNP marker is also significantly associated with Days to Flower Initiation after Flip under 12/12; Third column, Fisher Exact test p-value; Fourth column, beneficial genotype for early flowering under 18/6 (A = homozygous for reference allele, B = homozygous for alternative allele, X = heterozygous); Fifth column, reference allele call; Sixth column, alternative allele call; Seventh column, Abacus reference genome position in bp on chromosome 8; Eighth column, left flanking SNP of haplotype surrounding SNP marker; Ninth column, right flanking SNP of haplotype surrounding SNP marker; Tenth column, Abacus reference genome position in bp for left flanking SNP of haplotype surrounding SNP marker; Eleventh column, Abacus reference genome position in bp for right flanking SNP of haplotype surrounding SNP marker.

TABLE 5
Significant Fisher Exact test associations for Flower Initiation (=the ability to initiate flowers) under 18/6 for the set of 23ALV1 F2 accessions.

| | | | | | | | | | Position | Position |
|---|---|---|---|---|---|---|---|---|---|---|
| | | | | | | | | | left | right |
| | | | | | | Abacus | Left | Right | flanking | flanking |
| | | | | | | reference | flanking | flanking | SNP of | SNP of |
| | | | | | | genome | SNP of | SNP of | marker | marker |
| | SNP marker | | Geno- | Ref | Alt | position | marker | marker | haplotype | haplotype |
| SEQ ID | name | p-value | type | call | call | (bp) | haplotype | haplotype | (bp) | (bp) |
| SEQ ID NO: 1 | 171_6189 | 1.25E−09 | B, X | G | A | 19,681 | 171_6189* | 171_11609 | 0 | 25,101 |
| SEQ ID NO: 2 | 171_31491 | 1.12E−08 | B, X | T | A | 44,981 | 171_11609 | 171_38099 | 25,101 | 51,589 |
| SEQ ID NO: 49 | 171_48451 | 5.31E−09 | B, X | G | C | 63,750 | 171_38099 | 171_62360 | 51,589 | 77,659 |
| SEQ ID NO: 4 | Cannabis.v1_scf1780- | 1.25E−09 | B, X | A | G | 87,904 | 171_69402 | 171_98448 | 84,701 | 113,796 |
| | 26461_101 | | | | | | | | | |
| SEQ ID NO: 50 | 171_80245 | 1.25E−09 | B, X | T | C | 95,593 | Cannabis.v1_{—} | 171_85256 | 84,701 | 113,796 |
| | | | | | | | scf1780- | | | |
| | | | | | | | 26461_101 | | | |
| SEQ ID NO: 51 | 171_85256 | 5.43E−10 | B, X | C | T | 100,604 | 171_80245 | 171_94326 | 84,701 | 113,796 |
| SEQ ID NO: 52 | 171_94326 | 4.57E−10 | B, X | T | A | 109,674 | 171_85256 | 171_98448 | 84,701 | 113,796 |
| SEQ ID NO: 53 | 171_130678 | 3.52E−07 | A, X | A | C | 146,257 | 171_98448 | 171_137149 | 113,796 | 152,728 |
| SEQ ID NO: 5 | 171_143517 | 1.44E−19 | A, X | G | A | 159,096 | 171_137149 | 171_168508 | 152,728 | 189,086 |
| SEQ ID NO: 54 | 171_219947\hat{ } | 1.79E−13 | A, X | A | G | 241,017 | 171_215744 | 171_297723 | 236,814 | 330,022 |
| SEQ ID NO: 55 | 171_289630\hat{ } | 8.51E−21 | A, X | G | A | 321,930 | 171_215744 | 171_297723 | 236,814 | 330,022 |
| SEQ ID NO: 56 | 171_302378 | 1.85E−21 | A, X | T | A | 334,676 | 171_297723 | 171_339402 | 330,022 | 371,700 |
| SEQ ID NO: 57 | 171_319576\hat{ } | 6.58E−23 | A, X | A | T | 351,874 | 171_297723 | 171_339402 | 330,022 | 371,700 |
| SEQ ID NO: 58 | 171_363441 | 2.52E−19 | A, X | C | A | 395,213 | 171_353618 | 171_370217 | 385,390 | 401,990 |
| SEQ ID NO: 59 | 171_381570 | 7.67E−08 | B, X | A | G | 413,345 | 171_370217 | 149040_3510 | 401,990 | 430,259 |
| SEQ ID NO: 60 | 171_429974\hat{ } | 6.58E−23 | A, X | T | | 479,822 | 149040_3510 | 171_453600 | 430,259 | 503,453 |
| SEQ ID NO: 61 | 171_441217 | 3.23E−09 | A, X | T | A | 491,066 | 149040_3510 | 171_453600 | 430,259 | 503,453 |
| SEQ ID NO: 62 | 171_448590\hat{ } | 2.47E−20 | B, X | C | T | 498,442 | 149040_3510 | 171_453600 | 430,259 | 503,453 |
| SEQ ID NO: 63 | 171_462731 | 8.82E−09 | B, X | G | A | 512,584 | 171_453600 | 171_501931 | 503,453 | 555,854 |
| SEQ ID NO: 64 | 171_477640\hat{ } | 6.69E−22 | A, X | A | C | 531,560 | 171_453600 | 171_501931 | 503,453 | 555,854 |
| SEQ ID NO: 65 | 171_483960\hat{ } | 2.63E−22 | A, X | A | G | 537,879 | 171_453600 | 171_501931 | 503,453 | 555,854 |
| SEQ ID NO: 13 | 133361_6612\hat{ } | 7.52E−18 | B, X | G | A | 568,544 | 133361_2702 | 132292_12607 | 564,634 | 581,624 |
| SEQ ID NO: 66 | 123467_5978\hat{ } | 3.86E−17 | B, X | G | A | 606,518 | 132292_5522 | 171_565345 | 598,071 | 631,105 |
| SEQ ID NO: 67 | 171_573381\hat{ } | 1.21E−15 | A, X | A | G | 639,258 | 171_565345 | 171_600372 | 631,105 | 666,249 |
| SEQ ID NO: 68 | 171_606839\hat{ } | 6.03E−17 | B, X | G | A | 673,171 | 171_600372 | 171_619793 | 666,249 | 686,125 |
| SEQ ID NO: 69 | 171_624259\hat{ } | 2.84E−17 | A, X | C | T | 690,591 | 171_619793 | 142235_4818 | 686,125 | 759,657 |
| SEQ ID NO: 70 | 171_636730\hat{ } | 1.37E−16 | A, X | A | C | 703,062 | 171_619793 | 142235_4818 | 686,125 | 759,657 |
| SEQ ID NO: 71 | 171_643517\hat{ } | 1.26E−16 | B, X | A | C | 709,849 | 171_619793 | 142235_4818 | 686,125 | 759,657 |
| SEQ ID NO: 72 | 171_648249\hat{ } | 1.06E−16 | B, X | G | A | 714,581 | 171_619793 | 142235_4818 | 686,125 | 759,657 |
| SEQ ID NO: 14 | 171_654206 | 7.14E−16 | A, X | T | G | 720,538 | 171_619793 | 142235_4818 | 686,125 | 759,657 |
| SEQ ID NO: 73 | 171_702968\hat{ } | 1.35E−15 | A, X | G | A | 784,607 | 142235_4818 | 171_714874 | 759,657 | 796,514 |
| SEQ ID NO: 74 | 171_724002\hat{ } | 5.80E−18 | A, X | C | T | 805,642 | 171_720138 | 171_731565 | 801,778 | 813,205 |
| SEQ ID NO: 75 | 171_745360\hat{ } | 5.80E−18 | A, X | T | C | 827,002 | 171_731565 | 171_758485 | 813,205 | 840,127 |
| SEQ ID NO: 76 | 171_778207 | 5.30E−12 | A, X | G | A | 859,843 | 171_774312 | 171_787715 | 855,948 | 869,351 |
| SEQ ID NO: 77 | 171_835887 | 4.09E−12 | A, X | A | G | 935,803 | 171_817649 | Cannabis.v1_{—} | 917,565 | 936,746 |
| | | | | | | | | scf4041- | | |
| | | | | | | | | 1538_100 | | |
| SEQ ID NO: 78 | 171_857167\hat{ } | 4.47E−16 | B, X | T | A | 957,083 | 171_849220 | 171_878186 | 949,136 | 978,102 |
| SEQ ID NO: 79 | 171_863359 | 5.30E−12 | A, X | C | T | 963,275 | 171_849220 | 171_878186 | 949,136 | 978,102 |
| SEQ ID NO: 80 | 171_868913 | 5.30E−12 | A, X | T | A | 968,829 | 171_849220 | 171_878186 | 949,136 | 978,102 |
| SEQ ID NO: 81 | 171_873049 | 3.72E−12 | A, X | C | A | 972,965 | 171_849220 | 171_878186 | 949,136 | 978,102 |
| SEQ ID NO: 83 | 171_908334\hat{ } | 1.39E−17 | A, X | T | A | 1,008,257 | 171_899982 | 171_912691 | 999,905 | 1,012,614 |
| SEQ ID NO: 84 | 171_948145 | 2.48E−12 | A, X | C | T | 1,048,013 | 171_942768 | 171_952667 | 1,042,634 | 1,052,535 |
| SEQ ID NO: 85 | 171_976341 | 2.12E−10 | A, X | G | T | 1,076,208 | 171_960166 | 171_988338 | 1,060,034 | 1,088,206 |
| SEQ ID NO: 86 | 171_978291 | 7.39E−11 | A, X | A | G | 1,078,159 | 171_960166 | 171_988338 | 1,060,034 | 1,088,206 |
| SEQ ID NO: 87 | 171_1008238\hat{ } | 1.37E−16 | B, X | G | A | 1,108,104 | 171_988338 | 171_1024475 | 1,088,206 | 1,134,931 |
| SEQ ID NO: 90 | 171_1052176 | 8.22E−08 | B, X | C | G | 1,162,631 | 171_1043983 | 171_1074370 | 1,154,438 | 1,184,825 |
| SEQ ID NO: 19 | 171_1080819 | 8.22E−08 | A, X | T | C | 1,191,275 | 171_1074370 | 171_1091205 | 1,184,825 | 1,201,662 |
| SEQ ID NO: 91 | 171 1082805 | 2.98E−17 | A, X | G | A | 1,193,261 | 171 1074370 | 171_1091205 | 1,184,825 | 1,201,662 |
| SEQ ID NO: 20 | 171_1089602 | 3.98E−07 | A, X | G | A | 1,200,057 | 171_1074370 | 171_1091205 | 1,184,825 | 1,201,662 |
| SEQ ID NO: 21 | 171_1100713 | 1.37E−16 | A, X | C | T | 1,211,168 | 171_1094760 | 171_1106446 | 1,205,216 | 1,216,900 |
| SEQ ID NO: 92 | 171_1117836 | 9.18E−08 | B, X | T | C | 1,228,323 | 171_1115181 | 137390_1244 | 1,225,668 | 1,257,300 |
| SEQ ID NO: 93 | un29717_60_61 | 1.21E−06 | B, X | C | T | 1,273,758 | 137390_1244 | 171_1147276 | 1,257,300 | 1,277,500 |
| SEQ ID NO: 24 | 171_1147276 | 1.07E−08 | A, X | G | T | 1,277,500 | un29717_60_61 | 171_1156042 | 1,273,758 | 1,286,264 |
| SEQ ID NO: 94 | 171_1151293 | 1.20E−08 | A, X | A | C | 1,281,517 | un29717_6061 | 171_1156042 | 1,273,758 | 1,286,264 |
| SEQ ID NO: 25 | 171_1177247 | 4.76E−15 | B, X* | T | C | 1,305,959 | 171_1173237 | 171_1184007 | 1,301,949 | 1,312,722 |
| SEQ ID NO: 95 | 171_1190666\hat{ } | 5.77E−16 | B, X | G | A | 1,319,381 | 171_1184007 | 171_1207043 | 1,312,722 | 1,342,980 |
| SEQ ID NO: 96 | 171_1219675\hat{ } | 2.73E−16 | B, X | A | G | 1,355,612 | Cannabis.v1_{—} | 171_1230531 | 1,354,996 | 1,366,469 |
| | | | | | | | scf102- | | | |
| | | | | | | | 278325_101 | | | |
| SEQ ID NO: 97 | 171_1240501\hat{ } | 1.21E−15 | B, X | A | G | 1,376,341 | 171_1230531 | 171_1251819 | 1,366,469 | 1,387,657 |
| SEQ ID NO: 98 | 171_1357163\hat{ } | 1.21E−15 | A, X | C | T | 1,511,014 | 171_1349123 | 171_1359330 | 1,502,974 | 1,513,180 |
| SEQ ID NO: 99 | 129515_2372 | 2.73E−11 | A, X | G | T | 1,557,156 | 171_1397805 | 171_1418329 | 1,551,663 | 1,577,268 |
| SEQ ID NO: 100 | 171_1436811\hat{ } | 2.23E−15 | A, X | A | G | 1,595,751 | 171_1434408 | 171_1458503 | 1,593,348 | 1,617,208 |
| SEQ ID NO: 101 | 171_1441354\hat{ } | 1.21E−15 | A, X | A | G | 1,600,294 | 171_1434408 | 171_1458503 | 1,593,348 | 1,617,208 |
| SEQ ID NO: 102 | 171_1459473\hat{ } | 1.84E−14 | A, X | T | C | 1,618,178 | 171_1458503 | 171_1469787 | 1,617,208 | 1,628,490 |
| SEQ ID NO: 103 | 171_1463835\hat{ } | 4.96E−14 | A, X | G | A | 1,622,540 | 171_1458503 | 171_1469787 | 1,617,208 | 1,628,490 |
| SEQ ID NO: 33 | 171_1500507 | 4.13E−14 | B, X | A | C | 1,659,209 | 171_1497741 | 171_1518577 | 1,656,443 | 1,686,562 |
| SEQ ID NO: 34 | 171_1522309 | 1.36E−08 | A, X | A | G | 1,690,294 | 171_1518577 | 171_1525133 | 1,686,562 | 1,693,118 |
| SEQ ID NO: 104 | 171_1539058 | 4.12E−08 | A, X | T | C | 1,707,047 | 171_1525133 | Cannabis.v1_{—} | 1,693,118 | 1,710,154 |
| | | | | | | | | scf3448- | | |
| | | | | | | | | 27829_100 | | |
| SEQ ID NO: 106 | 171_1615710 | 1.37E−16 | B, X | A | G | 1,783,885 | 171_1607775 | 171_1628231 | 1,772,967 | 1,796,406 |
| SEQ ID NO: 107 | 171_1622388 | 2.23E−15 | B, X | A | G | 1,790,563 | 171_1607775 | 171_1628231 | 1,772,967 | 1,796,406 |
| SEQ ID NO: 108 | 171_1634072 | 2.88E−15 | A, X | A | G | 1,802,247 | 171_1632871 | 171_1638524 | 1,801,046 | 1,806,699 |
| SEQ ID NO: 109 | 171_1642347 | 8.01E−15 | B, X | C | A | 1,810,522 | 171_1638524 | 171_1647947 | 1,806,699 | 1,813,955 |
| SEQ ID NO: 110 | 171_1645831 | 2.70E−14 | A, X | C | A | 1,813,955 | 171_1638524 | 171_1647947 | 1,810,522 | 1,816,071 |
| SEQ ID NO: 38 | 171_1647947 | 1.15E−07 | B, X | T | A | 1,816,071 | 171_1645831 | 171_1654428 | 1,813,955 | 1,822,552 |
| SEQ ID NO: 111 | 171_1667193 | 2.70E−14 | A, X | C | G | 1,835,318 | 171_1661819 | 171_1676674 | 1,829,943 | 1,844,801 |
| SEQ ID NO: 113 | 171_1704395 | 2.10E−14 | B, X | T | G | 1,872,522 | Cannabis.v1_{—} | 171_1709761 | 1,849,570 | 1,877,889 |
| | | | | | | | scf2574- | | | |
| | | | | | | | 4059_101 | | | |
| SEQ ID NO: 114 | 171_1741089 | 4.42E−07 | B, X | C | T | 1,909,161 | 171_1737356 | 171_1743902 | 1,905,430 | 1,911,974 |
| SEQ ID NO: 115 | 171_1743902 | 6.58E−08 | B, X | A | G | 1,911,974 | 171_1741089 | 171_1748030 | 1,909,161 | 1,916,102 |
| SEQ ID NO: 117 | 171_1784261 | 1.36E−08 | A, X | G | A | 1,956,392 | 171_1780817 | 171_1790914 | 1,948,922 | 1,963,045 |
| SEQ ID NO: 118 | 171_1798950 | 3.54E−08 | A, X | C | T | 1,971,081 | 171_1790914 | 171_1801885 | 1,963,045 | 1,974,016 |
| SEQ ID NO: 119 | 171_1801885 | 3.54E−08 | A, X | T | C | 1,974,016 | 171_1798950 | 171_1811417 | 1,971,081 | 1,983,548 |
| SEQ ID NO: 41 | 171_1821517 | 1.84E−14 | B, X | C | T | 1,993,648 | 171_1811417 | 171_1865085 | 1,983,548 | 2,037,226 |
| SEQ ID NO: 120 | 171_1825490 | 1.44E−14 | A, X | T | C | 1,997,626 | 171_1811417 | 171_1865085 | 1,983,548 | 2,037,226 |
| SEQ ID NO: 42 | 171 1830089 | 6.39E−14 | B, X | C | A | 2,002,233 | 171 1811417 | 171_1865085 | 1,983,548 | 2,037,226 |
| SEQ ID NO: 43 | 171_1848136 | 3.01E−14 | A, X | A | G | 2,020,279 | 171_1811417 | 171_1865085 | 1,983,548 | 2,037,226 |
| SEQ ID NO: 121 | 171_1889481 | 1.84E−14 | B, X | T | A | 2,061,626 | 171_1875695 | 171_1895548 | 2,047,839 | 2,067,693 |
| SEQ ID NO: 122 | 171_1907238 | 1.36E−08 | B, X | T | C | 2,079,392 | 171_1895548 | 171_1934495 | 2,067,693 | 2,106,657 |
| SEQ ID NO: 123 | 171_1907774 | 3.48E−09 | B, X | T | G | 2,079,928 | 171_1895548 | 171_1934495 | 2,067,693 | 2,106,657 |
| SEQ ID NO: 124 | 171_1915168 | 1.78E−08 | A, X | T | G | 2,087,331 | 171_1895548 | 171_1934495 | 2,067,693 | 2,106,657 |
| SEQ ID NO: 125 | 171_1927464 | 1.79E−12 | B, X* | T | C | 2,099,627 | 171_1895548 | 171_1934495 | 2,067,693 | 2,106,657 |
| SEQ ID NO: 126 | 171_1929444 | 1.78E−08 | B, X | A | T | 2,101,607 | 171_1895548 | 171_1934495 | 2,067,693 | 2,106,657 |
| SEQ ID NO: 127 | 171_1949435 | 4.73E−08 | A, X | G | A | 2,127,039 | 171_1941961 | 171_1952324 | 2,114,122 | 2,129,927 |
| SEQ ID NO: 128 | 171_1974687 | 3.54E−08 | B, X | G | A | 2,160,732 | 171_1959081 | 171_1981946 | 2,145,070 | 2,167,991 |
| SEQ ID NO: 130 | 171_2080692 | 5.70E−08 | B, X | C | T | 2,267,196 | 171_2071383 | 171_2086176 | 2,257,882 | 2,272,681 |
| SEQ ID NO: 132 | 171_2195254 | 1.43E−09 | B, X | T | C | 2,383,741 | 171_2189445 | 171_2203055 | 2,377,932 | 2,391,542 |
| SEQ ID NO: 134 | un245831_73_74 | 4.42E−07 | A, X | A | G | 2,504,080 | 171_2307670 | 171_2315250 | 2,503,244 | 2,510,824 |
| SEQ ID NO: 137 | 171_2599642 | 1.46E−07 | B, X | A | G | 2,828,279 | 171_2596893 | 171_2652789 | 2,825,530 | 2,881,461 |
| SEQ ID NO: 138 | 171_2604364 | 2.17E−08 | B, X | G | A | 2,833,001 | 171_2596893 | 171_2652789 | 2,825,530 | 2,881,461 |
| SEQ ID NO: 139 | 171_2617799 | 5.37E−08 | A, X | C | G | 2,846,467 | 171_2596893 | 171_2652789 | 2,825,530 | 2,881,461 |
| SEQ ID NO: 140 | 171_2633941 | 6.65E−09 | A, X | T | A | 2,862,613 | 171_2596893 | 171_2652789 | 2,825,530 | 2,881,461 |
| SEQ ID NO: 141 | Cannabis.v1_scf1670- | 2.99E−08 | B, X | C | T | 2,912,209 | 171_2679500 | 171_2705673 | 2,908,180 | 2,934,321 |
| | 75982_101 | | | | | | | | | |
| SEQ ID NO: 142 | 171_2867392 | 1.35E−07 | B, X | C | T | 3,098,435 | 171_2859306 | 171_2888104 | 3,090,350 | 3,118,845 |
| SEQ ID NO: 143 | 171_3528035 | 3.12E−07 | B, X | T | G | 3,794,786 | 171_3509913 | 171_3530863 | 3,776,664 | 3,797,614 |

First column, SNP marker number; Second column, SNP marker name,
\hat{ }= SNP marker is also significantly associated with Days to Flower Initiation after Flip under 12/12; Third column, Fisher Exact test p-value; Fourth column, beneficial genotype for early flowering under 18/6 (A = homozygous for reference allele, B = homozygous for alternative allele, X = heterozygous),
*= B inferred based on segregation patterns; Fifth column, reference allele call; Sixth column, alternative allele call; Seventh column, Abacus reference genome position in bp on chromosome 8; Eight column, left flanking SNP of haplotype surrounding SNP marker,
*= SNP marker is the first on chromosome 8, therefore no left flanking marker other than the marker itself could be identified, the start of chromosome 8 is listed as the left flanking marker; Ninth column, right flanking SNP of haplotype surrounding SNP marker; Tenth column, Abacus reference genome position in bp for left flanking SNP of haplotype surrounding SNP marker; Eleventh column, Abacus reference genome position in bp for right flanking SNP of haplotype surrounding SNP marker.

TABLE 6
Significant NAM associations for Days to Flower Initiation under 18/6 for the set of 23ALV1
F2 accessions that were able to form clusters of flowers with at least six pistils.

| | | | | | | Abacus | Left | Right | Position left | Position right |
|---|---|---|---|---|---|---|---|---|---|---|
| | | | | | | reference | flanking | flanking | flanking SNP | flanking SNP |
| | SNP | | | | | genome | SNP of | SNP of | of marker | of marker |
| | marker | | Geno- | Ref | Alt | position | marker | marker | haplotype | haplotype |
| SEQ ID | name | p-value | type | call | call | (bp) | haplotype | haplotype | (bp) | (bp) |
| SEQ ID NO: 54 | 171_219947\hat{ } | 2.04E−09 | A, X | A | G | 241,017 | 171_178548 | 171_297723 | 199,126 | 330,022 |
| SEQ ID NO: 55 | 171_289630\hat{ } | 9.55E−09 | A, X | G | A | 321,930 | 171_178548 | 171_297723 | 199,126 | 330,022 |
| SEQ ID NO: 56 | 171_302378 | 1.00E−06 | A, X | T | A | 334,676 | 171_297723 | 171_328747 | 330,022 | 361,045 |
| SEQ ID NO: 57 | 171_319576\hat{ } | 9.55E−09 | A, X | A | T | 351,874 | 171_297723 | 171_328747 | 330,022 | 361,045 |
| SEQ ID NO: 60 | 171_429974\hat{ } | 1.41E−06 | A, X | T | C | 479,822 | 171_399703 | 171_441217 | 447,673 | 491,066 |
| SEQ ID NO: 62 | 171_448590\hat{ } | 6.17E−07 | B, X | C | T | 498,442 | 171_441217 | 171_462731 | 491,066 | 512,584 |
| SEQ ID NO: 64 | 171_477640\hat{ } | 1.17E−06 | A, X | A | C | 531,560 | 171_467459 | 133361_2702 | 517,316 | 564,634 |
| SEQ ID NO: 65 | 171_483960\hat{ } | 8.51E−07 | A, X | A | G | 537,879 | 171_467459 | 133361_2702 | 517,316 | 564,634 |
| SEQ ID NO: 66 | 123467_5978\hat{ } | 1.70E−06 | B, X | G | A | 606,518 | 133361_6612 | 171_565345 | 568,544 | 631,105 |
| SEQ ID NO: 67 | 171_573381\hat{ } | 2.45E−07 | A, X | A | G | 639,258 | 171_565345 | 171_585500 | 631,105 | 651,377 |
| SEQ ID NO: 68 | 171_606839\hat{ } | 6.92E−07 | B, X | G | A | 673,171 | 171_585500 | 171_619793 | 651,377 | 686,125 |
| SEQ ID NO: 70 | 171_636730\hat{ } | 1.55E−06 | A, X | A | C | 703,062 | 171_624259 | 142235_4818 | 690,591 | 759,657 |
| SEQ ID NO: 71 | 171_643517\hat{ } | 2.40E−07 | B, X | A | C | 709,849 | 171_624259 | 142235_4818 | 690,591 | 759,657 |
| SEQ ID NO: 72 | 171_648249\hat{ } | 1.55E−06 | B, X | G | A | 714,581 | 171_624259 | 142235_4818 | 690,591 | 759,657 |
| SEQ ID NO: 73 | 171_702968\hat{ } | 5.13E−08 | A, X | G | A | 784,607 | 171_698486 | 171_724002 | 780,125 | 805,642 |
| SEQ ID NO: 95 | 171_1190666\hat{ } | 1.66E−06 | B, X | G | A | 1,319,381 | 171_1184007 | 171_1207043 | 1,312,722 | 1,342,980 |
| SEQ ID NO: 97 | 171_1240501\hat{ } | 1.23E−06 | B, X | A | G | 1,376,341 | 171_1219675 | 171_1251819 | 1,355,612 | 1,387,657 |

First column, SNP marker number; Second column, SNP marker name,
\hat{ }= SNP marker is also significantly associated with Days to Flower Initiation after Flip under 12/12; Third column, Fisher Exact test p-value; Fourth column, beneficial genotype for early flowering under 18/6 (A = homozygous for reference allele, B = homozygous for alternative allele, X = heterozygous); Fifth column, reference allele call; Sixth column, alternative allele call; Seventh column, Abacus reference genome position in bp on chromosome 8; Eight column, left flanking SNP of haplotype surrounding SNP marker; Ninth column, right flanking SNP of haplotype surrounding SNP marker; Tenth column, Abacus reference genome position in bp for left flanking SNP of haplotype surrounding SNP marker; Eleventh column, Abacus reference genome position in bp for right flanking SNP of haplotype surrounding SNP marker.

TABLE 7
Significant NAM associations for Days to Flower Initiation under 18/6 for the combined set of 22ALV1,
22ALV2, and 23ALV1 F2 accessions that were able to form clusters of flowers with at least 6 pistils.

| | | | | | | | | | Position | Position |
|---|---|---|---|---|---|---|---|---|---|---|
| | | | | | | | | | left | right |
| | | | | | | Abacus | Left | Right | flanking | flanking |
| | | | | | | reference | flanking | flanking | SNP of | SNP of |
| | | | | | | genome | SNP of | SNP of | marker | marker |
| | SNP marker | | Geno- | Ref | Alt | position | marker | marker | haplotype | haplotype |
| SEQ ID | name | p-value | type | call | call | (bp) | haplotype | haplotype | (bp) | (bp) |
| SEQ ID NO: | 171_11609\hat{ } | 2.75E−07 | A, X | G | A | 25,101 | 171_6189 | 171_20281 | 19,681 | 33,771 |
| 48 | | | | | | | | | | |
| SEQ ID NO: | 171_219947\hat{ } | 1.58E−11 | A, X | A | G | 241,017 | 171_215744 | 171_289630 | 236,814 | 321,930 |
| 54 | | | | | | | | | | |
| SEQ ID NO: | 171_319576\hat{ } | 2.95E−11 | A, X | A | T | 351,874 | 171_297723 | 171_328747 | 330,022 | 361,045 |
| 57 | | | | | | | | | | |
| SEQ ID NO: | 171_429974\hat{ } | 1.78E−08 | A, X | T | C | 479,822 | 171_399703 | 171_441217 | 447,673 | 491,066 |
| 60 | | | | | | | | | | |
| SEQ ID NO: | 171_477640\hat{ } | 5.13E−07 | A, X | A | C | 531,560 | 171_467459 | 171_504713 | 517,316 | 558,636 |
| 64 | | | | | | | | | | |
| SEQ ID NO: | 171_483960\hat{ } | 7.59E−08 | A, X | A | G | 537,879 | 171_467459 | 171_504713 | 517,316 | 558,636 |
| 65 | | | | | | | | | | |
| SEQ ID NO: | 123467_5978\hat{ } | 8.13E−10 | B, X | G | A | 606,518 | 132292_5522 | 171_619793 | 598,071 | 686,125 |
| 66 | | | | | | | | | | |
| SEQ ID NO: | 171_573381\hat{ } | 3.16E−08 | A, X | A | G | 639,258 | 132292_5522 | 171_619793 | 598,071 | 686,125 |
| 67 | | | | | | | | | | |
| SEQ ID NO: | 171_606839\hat{ } | 3.55E−08 | B, X | G | A | 673,171 | 132292_5522 | 171_619793 | 598,071 | 686,125 |
| 68 | | | | | | | | | | |
| SEQ ID NO: | 171_702968\hat{ } | 2.34E−09 | A, X | G | A | 784,607 | 171_698486 | 171_714874 | 780,125 | 796,514 |
| 73 | | | | | | | | | | |
| SEQ ID NO: | 171_724002\hat{ } | 3.98E−07 | A, X | C | T | 805,642 | 171_714874 | 171_731565 | 796,514 | 813,205 |
| 74 | | | | | | | | | | |
| SEQ ID NO: | 171_1082805\hat{ } | 6.31E−08 | B, X | G | A | 1,193,261 | 171_1080819 | 171_1089602 | 1,191,275 | 1,200,057 |
| 91 | | | | | | | | | | |
| SEQ ID NO: | 171_1219675\hat{ } | 1.48E−06 | B, X | A | G | 1,355,612 | Cannabis.v1_scf102- | 171_1251819 | 1,354,996 | 1,387,657 |
| 96 | | | | | | | 278325_101 | | | |
| SEQ ID NO: | 171_1240501\hat{ } | 4.57E−07 | B, X | A | G | 1,376,341 | Cannabis.v1_scf102- | 171_1251819 | 1,354,996 | 1,387,657 |
| 97 | | | | | | | 278325_101 | | | |
| SEQ ID NO: | 171_4503268\hat{ } | 1.20E−06 | B, X | T | C | 4,801,680 | 171_4490650 | 171_4510724 | 4,785,390 | 4,809,136 |
| 144 | | | | | | | | | | |

First column, SNP marker number; Second column, SNP marker name,
\hat{ }= SNP marker is also significantly associated with Days to Flower Initiation after Flip under 12/12; Third column, Fisher Exact test p-value; Fourth column, beneficial genotype for early flowering under 18/6 (A = homozygous for reference allele, B = homozygous for alternative allele, X = heterozygous); Fifth column, reference allele call; Sixth column, alternative allele call; Seventh column, Abacus reference genome position in bp on chromosome 8; Eight column, left flanking SNP of haplotype surrounding SNP marker; Ninth column, right flanking SNP of haplotype surrounding SNP marker; Tenth column, Abacus reference genome position in bp for left flanking SNP of haplotype surrounding SNP marker; Eleventh column, Abacus reference genome position in bp for right flanking SNP of haplotype surrounding SNP marker.

TABLE 8
Significant Fisher Exact test associations for early formation of clusters of flowers with at
least six pistils for the combined sets of 22TRP2 and 23TRP1 accessions evaluated under 12/12.

| | | | | | | Abacus | Left | Right | Position left | Position right |
|---|---|---|---|---|---|---|---|---|---|---|
| | | | | | | reference | flanking | flanking | flanking SNP | flanking SNP |
| | | | | | | genome | SNP of | SNP of | of marker | of marker |
| | SNP marker | | Geno- | Ref | Alt | position | marker | marker | haplotype | haplotype |
| SEQ ID | name | p-value | type | call | call | (bp) | haplotype | haplotype | (bp) | (bp) |
| SEQ ID NO: | 171_289630 | 1.04E−09 | A, X | G | A | 321,930 | 171_219947 | 171_297723 | 241,017 | 330,022 |
| 55 | | | | | | | | | | |
| SEQ ID NO: | 171_319576 | 2.29E−09 | A, X | A | T | 351,874 | 171_302378 | 171_346595 | 334,676 | 378,368 |
| 57 | | | | | | | | | | |
| SEQ ID NO: | 171_429974 | 2.29E−09 | A, X | T | C | 479,822 | 171_381570 | 171_441217 | 413,345 | 491,066 |
| 60 | | | | | | | | | | |
| SEQ ID NO: | 171_448590 | 5.83E−09 | B, X | C | T | 498,442 | 171_441217 | 171_462731 | 491,066 | 512,584 |
| 62 | | | | | | | | | | |
| SEQ ID NO: | 171_477640 | 5.43E−09 | A, X | A | C | 531,560 | 171_467459 | 133361_2702 | 517,316 | 564,634 |
| 64 | | | | | | | | | | |
| SEQ ID NO: | 171_483960 | 2.31E−09 | A, X | A | G | 537,879 | 171_467459 | 133361_2702 | 517,316 | 564,634 |
| 65 | | | | | | | | | | |
| SEQ ID NO: | 133361_6612 | 2.13E−09 | B, X | G | A | 568,544 | 133361_2702 | 171_619793 | 564,634 | 686,125 |
| 13 | | | | | | | | | | |
| SEQ ID NO: | 123467_5978 | 6.62E−09 | B, X | G | A | 606,518 | 133361_2702 | 171_619793 | 564,634 | 686,125 |
| 66 | | | | | | | | | | |
| SEQ ID NO: | 171_573381 | 5.83E−09 | A, X | A | G | 639,258 | 133361_2702 | 171_619793 | 564,634 | 686,125 |
| 67 | | | | | | | | | | |
| SEQ ID NO: | 171_606839 | 1.14E−09 | B, X | G | A | 673,171 | 133361_2702 | 171_619793 | 564,634 | 686,125 |
| 68 | | | | | | | | | | |
| SEQ ID NO: | 171_624259 | 2.09E−09 | A, X | C | T | 690,591 | 171_619793 | 171_731565 | 686,125 | 813,205 |
| 69 | | | | | | | | | | |
| SEQ ID NO: | 171_636730 | 2.90E−09 | A, X | A | C | 703,062 | 171_619793 | 171_731565 | 686,125 | 813,205 |
| 70 | | | | | | | | | | |
| SEQ ID NO: | 171_643517 | 1.12E−08 | B, X | A | C | 709,849 | 171_619793 | 171_731565 | 686,125 | 813,205 |
| 71 | | | | | | | | | | |
| SEQ ID NO: | 171_648249 | 2.54E−08 | B, X | G | A | 714,581 | 171_619793 | 171_731565 | 686,125 | 813,205 |
| 72 | | | | | | | | | | |
| SEQ ID NO: | 171_702968 | 1.20E−08 | A, X | G | A | 784,607 | 171_619793 | 171_731565 | 686,125 | 813,205 |
| 73 | | | | | | | | | | |
| SEQ ID NO: | 171_724002 | 3.11E−08 | A, X | C | T | 805,642 | 171_619793 | 171_731565 | 686,125 | 813,205 |
| 74 | | | | | | | | | | |
| SEQ ID NO: | 171_745360 | 1.69E−09 | A, X | T | C | 827,002 | 171_731565 | 171_774312 | 813,205 | 855,948 |
| 75 | | | | | | | | | | |
| SEQ ID NO: | 171_857167 | 2.42E−08 | B, X | T | A | 957,083 | 171_849220 | 171_863359 | 949,136 | 963,275 |
| 78 | | | | | | | | | | |

First column, SNP marker number; Second column, SNP marker name; Third column, Fisher Exact test combined p-value; Fourth column, beneficial genotype for early flowering under 12/12 (A = homozygous for reference allele, B = homozygous for alternative allele, X = heterozygous); Fifth column, reference allele call; Sixth column, alternative allele call; Seventh column, Abacus reference genome position in bp on chromosome 8; Eighth column, left flanking SNP of haplotype surrounding SNP marker; Ninth column, right flanking SNP of haplotype surrounding SNP marker; Tenth column, Abacus reference genome position in bp for left flanking SNP of haplotype surrounding SNP marker; Eleventh column, Abacus reference genome position in bp for right flanking SNP of haplotype surrounding SNP marker.

TABLE 9
50 bp flanking sequences surrounding SNP markers. First column: SNP marker number; second
column: SNP marker name; third column: 101 bp sequence with the SNP marker at position 51 bp,
sequence is from the Abacus reference genome (version Csat_AbacusV2, NCBI assembly accession
GCA_025232715).

| | SNP marker | 50 bp |
|---|---|---|
| SEQ ID | name | flanking sequences (101 bp sequence with SNP at position 51 bp) |
| SEQ ID NO: 1 | 171_6189 | AAACCATTTGCAGCAAGTTGGCCCCTTGGGTCAATGAAGGACCTCTCCACGAGTAGTGCTGCCA |
| | | TGGGCCTTTCTTCAACAGTTAAAGACAAAGATCTTTC |
| SEQ ID NO: 2 | 171_31491 | CGATTTCTGAAATCTTGAAACCCTAGTTTTTCTCTCTTTTGGAAGAGACATAGTCTTGAGGTGTTT |
| | | CTCGAATCTTTAGACTGAGTGATCAATTTGTTTTT |
| SEQ ID NO: 3 | 171_38099 | TGGAGGATGTAGTATATTGATATAGTGCTGCAATAATATTCATTCTATTCTTCTGGATAAATTAT |
| | | ATATTCTCATCCTCATCCTCTGGATCGATCCATACT |
| SEQ ID NO: 4 | Cannabis. | TATTAATTAGAATGTCAGAATGGCCTCAGTACTAGTTTTACAATGCTATTACTTTTGCTGGACAC |
| | v1_scf | AATTAAAATTAAGTTCTCTTGGAGCATTTATACTGG |
| | 1780-26461_ | |
| | 101 | |
| SEQ ID NO: 5 | 171_143517 | GGGTCAATCTCAAAGAGCTTCTCCACAATCTTTGTGTAGGCAGTTTCATGGCGTTTCTCATCGGC |
| | | AGCAATTGTGCCGCATATCTGCGCCAGTTTCAGGTC |
| SEQ ID NO: 6 | 171_178548 | AAACTGTGCCAAGTTGGCAAACTTGCCAATCTAAGGATTGTCTCACTGACGACCTCATCTTCCAA |
| | | ACTCTGCGAAACAATACCATTGTTAAAAAAACTATC |
| SEQ ID NO: 7 | 171_339402 | CTCGTTAATGTGTGTATCTTTAGTTTTATTTGGTTTTTCTTTGTAATAATGGTTGTATCCTAGGTTT |
| | | TATGTGGTGTTGGCTTTGGTCGTTTTGTAAGGTT |
| SEQ ID NO: 8 | 171_346595 | GTAGGATTTGATGGTGTTTATATAGAGATATTAGAGAATGGAATTATGGGGTACTTGAGTTGAGT |
| | | TCCTATAGCATCTTTCTATAGCATATGATTATGCGG |
| SEQ ID NO: 9 | 171_353618 | AAAGCCGATCTAGTCTACTACAGCGAATCTCAAAATAAATTTATGCTTTGGAGTAATTAATCGAC |
| | | CGTGCCCATACCTAACCATACCACCCAAAATAAAAA |
| SEQ ID NO: 10 | 171_399703 | ATCTTTCCTTCGCCGTAAGTGAGGACTCCCAATACATTTGGGAGGAGGTCACTCATGAACATGTG |
| | | AACTTTGTCTCTTTTTCAACAACAAAAAAAATAAAA |
| SEQ ID NO: 11 | 171_501931 | GGAGGAGCCAGTGGAATAGGCCAAAGAAGTGCTGAAATTTTTGCCCAACATGGAGCCAAATTA |
| | | GTCATAGCCGATGTTCAAGACGAATTAGGTCACTCCCT |
| SEQ ID NO: 12 | 133361_2702 | TTTAAGATCATCATCATTTCAATTATGTTATATCATCACTTCTCTCTCCCTTCATATATACTATACT |
| | | ATTTCTTTTCTTATTTAATTTTGTGGTGGTTATT |
| SEQ ID NO: 13 | 133361_6612 | ATGTTGCACCACTTATCTTAGCATCTTTAACATGTGTACTAATACTCCATGGTCGAAGGTTAGCC |
| | | TGAATGTCATGATTATTGCCAACATCAACTCTCATC |
| SEQ ID NO: 14 | 171_654206 | TATTTGTTTGTTCAAGACTCATGAGAATTTAATTGTTTGTTATTTGATTTTCCATTTGTAATAAAT |
| | | AAAGTTATAGTTTTTTATTTTTTGATTTTGTATTA |
| SEQ ID NO: 15 | Cannabis. | TGATCTGACTGAAATGTTCCTCAACTTGTGGCTGCAGGCTTTTACGAGACACGAATTGCATATTA |
| | v1_scf | TGCGTCCAGAGGTGCCAATGGTTATCGTTTTGCTTC |
| | 4041-1538_ | |
| | 100 | |
| SEQ ID NO: 16 | 171_942768 | TTGGATTGACCACCGTCTTGTTGAGAGAGAAGATTGTCGTTCTTTGGTTTGTGGGTAATTGGGTC |
| | | AATTCCCATTTTGGCTAAACGTTTCTTTAGATGTGT |
| SEQ ID NO: 17 | 171_988338 | GGCAATCTTGGTATTGCTAGAAAGCTTTTTGATCGAATGCCTGAAAGAGATTTGGTTTCTTGGAA |
| | | TGTGATGATTACTGGGTATGCAAAGCAAGGTGAAAT |
| SEQ ID NO: 18 | 171_1037290 | ACAAACACAGCAGCACTAAGCACAGCATTATTCAACGAGGGATTAAGTTGCGGTGCATGTTTCC |
| | | AAATAAGGTGCGCCAACGACCCTCAATGGTGCCTCCC |
| SEQ ID NO: 19 | 171_1080819 | ATAGTCACGCTACTAACACGAACAAACAAGCTTTGGCCCAATTGATATTCTCTCAATGTCTAAAA |
| | | AAAAAACAAGACTAAAATCTCCTAGCATGAATGGAT |
| SEQ ID NO: 20 | 171_1089602 | TATATCGGATTCTAGTCTTTCTGGGCATAGGTATTTGTTTTCTGGGTACAGTTTCTCTGTCTCCAG |
| | | AAGGAAATGGAGGAACCCTTTTGTTCAGATTAGGT |
| SEQ ID NO: 21 | 171_1100713 | TCTCACTTTCACTAAACTTTCTGCTAGCAACAGAAGGCGGGGTTTCTCTCCTCTTTTCTTTAGAAG |
| | | AAAGGACATAGGTGCTGAAAGGGATTCTGTCGTTT |
| SEQ ID NO: 22 | 171_1106446 | TATATTAGCAGCTAACTATACATACTCATTCCACAAGTTCTCTCATAGTTTAGTACATCAAACCA |
| | | TCTCATTCTCTCAGCATAAATCTTGTAGAATAATCC |
| SEQ ID NO: 23 | 171_1108332 | CGTACGTATATATTTATATAAATATATAAATTAATTTATAATATTATGATAGAAAAGTCATCCAA |
| | | ATCAACTCTTGGGATTGGCTGTTGGAAGAGTGTTAG |
| SEQ ID NO: 24 | 171_1147276 | CATTTATCCTTCTATTTGTAATTTATTTATTTAATCAAAACAAAATAAATGTTACGCAATCCAACA |
| | | TTCACAAGATATATAACATTAAACTCACTCACTTA |
| SEQ ID NO: 25 | 171_1177247 | GTGGGTCATATCTCTGGTGCACACATGAATCCTGCTGTTACTCTAGCTTTTGCTACTTTTCTACAT |
| | | TTTCCATGGAAACTGGTACATTATTTTTTTATTTC |
| SEQ ID NO: 26 | 171_1261218 | AAAGGGGTTCTTAAAGTCTGGATGCATAATAACAAGAACACACTTGGGAAAAAAGATATTAATG |
| | | GCGGAGGAGCCAGTACAGGTGGAAGAAGCAACAATAA |
| SEQ ID NO: 27 | 171_1342861 | GATCCTTACGTGTGGCGCCAATACAATGAAGTTGCCAATGAATTTCAACAATTTCAGGTTCTCTT |
| | | ACACAGGTTTGTAGAGAATGAACCTTTTCAAGGTCC |
| SEQ ID NO: 28 | 171_1361793 | AACTCAACACCCACTTTTACAGCTCAAGAAATCTCTCCTTACAAGCCTCCTATTAACTCTTTTCCA |
| | | AATGGAGAGTTTCCCAATGAATTTATGTTCTCTCC |
| SEQ ID NO: 29 | 171_1372205 | AAAATAAAACTCAAGCCGTAGATCACTACACACAACATATTAACAACAATATATCAGCATGGCT |
| | | TCTTTCGATGCAGTTGCCTCTCTGAATTTGAGATTAC |
| SEQ ID NO: 30 | 171_1418329 | TCTCCCTCATCATCATCGTCACCTTCGGCAGATTCTGCCCCAACTTCCTCATAGTCCTTCTCAAGG |
| | | GCAGCCAAGTCTTCTCTCGCTTCAGAAAACTCACC |
| SEQ ID NO: 31 | 171_1423189 | AATAGTACTTTGTGTTATTTTTTATTTATTTAGTCTTTTGAAAAAAAGAGGTTAATTGTCTTTTCCT |
| | | TATTCAAAATTTGCGGCCATAACAAACACTAGCT |
| SEQ ID NO: 32 | 171_1458503 | TTTGATAAAGAAAAATGGGAAAAGAGTTTGGGATAACAGAGGATTAATATGTAATATGATCACA |
| | | TGGTGTTGCACATTGAGACATGTACATGAAGGCCCAC |
| SEQ ID NO: 33 | 171_1500507 | TAATTAGATGGGAAATTTAATGTAATTAAACATACCTAATTCTAGAAAATATGGCTGCACCATAT |
| | | GGAGTCCGTGAATCTATTGTATGTATTTTTTTTACC |
| SEQ ID NO: 34 | 171_1522309 | GAGCGATCTGGCTCGGCTACTAGAATGCAGGCAGGGCTTGCTAGGAATGGACAACCAACAAGA |
| | | AGCCTGTCAGCATCAAATGCTCAGCAGGCTTCTCCGTA |
| SEQ ID NO: 35 | 171_1557279 | TTACAGGAAGCAGGACCGGGAGGACTGGGTGGAGGAGGTATGTGGGGTTCGGCCACTGATGAA |
| | | AGGAGTGTCTACACTAACATTGCCAACAGTCAAAAGAA |
| SEQ ID NO: 36 | 171_1561630 | TTTCCTTTGGAAGTTTGGATTTAGATGGTAATTTCTTCCCAAAAAGCAGACTCTTGAGCCACTTTC |
| | | CTGGAGATTTTCCCATTGCAAGAATTATGTGTTCT |
| SEQ ID NO: 37 | 171_1567853 | ATAATACCTGTTATAAACAAGATTGATCAGCCAACTGCTGATCCTGACCGTGTTATAGCTCAATT |
| | | AAAGTCCATGTTTGATCTTGACCCTGCTGATGCTCT |
| SEQ ID NO: 38 | 171_1647947 | TAATCACTTACTGCATCTAAAAGAATATTAGAAGTCTTGAAATCACGATATATTACAGGCCTTTG |
| | | AACTTCTTCATGCAAAAAAGCAAGACCCTTTGCAGC |
| SEQ ID NO: 39 | 171_1709761 | ACTGAAACTAGGATTCCTGTCATACCCTACCGTATTGAGGAACATGACACAATAGTAATTGCTAC |
| | | ATAGATACTCACAACTCACGATAGACTCAAACCTTA |
| SEQ ID NO: 40 | 171_1765646 | TTAATTATAATTAAATAGGCGCCATCTCTCTTGCGACAGTAGTCCCTTTACTTCTCTATTCATTAT |
| | | TCCTCTCATATCTTAATTATTAAAATTAAGGATTA |
| SEQ ID NO: 41 | 171 1821517 | GGTATCCACAGCCCCTCGACCCAAATCAACACTCAAATCTTCATTACTGACCTCAACGCAACCTT |
| | | CTACCATGGACTTCGGACCCGCGAGATTATGCGTTG |
| SEQ ID NO: 42 | 171_1830089 | TATTATTTTATTTGGTTGTCAGAGTCGCTTTACGTATCGGTGTATTTAGGCCTATATTATTTTGGG |
| | | ATTCAAAATAAAAAATAAATGTATTATTAGATTTT |
| SEQ ID NO: 43 | 171_1848136 | ATCACTAACTATACATGTGTGTCATAGTGTTGATTTTGTATAACTACATAAAATTTCATTACAAA |
| | | ATGTTTTCTATTTTGGTTGAAACATATTCATTTTAT |
| SEQ ID NO: 44 | 171_2067142 | TTTGATAGTATTATTGGACACTAGTTAGTGAAAATATTCATTTACTATGTCCCACAAATTTCATCA |
| | | CCATATAATTATATATAGTGTGCGGTTGTTAATGG |
| SEQ ID NO: 45 | 171_2092878 | ATCCAACTCAAAAGGATGAAAATGACCAGACAGACAGTAGACACGGTTACCTAAATTAGTACTG |
| | | TCTTTGACCATTCTAAGCAAATAAATAATAAAAAGAG |
| SEQ ID NO: 46 | 171_2175812 | TATCTTCACAAAAACAACAACTTGATAGGTTGTGTTTATTGCAACAACAAACCAAACACAACAC |
| | | AACACAAGAGCAACTTTGCGATTCGAATGGCATTTCG |
| SEQ ID NO: 47 | 125705_984 | TTGATTTCTACACTAATCGAAATTCCTGTTGTTTGTGCATAAACCATCCAAAATTTTAGATGGCG |
| | | CAGGATCCAAATTCCAAGGCCCCTTTGATAATGTTT |
| SEQ ID NO: 48 | 171_11609 | ATATTCAACAAACCATATGGCCCAGAGCTGCTGCTGCTGCAGGTATGTAAGCTTACATTGCAATG |
| | | CATTAATGAATTTCCATCATTGATTATGATGAGGGG |
| SEQ ID NO: 49 | 171 48451 | TAGTAGTGGATGTGTTGGGTGGTCTGGAGAGTGTTGGGGAGCAGGATTGTGTGGTCATCATCAT |
| | | CATCCTTGCTGCCCTATTTGGGTTATTGGTAGTAGGC |
| SEQ ID NO: 50 | 171_80245 | ATTCTCAACAAAATCTTTTCGGTTTATCATTGCTGCACTGCATCCCTTTGTGATATCTGTTTTTGCT |
| | | TTCCAAGTTTTATTTGTCTAAGAAGGTTTGTGTC |
| SEQ ID NO: 51 | 171_85256 | CCAAACAAACACACAAGAGGGAATGTGAAAATTTTCGATGACGTCACCTTCACCAGCCAGCGCA |
| | | CCAAATAAATTAAAAAATTAAAAACGACGAGTCCAAA |
| SEQ ID NO: 52 | 171_94326 | AGAAAGATAATGGCAATTTACATGCCTCCCTTGATGTGGCATCTGTGGAATCTGAGGGGTATGA |
| | | TATTAATATGCTTCCTGGGTCTGGAGATCAAGCTGTG |
| SEQ ID NO: 53 | 171_130678 | ACTTAATTTTTTTATAATATTATTAAATATAGTATTCTTAGATAGAAATTATACTGTATAACTAAT |
| | | GATATGTGTACGTAAACATGACCCGGACCGACACG |
| SEQ ID NO: 54 | 171 219947 | ATTGTACGAATTAGAAGATGAAGAAGAAGAAGTAATGGTAGAAGATGATCATCTTCAAGAAAT |
| | | GTTGGTGTTCAACACCAAAACGACGTCGGTTAACAAGG |
| SEQ ID NO: 55 | 171_289630 | AGTCAATGTACTAGAAAAGCATCAACTTGAGGAATTGAATTTCATATGGAGTATTAATTATGTTG |
| | | GTTTTGAACCAAAACGTGGAGAGGGTGTTCTTGAAG |
| SEQ ID NO: 56 | 171_302378 | TATATTTTCTTTTTTAGAAAACCACCCGCCAAATCCAAATATTTCTCAAATTTTTAAAATCCAACA |
| | | TAACAAGTCAACGATCCTTGTCATAATCCAATCTC |
| SEQ ID NO: 57 | 171_319576 | TCGTTAAAATACTCACAACATGACATTAAAATATAATAACATAATAATAGATTACATACATGGG |
| | | AGGCTTGGTGGCATTAGCTCTTTCCCAACCATTCTCA |
| SEQ ID NO: 58 | 171_363441 | ATATTAAGCAAAACCATAAAAAACAAAAAACAAAAGTCATGGTTTTTTGTCTTGGAATTGGACC |
| | | CCCTCCTCCTACCCTAGCCTTATATATACATTTTCTT |
| SEQ ID NO: 59 | 171_381570 | CGTTAAAACGACATCATTTTCACAAAACGACATTATCCAGTAAAAGGACAATAATTACAATATG |
| | | AAGCCAAACTGTTCGATAAAAGTCCAAGCCATGGCAT |
| SEQ ID NO: 60 | 171_429974 | AAATCAGTTCCATTTGGATCAAGAGAAGAAGCATTAAAGAATGGAAGTAGTTTGGGTTATTTTA |
| | | ACTCAGCTCAAGCCTTAGCTGACTATGCTCACATTCT |
| SEQ ID NO: 61 | 171_441217 | TTATATTAAGAAATACCATTGTCAATGGTGTCTTCACTGCAGATATATATTCCTCATTGACAAAT |
| | | TCGGTAGAATAAGATGGCAAGGCTATGGATTGGCAA |
| SEQ ID NO: 62 | 171 448590 | TTTTTCAATTTTGAAAAGTTGGTCAATTTCATCCAATTAAACTAATCATACCATGTGTCCGTACAA |
| | | TATAAAAAGTCCTTACTTCATACTCTAGTCTAGTC |
| SEQ ID NO: 63 | 171_462731 | TCATCCAATGCAAGATTTTTTTTTTATTTCATTCTTACTCCTATGTATTCGTTCATTCTAACCGAGC |
| | | ATACCCTAAGTTTCGGTAAGCAGTCATTAACACT |
| SEQ ID NO: 64 | 171_477640 | TAAATTTTGAACTTTTGAAGTGTTATACATCTCTTATCTATATTTTAAAAAATATTGGCGCGTGCA |
| | | ATACACCTCGTTAGTTGAAAGAAAGAGGGAGAGTC |
| SEQ ID NO: 65 | 171 483960 | TTTGTTAGTCAGAGAATTTTCCTCTTCCTGAGGTAAGAATACATTAGGAAAAATCCTACAACAGC |
| | | TCCAACGATTATCCCAGCTGTTGTGAGCCAGAATGC |
| SEQ ID NO: 66 | 123467_5978 | TTGCTAATTACCGGGCAACGTTCGCCATAGAAATCTAACATCTCCATAGCGCATGATCCTAGGTT |
| | | GCAGTTCCCCGTGTAATTTTGAGGTACATTAGTGCT |
| SEQ ID NO: 67 | 171_573381 | TTTCTTTTCGGAGATTCAGTACTCGATGTTGGTAACAATAATAATTTAGCAACGGTTGTTAAAGC |
| | | CAATTTTCCTCCTTATGGACAGGACTTCAAAAATCA |
| SEQ ID NO: 68 | 171_606839 | TCTTCCAAATTTTTCGTTGTGGCAATATCTACCGATGATACATCGTTCTCGAGAGTATCATCCTAT |
| | | CACAAAATATACATATATATTTCATTATTTATATT |
| SEQ ID NO: 69 | 171_624259 | AAAATATTTCGTAAAGCATTATAATAAAAATTGCCAACCTTGCCACCACTCGAGGGTAGTGTATT |
| | | GAGAGAGTGCTAATTATAGCATTTTATTATAAAATA |
| SEQ ID NO: 70 | 171_636730 | GCCCACTCTCTCAGTTTCTTTAGATAAATACCGACCCTTCGTTTCCCTTTACAAACCCAAAAATCT |
| | | CTCACTGTCGTTCCTCACTAACACACACCCCCGCG |
| SEQ ID NO: 71 | 171_643517 | AAAAATCTCCTATTAATATACATTTTAGGGTTAATAAACTTGAACAAATTAACAAGCTATAGATC |
| | | TTTGCACCCAAAATTCGAAACAATTATTCTACATGC |
| SEQ ID NO: 72 | 171_648249 | AAAGTTGCTAAGTTGATCGATAGCTACATTGCAGAGGTTGCTTCTGATGTGAATTTAAAGCCCAC |
| | | AAAGATTCGTTCTTTAGCTGAGGCTATACCAGGGTC |
| SEQ ID NO: 73 | 171_702968 | GAGGAGGAGGAAAAGGAGGAGAAGCCGCGATATCTACTATCAACATAAGCGGCCCTTAAGGTG |
| | | GGCCGCTGAGATGAAGATCTTGAATGGAGGCTATTATT |
| SEQ ID NO: 74 | 171_724002 | CCCTTTTTACAACCGTTTCGAAGTGATTCGCCGCTCTTCTCACCTTCTTTCGCCTCTCCTCTCTTCA |
| | | CTCTCTCCACCTCTTTCTGCTCTTATCACAGACA |
| SEQ ID NO: 75 | 171_745360 | TGCGTGTTTCTCCATGTGAGTTTCGGGTTTTCTCAGAGAGTTGAGAGGTTTTGATATACTGTTATG |
| | | AACTGAATCTTAAGAAATTCTTGTATAATCTTAGT |
| SEQ ID NO: 76 | 171_778207 | TCCGAGTTCTTCCACATCTGTGATAGTGTATATGATCCCGCCAACTTGTAGAACATAAGCATACT |
| | | CGTCTAGCAAGTGTGGACTGATCACCCTACGGCGAT |
| SEQ ID NO: 77 | 171_835887 | CCGTCTTCCTACTGATGACAATTATAATGATTATGGTATTGGAGGACAACATGACCGTAGGTTTG |
| | | ATTACTCTCCAAAAGCATTTGACAAAATTCCATGGA |
| SEQ ID NO: 78 | 171_857167 | CCATTTCGTGTATATCGAGTAGACGACATAGATGGATCTTTGTATGCATCTACTAGCTTATCATA |
| | | AAATTCGTCCCTAGCAGGATTATTCTGGTCCTCTTT |
| SEQ ID NO: 79 | 171_863359 | TTAATAAATGGAAGTGGTGTGGTTTGGTTTTGGTTATGATTTGATGAAATCGAGTGTGGTGTGGT |
| | | GAGTCAGTGTCGTACTTTTTTGGGATTGGTGTGACG |
| SEQ ID NO: 80 | 171_868913 | TCGTCCATTAGCTCTCTACACCATGTCACTCGCTTTGGAGGGCAACTACCTGGTCCGAAACACCT |
| | | GCAAGTTAACCAATTCAAGCTTTAGTTTTGGCTGCA |
| SEQ ID NO: 81 | 171_873049 | GTGTAAGATTTTTTATAATCCCATTAATTTATAAACCCGACCCGGAAATCCACAAACCCACTTTC |
| | | CTTTCCATAATTTCCATGCACACATAAACGACGCCG |
| SEQ ID NO: 82 | 171_878186 | GTCTTCAAATGCCAGAACTGCAAACCAGCAACCGATAAGCAAACCAACAGAGAAAGAAGACTA |
| | | AAGGTAGCCATTTGTGAGTTAGTCTTTCTATTAAGTTG |
| SEQ ID NO: 83 | 171_908334 | AATGCAGTCAAAGTCATACAGAGCGGGAACTCTCTTGCTCAGTCTTCTTCTCCGTTCTGAGAAGT |
| | | GTTGGGAGTACTTGAAGCGGAGTTTTCAGATGTTAC |
| SEQ ID NO: 84 | 171_948145 | CAATTAAGCACGATAATAATTACATGCATACAATTATAGTAAAGTAATAACTGTAATAATAACG |
| | | TGTTGATTTAGATAACAAAGTGTTTATCTATTTAATT |
| SEQ ID NO: 85 | 171_976341 | AATTAACCTAATACAAATACAAATACATTTTACTCAAGCTTAAGCTAAAAGGTTAGCTGTAAATT |
| | | TTGGTCATTCATTAGGTAAAGTCTGAAAAGTTATAC |
| SEQ ID NO: 86 | 171_978291 | CGCTTTAGCCCACGTCTTACCCTGCGAACCACATGAAGGGTCATATGAGGACACATGACCCCAC |
| | | TCAATTTATTATAAATTATATTTTGTATAAAAAATTT |
| SEQ ID NO: 87 | 171_1008238 | GCACTAACGATGGAGTGTTCTTTTCTTCTTCCTATGGTAAATTGAGAATCGAGTGTTCTTTTTCTT |
| | | ATTTCTAGTGAAAAAATGAGAATAGCACATAAAGT |
| SEQ ID NO: 88 | 171_1024475 | AAGAAAAACTGAGTTTACTATAATAGCATTATGTAATAAGTGGTATGTGTAGTGTTATGGTTGGC |
| | | CTACCAACTAAAACTTTCAATTATGAAATGTCCATT |
| SEQ ID NO: 89 | 171_1043983 | ATAATAATAAAAACTGATTGATGAATAAATAAAAGTTAAGTACAGTCATTAGATGTCAATTTAA |
| | | GTAGTTCATAATTAACCAATCCATGGATTTAAAAGTT |
| SEQ ID NO: 90 | 171_1052176 | ACACCATGAAAATAACTCCAGCGGACCAAGGATTGAATAGGGATGTAAACCGGGTTGGGCGGG |
| | | TGCGGGGATTGCTCTCCCACCCCCATACTCGCTTCTTA |
| SEQ ID NO: 91 | 171_1082805 | AAAAACTCAGCTTCATCATCATCTTCTTCAATGGTTAAAGATGGAGTCAAGAAGGGTTTATTTGA |
| | | ATTGCCTCGTGATCACCACCGTGAATTGGAAGCTGA |
| SEQ ID NO: 92 | 171_1117836 | ACCCTTCTCTTTCGTACCCAAATATCACGCCCTTCTTCATGGAATTTCCTTATCAGAGCCTACGCT |
| | | TCCAGCCATTCACCCAGAGAAGCCATCTGGGTGTT |
| SEQ ID NO: 93 | un29717_ | CATTGTATGCAGCAGGTCAAGTTATAGGTGCAATTTTAGGAGCATTTACACTACGTGAAGTGCTA |
| | 60_61 | GACCCAATTCAAGATATTGGAACCACAACACCACAT |
| SEQ ID NO: 94 | 171_1151293 | CAATAATGCTTTATCAGGTTCTTCTTCTTCCTCTTCTTTCATCCCCTATTAAAATTTATATTTAATT |
| | | TCAAGATTATGATGATAATTTGAGATATTTTTAT |
| SEQ ID NO: 95 | 171_1190666 | CATCTAATCACAATCACCAACAAATTTAAGTGCTAAAGAACTATATCTTTGGTAAGAAAGACAC |
| | | AAACAAGTACATATCATTATGTCATGGTTAGTTTTAT |
| SEQ ID NO: 96 | 171_1219675 | TTATTGGAAATTCTCCAAGTTTCCTAAGGGCATTTAGGGCCTCCTTCCTTAGTCTTCCAGTTGTTG |
| | | TAGCAAGGGCACACAGCTTTATTACCATAATACCC |
| SEQ ID NO: 97 | 171 1240501 | GAAGCACTAATCCACGAAACCCCTCAATATGGGTCTAAAGATAAAGACATAAGGCTACATAATC |
| | | CAACAACCTTTAGAACCAGTTAAAACCCAAATTAAAC |
| SEQ ID NO: 98 | 171_1357163 | TCAGTATGAAATGAATGAGTTTGATTTGAACGAGGAAGAAGCTACACACTCCAAGCCCTAGACA |
| | | AGCTACATGCACAAACAACATTGTATTCTATTATTAT |
| SEQ ID NO: 99 | 129515_2372 | GTCTTTGTTGATTAGTGATGCAAATTAGTATTGGTTGGCAAATATTTTAGGTTTAAGTTAAAAGT |
| | | CAATATATACCAAACCAATTTTATAGCAATATTAGA |
| SEQ ID NO: 100 | 171_1436811 | ATACACTATCTGTCAACTTTTCTGGCTGGACTGGTGGTTGGTTTTGTCTCAGCATGGCGGCTAGCT |
| | | CTGCTGAGTGTGGCGGTGATTCCCGGCATTGCCTT |
| SEQ ID NO: 101 | 171 1441354 | TTGATAATGTTACAGCAATTGTCCCTCAAAGGATTTGCCGGAGACACAGCAAAAGCTCATGCAA |
| | | AGACAAGTATGATTGCCGGGGAAGGTGTGAGCAACAT |
| SEQ ID NO: 102 | 171_1459473 | AAAAACTATATATAACTGTTATTTTGGGCCTCTTGGGTCTTTGCTGTATATATAGTCTAAATTTTG |
| | | ACCCCGGGTATCCAGGGACAGACTACAGAGTACAT |
| SEQ ID NO: 103 | 171_1463835 | GATATTCTTCATTAGCATTTTCCCATGTTTTGCTTATAGCTACAAGATATGTAATTATTTAAGATT |
| | | ATAGTTACAAGATATGTAATTATTAAAAAAAAATT |
| SEQ ID NO: 104 | 171 1539058 | AAATTCACCGCATTGACTCGAAATGGTCTGCTGGCAATGTAATTTGACTCTAAGCATTCTGAGTG |
| | | AACAAGGAACAGCCCTCTAGCAAATGATCCAACCTA |
| SEQ ID NO: 105 | 171_1585716 | CTTGTCTTCTTACAAGTGGGGTCACCCCTAGACTTATTTAAACCCCATTTTGGGGTAGTTGGGGG |
| | | AGATTGTATTGTATGTCTCTAAAACTCTAATAGCAT |
| SEQ ID NO: 106 | 171_1615710 | GAGTTCTTCTCCACCAATTCTCTAAACATCGAAGATTGAAGAAGGAGGCCAAGAGCAGAAGGAG |
| | | AAGACTTGCTGCGAGTCATACTACTGCCAATTAGAGG |
| SEQ ID NO: 107 | 171_1622388 | AAGAAACAAGGTGGTGGTAAAGCTGTTGAAGTTGATAAAGCTGACATTGTAGGGGTAACATGG |
| | | ATGAAGGTCCCAAGAACAAATCAACTTGGTGTTCGGAT |
| SEQ ID NO: 108 | 171_1634072 | TAATAATTATTCAAAAGAAAAGAGCCTAGAAAAGGTTTTAGACTTACCATATCAGCCTACATAG |
| | | CCTTCCTTTCTCTAAAGAACATAGCCACAAGAAAACT |
| SEQ ID NO: 109 | 171 1642347 | GGTCACCATGGTAGGGTTCACCATTGAGGAGCTTAGCATAGTAGTCCGTTCAGATTTCAGACTGA |
| | | CTTCTCTTTAATTCAAATTGTGGATTCTGTTTTTTT |
| SEQ ID NO: 110 | 171_1645831 | TAGCACTAGAGAGAAACATACATATCATCAAAAGACATAGGCTTGGATCTCTGAGTTAGAATTG |
| | | GTGAGCTTGTAGAAAGCATCTGCTGCTTTCCCATGAA |
| SEQ ID NO: 111 | 171_1667193 | CCCAAAACAAAAAAAATGCTGAGGCGAGTGCTGCAACGAGCTTCCACTGGCCGCCATATTGCCA |
| | | AATCATACTACTTGGCTTCTCATCTTTCACCTTCTTT |
| SEQ ID NO: 112 | 171_1676674 | TAATCGAAATAGAGTGGAGGGGAGTGGCCCGTTGAGAATGTTACTAAACAAATTAAGATAGAGT |
| | | AAGTTCTCTAAGTTTCCTATACTGATAGGAATGGATC |
| SEQ ID NO: 113 | 171_1704395 | TTAAAGAATCGGAATATTTCAAATTACGAGAATGAAAAGATGAAGATAAGTTGGTCTTTGAAAA |
| | | TTGAAAAGGGAGAAGAAGCCTTGTTACTTTCCACTGG |
| SEQ ID NO: 114 | 171 1741089 | GGCCTTACAACCCTATACAAACCATTGCTTGACAATATAAAGATGTCTTTCCTGTTATCTTCACC |
| | | AAAAGAGAATATGAAACCAAGTCCAGAGACTGAGCT |
| SEQ ID NO: 115 | 171_1743902 | CATGTAAAGAAACTATACTAAAAGATAGAACTTACTCTTATTAGTACAAAAAGGATACGAAGAA |
| | | GAGTAATCAAAAATCAGCAGAAAGTGACATAACATAG |
| SEQ ID NO: 116 | 171_1776686 | CTTCCCTTTCTGATTAGAGCAGGGTCAAGTTTTTCTACATAATTAGTAGTAAAAACTATGATCCTT |
| | | TCACCACCACAAGCTGACCAAATCCCATCAATGAA |
| SEQ ID NO: 117 | 171_1784261 | AAACTAGCCGGATGCTCAAAAAGGACATGACTCCACTTAGTCCGATTCCCGCCATAAAAGCTCT |
| | | CACCAGATCCATTCGTGTAAAGCTTTCGTTGCCGATT |
| SEQ ID NO: 118 | 171_1798950 | TTTTATTACATTTGTTATTATGTCAAATCAAATAGGGGTCTATTATCTCTCAGAAGAAAATAAAA |
| | | TAGGGGTCTACCACATATAGAAATTTCCAAGACTCC |
| SEQ ID NO: 119 | 171_1801885 | AAAATGATAGTTGTTGTAGATATTCTTGGACTAGCTTTGATGGCACGTGCTAACACGAAAGTGCC |
| | | TCTTTACGTAGACTCAAGTTCGTATGAGTCATTTGG |
| SEQ ID NO: 120 | 171_1825490 | GGCACTGTCTGCATCAGAAGGCGGTGGTGGCCCACTCAAACTCGTTTGCCTCGTCAACTTAATAA |
| | | AAGTTCGCCTGAGCCTGCGAAACTCAAGCAAGCCAA |
| SEQ ID NO: 121 | 171_1889481 | TTTGCGGTGTTCTTATAAAGAAAGATAATTATGCAAAATCTGATCTTGATTTATGTGCAGATTTT |
| | | GTGTCCGAAAATGGGTCGAGATTGAGTGTTTCATCG |
| SEQ ID NO: 122 | 171_1907238 | TTCAACCACGGCAGCAACAATACCCTTCACTGTCTCCTGCATAACATTGATAAAGATCACGAAC |
| | | AATGAGTTATCAAAATTATCTAACTAGTCTAATTAAT |
| SEQ ID NO: 123 | 171_1907774 | AAGGGAACTTAGTGAAAGAGCTGTTGTTGTTATTACAAAAGGAGAGTAATTTTACATACCTAGC |
| | | TTTAAGTATTGTTCCCATAGTTCCTATTTGAGTTGCA |
| SEQ ID NO: 124 | 171_1915168 | CCATTTACATACACAGGGCCTTGAGAACTTACCTCAAGATGAAAAGTCTCTAACCATTTGCATGC |
| | | TTCTCCCATATTCTCTCTCACCCCCTATTAATCATT |
| SEQ ID NO: 125 | 171_1927464 | TAAAGAGCTTATTAGGAGAATACTCCCTTAATGTAGATCCGATTAGTCTATGTGGGGCCTCTAGC |
| | | TTGAGAATGACCCCTCAATCGTGATAACAATTATTT |
| SEQ ID NO: 126 | 171_1929444 | GAAAAAAACAAGTAACGAGTAACAAATGATAGGAGAATATGAGGCTAATAATTCATTATGGTCT |
| | | CTCTTTCACGAAATGTAAGAGCCAAGAGGGTTGAGGA |
| SEQ ID NO: 127 | 171 1949435 | ACTAAAAAATTATCACTAAAATTTAAAAATGATTATTTTTTGTAGTGATAGGTGAACTTTCTACA |
| | | TTCAAAGTAGAAGTTTTGGCTTTGTTGCATGCTATT |
| SEQ ID NO: 128 | 171_1974687 | GGTGAGGTATTGGAGAATGTTTCAGGGGAAGTTGAATTCAAACACGTTGAGTTTGCTTACCCTTC |
| | | TAGACCCGAAAGCATAATTTTCAAGGATTTTTGCTT |
| SEQ ID NO: 129 | Cannabis. | AATCTCCCACGACCATAATTATACTGATTATTAGCAGGCAATCTTGATATATACGAAGCATATAG |
| | v1_scf | TCCAATAGCAAGAGCTGCACAATCAAACAACATGTG |
| | 2769-57335_ | |
| | 101 | |
| SEQ ID NO: 130 | 171_2080692 | TATTAGGAGACCCAGAGCTACCAGACCCATTCTGACCACAATCTTTTCCACTTCCTGAACCCAAA |
| | | GCTACCAAAAACTTCTCATTCCATCTGCGACCACCA |
| SEQ ID NO: 131 | 171_2180533 | TGGATGAAAGGTTACAATGACTGTGTCCACTGGTGCATGCCAGGTCCTGTGGATCATTGGAGCC |
| | | ATTTTTTGATGGCTGTTATGAGGAAGGAATTGGGTTT |
| SEQ ID NO: 132 | 171 2195254 | GAGAAGAAGGGTGCCCAAAGAATAAGAATAGGCCATGAAAACAAAGAAGGTGAAGCCTTTCAA |
| | | AGTAGCTGATTTATACAGAATATGTAGACCAACCATAC |
| SEQ ID NO: 133 | 171_2307670 | TTATTATTATTATCATCATCATTTTACCTTTAATGGCAGTTCTGCAACGAATATGCGGTACCTAAG |
| | | GACAAGCTAGACAACTTCAAAAATGTCACAGCACA |
| SEQ ID NO: 134 | Jun245831_ | CGGATGCTTTTGAAAATTTCCAATTAAAAACATA |
| | 73_74 | TTCATTCTCTAACTATTTTTAATTCTTCTATCCTTGTACATTCATTTTGTATAGGTGGGAGCTATCT |
| SEQ ID NO: 135 | 171_2315250 | CTGAAATCTTACCTCATTAAGAATCGAAGAAAGAAGCTGGCTTTCAAAGTAGTTGCGCAGCTTG |
| | | CACTAGATCTTGCAAGAGGGTTGGATTTGCTTTCTTC |
| SEQ ID NO: 136 | 171 2588227 | CTTACATGTAGACACAAGAAACGTTTGTGGTCTGTGGAGTATGGAGCTAACCATATACCCCGTGT |
| | | AAGAGACACTACAGCCCTGCCAAATGATGAGGGTAA |
| SEQ ID NO: 137 | 171_2599642 | TTGACCAGAGAGTCAGACTAATGTACTTGGCTAACGAAGGAGATATAGATAGTATTAACGAGCT |
| | | GCTTGATGAAGGTACTGATGTTAATTTCAAGGACATC |
| SEQ ID NO: 138 | 171_2604364 | TGGGGTTGGAGTCAATGCGTGTAGATGTTGTGCTTCTTTTTGCTTGGAACGATATTTTCTTTGGGT |
| | | TATTTTAGCAGTAGGAACCAAAATGTATCGACTCT |
| SEQ ID NO: 139 | 171_2617799 | CACAATTGTCGTTATTGGAAAACTATAGGCACTCAAAATAGGGGACAAAACGTTGTTTTCAATTC |
| | | CCTAAGAAATGACTATTTTTCTTAATTTTAACATAA |
| SEQ ID NO: 140 | 171_2633941 | ATTGCTGAAAATTTCAAATCCCCTCGATTCATCTAGCTAGAATCGTTTCATGGTTTCTGGGTTTTT |
| | | TCAATAATTAATAACTCAATATTCCTCCTCTTCTT |
| SEQ ID NO: 141 | Cannabis. | GATCAATCTGCTTCCCAATCCTATGACCATCACAACTTTCTTCCCGTAACCCTTATGGACTCCAAT |
| | v1_scf | CCCCACTACTCTCACTCCCATCGCCCTGACCACAC |
| | 1670-75982_ | |
| | 101 | |
| SEQ ID NO: 142 | 171_2867392 | AATTGTTGGAATATTCAAGGGCTGTGATCATTTTGATTTATTTCATGATACTGACTACTCTTTTCA |
| | | CTAACAGTTCCCAAACTTAATGCCTATATCAATGA |
| SEQ ID NO: 143 | 171_3528035 | ACACATACATAGAATTGTTTACGTGCAAATAGTTACTTTAATCTACCTGTTGGGATCAGAACCAC |
| | | TTTTATGTCTCACATGTTTATTTAATTTATTCATAT |
| SEQ ID NO: 144 | 171_4503268 | ACTTCCAATGCATTTTTGACCCCTGATCTCGATGAGGCAAAGGAATATTTTGATAAGGCTGCAGT |
| | | GTACTTTCAGCAAGCATCCGATGAGGTTTTTCTCCT |

### Example 2

Discovery of Gene Sequences Associated with Flower Initiation

Seven genes, ELF9, FT, AGO5, CDF2, GASA4, CLPS3, and PI, located within the haplotypes of SNP markers 17194326, 171_143517, 171_219947, 171_1043983, 171_1219675, 171_1676674, and 171_2195254, respectively, were explored for putative causative SNPs for the mapped flower initiation traits.

SNP marker 171_94326, which was mapped based on Fisher Exact test of Flower Initiation of 22ALV2 and 23ALV1 data, is located at position 109,674 bp on chromosome 8 of the Abacus reference genome (version Csat_AbacusV2; NCBI assembly accession GCA_025232715.1); it is located 38.1 Kbp upstream of ELF9 (EARLY FLOWERING 9; AT5G16260; based on homology with Arabidopsis thaliana). The coding sequence (CDS) of ELF9 is located between 147,820-152,795 bp on the Abacus reference genome. Its CBDRx (version cs10) homolog is LOC115699158 (annotated as splicing factor U2AF-associated protein 2 isoform X2; referred to as CsELF9 by Dowling, Caroline A., et al. “A FLOWERING LOCUS T ortholog is associated with photoperiod-insensitive flowering in hemp (Cannabis sativa L.).” bioRxiv (2023): 2023-04) and is located between 64,398,177-64,403,436 bp on chromosome 8 of the CBDRx reference genome.

SNP marker 171_143517, which was mapped based on both Fisher Exact test of Flower Initiation and NAM of Days to Flower Initiation of 22ALV1 data as well as Fisher Exact test of Flower Initiation of 23ALV1 data, is located at position 159,096 bp on chromosome 8 of the Abacus reference genome (version Csat_AbacusV2; NCBI assembly accession GCA_025232715.1); it is located 12.8 Kbp downstream of FT (FLOWERING LOCUS T; AT1G65480; based on homology with Arabidopsis thaliana). The CDS of FT is located between 174,846-171,863 bp on the Abacus reference genome. Its CBDRx (version cs10) homolog is LOC115700781 (annotated as HEADING DATE 3A-like; referred to as CsFT1 by Dowling, Caroline A., et al. “A FLOWERING LOCUS T ortholog is associated with photoperiod-insensitive flowering in hemp (Cannabis sativa L.).” bioRxiv (2023): 2023-04) and is located between 64,421,284-64,423,641 bp on chromosome 8 of the CBDRx reference genome. In addition, 171_143517 is located 6.3 Kbp downstream of ELF9.

SNP marker 171_219947, which was mapped based on both Fisher Exact test of Flower Initiation and NAM of Days to Flower Initiation of 23ALV1 data, is located at position 241,017 bp on chromosome 8 of the Abacus reference genome (version Csat_AbacusV2; NCBI assembly accession GCA_025232715.1); it is located 23.0 Kbp upstream of AGO5 (ARGONAUTE 5; AT2G23850; based on homology with Arabidopsis thaliana). The CDS of AGO5 is located between 217,974-213,480 bp on the Abacus reference genome. Its CBDRx (version cs10) homolog is LOC1 15698998 (annotated as protein argonaute 5; referred to as CsAGO5a by Dowling, Caroline A., et al. “A FLOWERING LOCUS T ortholog is associated with photoperiod-insensitive flowering in hemp (Cannabis sativa L.).” bioRxiv (2023): 2023-04) and is located between 64,618,208-64,622,176 bp on chromosome 8 of the CBDRx reference genome.

SNP marker 171_1043983, which was mapped based on Fisher Exact test of Flower Initiation of 22ALV2 data, is located at position 1,154,438 bp on chromosome 8 of the Abacus reference genome (version Csat_AbacusV2; NCBI assembly accession GCA_025232715.1); it is located 18.4 Kbp downstream of CDF2 (CYCLIC DOF FACTOR 2; AT5G39660; based on homology with Arabidopsis thaliana). The CDS of CDF2 is located between 1,175,270-1,172,823 bp on the Abacus reference genome. Its CBDRx (version cs10) homolog is LOC115699551 (annotated as cyclic dof factor 2) and is located between 62,482,700-62,485,656 bp on chromosome 8 of the CBDRx reference genome.

SNP marker 171_1219675, which was mapped based on Fisher Exact test of Flower Initiation of 23ALV1 data and NAM of the combined set of 22ALV1, 22ALV2, and 23ALV1 data, is located at position 1,355,612 bp on chromosome 8 of the Abacus reference genome (version Csat_AbacusV2; NCBI assembly accession GCA_025232715.1); it is located inside GASA4 (GAST1 protein homolog 4; AT5G15230; based on homology with Arabidopsis thaliana). The CDS of GASA4 is located between 1,355,112-1,356,499 bp on the Abacus reference genome. Its CBDRx (version cs10) homolog is LOC115701397 (annotated as gibberellin-regulated protein 4) and is located between 62,291,905-62,293,013 bp on chromosome 8 of the CBDRx reference genome.

SNP marker 171_1676674, which was mapped based on Fisher Exact test of Flower Initiation of 22ALV2 data, is located at position 1,844,801 bp on chromosome 8 of the Abacus reference genome (version Csat_AbacusV2; NCBI assembly accession GCA_025232715.1); it is located 17.4 Kbp upstream of CLPS3 (CLP-SIMILAR PROTEIN 3; AT3G04680; based on homology with Arabidopsis thaliana). The CDS of CLPS3 is located between 1,824,429-1,827,375 bp on the Abacus reference genome. Its CBDRx (version cs10) homolog is LOC1 15698518 (annotated as protein CLP1 homolog) and is located between 60,848,469-60,851,731 bp on chromosome 8 of the CBDRx reference genome.

SNP marker 171_2195254, which was mapped based on Fisher Exact test of Flower Initiation of 23ALV1 data, is located at position 2,383,741 bp on chromosome 8 of the Abacus reference genome (version Csat_AbacusV2; NCBI assembly accession GCA_025232715.1); it is located 4.1 Kbp downstream of PI (PISTILLATA; AT5G20240; based on homology with Arabidopsis thaliana). The CDS of PI is located between 2,390,618-2,387,954 bp on the Abacus reference genome. Its CBDRx (version cs10) homolog is LOC115699531 (annotated as floral homeotic protein PMADS 2 isoform X1) and is located between 60,341,888-60,344,815 bp on chromosome 8 of the CBDRx reference genome.

ELF9, FT, AGO5, CDF2, GASA4, CLPS3, and PI, were evaluated for coding sequence variation in Cannabis accessions varying for their ability to initiate flowers under 18/6 and the mapped markers as described above (Tables 2, 3, 4, 5, 6, and 8). Sequences were compared with CBDRx (NCBI assembly accession GCA_900626175.1) reference genome sequence. CBDRx reference genome marker genotypes were determined after performing a BLASTN search based on 50-100 bp flanking sequences surrounding each SNP marker in the Abacus reference genome (Table 10).

RNA was extracted from flower tissue from 20LCMP-1:1 (Abacus), 23TRC1-16-1 (Finola; selfed progeny of 22ALV1 mapping population parent 20GAQ-1229), 23TRC1-17-1 (selfed progeny of sib of 22ALV2 mapping population parent PTVOG-21-18). 22TRP2-2-2 (sib of 23ALV1, 22TRP2, and 23TRP1 mapping population parent 20GAQ-1072), and four accessions that are incapable of initiating flowers under 18/6: 23TRC1-15-1, 23PLP1-4-15, 23PLP1-5-8, and 23PLP1-7-1. Leaf tissue was collected at the stage where plants formed flower clusters with at least six pistils (Nucleospin RNA Plant and Fungi kit, Macherey-Nagel). After concentration adjustment and treatment with DNAse, the RNA was used directly for RT-PCR (OneTaq® One-Step RT-PCR Kit, New England Biolabs). Sanger sequencing of coding sequence (CDS) was performed based on RT-PCR product. Primers for amplification and sequencing of ELF9, FT, AGO5, CDF2, GASA4, CLPS3, and PI can be found in Table 13 (SEQ ID NOs: 145-170). For both Abacus and Finola reference genome sequences (Abacus: NCBI assembly accession GCA_025232715.1; Finola: NCBI assembly accession GCA_003417725.2) were used in combination with RT-PCR sequencing data to identify correct splice sites. For CBDRx reference genome sequences (NCBI assembly accession GCA_900626175.1) were used and splice sites were predicted based on alignment with RT-PCR sequences.

Alignment of Sanger sequenced fragments was performed per accession for all seven genes. The resulting consensus sequences were subsequently aligned per gene. Functional CDS were translated to protein sequences, which were subsequently aligned with Arabidopsis thaliana protein sequence to identify functional domains. Functional domains were explored further in the protein sequence alignments for amino acid substitutions that would alter these domains.

### ELF9

Alignment of ELF9 CDS and protein sequence of Abacus/20LCMP-1:1, Finola/23TRC1-16-1, 22TRP2-2-2, 23TRC1-17-1, 23PLP1-4-15, 23TRC1-15-1, and CBDRx revealed two amino acid substitutions which in combination distinguish between accessions that differ for their ability to form flower clusters with at least six pistils at 18/6. These amino acid substitutions were not previously described as amino acid substitutions in ELF9 that are beneficial for flower initiation under 18/6 (Dowling, Caroline A., et al. “A FLOWERING LOCUS T ortholog is associated with photoperiod-insensitive flowering in hemp (Cannabis sativa L.).” bioRxiv (2023): 2023-04.).

The first amino acid substitution is a T (Threonine, observed in Abacus/20LCMP-1:1, 23TRC1-16-1, 22TRP2-2-2, 23TRC1-17-1) to I (Isoleucine, observed in 23PLP1-4-15, 23TRC1-15-1, and CBDRx) at amino acid position 86 in Abacus/20LCMP-1:1 (T86I; SEQ ID NO: 173) and 23TRC1-15-1 (SEQ ID NO: 174), caused by a C to T nucleotide substitution (Abacus/20LCMP-1:1, Finola/23TRC1-16-1, 22TRP2-2-2, 23TRC1-17-1: C; 23PLP1-4-15, 23TRC1-15-1, and CBDRx: T) at CDS position 257 bp of 23TRC1-15-1 (SEQ ID NO: 172), and Abacus (SEQ ID NO: 171; located at position 51 bp for SEQ ID NO: 175; Table 13).

The second amino acid substitution is an N (Asparagine, observed in Abacus/20LCMP-1:1) to D (Aspartic Acid, observed in 23TRC1-16-1, 22TRP2-2-2, 23TRC1-17-1, 23PLP1-4-15, 23TRC1-15-1, and CBDRx) at amino acid position 303 in Abacus (N303D; SEQ ID NO: 173) and 23TRC1-15-1 (SEQ ID NO: 174), caused by a A to G nucleotide substitution (Abacus/20LCMP-1:1: A. 23TRC1-16-1, 22TRP2-2-2, 23TRC1-17-1, 23PLP1-4-15, 23TRC1-15-1, and CBDRx: G) at CDS position 907 bp of 23TRC1-15-1 (SEQ ID NO: 172) and Abacus (SEQ ID NO: 171; located at position 51 bp for SEQ ID NO: 176; Table 13).

NCBI conserved domain search (Marchler-Bauer, Aron, et al. “CDD/SPARCLE: functional classification of proteins via subfamily domain architectures.” Nucleic acids research 45.D1 (2017): D200-D203.) identified three domains in the Abacus protein sequence of ELF9: 1. Domain of Unknown Function (DUF4339) between amino acid positions 34-84; 2. RNA recognition motif 1 (RRM1) between amino acid positions 260-353; 3. RNA recognition motif 3 (RRM3) between positions 390-462. The location of the first amino acid substitution, T86I, is located in close proximity to the DUF4339 domain, which was annotated as a GYF domain by Dowling et al. (Dowling, Caroline A., et al. “A FLOWERING LOCUS T ortholog is associated with photoperiod-insensitive flowering in hemp (Cannabis sativa L.).” bioRxiv (2023): 2023-04.). The second amino acid substitution, N303D, is located inside the RRM1 domain, which was annotated as an RRM domain by Dowling et al. (Dowling, Caroline A., et al. “A FLOWERING LOCUS T ortholog is associated with photoperiod-insensitive flowering in hemp (Cannabis sativa L.).” bioRxiv (2023): 2023-04.). T-DNA knock-outs of ELF9 in Arabidopsis thaliana, where the T-DNA insertion was located in the RRM2 domain, resulted in earlier flowering in short days, which are normally not inducive to flowering in Arabidopsis (Song, Hae-Ryong, et al. “The RNA binding protein ELF9 directly reduces SUPPRESSOR OF OVEREXPRESSION OF CO1 transcript levels in Arabidopsis, possibly via nonsense-mediated mRNA decay.” The Plant Cell 21.4 (2009): 1195-1211.). Therefore, the T and D amino acids of these two amino acid substitutions, T86I and N303D, in the ELF9 protein sequence may contribute to the ability to form flower clusters with at least six pistils under 18/6.

### FT

Alignment of FT CDS and protein sequence of Abacus/20LCMP-1:1, Finola/23TRC1-16-1, 22TRP2-2-2, 23TRC1-17-1, 23PLP1-7-1, 23PLP1-4-15, and CBDRx revealed an indel and an amino acid substitution which were not previously described (Dowling, Caroline A., et al. “A FLOWERING LOCUS T ortholog is associated with photoperiod-insensitive flowering in hemp (Cannabis sativa L.).” bioRxiv (2023): 2023-04.). The indel distinguishes Abacus/20LCMP-1:1 and Finola/23TRC1-16-1 (171_143517 marker genotype 0/0) from CBDRx (171_143517 marker genotype 1/1). The amino acid substitution distinguishes two accessions that are able to form flower clusters from the other accessions.

The indel is a deletion of 12 nucleotides AATAATAATAAT between CDS position 45-46 bp of CBDRx (SEQ ID NO: 179) causing a deletion of NNNN in the CBDRx protein sequence (four instances of Asparagine; SEQ ID NO: 182), which are present in Abacus between amino acid positions 16-19 (N16del, N17del, N18del, N19del; SEQ ID NO: 180), corresponding with nucleotide positions 46-57 bp in Abacus CDS (SEQ ID NO: 177; located at position 51 bp for SEQ ID NO: 183; Table 13).

The amino acid substitution is an L (Leucine, observed in Abacus, 23TRC1-17-1, 23PLP1-7-1, and CBDRx) to V (Valine, observed in 23TRC1-16-1 and 22TRP2-2-2) at position 66 in Abacus (L66V; SEQ ID NO: 180) and 22TRP2-2-2 (SEQ ID NO: 181), caused by two nucleotide substitutions. The first one is a C to G nucleotide substitution (Abacus, 23TRC1-17-1, 23PLP1-7-1, and CBDRx: C; 23TRC1-16-1 and 22TRP2-2-2: G) at CDS position 196 bp of 22TRP2-2-2 (SEQ ID NO: 178), and Abacus (SEQ ID NO: 177; located at position 51 bp for SEQ ID NO: 184; Table 13). The second one is a C to T nucleotide substitutions (Abacus, 23TRC1-17-1, 23PLP1-7-1, and CBDRx: C; 23TRC1-16-1 and 22TRP2-2-2: T) at CDS position 198 bp of 22TRP2-2-2 (SEQ ID NO: 178), and Abacus (SEQ ID NO: 177; located at position 51 bp for SEQ ID NO: 185; Table 13).

NCBI conserved domain search (Marchler-Bauer, Aron, et at. “CDD/SPARCLE: functional classification of proteins via subfamily domain architectures.” Nucleic acids research 45.D1 (2017): D200-D203.) identified the location of a phosphatidylethanolamine-binding protein (PEBP) domain between amino acid positions 23-192 in the Abacus protein sequence. The L66V substitution is located in this PEBP domain, which is important for the function of the FT gene (Song, Cheng, et at. “Genome-wide analysis of PEBP genes in Dendrobium huoshanense: Unveiling the antagonistic functions of FT/TFL1 in flowering time.” Frontiers in Genetics 12 (2021): 687689.). The NNNN indel is located at the beginning of the first exon of FT and therefore might affect binding of transcription factors CIB4 (CRY2-interacting bHLH4) and CTBS (CRY2-interacting bHLH5), which function redundantly to activate the transcription of FT and flowering initiation (Liu, Yawen, et al. “Multiple bHLH proteins form heterodimers to mediate CRY2-dependent regulation of flowering-time in Arabidopsis.” PLoS genetics 9.10 (2013): e1003861L). Without being bound to any particular theory, because FT has a central position in mediating the onset of flowering, both the NNNN insertion and the V amino acid in the L66V amino acid substitution may have an effect on the ability and earliness of accessions to form flower clusters with at least six pistils under 18/6 (Kobayashi, Yasushi, et al. “A pair of related genes with antagonistic roles in mediating flowering signals.” Science 286.5446 (1999): 1960-1962.; Pin, P. A., and Ove Nilsson. “The multifaceted roles of FLOWERING LOCUS T in plant development.” Plant, cell & environment 35.10 (2012): 1742-1755.).

### AGO5

Alignment of AGO5 CDS and protein sequence of Abacus/20LCMP-1:1, 22TRP2-2-2, 23TRC1-17-1, 23PLP1-5-8, 23PLP1-7-1, 23TRC1-15-1, and CBDRx revealed 11 amino acid substitutions which were not previously described as amino acid substitutions in AGO5 that are beneficial for flower initiation under 18/6 (Dowling, Caroline A., et al. “A FLOWERING LOCUS T ortholog is associated with photoperiod-insensitive flowering in hemp (Cannabis sativa L.).” bioRxiv (2023): 2023-04.).

The first amino acid substitution is a V (Valine, observed in Abacus/20LCMP-1:1, 23TRC1-15-1, 23PLP1-7-1, and CBDRx) to I (Isoleucine, observed in 23TRC1-17-1) at amino acid position 263 in Abacus/20LCMP-1:1 (V263I; SEQ ID NO: 221) and 23TRC1-17-1 (SEQ ID NO: 222), caused by a G to A nucleotide substitution (Abacus/20LCMP-1:1, 23TRC1-15-1: C; 23PLP1-7-1, and CBDRx: G; 23TRC1-17-1: A) at CDS position 787 bp of 23TRC1-17-1 (SEQ ID NO: 219), and Abacus (SEQ ID NO: 218; located at position 51 bp for SEQ ID NO: 224; Table 13).

The second amino acid substitution is an A (Alanine, observed in Abacus/20LCMP-1:1 and CBDRx) to M (Methionine, observed in 23TRC1-17-1) at amino acid position 463 in Abacus/20LCMP-1:1 (A463M; SEQ ID NO: 221) and 23TRC1-17-1 (SEQ ID NO: 222), caused by a C to T nucleotide substitution (Abacus/20LCMP-1:1 and CBDRx: C; 23TRC1-17-1: T) at CDS position 1388 bp of 23TRC1-17-1 (SEQ ID NO: 219), and Abacus (SEQ ID NO: 218; located at position 51 bp for SEQ ID NO: 225; Table 13).

The third amino acid substitution is an E (Glutamic Acid, observed in Abacus/20LCMP-1:1 and CBDRx) to K (Lysine, observed in 23TRC1-17-1) at amino acid position 477 in Abacus/20LCMP-1:1 (E477K; SEQ ID NO: 221) and 23TRC1-17-1 (SEQ ID NO: 222), caused by a G to A nucleotide substitution (Abacus/20LCMP-1:1 and CBDRx: G; 23TRC1-17-1:A) at CDS position 1429 bp of 23TRC1-17-1 (SEQ ID NO: 219), and Abacus (SEQ ID NO: 218; located at position 51 bp for SEQ ID NO: 226; Table 13).

The fourth amino acid substitution is an N (Asparagine, observed in Abacus/20LCMP-1:1) to K (Lysine, observed in 23TRC1-17-1 and CBDRx) at amino acid position 494 in Abacus/20LCMP-1:1 (N494K; SEQ ID NO: 221) and 23TRC1-17-1 (SEQ ID NO: 222), caused by a T to A nucleotide substitution (Abacus/20LCMP-1:1: T; 23TRC1-17-1 and CBDRx: A) at CDS position 1482 bp of 23TRC1-17-1 (SEQ ID NO: 219), and Abacus (SEQ ID NO: 218; located at position 51 bp for SEQ ID NO: 227; Table 13).

The fifth amino acid substitution is an I (Isoleucine, observed in Abacus/20LCMP-1:1 and CBDRx) to M (Methionine, observed in 23TRC1-17-1) at amino acid position 523 in Abacus/20LCMP-1:1 (I523M; SEQ ID NO: 221) and 23TRC1-17-1 (SEQ ID NO: 222), caused by a T to G nucleotide substitution (Abacus/20LCMP-1:1 and CBDRx: T; 23TRC1-17-1: G) at CDS position 1569 bp of 23TRC1-17-1 (SEQ ID NO: 219), and Abacus (SEQ ID NO: 218; located at position 51 bp for SEQ ID NO: 228; Table 13).

The sixth amino acid substitution is an N (Isoleucine, observed in Abacus/20LCMP-1:1) to K (Methionine, observed in 23TRC1-17-1 and CBDRx) at amino acid position 531 in Abacus/20LCMP-1:1 (N531K; SEQ ID NO: 221) and 23TRC1-17-1 (SEQ ID NO: 222), caused by a C to A nucleotide substitution (Abacus/20LCMP-1:1: C; 23TRC1-17-1 and CBDRx: A) at CDS position 1593 bp of 23TRC1-17-1 (SEQ ID NO: 219), and Abacus (SEQ ID NO: 218; located at position 51 bp for SEQ ID NO: 229; Table 13).

The seventh amino acid substitution is an E (Glutamic Acid, observed in Abacus/20LCMP-1:1, 22TRP2-2-2, and CBDRx) to D (Aspartic Acid, observed in 23TRC1-17-1) at amino acid position 594 in Abacus/20LCMP-1:1 (E694D; SEQ ID NO: 221) and 23TRC1-17-1 (SEQ ID NO: 222), caused by a G to T nucleotide substitution (Abacus/20LCMP-1:1, 22TRP2-2-2, and CBDRx: G; 23TRC1-17-1: T) at CDS position 2082 bp of 23TRC1-17-1 (SEQ ID NO: 219), and Abacus (SEQ ID NO: 218; located at position 51 bp for SEQ ID NO: 230; Table 13).

The eighth amino acid substitution is an G (Glycine, observed in Abacus/20LCMP-1:1, 23TRC1-17-1, and CBDRx) to E (Glutamic Acid, observed in 22TRP2-2-2) at amino acid position 696 in Abacus/20LCMP-1:1 (G696E; SEQ ID NO: 221) and 22TRP2-2-2 (SEQ ID NO: 223), caused by a G to A nucleotide substitution (Abacus/20LCMP-1:1, 23TRC1-17-1, and CBDRx: G; 22TRP2-2-2: A) at CDS position 2087 bp of 22TRP2-2-2 (SEQ ID NO: 220), and Abacus (SEQ ID NO: 218; located at position 51 bp for SEQ ID NO: 231; Table 13).

The ninth amino acid substitution is an D (Aspartic Acid, observed in Abacus/20LCMP-1:1) to N (Asparagine, observed in 22TRP2-2-2 and CBDRx) at amino acid position 723 in Abacus/20LCMP-1:1 (D723N; SEQ ID NO: 221) and 22TRP2-2-2 (SEQ ID NO: 223), caused by a G to A nucleotide substitution (Abacus/20LCMP-1:1: G; 22TRP2-2-2 and CBDRx: A) at CDS position 2167 bp of 22TRP2-2-2 (SEQ ID NO: 220), and Abacus (SEQ ID NO: 218; located at position 51 bp for SEQ ID NO: 232; Table 13).

The tenth amino acid substitution is an N (Asparagine, observed in Abacus/20LCMP-1:1) to H (Histidine, observed in 22TRP2-2-2 and CBDRx) at amino acid position 740 in Abacus/20LCMP-1:1 (N740H; SEQ ID NO: 221) and 22TRP2-2-2 (SEQ ID NO: 223), caused by a A to C nucleotide substitution (Abacus/20LCMP-1:1: A; 22TRP2-2-2 and CBDRx: C) at CDS position 2218 bp of 22TRP2-2-2 (SEQ ID NO: 220), and Abacus (SEQ ID NO: 218; located at position 51 bp for SEQ ID NO: 233; Table 13).

The eleventh amino acid substitution is an H (Histidine, observed in Abacus/20LCMP-1:1) to Q (Glutamine, observed in 22TRP2-2-2 and CBDRx) at amino acid position 746 in Abacus/20LCMP-1:1 (H746Q; SEQ ID NO: 221) and 22TRP2-2-2 (SEQ ID NO: 223), caused by a C to A nucleotide substitution (Abacus/20LCMP-1:1: C; 22TRP2-2-2 and CBDRx: A) at CDS position 2238 bp of 22TRP2-2-2 (SEQ ID NO: 220), and Abacus (SEQ ID NO: 218; located at position 51 bp for SEQ ID NO: 234; Table 13).

NCBI conserved domain search (Marchler-Bauer, Aron, et al. “CDD/SPARCLE: functional classification of proteins via subfamily domain architectures.” Nucleic acids research 45.D1 (2017): D200-D203.) identified four domains: 1. N-terminal domain of argonaute (ArgoN) between amino acid positions 87-152 of the Abacus AGO5 protein sequence; 2. Argonaute linker 1 domain (ArgoL1) between amino acid positions 163-212 of the Abacus AGO5 protein sequence; 3. PAZ domain between amino acid positions 213-324 of the Abacus protein sequence; 4. PIWI domain between amino acid positions 371-876 of the Abacus AGO5 protein sequence. The V263 amino acid substitution is located in the PAZ domain, whereas all other amino acid substitutions are located in the PIWI domain. AGO5 controls age-dependent flowering through interaction with miR156; ago5 mutants are early flowering (Roussin-Léveillée, Charles, Guilherme Silva-Martins, and Peter Moffett. “ARGONAUTES represses age-dependent induction of flowering through physical and functional interaction with miR156 in Arabidopsis.” Plant and Cell Physiology 61.5 (2020): 957-966.). Without being bound to any particular theory, the combination of amino acids, I, M, K, K, M, K, D in 23TRP1-17-1 observed for amino acid substitutions V263I, A463M, E477K, N494K, I523M, N531K, and E694D, as well as the combination of amino acids E, N, H, Q, in 22TRP2-2-2 observed for amino acid substitutions G696E, D723N, N740H, and H746Q may contribute to the ability of these two accessions to form flower clusters with at least six pistils under 18/6. In addition, it is expected that some of these amino acids in 22TRP2-2-2 contribute to its ability to form flower clusters with six pistils earlier than 23TRC1-17-1.

### CDF2

Alignment of CDF2 CDS and protein sequence of Abacus/20LCMP-1:1, Finola/23TRC1-16-1, 22TRP2-2-2, 23TRC1-17-1, 23PLP1-4-15, 23PLP1-5-8, 23PLP1-7-1, 23TRC1-15-1, and CBDRx revealed eight amino acid substitutions, which in combination distinguish between accessions that are able to form flower clusters with at least six pistils under 18/6, Finola/23TRC1-16-1, 22TRP2-2-2, and 23TRP1-17-1, from accessions that are unable to form flower clusters with at least six pistils under 18/6, Abacus/20LCMP-1:1, 23TRC1-15-1, 23PLP1-4-15, 23PLP1-5-8, 23PLP1-7-1, and CBDRx.

The first amino acid substitution is a T (Threonine, observed in Abacus/20LCMP-1:1 and 23PLP1-4-15) to A (Alanine, observed in 23TRC1-16-1, 22TRP2-2-2, 23TRC1-17-1, 23TRC1-15-1, 23PLP1-5-8, 23PLP1-7-1, and CBDRx) at amino acid position 103 in Abacus (T103A; SEQ ID NO: 190) and 23TRC1-17-1 (SEQ ID NO: 191), caused by a A to G nucleotide substitution (Abacus/20LCMP-1:1 and 23PLP1-4-15: A, 23TRC1-16-1, 22TRP2-2-2, 23TRC1-17-1, 23TRC1-15-1, 23PLP1-5-8, 23PLP1-7-1, and CBDRx: G) at CDS position 307 bp of 23TRC1-17-1 (SEQ ID NO: 187), and Abacus (SEQ ID NO: 186; located at position 51 bp for SEQ ID NO: 194; Table 13).

The second amino acid substitution is a T (Threonine, observed in Abacus/20LCMP-1:1. 23TRC1-16-1, 23PLP1-4-15, 23TRC1-15-1, 23PLP1-5-8, 23PLP1-7-1, and CBDRx) to N (Asparagine, observed in 22TRP2-2-2, 23TRC1-17-1) at amino acid position 127 in Abacus (T127N; SEQ ID NO: 190) and 23TRC1-17-1 (SEQ ID NO: 191), caused by a C to A nucleotide substitution (Abacus/20LCMP-1:1, 23TRC1-16-1, 23PLP1-4-15, 23TRC1-15-1, 23PLP1-5-8, 23PLP1-7-1, and CBDRx: C, 22TRP2-2-2, 23TRC1-17-1: A) at CDS position 380 bp of 23TRC1-17-1 (SEQ ID NO: 187), and Abacus (SEQ ID NO: 186; located at position 51 bp for SEQ ID NO: 195; Table 13).

The third amino acid substitution is an M (Methionine, observed in Abacus/20LCMP-1:1, 22TRP2-2-2, 23PLP1-4-15, 23TRC1-15-1, 23PLP1-5-8, 23PLP1-7-1, and CBDRx) to L (Leucine, observed in 23TRC1-16-1 and 23TRC1-17-1) at amino acid position 265 in Abacus (M265L; SEQ ID NO: 190) and 23TRC1-17-1 (SEQ ID NO: 191), caused by a A to T nucleotide substitution (Abacus/20LCMP-1:1, 22TRP2-2-2, 23PLP1-4-15, 23TRC1-15-1, 23PLP1-5-8, 23PLP1-7-1, and CBDRx: A, 23TRC1-16-1 and 23TRC1-17-1: T) at CDS position 793 bp of 23TRC1-17-1 (SEQ ID NO: 187), and Abacus (SEQ ID NO: 186; located at position 51 bp for SEQ ID NO: 196; Table 13).

The fourth amino acid substitution is an A (Alanine, observed in Abacus/20LCMP-1:1, 23PLP1-4-15, 23PLP1-5-8, 23PLP1-7-1, and CBDRx) to P (Proline, observed in 23TRC1-16-1, 22TRP2-2-2, 23TRC1-17-1, and 23TRC1-15-1) at amino acid position 284 in Abacus (A284P; SEQ ID NO: 190) and 23TRC1-17-1 (SEQ ID NO: 191), caused by a G to C nucleotide substitution (Abacus/20LCMP-1:1, 23PLP1-4-15, 23PLP1-5-8, 23PLP1-7-1, and CBDRx: G. 23TRC1-16-1, 22TRP2-2-2, 23TRC1-17-1, and 23TRC1-15-1: C) at CDS position 850 bp of 23TRC1-17-1 (SEQ ID NO: 187), and Abacus (SEQ ID NO: 186; located at position 51 bp for SEQ ID NO: 197; Table 13).

The fifth amino acid substitution is an K (Lysine, observed in Abacus/20LCMP-1:1 and 23PLP1-4-15) to Q (Glutamine, observed in 23TRC1-16-1, 22TRP2-2-2, 23TRC1-17-1, 23TRC1-15-1, 23PLP1-5-8, 23PLP1-7-1, and CBDRx) at amino acid position 286 in Abacus (K286Q; SEQ ID NO: 190) and 23TRC1-17-1 (SEQ ID NO: 191), caused by a A to C nucleotide substitution (Abacus/20LCMP-1:1 and 23PLP1-4-15: A, 23TRC1-16-1, 22TRP2-2-2, 23TRC1-17-1, 23TRC1-15-1, 23PLP1-5-8, 23PLP1-7-1, and CBDRx: C) at CDS position 856 bp of 23TRC1-17-1 (SEQ ID NO: 187), and Abacus (SEQ ID NO: 186; located at position 51 bp for SEQ ID NO: 198; Table 13).

The sixth amino acid substitution is an N (Asparagine, observed in Abacus/20LCMP-1:1, 23PLP1-4-15, and 23PLP1-5-8) to H (Histidine, observed in 23TRC1-16-1, 22TRP2-2-2, 23TRC1-17-1, 23TRC1-15-1, 23PLP1-7-1, and CBDRx) at amino acid position 290 in Abacus (N290H; SEQ ID NO: 190) and 23TRC1-17-1 (SEQ ID NO: 191), caused by a A to C nucleotide substitution (Abacus/20LCMP-1:1, 23PLP1-4-15, and 23PLP1-5-8: A, 23TRC1-16-1, 22TRP2-2-2, 23TRC1-17-1, 23TRC1-15-1, 23PLP1-7-1, and CBDRx: C) at CDS position 868 bp of 23TRC1-17-1 (SEQ ID NO: 187), and Abacus (SEQ ID NO: 186; located at position 51 bp for SEQ ID NO: 199; Table 13).

The seventh amino acid substitution is an N (Asparagine, observed in Abacus/20LCMP-1:1, 23TRC1-16-1, 22TRP2-2-2, 23TRC1-17-1, 23TRC1-15-1 and 23PLP1-4-15) to I (Isoleucine, observed in 23PLP1-5-8, 23PLP1-7-1, and CBDRx) at amino acid position 293 in Abacus (N293I; SEQ ID NO: 190) and 23PLP1-5-8 (SEQ ID NO: 193), caused by a A to T nucleotide substitution (Abacus/20LCMP-1:1, 23TRC1-16-1, 22TRP2-2-2, 23TRC1-17-1, 23TRC1-15-1 and 23PLP1-4-15: A, 23PLP1-5-8, 23PLP1-7-1, and CBDRx: T) at CDS position 878 bp of 23PLP1-5-8 (SEQ ID NO: 189), and Abacus (SEQ ID NO: 186; located at position 51 bp for SEQ ID NO: 200; Table 13).

The eighth amino acid substitution is an A (Alanine, observed in Abacus/20LCMP-1:1, 22TRP2-2-2, 23TRC1-17-1, 23TRC1-15-1, 23PLP1-4-15, and 23PLP1-7-1) to T (Threonine, observed in 23TRC1-16-1, 23PLP1-5-8, and CBDRx) at amino acid position 394 in Abacus (A394T; SEQ ID NO: 190) and 23TRC1-16-1 (SEQ ID NO: 192), caused by a G to A nucleotide substitution (Abacus/20LCMP-1:1 and 23PLP1-4-15, 22TRP2-2-2, 23TRC1-17-1, 23TRC1-15-1, and 23PLP1-7-1: G, 23TRC1-16-1, 23PLP1-5-8, and CBDRx: A) at CDS position 1180 bp of 23TRC1-16-1 (SEQ ID NO: 188), and Abacus (SEQ ID NO: 186; located at position 51 bp for SEQ ID NO: 201; Table 13).

NCBI conserved domain search (Marchler-Bauer, Aron, et al. “CDD/SPARCLE: functional classification of proteins via subfamily domain architectures.” Nucleic acids research 45.D1 (2017): D200-D203.) identified three domains in the Abacus protein sequence of CDF2: 1. Superantigen-like protein SSL3 (PRK13335 super family) between amino acid positions 22-135; 2. Dof domain, zinc finger (zf-Dof) between amino acid positions 148-204; 3. Large tegument protein UL36 (PHA03247 super family) between amino acid positions 314-503. The first two amino acid substitutions, T103A and T127N, are located inside the SSL3 domain. The last amino acid substitution, A394T, is located in the UL36 domain. Amino acid substitutions T127N, M265L, and A284P were found conserved as T, L, and P, respectively, in Arabidopsis, which flowers under long day conditions. Without being bound to any particular theory, the observed amino acids may contribute to the ability to form flower clusters with at least six pistils under 18/6. It appears that having all amino acid substitutions, as observed in Finola/23TRC1-16-1, causes earlier flowering under 18/6, as compared to accessions with one or two fewer amino acid substitutions, such as 22TRP2-2-2 and 23TRC1-17-1, which flower later under 18/6.

### GASA4

Alignment of GASA4 CDS and protein sequence of Abacus/20LCMP-1:1. Finola/23TRC1-16-1, 22TRP2-2-2, 23TRC1-17-1, 23TRC1-15-1, 23PLP1-4-15, 23PLP1-5-8, 23PLP1-7-1, and CBDRx revealed one amino acid substitution distinguishing between an accession that is able to form flower clusters with at least six pistils under 18/6, 23TRC1-17-1, and the other accessions, Abacus/20LCMP-1:1, Finola/23TRC1-16-1, 22TRP2-2-2, 23TRC1-15-1, 23PLP1-4-15, 23PLP1-5-8, 23PLP1-7-1, and CBDRx.

This amino acid substitution is a K (Lysine, observed in Abacus/20LCMP-1:1, Finola/23TRC1-16-1, 22TRP2-2-2, 23TRC1-15-1, 23PLP1-4-15, 23PLP1-5-8, 23PLP1-7-1, and CBDRx) to E (Glutamic Acid, observed in 23TRC1-17-1) at amino acid position 38 in Abacus (K38E; SEQ ID NO: 204) and 23TRC1-17-1 (SEQ ID NO: 205), caused by an A to G nucleotide substitution (Abacus/20LCMP-1:1, Finola/23TRC1-16-1, 22TRP2-2-2, 23TRC1-15-1, 23PLP1-4-15, 23PLP1-5-8, 23PLP1-7-1, and CBDRx: A, 23TRC1-17-1: G) at CDS position 112 bp of 23TRC1-17-1 (SEQ ID NO: 203, and Abacus (SEQ ID NO: 202; located at position 51 bp for SEQ ID NO: 206; Table 13).

Alignment of Cannabis GASA4 protein sequence with Arabidopsis GASA4 protein sequence revealed that the K38E substitution is located in the chain region of the protein (annotated between amino acid positions 26-106 in Arabidopsis; Uniprot ID P46690). In Arabidopsis, over-expression of the GASA4 gene promotes flowering under short-day conditions, where Arabidopsis normally flowers under long-day conditions (Rubinovich, Lior, and David Weiss. “The Arabidopsis cysteine-rich protein GASA4 promotes GA responses and exhibits redox activity in bacteria and in planta.” The Plant Journal 64.6 (2010): 1018-1027.). Without being bound to any particular theory, the E amino acid of the K38E substitution in 23TRC1-17-1 may contribute to this accession's ability to form flower clusters with at least six pistils under 18/6.

### CLPS3

Alignment of CLPS3 CDS and protein sequence of Abacus/20LCMP-1:1, 23TRC1-16-1/Finola, 22TRP2-2-2, 23TRC1-17-1, 23TRC1-15-1, 23PLP1-4-15, 23PLP1-5-8, 23PLP1-7-1, and CBDRx revealed two amino acid substitutions which in combination distinguish between accessions that are able to form flower clusters with at least six pistils under 18/6 and accessions that are unable to form flower clusters with at least six pistils under 18/6.

The first amino acid substitution is a M (Methionine, observed in Abacus/20LCMP-1:1) to L (Leucine, observed in Finola/23TRC1-16-1 and CBDRx) at amino acid position 88 in Abacus (M88L; SEQ ID NO: 209) and Finola/23TRC1-16-1 (SEQ ID NO: 210), caused by a A to C nucleotide substitution (Abacus/20LCMP-1:1: A, Finola/23TRC1-16-1 and CBDRx: C) at CDS position 262 bp of Finola/23TRC1-16-1 (SEQ ID NO: 208), and Abacus (SEQ ID NO: 207; located at position 51 bp for SEQ ID NO: 211; Table 13).

The second amino acid substitution is a V (Valine, observed in Abacus/20LCMP-1:1) to I (Isoleucine, observed in 23TRC1-16-1, 22TRP2-2-2, 23TRC1-17-1, 23TRC1-15-1, 23PLP1-4-15, 23PLP1-5-8, 23PLP1-7-1, and CBDRx) at amino acid position 238 in Abacus (V238I; SEQ ID NO: 209) and 23TRC1-16-1 (SEQ ID NO: 210), caused by a G to A nucleotide substitution (Abacus/20LCMP-1:1: G. 23TRC1-16-1, 22TRP2-2-2, 23TRC1-17-1, 23TRC1-15-1, and 23PLP1-4-15, 23PLP1-5-8, 23PLP1-7-1, and CBDRx: A) at CDS position 712 bp of 23TRC1-16-1 (SEQ ID NO: 208), and Abacus (SEQ ID NO: 207; located at position 51 bp for SEQ ID NO: 212; Table 13).

NCBI conserved domain search (Marchler-Bauer, Aron, et al. “CDD/SPARCLE: functional classification of proteins via subfamily domain architectures.” Nucleic acids research 45.D1 (2017): D200-D203.) identified two domains in the Abacus protein sequence of CLPS3: 1. Predicted GTPase subunit of the pre-mRNA cleavage complex (CLP1 super family) between amino acid position 25-444; 2. mRNA cleavage and polyadenylation factor CLP1 P-loop (CLP1_P) between amino acid positions 140-327. The first amino acid substitution, M88L, is located in the first domain, whereas the second amino acid substitutions, V238I, is located in the second domain. Over-expression of CLPS3 causes early flowering time in both long-day and short-day growth conditions in Arabidopsis (Xing, Denghui, Hongwei Zhao, and Qingshun Quinn Li. “Arabidopsis CLP1-SIMILAR PROTEIN3, an ortholog of human polyadenylation factor CLP1, functions in gametophyte, embryo, and postembryonic development.” Plant physiology 148.4 (2008): 2059-2069.). Without being bound to any particular theory, the two amino acids L and I of the amino acid substitutions M88L and V238I may contribute to the ability of Finola/23TRC1-16-1 to form flower clusters with at least six pistils under 18/6.

### PI

Alignment of PI CDS and protein sequence of Abacus/20LCMP-1:1, 22TRP2-2-2, 23TRC1-17-1, 23TRC1-15-1, 23PLP1-4-15, 23PLP1-5-8, 23PLP1-7-1, and CBDRx revealed one amino acid deletion in an accession which is able to form flower clusters with at least six pistils under 18/6, 23TRP1-17-1.

This amino acid deletion is an N (Asparagine, observed in Abacus/20LCMP-1:1, 22TRP2-2-2, 23TRC1-17-1, 23TRC1-15-1, 23PLP1-4-15, 23PLP1-5-8, 23PLP1-7-1, and CBDRx) at amino acid position 177 in Abacus (N177Del; SEQ ID NO: 215) which is absent in 23TRC1-17-1 (SEQ ID NO: 216), caused by a deletion of three nucleotides, AAT, located between positions 529-531 bp in Abacus (SEQ ID NO: 213; located at position 51 bp for SEQ ID NO: 217), 22TRP2-2-2, 23TRC1-17-1, 23TRC1-15-1, 23PLP1-4-15, 23PLP1-5-8, 23PLP1-7-1, and CBDRx. In 23TRC1-17-1 the deletion is between nucleotide positions 528-529 bp (SEQ ID NO: 214; Table 13).

Alignment of Cannabis PI protein sequence with Arabidopsis PI protein sequence revealed that the N177del deletion is located near the K-box region (annotated between 84-170 AA in Arabidopsis; Uniprot ID P48007). In Arabidopsis, pi knock-out mutants have higher BNQ gene expression, which results in earlier flowering (Mara, Chloe D., Tengbo Huang, and Vivian F. Irish. “The Arabidopsis floral homeotic proteins APETALA3 and PISTILLATA negatively regulate the BANQUO genes implicated in light signaling.” The Plant Cell 22.3 (2010): 690-702.). Without being bound to any particular theory, the deletion of the N amino acid in 23TRC1-17-1 may contribute to its ability to initiate flowers under 18/6.

Combinations of SNP markers and amino acid substitutions associated with flower initiation under 18/6 Seven SNP markers near flower initiation candidate genes allow for the discrimination of accessions based on their ability to form flower clusters with at least six pistils (Table 10). Sequencing of these candidate genes provided a more granular view of putative causative SNPs causing amino acid substitutions in these genes. Nine amino acid substitutions in five Flower Initiation candidate genes allow for the discrimination of accessions based on their ability to form flower clusters with at least six pistils (Table 11). The combination of SNP markers as well as the combination of amino acid substitutions observed in Finola/23TRC1-16-1 is associated with early formation of flower clusters with at least six pistils under 18/6 (28-36 DAS), whereas the combination of SNP markers as well as the combination of amino acid substitutions observed in 22TRP2-2 and 23TRC1-17-1 are associated with later formation of clusters of flowers with at least six pistils under 18/6 (Table 11).

TABLE 10
Accessions used for gene sequencing and their SNP marker genotypes.

| | | Days to | Days to | | | | | | | |
|---|---|---|---|---|---|---|---|---|---|---|
| | Flower | Flower | Flower | 171_{—} | 171_{—} | 171_{—} | 171_{—} | 171_{—} | 171_{—} | 171_{—} |
| | Initiation | Initiation | Initiation | 94326 | 143517 | 219947 | 1043983 | 1219675 | 1676674 | 2195254 |
| Accession name | under 18/6 | under 18/6 | under 12/12 | (ELF9) | (FT, ELF9) | (AGO5) | (CDF2) | (GASA4) | (CLPS3) | (PI) |
| Beneficial genotype## | | | | B, X | A, X | A, X | B, X | B, X | B, X | B, X |
| Finola/23TRC1-16-1*# | yes | 28-41 | 5 | B | A | A | A | B | B | A |
| 22TRP2-2-2** | yes | 48-50 | 6 | X | A | A | A | B | A | X |
| 23TRC1-17-1*** | yes | >64 | 14 | B | A | A | B | B | B | A |
| Abacus/20LCMP-1:1# | no | NA | 8 | A | A | A | A | A | A | A |
| 23TRC1-15-1 | 00 | NA | 14 | A | B | B | A | B | A | X |
| 23PLP1-4-15 | no | NA | >20 | X | B | X | A | B | X | A |
| 23PLP1-5-8 | no | NA | >20 | A | B | X | A | A | A | X |
| 23PLP1-7-1 | no | NA | >20 | B | B | A | A | B | B | X |
| CBDRx | NA | NA | NA | A | B | B | A | A | A | B |

First column: accession name,
*selfed progeny of 20GAQ-1229,
**sib of 20GAQ-1072,
***selfed progeny of sib of PTVOG-21-18;
#Abacus and Finola splice variants of the CDS were determined based on Sanger sequencing of RNA;
##Beneficial genotype for ability to form clusters of flowers with at least six pistils under 18/6 and/or early formation of clusters of flowers with at least six pistils under 18/6; Second column: ability to form clusters of flowers with at least six pistils under 18/6, NA = data not available; Third column: days till the formation of clusters of flowers with at least six pistils under 18/6, NA = data not available; Fourth column: days till the formation of clusters with flowers with at least six pistils under 12/12, NA = data not available; Fifth-eleventh columns: genotypes for SNP markers (A = homozygous reference allele, X = heterozygous, B homozygous alternate allele).

TABLE 11
Key amino acid substitutions in candidate genes which in combination distinguish
between the ability to form flower clusters with at least 6 pistils under 18/6.

| | Flowers | | | | | | | | | | |
|---|---|---|---|---|---|---|---|---|---|---|---|
| | under | ELF9 | ELF9 | FT | AGO5 | AGO5 | CDF2 | CDF2 | CDF2 | CLPS3 | CLPS3 |
| Accession name | 18/6? | T86I | N303D | L66V | V263I | G696E | T127N | M265L | A284P | M88L | V238I |
| 23TRC1-16-1* | yes | T | D | V | NA | NA | T | L | P | L | I |
| 22TRP2-2-2** | yes | T | D | V | NA | E | N | M | P | NA | I |
| 23TRC1-17-1*** | yes | T | D | L | I | G | N | L | P | NA | I |
| 20LCMP-1:1 | no | T | N | L | V | G | T | M | A | M | V |
| 23TRC1-15-1 | no | I | D | NA | V | NA | T | M | P | NA | I |
| 23PLP1-4-15 | no | I | D | NA | NA | NA | T | M | A | NA | I |
| 23PLP1-5-8 | no | NA | NA | NA | NA | NA | T | M | A | NA | I |
| 23PLP1-7-1 | no | NA | NA | L | V | NA | T | M | A | NA | I |
| CBDRx | NA | I | D | L | V | G | T | M | A | L | I |

First column: accession; Second column: ability to form flower clusters with at least 6 pistils under 18/6; Third-Eleventh columns: amino acids observed for the sequenced candidate genes, NA = data not available.

Evaluation of the F2 progeny of crosses with the three accessions that are able to form flower clusters with at least six pistils under 18/6 shows that the trait from Finola/23TRC1-16-1 is inherited in a dominant manner as previously described (Dowling, Caroline A., et al. “A FLOWERING LOCUS T ortholog is associated with photoperiod-insensitive flowering in hemp (Cannabis sativa L.).” bioRxiv (2023): 2023-04.). The ability to form flower clusters with at least six pisitls under 18/6 is inherited from 22TRP2-2 in a dominant manner, but is inherited from 23TRC1-17-1 in a recessive manner. F1 progeny of 23TRC1-16-1 that are heterozygous for the flower initiation locus begin flowering later than the parent, which indicates that alleles are acting in a co-dominant manner with respect to time to flower initiation (i.e., early flower). Furthermore, F1 progeny of 22TRP2-2-2 with Finola/23TRC1-16-1 form flower clusters with at least six pistils under 18/6 on average at roughly the midpoint between the two parents and three days sooner as compared to the F1 progeny of an AF1 autoflowering accession (homozygous for the AF1 locus; Dowling, Caroline A., et al. “A FLOWERING LOCUS T ortholog is associated with photoperiod-insensitive flowering in hemp (Cannabis sativa L.) with Finola/23TRC1-16-1. This is indicative of an additive effect of the genetics from 22TRP2-2-2 in combination with those from Finola/23TRC1-16-1 (Table 12). In addition, it appears that the early flowering effect observed for the three mapping population parents (Finola/23TRC1-16-1 initiates flowers under 18/6 earlier as compared to 22TRP2-2-2 and 23TRC1-17-1, with 23TRC1-17-1 initiating flowers latest) has a similar early effect in F1 progeny with AF1 autoflowering accessions as the other parent (Table 12). The F1 progeny of photosensitive accessions (unable to form clusters with at least six pistils under 18/6) with AF1 autoflowering accessions are incapable of forming flower clusters with at least six pistils under 18/6, because AF1 autoflowering accessions do not carry the beneficial genotypes for the flower initiation markers at the beginning of chromosome 8 and because the AF1 trait is recessive as previously described (Dowling, Caroline A., et al. “A FLOWERING LOCUS T ortholog is associated with photoperiod-insensitive flowering in hemp (Cannabis sativa L.).” bioRxiv (2023): 2023-04.).

In conclusion, the presented results provide combinations of SNP markers and putative causative SNPs that can be used to select Cannabis plants that are able to initiate flowers at day lengths greater than 12 hours. Some of these SNP markers and putative causative SNP combinations can be used to select Cannabis plants that are able to initiate flowers earlier at day lengths greater than 12 hours. In addition, some of these SNP markers and putative causative SNP combinations can be used to select for earlier flowering Cannabis plants under 12 hours day length.

TABLE 12
Effect of combination of different parents that are
able to form clusters of flowers with at least six
pistols under 18/6 on Time to Flower Initiation.

| Germplasm | Generation | Time to Flower Initiation |
|---|---|---|
| 23TRC1-16-1 | Parent | 38 |
| 22TRP2-2-2 | Parent | 50 |
| 23TRC1-17-1 | Parent | >64 |
| AF1 | Parent | 25 |
| 23TRC1-16-1 × 22TRP2-2-2 | F1 | 43 |
| 23TRC1-16-1 × AF1 | F1 | 45 |
| 22TRP2-2-2 × AF1 | F1 | 50 |
| 23TRC1-17-1 × AF1 | F1 | 51 |
| PS × AF1 | F1 | No initiation* |

First column: germplasm evaluated, AF1 = autoflowering accessions homozygous for the AF1 locus (Dowling, Caroline A., et al. “A FLOWERING LOCUS T ortholog is associated with photoperiod-insensitive flowering in hemp (Cannabis sativa L.).” bioRxiv (2023): 2023-04.).;
Second column: generation evaluated; Third column: average Time to Flower Initiation (DAS) across four plants grown from seed,
*= unable to form flower clusters with at least six pistils under 18/6

TABLE 13
provides additional sequence information. First column: corresponding SEQ ID No; Second column: sequence description, *incomplete CDS
and protein sequence, coordinates in brackets show the start and end of the sequence as compared to the Abacus reference genome homologous
CDS and protein sequence, respectively; Third column: sequences (genomic DNA, CDS, or protein sequences as indicated in the second column
description of the sequences).

| SEQ ID NO | Description | Sequence |
|---|---|---|
| SEQ ID NO: 145 | COL1C_AF | GACAACACCGACCAACAACA |
| SEQ ID NO: 146 | COL1C_AR | AACCCATCATCCTCACTTGC |
| SEQ ID NO: 147 | COL1C_CF | AGGTCTTTTCGACTGCCAAT |
| SEQ ID NO: 148 | COL1C_DR | GGAGGCTTATTGGGTTCCTT |
| SEQ ID NO: 149 | FTC_AF | AGGGACTCTCTTGTTGTTGGT |
| SEQ ID NO: 150 | FTC_ARW | CTTCCACCWGAGCCAGTTTC |
| SEQ ID NO: 151 | AGO5C_1AF | CCCGGTTATGGTATTGTTGG |
| SEQ ID NO: 152 | AGO5C_1AR | ACGGTCGTTGACAAGTTTCC |
| SEQ ID NO: 153 | AGO5C_2AF | GAAGTGCAACAAAGCCGATT |
| SEQ ID NO: 154 | AGO5C_2AR | AATCCATCGAGGCAACCAC |
| SEQ ID NO: 155 | AGO5C_3AF | GTTGGTGGGCGAAATACAGT |
| SEQ ID NO: 156 | AGO5C_3AR | TGTAGCAACGAGCACGAATC |
| SEQ ID NO: 157 | DOF2C_AF | ACTATACCGGTGGCGGAAGT |
| SEQ ID NO: 158 | DOF2C_AR | TCTTTGGTTGGAAAGCCTTG |
| SEQ ID NO: 159 | DOF2C_CR | ACACGCCTCATCCTTTGAAT |
| SEQ ID NO: 160 | DOF2C_DF | GTGGGACGATGAGGAATGTT |
| SEQ ID NO: 161 | GASA4C_BF | GCCTTCATGATCTTGGCTTT |
| SEQ ID NO: 162 | GASA4C_AR | CATTTAGGGCCTCCTTCCTT |
| SEQ ID NO: 163 | CLPS3C_BF | CCAGGCTCAAATTTGCTGTT |
| SEQ ID NO: 164 | CLPS3C_BR | TGGAGCAAATCCTGGTTGAT |
| SEQ ID NO: 165 | CLPS3C_HF | GCCATATTGGATGGTCGAAG |
| SEQ ID NO: 166 | CLPS3C_HR | GCGGGGAGAGATCGTTTATC |
| SEQ ID NO: 167 | PIC_AF | ATGGGAAGGGGTAAGATTGA |
| SEQ ID NO: 168 | PIC_AR | TCCCTCAAATTCAGCCTCTG |
| SEQ ID NO: 169 | PIC_CF | TGCTAAGAGAAAAGCTGGAATTT |
| SEQ ID NO: 170 | PIC_CR | TCTTGGAGATTTGGCTGCAT |
| SEQ ID NO: 171 | Abacus ELF9 CDS (1-1431 bp) | ATGTCGTCTCAGGACAACACCGACCAACAACACCAATCCACTGGTGTG |
| | | GAACTACACAACGTTGTTGATGGGAACTCTCAATCCAGTATTGAAGTA |
| | | GGATGGTATATTCTCGGTGATAATCAAGAGCATGTTGGTCCCTATGTTT |
| | | CTTCTGAATTGCTTGAGCATTTCTTGAATGGGTATCTCACCCCGAGTAC |
| | | ACTCGTGTGGTCTGAAGGAAGGACTGAATGGCAGCCATTATCCTCAAT |
| | | CCCCGAGTTGATGACATACATACCTCAACAAGGAGCCGATGATTCTAT |
| | | GCCCTCCAATGATGAAGAGTTTTTGAAATGGCAGAAGGAGATTCAAGA |
| | | GGCTGAGGCTGAGGCAGAAGCAGAGGCAAAAAATGGCTCTACTGATT |
| | | TCGGCGGAGCCTCGATGGATAATGATGACAGACCTTCAACCCCACCAG |
| | | AGGGTGAAGAAGAATTCACAGACGATGACGGGACTACCTATAAATGG |
| | | GATCGAGGTCTGAGGGTTTGGGTTCCTCAGGAGGACATAGATAGCAAA |
| | | GGTGGAGAATATAAGTTAGAAGACATGATTTTCTTAAAAGAAGAAGA |
| | | GGTCTTTTCGACTGCCAATATTTCTAATGTTCCTGCAAGTGAAGAAGAT |
| | | AATACATCTAAGCAACCTGATTCCTCTACAAAACAAGTTGATGATAGC |
| | | AATGAGGCAGTGGAAGGAAAAAATGCTAAGAGAAAATTGCCTGATGA |
| | | GGAAGCTGCTAAGAAGGAACCCAATAAGCCTCCTGATAGCTGGTTTGA |
| | | ATTAAAAGTAAATACTCATGTGTATGTGACTGGGTTGCCCGAGGACGT |
| | | GACAGTTGATGAAGTGGTAGAAGTATTCTCCAAGTGCGGGATAATAAA |
| | | GGAGGATACAGAAACCAAAAAGCCCCGTATAAAGCTTTATGTTAACA |
| | | AAGAGACTGGAAGAAAGAAGGGAGATGCACTTGTTACATATCTGAAG |
| | | GAACCTTCAGTTGCTCTGGCTTTGCAAATTCTGGATGGAGCACCTTTTC |
| | | GTCCTGGTGGGATTCCAATGACAGTCACCCCAGCCAAATTTGAGCAGA |
| | | AAGGGAATACATTTATTTCCAAGAAAGTTGACAACAAGAAGAAAAAG |
| | | AAACTTAAGAAGGTGGAAGATAAGATGCTAGGATGGGGTGGTCGTGA |
| | | TGATGCGAAGTTGTGTATTCCAGCTACTGTTGTTCTTCGTTATATGTTC |
| | | ATGCCTGCTGAAATGAGGGCCGATGAAAACTTACGTTCTGAGCTAGAG |
| | | GCTGACATACAGGAGGAATGTTCAAAGCTTGGTCCACTGGACAATGTT |
| | | AAGGTGTGCGAGAATCACCCTCAAGGTGTTGTTTTAGTAAGATACAAG |
| | | GACAGGAAGGATGCTCAAAAGTGCATAGAGCTGATGAATGGAAGATG |
| | | TTTGGCGGTAGACAGATACATGCAAGTGAGGATGATGGGTTAG |
| SEQ ID NO: 172 | 23TRC1-15-1 CDS (28-1375 bp)* | CAACACCAATCCACTGGTGTGGAACTACACAACGTTGTTGATGGGAAC |
| | | TCTCAATCCAGTATTGAAGTAGGATGGTATATTCTCGGTGATAATCAA |
| | | GAGCATGTTGGTCCCTATGTTTCTTCTGAATTGCTTGAGCATTTCTTGA |
| | | ATGGGTATCTCACCCCGAGTACACTCGTGTGGTCTGAAGGAAGGACTG |
| | | AATGGCAGCCATTATCCTCAATCCCCGAGTTGATGATATACATACCTC |
| | | AACAAGGAGCCGATGATTCTATGCCCTCCAATGATGAAGAGTTTTTGA |
| | | AATGGCAGAAGGAGATTCAAGAGGCTGAGGCTGAGGCAGAAGCAGAG |
| | | GCAAAAAATGGCTCTACTGATTTCGGCGGAGCCTCGATGGATAATGAT |
| | | GACAGACCTTCAACCCCACCAGAGGGTGAAGAAGAATTCACAGACGA |
| | | TGACGGGACTACCTATAAATGGGATCGAGGTCTGAGGGTTTGGGTTCC |
| | | TCAGGAGGACATAGATAGCAAAGGTCGAGAATATAAGTTAGAAGACA |
| | | TGATTTTCTTAAAAGAAGAAGAGGTCTTTTCGACTGCCAATATTTCTAA |
| | | TGTTCCTGCAAGTGAAGAAGATAATACATCTAAGCAACCTGATTCCTC |
| | | TACAAAACAAGTTGATGATAGCAATGAGGCAGTGGAAGGAAAAAATG |
| | | CTAAGAGAAAATTGCCTGATGAGGAAGCTGCTAAGAAGGAACCCAAT |
| | | AAGCCTCCTGATAGCTGGTTTGAATTAAAAGTAAATACTCATGTGTAT |
| | | GTGACTGGGTTGCCCGAGGACGTGACAGTTGATGAAGTGGTAGAAGTA |
| | | TTCTCCAAGTGCGGGATAATAAAGGAGGATACAGAAACCAAAAAGCC |
| | | CCGTATAAAGCTTTATGTTGACAAAGAGACTGGAAGAAAGAAGGGAG |
| | | ATGCACTTGTTACATATCTGAAGGAACCTTCAGTTGCTCTGGCTTTGCA |
| | | AATTCTGGATGGAGCACCTTTTCGTCCTGGTGGGATTCCAATGACAGTC |
| | | ACCCCAGCCAAATTTGAGCAGAAAGGGAATACATTTATTTCCAAGAAA |
| | | GTTGACAACAAGAAGAAAAAGAAACTTAAGAAGGTGGAAGATAAGAT |
| | | GCTAGGATGGGGTGGTCGTGATGATGCGAAGTTGTGTATTCCAGCTAC |
| | | TGTTGTTCTTCGTTATATGTTCATGCCTGCTGAAATGAGGGCCGATGAA |
| | | AACTTACGTTCTGAGCTAGAGGCTGACATACAGGAGGAATGTTCAAAG |
| | | CTTGGTCCGCTGGACAATGTTAAGGTGTGCGAGAATCACCCTCAAGGT |
| | | GTTGTTTTAGTAAGATACAAGGACAGGAAGGATGCTCAAAAGTGCATA |
| | | GAACTGA |
| SEQ ID NO: 173 | Abacus ELF9 protein sequence | MSSQDNTDQQHQSTGVELHNVVDGNSQSSIEVGWYILGDNQEHVGPYVS |
| | (1-476 AA) | SELLEHFLNGYLTPSTLVWSEGRTEWQPLSSIPELMTYIPQQGADDSMPSN |
| | | DEEFLKWQKEIQEAEAEAEAEAKNGSTDFGGASMDNDDRPSTPPEGEEEF |
| | | TDDDGTTYKWDRGLRVWVPQEDIDSKGGEYKLEDMIFLKEEEVESTANIS |
| | | NVPASEEDNTSKQPDSSTKQVDDSNEAVEGKNAKRKLPDEEAAKKEPNK |
| | | PPDSWFELKVNTHVYVTGLPEDVTVDEVVEVFSKCGIIKEDTETKKPRIKL |
| | | YVNKETGRKKGDALVTYLKEPSVALALQILDGAPFRPGGIPMTVTPAKFE |
| | | QKGNTFISKKVDNKKKKKLKKVEDKMLGWGGRDDAKLCIPATVVLRYM |
| | | FMPAEMRADENLRSELEADIQEECSKLGPLDNVKVCENHPQGVVLVRYK |
| | | DRKDAQKCIELMNGRCLAVDRYMQVRMMG* |
| SEQ ID NO: 174 | 23TRC1-15-1 ELF9 protein | QHQSTGVELHNVVDGNSQSSIEVGWYILGDNQEHVGPYVSSELLEHFLNG |
| | sequence (10-458 AA)* | YLTPSTLVWS |
| | | EGRTEWQPLSSIPELMIYIPQQGADDSMPSNDEEFLKWQKEIQEAEAEAEA |
| | | EAKNGSTDF |
| | | GGASMDNDDRPSTPPEGEEEFTDDDGTTYKWDRGLRVWVPQEDIDSKGR |
| | | EYKLEDMIFLK |
| | | EEEVFSTANISNVPASEEDNTSKQPDSSTKQVDDSNEAVEGKNAKRKLPD |
| | | EEAAKKEPNK |
| | | PPDSWFELKVNTHVYVTGLPEDVTVDEVVEVFSKCGIIKEDTETKKPRIKL |
| | | YVDKETGRK |
| | | KGDALVTYLKEPSVALALQILDGAPFRPGGIPMTVTPAKFEQKGNTFISKK |
| | | VDNKKKKKL |
| | | KKVEDKMLGWGGRDDAKLCIPATVVLRYMFMPAEMRADENLRSELEAD |
| | | IQEECSKLGPLDNVKVCENHPQGVVLVRYKDRKDAQKCIEL |
| SEQ ID NO: 175 | Abacus ELF9 genomic DNA | TGAAGGAAGGACTGAATGGCAGCCATTATCCTCAATCCCCGAGTTGAT |
| | sequence flanking causative SNP | GACATACATACCTCAACAAGGAGCCGATGATTCTAGTAAGGGTGGAGT |
| | (C/T substitution located at position | ATTTT |
| | 257 bp in Abacus CDS) located at | |
| | position 51 bp (Abacus AA | |
| | substitution T861) | |
| SEQ ID NO: 176 | Abacus ELF9 genomic DNA | AAAAAAATTAGGATACAGAAACCAAAAAGCCCCGTATAAAGCTTTAT |
| | sequence flanking causative SNP | GTTAACAAAGAGACTGGAAGAAAGAAGGGAGATGCACTTGTTACATA |
| | (A/G substitution located at position | TCTGAAG |
| | 907 bp in Abacus CDS) located at | |
| | position 51 bp (Abacus AA | |
| | substitution N303D) | |
| SEQ ID NO: 177 | Abacus FT CDS (1-585 bp) | ATGGCTAGAGTACTACTTAATAATAATAATAATAATAATAATAATAAT |
| | | AATAATAATAATAATAGTAGGGACTCTCTTGTTGTTGGTAGAGTAATA |
| | | GGAGATGTTTTGGATCCTTTTACAAGATCAGTCCCTATGAGAGTGTATT |
| | | ATGGTAACAGAGAGGTCAACAATGGCTATGAGTTCAAACCTTCTCAAC |
| | | TTCTCAACCAACCTCGAGTTCATATTGGTGGTCAAGATCTTAGGACCTT |
| | | CTACACTTTGCTTATGGTGGATCCGGATGCTCCTAACCCTAGTGACCCC |
| | | AATCTAAGGGAGTATTTGCATTGGTTGGTGATTGATATTCCAGGAAGT |
| | | ACAGGACCAAGTTTCGGAGAAGAGGTGGTGTGCTATGAGAATCCAAG |
| | | GCCAAGTATGGGAATTCATAGGTTTGTGTTTGTGTTGTTCAAGCAATTG |
| | | GGAAGACAGACAGTGTATGCACCGGGATGGCGTCAAAATTTCAACAC |
| | | CAGAGATTTTGCTGAGCTTTATAATCTTGGTGCCCCTGTTGCTGCTCTC |
| | | TATTTCAATTGTCAGAGGGAAACTGGCTCAGGTGGAAGGAGACCTTCC |
| | | AACTAA |
| SEQ ID NO: 178 | 22TRP2-2-2 FT CDS (124-515 bp) | TCAGTCCCTATGAGAGTGTATTATGGTAACAGAGAGGTCAACAATGGC |
| | | TATGAATTCAAACCTTCTCAACTTGTTAACCAACCTCGTGTTCATGTTG |
| | | GTGGTCAAGATCTTAGGACCTTCTACACTTTGCTTATGGTGGATCCGGA |
| | | TGCTCCTAACCCTAGTGACCCCAATCTAAGGGAGTATTTGCATTGGTTG |
| | | GTGATCGATATTCCAGGAACTACAGGGCCAAGTTTCGGAGAAGAGGTG |
| | | GTGTGCTATGAGAATCCAAGGCCAAGTATGGGAATTCATAGGTTTGTG |
| | | TTTGTGTTGTTCAAGCAATTGGGACGGCAGACAGTGTATGCACCGGGA |
| | | TGGCGTCAAAATTTCAACACCAGAGATTTTGCTGAGCTTTATAATCTTG |
| | | GTGC |
| SEQ ID NO: 179 | CBDRx FT CDS (1-573 bp) | ATGGCTAGAGTACTACTTAATAATAATAATAATAATAATAATAATAAT |
| | | AATAGTAGGGACTCTCTTGTTGTTGGTAGAGTAATAGGAGATGTTTTG |
| | | GATCCTTTTACAAGATCAGTCCCTATGAGAGTGTATTATGGTAACAGA |
| | | GAGGTCAACAATGGCTATGAGTTCAAACCTTCTCAACTTCTCAACCAA |
| | | CCTCGTGTTCATATTGGTGGTCAAGATCTTAGGACCTTCTACACTTTGC |
| | | TTATGGTGGATCCGGATGCTCCTAACCCTAGTGACCCCAATCTAAGGG |
| | | AGTATTTGCATTGGTTGGTGATTGATATTCCAGGAAGTACAGGACCAA |
| | | GTTTCGGAGAAGAGGTGGTGTGCTATGAGAATCCAAGGCCAAGTATGG |
| | | GAATTCATAGATTTGTGTTTGTGTTGTTTAAACAATTGGGAAGGCAGA |
| | | CAGTGTATGCACCAGGATGGCGTCAAAATTTCAACACCAGAGACTTTG |
| | | CTGAGCTTTACAACCTTGGTGCCCCTGTTGCTGCTCTCTATTTCAACTG |
| | | CCAGAGGGAAACTGGCTCTGGTGGAAGGAGACCTTCCAACTAA |
| SEQ ID NO: 180 | Abacus FT protein sequence | MARVLLNNNNNNNNNNNNNNNSRDSLVVGRVIGDVLDPFTRSVPMRVY |
| | (1-194 AA) | YGNREVNNGYEFKPSQLLNQPRVHIGGQDLRTFYTLLMVDPDAPNPSDPN |
| | | LREYLHWLVIDIPGSTGPSFGEEVVCYENPRPSMGIHRFVFVLFKQLGRQT |
| | | VYAPGWRQNFNTRDFAELYNLGAPVAALYFNCQRETGSGGRRPSN |
| SEQ ID NO: 181 | 22TRP2-2-2 FT protein sequence | SVPMRVYYGNREVNNGYEFKPSQLVNQPRVHVGGQDLRTFYTLLMVDP |
| | (42-171) | DAPNPSDPNLREYLHWLVIDIPGTTGPSFGEEVVCYENPRPSMGIHRFVFV |
| | | LFKQLGRQTVYAPGWRQNENT RDFAELYNLG |
| SEQ ID NO: 182 | CBDRx FT protein sequence | MARVLLNNNNNNNNNNNSRDSLVVGRVIGDVLDPFTRSVPMRVYYGNR |
| | (1-190 AA) | EVNNGYEFKPSQLLNQPRVHIGGQDLRTFYTLLMVDPDAPNPSDPNLREY |
| | | LHWLVIDIPGSTGPSFGEEVVCYENPRPSMGIHRFVFVLFKQLGRQTVYAP |
| | | GWRQNFNTRDFAELYNLGAPVAALYFNCQRETGSGGRRPSN |
| SEQ ID NO: 183 | Abacus FT genomic DNA sequence | TTATTATGGCTAGAGTACTACTTAATAATAATAATAATAATAATAATA |
| | flanking causative indel | ATAATAATAATAATAATAATAGTAGGGACTCTCTTGTTGTTGGTAGAG |
| | (AATAATAAT/------- indel | TAATAGGAGATGT |
| | located between positions 46-57 | |
| | bp in Abacus CDS) located at | |
| | positions 51-62 bp (Abacus AA | |
| | substitution N16del, N17del, | |
| | N18del, N19del) | |
| SEQ ID NO: 184 | Abacus FT genomic DNA sequence | TGGTAACAGAGAGGTCAACAATGGCTATGAGTTCAAAC |
| | flanking causative SNP (C/G | CTTCTCAACTTCTCAACCAACCTCGAGTTCATATTGGTGGTCAAGATCT |
| | substitution located at position 196 | TAGGACCTTCTAC |
| | bp in Abacus CDS) located at | |
| | position 51 bp (Abacus AA | |
| | substitution L66V) | |
| SEQ ID NO: 185 | Abacus FT genomic DNA sequence | GTAACAGAGAGGTCAACAATGGCTATGAGTTCAAAC |
| | flanking causative SNP (C/T | CTTCTCAACTTCTCAACCAACCTCGAGTTCATATTGGTGGTCAAGATCT |
| | substitution located at position 198 | TAGGACCTTCTACAC |
| | bp in Abacus CDS) located at | |
| | position 51 bp (Abacus AA | |
| | substitution L66V) | |
| SEQ ID NO: 186 | Abacus CDF2 CDS (1-1536 bp) | ATGAGAGCACAAATGTCGGAGCCTAAAGACCCGGCGATCAAGCTCTTC |
| | | GGCAAGACTATACCGGTGGCGGAAGTTCCGGTTACTTCCGGTGGGTCT |
| | | CAAGGACCTTCAGTTTCGGCTTCTGCGACTTCTATGGACGATAATATGG |
| | | ATCAGGAACCTAAACCAAACTCTTCTTCTTCTGAGGTCATTACCAGTAA |
| | | AGATGAGGATGATAGAGACACAGAAAAGGCAACAACAAATGATGATC |
| | | CCACAGAGACTAAAGAGGAAGATGGAAACCAACCTATGTCTCCTGAA |
| | | GAGCCAAGGAATTCGGGTACAACTTCTGGAAGTTCTGAGAACCCGACA |
| | | ACACCTGTTGATAAGGAGAGTGTTACACCGAAAGCTTCAAAGACCGAA |
| | | GAAGAGCAGAGCGAGGGAAATAATTCACAAGAAAAGACCTTGAAAAA |
| | | ACCAGACAAGATACTTCCATGCCCCCGGTGTAATAGCATGGACACCAA |
| | | GTTCTGTTACTACAACAATTACAACGTTAACCAACCCAGACACTTCTGC |
| | | AAGAAATGCCAAAGATATTGGACTGCTGGTGGGACGATGAGGAATGT |
| | | TCCTGTGGGTGCTGGTCGTCGTAAGAACAAGAACTCTGCTTCTCACTAC |
| | | CGTCACATAACTGTTTCTGAAGCTCTTCAAAATGGAAGAACTGATGTTT |
| | | CAAGTGGAATTCACTCTCCTCATCCTTCAATGAAATCTAATGGTACTGT |
| | | CCTGACATTTGGCTCTGATGCACCCTTATGTGAATCAATGGCATCTGTT |
| | | CTGAATCTTGCTGATAAAAACATGAGGAACTGCACACAGAATGGTTTT |
| | | CTTAAACCCGAAGAAGTAAGGACTCCTGTAGCTTTTAAAAGTGTAGAA |
| | | AATGGAGATAACAATGTTGATGGATCTTCAATTACAACATCGAATTCA |
| | | AAGGATGAGGCGTGTAACAATGTTTCACAAGAACAAGTCATACAAAA |
| | | TCCTCAGGGTGTTCCTCCCCAAATGCCATGCTTTCCTGGGCCTCCTTGG |
| | | CCGTACCCTTGGAATGCTGCTCAGTGGAACCCTTCGGTTCCAATGCCCG |
| | | CTTTCTGTCCTCCAGGATATCCTATGCCATTTTACCCTGCACCTGCTTAT |
| | | TGGGGTTGTAGTGTACCAGGGACATGGAACATCCCTTGGCTTCCAATG |
| | | CCAACATCTCAAAACCCAGCCGCCCCGAGCTCTGGTCCTAACTCTCCT |
| | | AACTCCCCAACATTGGGAAAACATTCAAGGGATGAAAACACAGTTAA |
| | | ATCAAATTCCTCAGAAGAGGAACAACCAAAAGAAAGCAATTCTGAGA |
| | | GATGCCTTTGGATTCCAAAAACATTGAGGATCGACGATCCAGGTGAAG |
| | | CTGCTAGGAGCTCTATATGGGCAACATTGGGGATAAAGAATGATAAAG |
| | | CCGATTCAATCAGCGGGGGAGGACTTTTCAAGGCTTTCCAACCAAAGA |
| | | ACGACGAAAAGAACCACAGAGCAGAAAACTCAGTCTTACAAGCGAAT |
| | | CCAGCCGCATTGTCAAGATCGCTAAGCTTTCAAGAGAGGTCGTAA |
| SEQ ID NO: 187 | 23TRC1-17-1 CDF2 CDS | GAGGTCATTACCAGTAAAGATGAGGATGATAGAGACACAGAAAAGGC |
| | 1(178-399 bp) | AACAACAAATGATGATCCCACAGAGACTAAAGAGGAAGATGGAAACC |
| | | AACCCATGTCTCCTGAAGAGCCAAGAAATTCGGGTGCAACTTCTGGAA |
| | | GTTCTGAGAACCCAACAACACCTGTTGATAAGGAGAGTGTTACACCGA |
| | | AAGCTTCAAAGAACGAAGAAGAGCAGAGCGAGGGAAATAATTCACAA |
| | | GAAAAGACCTTGAAGAAACCAGACAAGATACTTCCATGCCCCCGGTGT |
| | | AATAGCATGGACACCAAGTTCTGTTACTACAACAATTACAACGTTAAC |
| | | CAACCCCGACACTTCTGCAAGAAATGCCAAAGATATTGGACTGCTGGT |
| | | GGGACGATGAGGAATGTTCCTGTGGGTGCTGGTCGTCGTAAGAACAAG |
| | | AACTCTGCTTCTCACTACCGTCACATAACTGTTTCTGAAGCTCTTCAAA |
| | | ATGGAAGAACTGATGTTTCAAGTGGAATTCACTCTCCTCATCCTTCAAT |
| | | GAAATCTAATGGTACTGTCCTGACATTTGGCTCTGATGCACCCTTATGT |
| | | GAATCAATGGCATCTGTTCTGAATCTTGCTGATAAAAACTTGAGGAAC |
| | | TGCACACAGAATGGTTTTCTTAAACCCGAAGAAGTAAGGACTCCCGTA |
| | | CCTTTTCAAAGTGTAGAACATGGAGATAACAATGTTGATGGATCTTCA |
| | | ATTACAACATCGAATTCAAAGGATGAGGCGTGTAACAATGTTTCACAA |
| | | GAACAAGTCATACAAAATCCTCAGGGTGTTCCTCCCCAAATGCCATGC |
| | | TTTCCTGGGCCTCCTTGGCCGTACCCTTGGAATGCTGCTCAGTGGAACC |
| | | CTTCGGTTCCAATGCCCGCTTTCTGTCCTCCAGGATATCCTATGCCATT |
| | | TTACCCTGCACCTGCTTATTGGGGTTGTAGTGTACCAGGGACATGGAA |
| | | CATCCCTTGGCTTCCAATGCCAACATCTCAAAACCCAGCCGCCCCGAG |
| | | CTCTGGTCCTAACTCTCCTAACTCCCCAACATTGGGAAAACATTCAAG |
| | | GGATGAAAACACAGTTAAATCAAATTCCTCAGAAGAGGAACAACCAA |
| | | AAGAAAGCAATTCTGAGAGATGCCTTTGGATTCCAAAAACATTGAGGA |
| | | TCGACGATCCAGGTGAAGCTGCTAGGAGCTCTATATGGGCAACATTGG |
| | | GGATAAAGAATGATAAAGCCG |
| SEQ ID NO: 188 | 23TRC1-16-1 CDF2 CDS | TCGGCTTCTGCGACTTCTATGGACGATAATATGGATCAGGAACCTAAA |
| | (112-1381 bp) | CCGAACTCTTCTTCTTCTGAGGTCATTACCAGTAAAGATGAGGATGAT |
| | | AGAGACACAGAAAAGGCAACAACAAATGATGATCCCACAGAGACTAA |
| | | AGAGGAAGATGGAAACCAACCTATGTCTCCTGAAGAGCCAAGAAATT |
| | | CGGGTGCAACTTCTGGAAGTTCTGAGAACCCGACAACACCTGTTGATA |
| | | AGGAGAGTGTTACACCGAAAGCTTCAAAGACCGAAGAAGAGCAGAGC |
| | | GAGGGAAATAATTCACAAGAAAAGACCTTGAAGAAACCAGACAAGAT |
| | | ACTTCCATGCCCCCGGTGTAATAGCATGGACACCAAGTTCTGTTACTAC |
| | | AACAATTACAACGTTAACCAACCCCGACACTTCTGCAAGAAATGCCAA |
| | | AGATATTGGACTGCTGGTGGGACGATGAGGAATGTTCCTGTGGGTGCT |
| | | GGTCGTCGTAAGAACAAGAACTCTGCTTCTCACTACCGTCACATAACT |
| | | GTTTCTGAAGCTCTTCAAAATGGAAGAACTGATGTTTCAAGTGGAATT |
| | | CACTCTCCTCATCCTTCAATGAAATCTAATGGTACTGTCCTGACATTTG |
| | | GCTCTGATGCACCCTTATGTGAATCAATGGCATCTGTTCTGAATCTTGC |
| | | TGATAAAAACTTGAGGAACTGCACACAGAATGGTTTTCTTAAACCCGA |
| | | AGAAGTAAGGACTCCCGTACCTTTTCAAAGTGTAGAACATGGAGATAA |
| | | CAATGTTGATGGATCTTCAATTACAACATCGAATTCAAAGGATGAGGC |
| | | GTGTAACAATGTTTCACAAGAACAAGTCATACAAAATCCTCAGGGTGT |
| | | TCCTCCCCAAATGCCATGCTTTCCTGGGCCTCCTTGGCCGTACCCTTGG |
| | | AATGCTGCTCAGTGGAACCCTTCGGTTCCAATGCCAGCTTTCTGTCCTC |
| | | CAGGATATCCTATGCCATTTTACCCTGCACCTGCTTATTGGGGTTGTAG |
| | | TGTACCAGGGACATGGAACATCCCTTGGCTTCCAATGCCAACATCTCA |
| | | AAACCCAGCCACCCCGAGCTCTGGTCCTAACTCTCCTAACTCCCCAAC |
| | | ATTGGGAAAACATTCAAGGGAGGAAAACACAGTTAAATCAAATTCCTC |
| | | AGAAGAAGAACAACCAAAAGAAAGCAATTCTGAGAGATGCCTTTGGA |
| | | TTCCAAAAACATTGAGGATCGACGATCCAGGTGAAGCTGCTAGGAGCT |
| | | CTATATGGGCAACATTGGGGA |
| SEQ ID NO: 189 | 23PLP1-5-8 CDF2 CDS (154-1366 | CCTAAACCAAACTCTTCTTCTTCTGAGGTCATTACCAGTAAAGATGAG |
| | bp) | GATGATAGAGACACAGAAAAGGCAACAACAAATGATGATCCCACAGA |
| | | GACTAAAGAGGAAGATGGAAACCAACCTATGTCTCCTGAAGAGCCAA |
| | | GGAATTCGGGTGCAACTTCTGGAAGTTCTGAGAACCCGACAACACCTG |
| | | TTGATAAGGAGAGTGTTACACCGAAAGCTTCAAAGACCGAAGAAGAG |
| | | CAGAGCGAGGGAAATAATTCACAAGAAAAGACCTTGAAAAAACCAGA |
| | | CAAGATACTTCCATGCCCCCGGTGTAATAGCATGGACACCAAGTTCTG |
| | | TTACTACAACAATTACAACGTTAACCAACCCCGACACTTCTGCAAGAA |
| | | ATGCCAAAGATATTGGACTGCTGGTGGGACGATGAGGAATGTTCCTGT |
| | | GGGTGCTGGTCGTCGTAAGAACAAGAACTCTGCTTCTCACTACCGTCA |
| | | CATAACTGTTTCTGAAGCTCTTCAAAATGGAAGAACTGATGTTTCAAG |
| | | TGGAATTCACTCTCCTCATCCTTCAATGAAATCTAATGGTACTGTCCTG |
| | | ACATTTGGCTCTGATGCACCCTTATGTGAATCAATGGCATCTGTTCTGA |
| | | ATCTTGCTGATAAAAACATGAGGAACTGCACACAGAATGGTTTTCTTA |
| | | AACCCGAAGAAGTAAGGACTCCTGTAGCTTTTCAAAGTGTAGAAAATG |
| | | GAGATATCAATGTTGATGGATCTTCAATTACAACATCGAATTCAAAGG |
| | | ATGAGGCGTGTAACAATGTTTCACAAGAACAAGTCATACAAAATCCTC |
| | | AGGGTGTTCCTCCCCAAATGCCATGCTTTCCTGGGCCTCCTTGGCCGTA |
| | | CCCTTGGAATGCTGCTCAGTGGAACCCTTCGGTTCCAATGCCCGCTTTC |
| | | TGTCCTCCAGGATATCCTATGCCATTTTACCCTGCACCTGCTTATTGGG |
| | | GTTGTAGTGTACCAGGGACATGGAACATCCCTTGGCTTCCAATGCCAA |
| | | CATCTCAAAACCCAGCCACCCCGAGCTCTGGTCCTAACTCTCCTAACTC |
| | | CCCAACATTGGGAAAACATTCAAGGGAGGAAAACACAGTTAAATCAA |
| | | ATTCCTCAGAAGAAGAGCAACCAAAAGAAAGCAATTCTGAGAGATGC |
| | | CTTTGGATTCCAAAAACATTGAGGATCGACGATCCAGGTGAAGCTGCT |
| | | AGGAGCTCTATAT |
| SEQ ID NO: 190 | Abacus CDF2 protein sequence (1- | MRAQMSEPKDPAIKLFGKTIPVAEVPVTSGGSQGPSVSASATSMDDNMDQ |
| | 511 AA) | EPKPNSSSSEVITSKDEDDRDTEKATTNDDPTETKEEDGNQPMSPEEPRNS |
| | | GTTSGSSENPTTPVDKESVTPKASKTEEEQSEGNNSQEKTLKKPDKILPCPR |
| | | CNSMDTKFCYYNNYNVNQPRHFCKKCQRYWTAGGTMRNVPVGAGRRK |
| | | NKNSASHYRHITVSEALQNGRTDVSSGIHSPHPSMKSNGTVLTFGSDAPLC |
| | | ESMASVLNLADKNMRNCTQNGFLKPEEVRTPVAFKSVENGDNNVDGSSI |
| | | TTSNSKDEACNNVSQEQVIQNPQGVPPQMPCFPGPPWPYPWNAAQWNPS |
| | | VPMPAFCPPGYPMPFYPAPAYWGCSVPGTWNIPWLPMPTSQNPAAPSSGP |
| | | NSPNSPTLGKHSRDENTVKSNSSEEEQPKESNSERCLWIPKTLRIDDPGEA |
| | | ARSSIWATLGIKNDKADSISGGGLFKAFQPKNDEKNHRAENSVLQANPAA |
| | | LSRSLSFQERS |
| SEQ ID NO: 191 | 23TRC1-17-1 CDF2 protein | EVITSKDEDDRDTEKATTNDDPTETKEEDGNQPMSPEEPRNSGATSGSSEN |
| | sequence (60-466 AA) | PTTPVDKESVTPKASKNEEEQSEGNNSQEKTLKKPDKILPCPRCNSMDTKF |
| | | CYYNNYNVNQPRHFCKKCQRYWTAGGTMRNVPVGAGRRKNKNSASHY |
| | | RHITVSEALQNGRTDVSSGIHSPHPSMKSNGTVLTFGSDAPLCESMASVLN |
| | | LADKNLRNCTQNGFLKPEEVRTPVPFQSVEHGDNNVDGSSITTSNSKDEA |
| | | CNNVSQEQVIQNPQGVPPQMPCFPGPPWPYPWNAAQWNPSVPMPAFCPP |
| | | GYPMPFYPAPAYWGCSVPGTWNIPWLPMPTSQNPAAPSSGPNSPNSPTLG |
| | | KHSRDENTVKSNSSEEEQPKESNSERCLWIPKTLRIDDPGEAARSSIWATL |
| | | GIKNDKA |
| SEQ ID NO: 192 | 23TRC1-16-1 CDF2 protein | SASATSMDDNMDQEPKPNSSSSEVITSKDEDDRDTEKATTNDDPTETKEE |
| | sequence (38-460 AA) | DGNQPMSPEEPRNSGATSGSSENPTTPVDKESVTPKASKTEEEQSEGNNSQ |
| | | EKTLKKPDKILPCPRCNSMDTKFCYYNNYNVNQPRHFCKKCQRYWTAGG |
| | | TMRNVPVGAGRRKNKNSASHYRHITVSEALQNGRTDVSSGIHSPHPSMKS |
| | | NGTVLTFGSDAPLCESMASVLNLADKNLRNCTQNGFLKPEEVRTPVPFQS |
| | | VEHGDNNVDGSSITTSNSKDEACNNVSQEQVIQNPQGVPPQMPCFPGPPW |
| | | PYPWNAAQWNPSVPMPAFCPPGYPMPFYPAPAYWGCSVPGTWNIPWLP |
| | | MPTSQNPATPSSGPNSPNSPTLGKHSREENTVKSNSSEEEQPKESNSERCL |
| | | WIPKTLRIDDPGEAARSSIWA TLG |
| SEQ ID NO: 193 | 23PLP1-5-8 CDF2 protein sequence | PKPNSSSSEVITSKDEDDRDTEKATTNDDPTETKEEDGNQPMSPEEPRNSG |
| | (52-455 AA) | ATSGSSENPTTPVDKESVTPKASKTEEEQSEGNNSQEKTLKKPDKILPCPR |
| | | CNSMDTKFCYYNNYNVNQPRHFCKKCQRYWTAGGTMRNVPVGAGRRK |
| | | NKNSASHYRHITVSEALQNGRTDVSSGIHSP |
| | | HPSMKSNGTVLTFGSDAPLCESMASVLNLADKNMRNCTQNGFLKPEEVR |
| | | TPVAFQSVENGDINVDGSSITTSNSKDEACNNVSQEQVIQNPQGVPPQMPC |
| | | FPGPPWPYPWNAAQWNPSVPMPAFCPPGYPMPFYPAPAYWGCSVPGTW |
| | | NIPWLPMPTSQNPATPSSGPNSPNSPTLGKHSREENTVKSNSSEEEQPKESN |
| | | SERCLWIPKTLRIDDPGEAARSSI |
| SEQ ID NO: 194 | Abacus CDF2 genomic DNA | AGGAAGATGGAAACCAACCTATGTCTCCTGAAGAGCCAAGGAATTCG |
| | sequence flanking causative SNP | GGTACAACTTCTGGAAGTTCTGAGAACCCGACAACACCTGTTGATAAG |
| | (A/G substitution located at position | GAGAGT |
| | 307 bp in Abacus CDS) located at | |
| | position 51 bp (Abacus AA | |
| | substitution T103A) | |
| SEQ ID NO: 195 | Abacus CDF2 genomic DNA | CCCGACAACACCTGTTGATAAGGAGAGTGTTACACCGAAAGCTTCAAA |
| | sequence flanking causative SNP | GACCGAAGAAGAGCAGAGCGAGGGAAATAATTCACAAGAAAAGACCT |
| | (C/A substitution located at position | TGAAAA |
| | 380 bp in Abacus CDS) located at | |
| | position 51 bp (Abacus AA | |
| | substitution T127N) | |
| SEQ ID NO: 196 | Abacus CDF2 genomic DNA | CACCCTTATGTGAATCAATGGCATCTGTTCTGAATCTTGCTGATAAAAA |
| | sequence flanking causative SNP | CATGAGGAACTGCACACAGAATGGTTTTCTTAAACCCGAAGAAGTAAG |
| | (A/T substitution located at position | GACT |
| | 793 bp in Abacus CDS) located at | |
| | position 51 bp (Abacus AA | |
| | substitution M265L) | |
| SEQ ID NO: 197 | Abacus CDF2 genomic DNA | ACTGCACACAGAATGGTTTTCTTAAACCCGAAGAAGTAAGGACTCCTG |
| | sequence flanking causative SNP | TAGCTTTTAAAAGTGTAGAAAATGGAGATAACAATGTTGATGGATCTT |
| | (G/C substitution located at position | CAATT |
| | 850 bp in Abacus CDS) located at | |
| | position 51 bp (Abacus AA | |
| | substitution A284P) | |
| SEQ ID NO: 198 | Abacus CDF2 genomic DNA | CACAGAATGGTTTTCTTAAACCCGAAGAAGTAAGGACTCCTGTAGCTT |
| | sequence flanking causative SNP | TTAAAAGTGTAGAAAATGGAGATAACAATGTTGATGGATCTTCAATTA |
| | (A/C substitution located at position | CAACA |
| | 856 bp in Abacus CDS) located at | |
| | position 51 bp (Abacus AA | |
| | substitution K286Q) | |
| SEQ ID NO: 199 | Abacus CDF2 genomic DNA | TTTCTTAAACCCGAAGAAGTAAGGACTCCTGTAGCTTTTAAAAGTGTA |
| | sequence flanking causative SNP | GAAAATGGAGATAACAATGTTGATGGATCTTCAATTACAACATCGAAT |
| | (A/C substitution located at position | TCAAA |
| | 868 bp in Abacus CDS) located at | |
| | position 51 bp (Abacus AA | |
| | substitution N290H) | |
| SEQ ID NO: 200 | Abacus CDF2 genomic DNA | CGAAGAAGTAAGGACTCCTGTAGCTTTTAAAAGTGTAGAAAATGGAG |
| | sequence flanking causative SNP | ATAACAATGTTGATGGATCTTCAATTACAACATCGAATTCAAAGGATG |
| | (A/T substitution located at position | AGGCGT |
| | 878 bp in Abacus CDS) located at | |
| | position 51 bp (Abacus AA | |
| | substitution N293I) | |
| SEQ ID NO: 201 | Abacus CDF2 genomic DNA | GGACATGGAACATCCCTTGGCTTCCAATGCCAACATCTCAAAACCCAG |
| | sequence flanking causative SNP | CCGCCCCGAGCTCTGGTCCTAACTCTCCTAACTCCCCAACATTGGGAA |
| | (G/A substitution located at position | AACAT |
| | 1180 bp in Abacus CDS) located at | |
| | position 51 bp (Abacus AA | |
| | substitution A394T) | |
| SEQ ID NO: 202 | Abacus GASA4 CDS (1-327 bp) | ATGGCCATGACTAAGTTTCTGGCCTTCATGATCTTGGCTTTCATCGCCA |
| | | TTTCCATGATCCAAACAACTGTTATGGCTTCTAATGGCAATGGAGGTC |
| | | ATCACAACAACAAGAAAGGATATGGACCAGGGAGTCTGAAGAGTTAC |
| | | CAATGCCCATCTGAATGCGTGAGGAGGTGTGGAAGAACTCAGTACCAC |
| | | AAGCCGTGCATGTTCTTCTGCCAGAAATGTTGTAAGACCTGCCTGTGTG |
| | | TGCCACCGGGGTATTATGGTAATAAAGCTGTGTGCCCTTGCTACAACA |
| | | ACTGGAAGACTAAGGAAGGAGGCCCTAAATGCCCTTAG |
| SEQ ID NO: 203 | 23TRC1-17-1 GASA4 CDS (37- | GCTTTCATCGCCATTTCCATGATCCAAACAACTGTTATGGCTTCTAATG |
| | 312 bp)* | GCAATGGAGGTCATCACAACAACAAGGAAGGATATGGACCAGGGAGT |
| | | CTCAAGAGTTACCAATGCCCATCTGAATGCGTGAGGAGGTGTGGAAGA |
| | | ACCCAGTACCACAAGCCGTGCATGTTCTTCTGCCAGAAATGTTGTAAG |
| | | ACCTGCCTGTGTGTGCCACCGGGGTATTATGGTAATAAAGCTGTGTGC |
| | | CCTTGCTACAACAACTGGAAGACCAAGGAAGGAGGC |
| SEQ ID NO: 204 | Abacus GASA4 protein sequence | MAMTKFLAFMILAFIAISMIQTTVMASNGNGGHHNNKKGYGPGSLKSYQ |
| | (1-108 AA) | CPSECVRRCGRTQYHKPCMFFCQKCCKTCLCVPPGYYGNKAVCPCYNN |
| | | WKTKEGGPKCP |
| SEQ ID NO: 205 | 23TRC1-17-1 GASA4 protein | AFIAISMIQTTVMASNGNGGHHNNKEGYGPGSLKSYQCPSECVRRCGRTQ |
| | sequence (13-104 AA)* | YHKPCMFFCQKCCKTCLCVPPGYYGNKAVCPCYNNWKTKEGG |
| SEQ ID NO: 206 | Abacus GASA4 genomic DNA | TTGCATTGTAATTAATTAATTAATTATTTTTTTTAAAATTTTTATTTTAG |
| | sequence flanking causative SNP | AAAGGATATGGACCAGGGAGTCTGAAGAGTTACCGTAAGTATTTTGTT |
| | (A/G substitution located at position | ATT |
| | 112 bp in Abacus CDS) located at | |
| | position 51 bp (Abacus AA | |
| | substitution K38E) | |
| SEQ ID NO: 207 | Abacus CLPS3 CDS (1-1338 bp) | ATGGCTTATGGAGGTACACCACTGGGTCCCCCACCAATGGCTGCTGCT |
| | | GCTGCTTCATCATCAGCCTCCACAGTTCGCCAGGTCAAGTTGGACCGA |
| | | GAGAGCGAGCTTCGAATCGAAGTTGGATACGACTCCTCCCTCAGGCTC |
| | | CGACTTCTAAACGGCACGGCTGAGATATTCGGCACCGAACTCCCACCT |
| | | GAAATCTGGCTCACTTTCCCTCCCAGGCTCAAATTTGCTGTTTTCACTT |
| | | GGTATGGAGCCACAATTGAAATGGATGGTAGTACTGAAACTGACTACA |
| | | CTGCAGATGAGACCCCTATGATTAGCTACGTGAATGTCCATGCCATAT |
| | | TGGATGGTCGAAGAAACCGTGCTAAAGCTTCGTCCTCTGATGGTTCCA |
| | | GTACGGTTCAGGGGCCTCGGGTCATTGTTGTGGGACCTACAGATTCTG |
| | | GGAAGAGTACATTGTCAAGAATGCTTCTTAGTTGGGCAGCTAAACAGG |
| | | GTTGGAAACCTACTTTTGTGGACTTGGATATTGGTCAAGGATCCATAA |
| | | CTCTACCGGGATGTATTGCTGCTACTCCTATTGAAATGCCTATTGACCC |
| | | AGTTGAAGGGATTCCTCTTGAGATGCCTTTGGTTTACTTCTATGGACAC |
| | | ACCACTCCCAGTAACAATGTAGAACTATACAAAATCCTTGTGAGGGAG |
| | | CTTTCTCAACTGCTTGAGAGACAATTTGCGGGTAATGTTGAATCTCGAG |
| | | CTGCTGGCATGGTGATCAATACCATGGGTTGGATAGAAGGAATAGGAT |
| | | ATGAGTTGCTTCTTCATGCAATCGACACATTTAATATCAACGTTGTATT |
| | | GGTTCTGGGTCAGGAAAAACTTTGCAGTATGCTCAGAGATGTTCTGAA |
| | | AAACAAGCCTGATGTGGATGTTGTAAAACTTCAAAGATCCGGAGGAGT |
| | | TGTATCGAGGAATGTCAAATATCGTCAAAAGGCTAGGAGTTTGAGGAT |
| | | AAGGGAATATTTTTATGGACGGATAAACGATCTCTCCCCGCATTCTAA |
| | | TATTGCGAGTTTTAGTGATTTATTGGTCTATCGAATTGGTGGGGGACCT |
| | | CAGGCACCCCGGTCAGCCCTGCCAATTGGTGCAGAGCCTGCAGCAGAT |
| | | CCTACAAGATTAGTCCCTGTGAATATCAACCAGGATTTGCTCCATCTAG |
| | | TTCTTGCTGTTTCATTTGCGAAAGAGCCTGAGGAAATTCTTTCAAGTAA |
| | | CGTTGCCGGGTTTATATACATTACTGACATTGACATTCAGAGGAAGAA |
| | | GATCACATATCTTGCACCAGCTGCTGGTGATCTCCCGAGCAAATATCT |
| | | GATTGTGGGAACCTTGACATGGATCGAAACATAG |
| SEQ ID NO: 208 | Finola/23TRC1-16-1 CLPS3 CDS | ATGGCTTATGGAGGTACACCACTGGGTCCCCCACCAATGGCAGCTGCT |
| | (1-1338 bp) | GCTGCTTCATCATCAGCCTCCACAGTTCGCCAGGTCAAGTTGGACCGA |
| | | GAGAGCGAGCTTCGAATCGAAGTTGGATACGACTCCTCCCTCAGGCTC |
| | | CGACTTCTAAACGGCACAGCTGAGATATTCGGCACCGAACTCCCACCT |
| | | GAAATCTGGCTCACTTTCCCTCCCAGGCTCAAATTTGCTGTTTTCACTT |
| | | GGTATGGAGCCACAATTGAACTGGATGGTAGTACTGAAACTGACTACA |
| | | CTGCAGATGAGACCCCTATGATTAGCTACGTGAATGTCCATGCCATAT |
| | | TGGATGGTCGAAGAAACCGTGCTAAAGCTTCGTCCTCTGATGGTTCCA |
| | | GTACGGTTCAGGGGCCTCGGGTCATTGTTGTGGGACCTACAGATTCTG |
| | | GGAAGAGTACATTGTCAAGAATGCTTCTTAGTTGGGCAGCTAAACAGG |
| | | GTTGGAAACCTACTTTTGTGGACTTGGATATTGGTCAAGGATCCATAA |
| | | CTCTACCGGGATGTATTGCTGCTACTCCTATTGAAATGCCTATTGACCC |
| | | AGTTGAAGGGATTCCTCTTGAGATGCCTTTGGTTTACTTCTATGGACAC |
| | | ACCACTCCCAGTAACAATGTAGAACTATACAAAATCCTTGTGAGGGAG |
| | | CTTTCTCAACTGCTTGAGAGACAATTTGCGGGTAATATTGAATCTCGAG |
| | | CTGCTGGCATGGTGATCAATACCATGGGTTGGATAGAAGGAATAGGAT |
| | | ATGAGTTGCTTCTTCATGCAATCGACACATTTAATATCAACGTTGTATT |
| | | GGTTCTGGGTCAGGAAAAACTTTGCAGTATGCTCAGAGATGTTCTGAA |
| | | AAACAAGCCTGATGTGGATGTTGTAAAACTTCAAAGATCTGGAGGAGT |
| | | TGTATCGAGGAATGTCAAATATCGTCAGAAGGCTAGGAGTTTGAGGAT |
| | | AAGGGAATATTTTTATGGAAGGATAAACGATCTCTCCCCGCATTCTAA |
| | | TATTGCGAGTTTTAGTGATTTATTGGTCTATCGAATTGGTGGGGGACCT |
| | | CAGGCACCCCGGTCAGCCCTGCCAATTGGTGCAGAGCCTGCAGCAGAT |
| | | CCTACAAGATTAGTCCCTGTGAATATCAACCAGGATTTGCTCCATCTAG |
| | | TTCTTGCTGTTTCATTTGCGAAAGAGCCTGAGGAAATTCTTTCAAGCAA |
| | | CGTTGCCGGGTTTATATACATTACTGACATTGACATTCAGAGGAAGAA |
| | | GATTACATATCTTGCACCAGCTGCTGGTGATCTCCCGAGCAAATATCTG |
| | | ATTGTGGGAACCTTGACATGGATCGAAACATAG |
| SEQ ID NO: 209 | Abacus CLPS3 protein sequence | MAYGGTPLGPPPMAAAAASSSASTVRQVKLDRESELRIEVGYDSSLRLRL |
| | (1-445 AA) | LNGTAEIFGTELPPEIWLTFPPRLKFAVFTWYGATIEMDGSTETDYTADET |
| | | PMISYVNVHAILDGRRNRAKASSSDGSSTVQGPRVIVVGPTDSGKSTLSR |
| | | MLLSWAAKQGWKPTFVDLDIGQGSITLPGCIAATPIEMPIDPVEGIPLEMPL |
| | | VYFYGHTTPSNNVELYKILVRELSQLLERQFAGNVESRAAGMVINTMGWI |
| | | EGIGYELLLHAIDTFNINVVLVLGQEKLCSMLRDVLKNKPDVDVVKLQRS |
| | | GGVVSRNVKYRQKARSLRIREYFYGRINDLSPHSNIASFSDLLVYRIGGGP |
| | | QAPRSALPIGAEPAADPTRLVPVNINQDLLHLVLAVSFAKEPEEILSSNVAG |
| | | FIYITDIDIQRKKITYLAPAAGDLPSKYLIVGTLTWIET |
| SEQ ID NO: 210 | Finola/23TRC1-16-1 CLPS3 protein | MAYGGTPLGPPPMAAAAASSSASTVRQVKLDRESELRIEVGYDSSLRLRL |
| | sequence (1-445 AA) | LNGTAEIFGTELPPEIWLTFPPRLKFAVFTWYGATIELDGSTETDYTADETP |
| | | MISYVNVHAILDGRRNRA |
| | | KASSSDGSSTVQGPRVIVVGPTDSGKSTLSRMLLSWAAKQGWKPTFVDL |
| | | DIGQGSITLPGCIAATPIEMPIDPVEGIPLEMPLVYFYGHTTPSNNVELYKIL |
| | | VRELSQLLERQFAGNIES |
| | | RAAGMVINTMGWIEGIGYELLLHAIDTFNINVVLVLGQEKLCSMLRDVLK |
| | | NKPDVDVVKLQRSGGVVSRNVKYRQKARSLRIREYFYGRINDLSPHSNIA |
| | | SFSDLLVYRIGGGPQAPRSA |
| | | LPIGAEPAADPTRLVPVNINQDLLHLVLAVSFAKEPEEILSSNVAGFIYITDI |
| | | DIQRKKITYLAPAAGDLPSKYLIVGTLTWIET |
| SEQ ID NO: 211 | Abacus CLPS3 genomic DNA | GGATGTTTATTCTGTGAAAGGTTTTCACTTGGTATGGAGCCACAATTGA |
| | sequence flanking causative SNP | AATGGATGGTAGTACTGAAACTGACTACACTGCAGATGAGGTATTATT |
| | (A/C substitution located at position | TATC |
| | 262 bp in Abacus CDS) located at | |
| | position 51 bp (Abacus AA | |
| | substitution M88L) | |
| SEQ ID NO: 212 | Abacus CLPS3 genomic DNA | TCCTTGTGAGGGAGCTTTCTCAACTGCTTGAGAGACAATTTGCGGGTA |
| | sequence flanking causative SNP | ATGTTGAATCTCGAGCTGCTGGCATGGTGATCAATACCATGGGTTGGA |
| | (G/A substitution located at position | TAGAA |
| | 712 bp in Abacus CDS) located at | |
| | position 51 bp (Abacus AA | |
| | substitution V238I) | |
| SEQ ID NO: 213 | Abacus PI CDS (1-642 bp) | ATGGGAAGGGGTAAGATTGAGATTAAGAAAATAGAGAATACAAGTAA |
| | | TAGAGCAGTGACTTATGCTAAGAGAAAAGCTGGAATTTTCAAGAAGGC |
| | | TAAGGAGATTGCTATTCTTTGTGATGTTAAAGTCTCACTCATCATCGTT |
| | | TCTGGATCTGGCAAAATGGATGCCATGTGTAGTCCTGATAATTCGTTG |
| | | AGTACCATATGTGAGGAATATCACAAACACTCCAAAGAGAAGTTATGG |
| | | CCTGCTAAGCATGAGAACCTGCACAATGAAATTGAAATGGTCAAAAA |
| | | AGAAAATGAGAACATGGTGATAGAGCTGAGGCACCTGAATGGACAGG |
| | | ATATCAATCTGAACCACTATGAACTGATACCTATTGAGGAGGCACTTG |
| | | AGAATGGTCTTATAAGTGTTCGTGCCAGAAAGTCGGAGTTAGTTAACA |
| | | TTCTTAGGCAAAATCTGGAAGCTAAAGAGGAAGAGCATAAGCGCCTC |
| | | ATTTGCGAGCTGCACCAAAAGATGGAAGTAGTAGGGGATCATGGGTAT |
| | | CATAATCATCATCATCATCATGATCAGAGGCTGAATTTGAGGGACTAC |
| | | AATAATGCCCATATGCCTTTAGCCTTCCGTGTCCAGCCTATGCAGCCAA |
| | | ATCTCCAAGAGAGAATTTAG |
| SEQ ID NO: 214 | 23TRC1-17-1 CDS (73-618 bp)* | AAAGCTGGAATTTTCAAGAAGGCTAAGGAGATTGCTATTCTTTGTGAT |
| | | GTTAAAGTCTCACTCATCATCGTTTCTGGATCTGGCAAAATGGATGCCA |
| | | TGTGTAGTCCTGATAATTCGTTGAGTACCATATGTGAGGAATATCACA |
| | | AACACTCCAAAGAGAAGTTATGGCCTGCTAAGCATGAGAACCTGCACA |
| | | ATGAAATTGAAATGGTCAAAAAAGAAAATGAGAACATGGTGATAGAG |
| | | CTGAGGCACCTGAATGGACAGGATATCAATCTGAACCACTATGAACTG |
| | | ATACCTATTGAGGAGGCACTTGAGAATGGCCTTATAAGTGTTCGTGCC |
| | | AGAAAGTCGGAGTTAGTTAACATTCTTAGGCAAAATCTGGAAGCTAAA |
| | | GAGGAAGAGCATAAGCGCCTCATTTGCGAGCTGCACCAAAAGATGGA |
| | | AGTAGTAGGGGATCATGGGTATCATCATCATCATCATCATGATCAGAG |
| | | GCTGAATTTGAGGGACTACAATAATGCCCATATGCCTTTAGCCTTCCGT |
| | | GTCCAGCCTATGCAGCCA |
| SEQ ID NO: 215 | Abacus PI protein sequence (1-213 | MGRGKIEIKKIENTSNRAVTYAKRKAGIFKKAKEIAILCDVKVSLIIVSGSG |
| | AA) | KMDAMCSPDNSLSTICEEYHKHSKEKLWPAKHENLHNEIEMVKKENENM |
| | | VIELRHLNGQDINLNHYELIPIEEALENGLISVRARKSELVNILRQNLEAKE |
| | | EEHKRLICELHQKMEVVGDHGYHNHHHHHDQRLNLRDYNNAHMPLAFR |
| | | VQPMQPNLQERI |
| SEQ ID NO: 216 | 23TRC1-17-1 protein sequence (25 - | KAGIFKKAKEIAILCDVKVSLIIVSGSGKMDAMCSPDNSLSTICEEYHKHSK |
| | 206 AA)* | EKLWPAKHENLHNEIEMVKKENENMVIELRHLNGQDINLNHYELIPIEEA |
| | | LENGLISVRARKSELVNILRQNLEAKEEEHKRLICELHQKMEVVGDHGYH |
| | | HHHHHDQRLNLRDYNNAHMPLAFRVQPM QP |
| SEQ ID NO: 217 | Abacus PI genomic DNA sequence | TATATATGCAGCACCAAAAGATGGAAGTAGTAGGGGATCATGGGTATC |
| | flanking causative indel (AAT/- | ATAATCATCATCATCATCATGATCAGAGGCTGAATTTGAGGGACTACA |
| | indel located between positions 529- | ATAATGC |
| | 531 bp in Abacus CDS) located at | |
| | positions 51-53 bp (Abacus AA | |
| | substitution N177del) | |
| SEQ ID NO: 218 | Abacus AGO5 CDS (1-2730 bp) | ATGTCTTCTTCTCCGGTGGATTATTCATCGAAATGTGTGATGTTTCCGG |
| | | CAAGACCCGGTTATGGTATTGTTGGTAGAAAGTGTTTGATCAAAGCTA |
| | | ATCACTTCACACTCCAACTTGTTACCACCATCCATAACTATAAGGGTCT |
| | | CTATCATTACCATGTATGGATAAGTCCTCAAGTAACATCCATGAAATT |
| | | AAACAGAGATATTATTAAAGAGTTGGGAACTTTGAACTATTTAGGTAA |
| | | TGTGAGAGTTGCCTATGATGGTAGAAATACTATCTACTCTGTTGGGGA |
| | | GTTACCATTTTCTTCAAATGAATTTATTCTTAAGTTTCCCACAACAAAA |
| | | ACAACAACAACCACCAGTGAATGTGAGTTTAAGGTTAGCATCAGGTTC |
| | | GTTTCAAGGATAGACTTATGTAATCCACAAAGCTTTTTACACACCATTC |
| | | AACCACTTAACGTTGCTCTCAGATCAGCTCCATCAGAAATGTATAGTG |
| | | TTGTGGGGAGGTCTTTTTATCATGACACAATGAGTAGAGCTGGTGAGC |
| | | TTGGAGATGGTGTAATGTATTGGAAAGGTTTCTATCAAAGTTTGAGGC |
| | | CAACTCAATTGGGGCTTTCTCTTAACATTGATGTGTCAGCAAAGGCTTT |
| | | TTATGAACCTATTGAGGTAACTGATTTTATCTTCAAAAACTTTAATGTG |
| | | AGATACGATTCGACGCCATTGGATGATCATGTTCGGGTTCAGCTAAGG |
| | | AAAAATTTAAGAGGGGTTAAAGTAACATGTAGACATTTGGATGATACA |
| | | ATGTTGTTCAAAGTTTTTGGGGTATCGACAGAGCCATTGAACAATTTA |
| | | ATGTTTGATCTTGATGGTAACACGATTTCGGTTGTTACTTATTTTCAAG |
| | | ACAAGTACGAAATTCAGCTCGAATATGTGACATGGCCTGCTCTGCAAG |
| | | TAGGAAGTGCAACAAAGCCGATTTACTTGCCCATAGAGGTTTGTAGAA |
| | | TTGTTGAGGGGCAAAGATGCTACAAGAAACTCAACCAAAGGCAAGTA |
| | | ACCAATCTATTAAGGGAAACTTGTCAACGACCGTACGAAAGGGAGGA |
| | | AACCATTAGGCAAATTTTTCGACAAAACAATTACAATGATCAAAATCT |
| | | TGTTCGCGATTTTGGAATTAATGTTGCAAACGACATGACATTGATTAAT |
| | | GCTCGAGTTTTGCCTCCACCATTGCTTTTATACCATGATACTGGGAATG |
| | | AAGCTACTGCCAATCCTCAACTCGGGCAATGGAATATGATCAATAAGA |
| | | AAATGATCAATGGTGGCCATGTAAAATCTTGGACTTGTGTGAACTTCT |
| | | CACATGAAAATCCAAATATTCACAATCGCTTTTGTGATGAACTAGTCA |
| | | ATATGTGCATCAGTAGAGGAATGGTAGGGTTACTCAATCTTTCTGTCTC |
| | | GTTATGTTTTTTTTCGCGAAATTGCATAACTGACGTTTTTATCATTTTGG |
| | | ATACTCGGAATAAGGCTTTCCAAACCGCGCCAGTAATTCCTTCGCGCG |
| | | CTTGGCCAGCTAACGAAATTGAGGAGGCCTTGAAATATGTTCATGACC |
| | | AATGCAACATGATACTCGGAAACAATCAACTTCAGTTGCTAATAGTTA |
| | | TCTTGCCTGATTTTTCGGGTTCTTATGGGACAATTAAACGAGTTTGTGA |
| | | AACTGAGCTTGGAATTGTTTCACAATGCTGTCGGCCTAACCATGCAGC |
| | | GAAGCTCAACAACAAGCAATACTTGGAGAATCTATCATTGAAAATCAA |
| | | TACCAAGGTTGGTGGGCGAAATACAGTGTTAAGTGATGCTCTTAATAA |
| | | GAGAATTCCTTTTGTGTCTGATGTCCCGACGATAATCTTTGGCGCGGCT |
| | | TTTACACATCCAAAGCCCGAAGATTACTCAAGCCCTTCAATAGCAGCT |
| | | GTGGTTGCCTCGATGGATTGGCCCGAGGTGACCAAATATAGAGCGTTG |
| | | GTATCAGCGCAACAGCACACCGAGGAGATCATCCAAGATCTTTATAAA |
| | | GTAACTACAGATGATAAAGGGAGCATAGTCCATGGAGGAATGATAAG |
| | | GCGAGTTAATCACCATTGTGTACATGCCGATCAACCGGAGCCAAACCT |
| | | CATCGGATTATATTCTACCGGTTATTTGTGTAGCACACTTGGTAAAATC |
| | | TTTAGTGGTTTTTCATTACTCTGCCTCTTAATTTACTCTGTTTTGTGCAG |
| | | AGATGGCGTTAGTGATGGACAGTTCAATCGAGTGTTGCTTTACGAAAT |
| | | GGATGCGATAAGAAAGGCTTGTTCATCGCTCGAGGAAGGATATCTTCC |
| | | TCGAGTGACATTTGTTGCTATTCAGAAAAGGTGTCATACTCGCCTTTTC |
| | | CCTGCTGATTACAACGATCGCGATTCAATGGACAAGAGTGGCAACATT |
| | | CTACCAGGCACTGTTGTTGATACTAATATCTGCCACCCAACTCACTTCG |
| | | ATTTTTACCTCAAAAGTCATGCTGGAATTCAGGGAACAAGTCGTCCCG |
| | | CTCACTACCATGTCTTGTTCGACGAGAACAACTTCACAGCTGATGCCTT |
| | | ACAAGTTCTCACAAACAATCTATGCTACACGTATGCAAGGTGCACGAA |
| | | ATCAGTGTCTATCGTTCCTCCTGCATATTATGCACATTTGGCTGCGATT |
| | | CGTGCTCGTTGCTACATTGGAGTAGCAGTTGATGAAGAAGAAACTGAT |
| | | GTTGGCCCAAGAACTTATCAACCCCTTCAGGTGATCAATGGGAACCTT |
| | | AAAGATGTGATGTTCTATGTCTAA |
| SEQ ID NO: 219 | 23TRC1-17-1 AGO5 CDS (109- | CTCCAACTTGTTACCACCATCCATAACTATAAGGGTCTCTATCATTACC |
| | 2124) | ATGTATGGATAAGTCCTCAAGTAACATCCATGAAATTAAACAGAGATA |
| | | TCGTTAAAGAGTTGGGAACTTTGAACTATTTAGGTAATGTGAGAGTTG |
| | | CCTATGATGGTAGAAACACTATCTACACTGTTGGGGAGTTACCATTTTC |
| | | TTCAAATGAATTTATTCTTAAGTTTCCCACAACAAAAACAACAACAAC |
| | | CACCAGTGAATGTGAGTTTAAGGTTAGCATCAGGTTCGTTTCAAGGAT |
| | | AGACTTATGTAATCCACAAAGCTTTTTACACACCATTCAACCACTTAAT |
| | | GTTGCTCTCAGATCAGCTCCATCAGAAATGTATAGTGTTGTGGGGAGG |
| | | TCTTTTTATCATGACACAATGAGTAGAGCTGGTGAGCTTGGAGATGGT |
| | | GTAATGTATTGGAAAGGTTTCTATCAAAGTTTGAGGCCAACTCAATTG |
| | | GGGCTTTCTCTTAACATTGATGTGTCAGCAAAGGCTTTTTATGAACCTA |
| | | TTGAGGTAACTGATTTTATCTTCAAAAACTTTAATGTGAGATACGATTC |
| | | GACGCCATTGGATGATCATGTTCGGGTTCAGCTAAGGAAAAATTTAAG |
| | | AGGGGTTAAAGTAACATGTAGACATTTGGATGATACAATGTTGTTCAA |
| | | AATTTTTGGGGTATCGACAGAGCCATTGAACAATTTAATGTTTGATCTT |
| | | GATGGTAACACGATTTCGGTTGTTACTTACTTTCAAGATAAGTACGAA |
| | | ATTCAGCTCGAATATGTGACATGGCCTGCTCTGCAAGTAGGAAGTGCA |
| | | ACAAAGCCGATTTACTTGCCCATAGAGGTTTGTAGAATTGTTGAGGGG |
| | | CAAAGATGCTACAAGAAACTCAACCAAAGGCAAGTAACCAATCTATT |
| | | AAGGGAAACTTGTCAACGACCGTACGAAAGGGAGGAAACCATTAGGC |
| | | AAATTTTTCGACAAAACAATTACAATGATCARAATCTTGTTCGCGATTT |
| | | TGGAATTAATGTTGCAAACGACATGACATTGATTAATGCTCGAGTTTT |
| | | GCCTCCACCATTGCTTTTATACCATGATACTGGGAATGAAGCTACTGCS |
| | | AATCCTCAACTCGGRCAATGGAATATGATCAATAARAAAATGATCAAT |
| | | GGTGGCCATGTAAAATCTTGGACTTGTGTGAACTTCTCACATGAAAAT |
| | | CCARATATTCACAATCGCTTTTGTGATGAACTAGTCAATATGTGCATCA |
| | | GTAGAGGAATGGCTTTCCARACCAYGCCAGTAATTCCTTCGCGCGCTT |
| | | GGCCAGCTAATGAAATCAAGGAGGCCTTGAAATATGTTCATGACCAAT |
| | | GCAACATGATACTTGGAAACAAACAACTTCAGTTGCTAATAGTTATCT |
| | | TGCCTGATTTTTCGGGTTCTTATGGGACRATTAAACGAGTTTGTGAAAC |
| | | TGAGCTWGGAATKGTTTCACAATGCTGYCGGCCTAAACATGCAGCGA |
| | | AGCTCAACAACAAGCMATACTTGGAGAATCTATCATTGAAAATCAATA |
| | | CCAAGGTTGGTGGGCGAAATACAGTGTTAAGTGATGCTCTTAATAAGA |
| | | GAATTCCTTTTGTGTCTGATGTCCCGACGATAATCTTTGGCGCGGCTTT |
| | | TACACATCCAAAGCCCGAAGATTACTCAAGCCCTTCAATAGCAGCTGT |
| | | GGTTGCCTCGATGGATTGGCCCGAGGTGACCAAATATAGAGCGTTGGT |
| | | ATCAGCGCAACAGCACACCGAGGAGATCATCCAAGATCTTTATAAAGT |
| | | AACTACAGATGATAAAGGGAGCATAGTCCATGGAGGAATGATAAGGG |
| | | ATCATTTAATTGCTTTCAGCCGATCAACCGGAGCCAAACCTCACCGGA |
| | | TTATATTCTACCGAGATGGTGTTAGTGATGGACAGTTCAATCGAGTGTT |
| | | GCTTTACGAAATGGATGCGATAAGAAAGGCTTGTTCATCGCTCGATGA |
| | | AGGATATCTTCCTCGAGTGACATTTGTTGCTATTCAGAAA |
| SEQ ID NO: 220 | 22TRP2-2-2 AGO5 CDS (1705 | CCTTTTGTGTCTGATGTCCCGACGATAATCTTTGGTGCGGCTTTTACAC |
| | 2367 bp) | ATCCAAAGCCGGAAGATTACTCAAGCCCTTCAATAGCAGCTGTGGTTG |
| | | CCTCGATGGATTGGCCCGAGGTGACCAAATATAGAGCGTTGGTATCAG |
| | | CGCAACAGCACACCGAGGAGATCATCCAAGATCTTTATAAAGTAACTA |
| | | CAGATGATAAAGGGAGCATAGTCCATGGAGGAATGATAAGGGATCAT |
| | | TTAATTGCTTTCAGCCGATCAACCGGAGCCAAACCTCACCGGATTATA |
| | | TTCTACCGAGATGGCGTTAGTGATGGACAGTTCAATCGAGTGTTGCTTT |
| | | ACGAAATGGATGCGATAAGAAAGGCTTGTTCATCGCTCGAGGAAGAA |
| | | TATCTTCCTCGAGTGACATTTGTTGCTATTCAGAAAAGGTGTCATACTC |
| | | GCCTTTTCCCTGCTGATTACAACGATCGCAATTCAATGGACAAGAGTG |
| | | GCAACATTCTACCAGGCACTGTTGTTGATACTCATATCTGCCACCCAAC |
| | | TCAATTCGATTTTTACCTCAAAAGTCATGCTGGAATTCAGGGAACAAG |
| | | TCGTCCCGCTCACTACCATGTCTTGTTCGACGAGAACAACTTCACAGCT |
| | | GATGCCTTACAAGTTCTCACAAACAATCTATGCTAC |
| SEQ ID NO: 221 | Abacus AGO5 protein sequence (1- | MSSSPVDYSSKCVMFPARPGYGIVGRKCLIKANHFTLQLVTTIHNYKGLY |
| | 909 AA) | HYHVWISPQVTSMKLNRDIIKELGTLNYLGNVRVAYDGRNTIYSVGELPF |
| | | SSNEFILKFPTTKTTTTTSECEFKVSIRFVSRIDLCNPQSFLHTIQPLNVALRS |
| | | APSEMYSVVGRSFYHDTMSRAGELGDGVMYWKGFYQSLRPTQLGLSLNI |
| | | DVSAKAFYEPIEVTDFIFKNFNVRYDSTPLDDHVRVQLRKNLRGVKVTCR |
| | | HLDDTMLFKVFGVSTEPLNNLMFDLDGNTISVVTYFQDKYEIQLEYVTWP |
| | | ALQVGSATKPIYLPIEVCRIVEGQRCYKKLNQRQVTNLLRETCQRPYEREE |
| | | TIRQIFRONNYNDQNLVRDFGINVANDMTLINARVLPPPLLLYHDTGNEA |
| | | TANPQLGQWNMINKKMINGGHVKSWTCVNFSHENPNIHNRFCDELVNM |
| | | CISRGMVGLLNLSVSLCFFSRNCITDVFIILDTRNKAFQTAPVIPSRAWPAN |
| | | EIEEALKYVHDQCNMILGNNQLQLLIVILPDFSGSYGTIKRVCETELGIVSQ |
| | | CCRPNHAAKLNNKQYLENLSLKINTKVGGRNTVLSDALNKRIPFVSDVPTI |
| | | IFGAAFTHPKPEDYSSPSIAAVVASMDWPEVTKYRALVSAQQHTEEIIQDL |
| | | YKVTTDDKGSIVHGGMIRRVNHHCVHADQPEPNLIGLYSTGYLCSTLGKI |
| | | FSGFSLLCLLIYSVLCRDGVSDGQFNRVLLYEMDAIRKACSSLEEGYLPRV |
| | | TFVAIQKRCHTRLFPADYNDRDSMDKSGNILPGTVVDTNICHPTHFDFYL |
| | | KSHAGIQGTSRPAHYHVLFDENNFTADALQVLINNLCYTYARCTKSVSIV |
| | | PPAYYAHLAAIRARCYIGVAVDEEETDVGPRTYQPLQVINGNLKDVMFY |
| | | V |
| SEQ ID NO: 222 | 23TRC1-17-1 AGO5 protein | LQLVTTIHNYKGLYHYHVWISPQVTSMKLNRDIVKELGTLNYLGNVRVA |
| | sequence (37-708 AA) | YDGRNTIYTVGELPFSSNEFILKFPTTKTTTTTSECEFKVSIRFVSRIDLCNP |
| | | QSFLHTIQPLNVALRSAPSEMYSVVGRSFYHDTMSRAGELGDGVMYWKG |
| | | FYQSLRPTQLGLSLNIDVSAKAFYEPIEVTDFIFKNFNVRYDSTPLDDHVRV |
| | | QLRKNLRGVKVTCRHLDDTMLFKIFGVSTEPLNNLMFDLDGNTISVVTYF |
| | | QDKYEIQLEYVTWPALQVGSATKPIYLPIEVCRIVEGQRCYKKLNQRQVT |
| | | NLLRETCQRPYEREETIRQIFRONNYNDQNLVRDFGINVANDMTLINARV |
| | | LPPPLLLYHDTGNEATANPQLGQWNMINKKMINGGHVKSWTCVNFSHEN |
| | | PXIHNRFCDELVNMCISRGMAFQTXPVIPSRAWPANEIKEALKYVHDQCN |
| | | MILGNKQLQLLIVILPDFSGSYGTIKRVCETELGXVSQCCRPKHAAKLNNK |
| | | XYLENLSLKINTKVGGRNTVLSDALNKRIPFVSDVPTIIFGAAFTHPKPEDY |
| | | SSPSIAAVVASMDWPEVTKYRALVSAQQHTEEIIQDLYKVTTDDK |
| | | GSIVHGGMIRDHLIAFSRSTGAKPHRIIFYRDGVSDGQFNRVLLYEMDAIR |
| | | KACSSLDEG YLPRVTFVAIQK |
| SEQ ID NO: 223 | 22TRP2-2-2 AGO5 protein | PFVSDVPTIIFGAAFTHPKPEDYSSPSIAAVVASMDWPEVTKYRALVSAQQ |
| | sequence (569-789 AA) | HTEEIIQDLYKVTTDDKGSIVHGGMIRDHLIAFSRSTGAKPHRIIFYRDGVS |
| | | DGQFNRVLLYEMDAIRKACSSLEEEYLPRVTFVAIQKRCHTRLFPADYND |
| | | RNSMDKSGNILPGTVVDTHICHPTQFDFYLKSHAGIQGTSRPAHYHVLFDE |
| | | NNFTADALQVLINNLCY |
| SEQ ID NO: 224 | Abacus AGO5 genomic DNA | GAGGGGTTAAAGTAACATGTAGACATTTGGATGATACAATGTTGTTCA |
| | sequence flanking causative SNP | AAGTTTTTGGGGTATCGACAGAGCCATTGAACAATTTAATGTAAGTAA |
| | (G/A substitution located at position | CATAA |
| | 787 bp in Abacus CDS) located at | |
| | position 51 bp (Abacus AA | |
| | substitution V263I) | |
| SEQ ID NO: 225 | Abacus AGO5 genomic DNA | AACTGACGTTTTTATCATTTTGGATACTCGGAATAAGGCTTTCCAAACC |
| | sequence flanking causative SNP | GCGCCAGTAATTCCTTCGCGC |
| | (C/T substitution located at position | GCTTGGCCAGCTAACGAAATTGAGGAGGCCT |
| | 1388 bp in Abacus CDS) located at | |
| | position 51 bp (Abacus AA | |
| | substitution A463M) | |
| SEQ ID NO: 226 | Abacus AGO5 genomic DNA | TCCAAACCGCGCCAGTAATTCCTTCGCGCGCTTGGCCAGCTAACGAAA |
| | sequence flanking causative SNP | TTGAGGAGGCCTTGAAATATGTTCATGACCAATGCAACATGATACTCG |
| | (G/A substitution located at position | GAAAC |
| | 1429 bp in Abacus CDS) located at | |
| | position 51 bp (Abacus AA | |
| | substitution E477K) | |
| SEQ ID NO: 227 | Abacus AGO5 genomic DNA | GAGGCCTTGAAATATGTTCATGACCAATGCAACATGATACTCGGAAAC |
| | sequence flanking causative SNP | AATCAACTTCAGTTGCTAATAGTTATCTTGCCTGATTTTTCGGGTTCTT |
| | (T/A substitution located at position | ATGG |
| | 1482 bp in Abacus CDS) located at | |
| | position 51 bp (Abacus AA | |
| | substitution N494K) | |
| SEQ ID NO: 228 | Abacus AGO5 genomic DNA | TTGCCTCTTGTAGGGACAATTAAACGAGTTTGTGAAACTGAGCTTGGA |
| | sequence flanking causative SNP | ATTGTTTCACAATGCTGTCGGCCTAACCATGCAGCGAAGCTCAACAAC |
| | (T/G substitution located at position | AAGCA |
| | 1569 bp in Abacus CDS) located at | |
| | position 51 bp (Abacus AA | |
| | substitution 1523M) | |
| SEQ ID NO: 229 | Abacus AGO5 genomic DNA | CGAGTTTGTGAAACTGAGCTTGGAATTGTTTCACAATGCTGTCGGCCTA |
| | sequence flanking causative SNP | ACCATGCAGCGAAGCTCAACAACAAGCAATACTTGGAGAATCTATCAT |
| | (C/A substitution located at position | TGAA |
| | 1593 bp in Abacus CDS) located at | |
| | position 51 bp (Abacus AA | |
| | substitution N531K) | |
| SEQ ID NO: 230 | Abacus AGO5 genomic DNA | CCATGGCAATATCTAATCTGTTTATAATTACAGGCTTGTTCATCGCTCG |
| | sequence flanking causative SNP | AGGAAGGATATCTTCCTCGAGTGACATTTGTTGCTATTCAGAAAAGGT |
| | (G/T substitution located at position | GTCA |
| | 2082 bp in Abacus CDS) located at | |
| | position 51 bp (Abacus AA | |
| | substitution E694D) | |
| SEQ ID NO: 231 | Abacus AGO5 genomic DNA | GCAATATCTAATCTGTTTATAATTACAGGCTTGTTCATCGCTCGAGGAA |
| | sequence flanking causative SNP | GGATATCTTCCTCGAGTGACATTTGTTGCTATTCAGAAAAGGTGTCATA |
| | (G/A substitution located at position | CTC |
| | 2087 bp in Abacus CDS) located at | |
| | position 51 bp (Abacus AA | |
| | substitution G696E) | |
| SEQ ID NO: 232 | Abacus AGO5 genomic DNA | TTCAGAAAAGGTGTCATACTCGCCTTTTCCCTGCTGATTACAACGATCG |
| | sequence flanking causative SNP | CGATTCAATGGACAAGAGTGGCAACATTCTACCAGGTTTTAGTGTGTG |
| | (G/A substitution located at position | AATC |
| | 2167 bp in Abacus CDS) located at | |
| | position 51 bp (Abacus AA | |
| | substitution D723N) | |
| SEQ ID NO: 233 | Abacus AGO5 genomic DNA | ATTTGATTGGTTTTACTATTTTGCATTTTGAAGGCACTGTTGTTGATACT |
| | sequence flanking causative SNP | AATATCTGCCACCCAACTCACTTCGATTTTTACCTCAAAAGTCATGCTG |
| | (A/C substitution located at position | GA |
| | 2218 bp in Abacus CDS) located at | |
| | position 51 bp (Abacus AA | |
| | substitution N740H) | |
| SEQ ID NO: 234 | Abacus AGO5 genomic DNA | TTTTGCATTTTGAAGGCACTGTTGTTGATACTAATATCTGCCACCCAAC |
| | sequence flanking causative SNP | TCACTTCGATTTTTACCTCAAAAGTCATGCTGGAATTCAGGTCCAAAAT |
| | (C/A substitution located at position | GTC |
| | 2238 bp in Abacus CDS) located at | |
| | position 51 bp (Abacus AA | |
| | substitution H746Q) | |

It will be apparent that the precise details of the methods or compositions described may be varied or modified without departing from the spirit of the present disclosure. We claim all such modifications and variations that fall within the scope and spirit of the claims below.

1. A method for producing one or more Cannabis plants that flower at increased daylight hours, the method comprising:
(i) obtaining a nucleic acid sample from a Cannabis plant or its germplasm;
(ii) detecting one or more markers that indicate flower initiation at increased daylight hours;
(iii) crossing the Cannabis plant comprising the one or more markers; and
(iv) obtaining one or more progeny plants comprising the one or more markers and that initiate flowering at increased daylight hours;
wherein increased daylight hours is a light period of 13 to 18 hours.

2. (canceled)

3. The method of claim 1, wherein the Cannabis plant comprising the one or more markers is crossed with a second plant that does not initiate flowering at increased daylight hours.

4. The method of claim 1, wherein the one or more markers comprises a polymorphism on chromosome 8 relative to a reference genome at nucleotide positions: 159,096; 109,674; 1,162,631; 241,017; 1,844,801; 1,355,612; 19,681; 44,981; 51,589; 87,904; 199,126; 371,700; 378,368; 385,390; 447,673; 555,854; 564,634; 568,544; 720,538; 936,746; 1,042,634; 1,088,206; 1,147,746; 1,191,275; 1,200,057; 1,211,168; 1,216,900; 1,218,786; 1,277,500; 1,305,959; 1,397,056; 1,496,730; 1,515,643; 1,526,023; 1,577,268; 1,582,128; 1,617,208; 1,659,209; 1,690,294; 1,725,273; 1,729,625; 1,735,848; 1,816,071; 1,877,889; 1,933,751; 1,993,648; 2,002,233; 2,020,279; 2,253,641; 2,279,386; 2,364,298; 2,404,096; 63,750; 95,593; 100,604; 146,257; 321,930; 334,676; 351,874; 395,213; 413,345; 479,822; 491,066; 498,442; 512,584; 531,560; 537,879; 606,518; 639,258; 673,171; 690,591; 703,062; 709,849; 714,581; 784,607; 805,642; 827,002; 859,843; 935,803; 957,083; 963,275; 968,829; 972,965; 978,102; 1,008,257; 1,048,013; 1,076,208; 1,078,159; 1,108,104; 1,134,931; 1,154,438; 1,193,261; 1,228,323; 1,273,758; 1,281,517; 1,319,381; 1,376,341; 1,511,014; 1,557,156; 1,595,751; 1,600,294; 1,618,178; 1,622,540; 1,707,047; 1,753,859; 1,783,885; 1,790,563; 1,802,247; 1,810,522; 1,813,955; 1,835,318; 1,872,522; 1,909,161; 1,911,974; 1,944,792; 1,956,392; 1,971,081; 1,974,016; 1,997,626; 2,061,626; 2,079,392; 2,079,928; 2,087,331; 2,099,627; 2,101,607; 2,127,039; 2,160,732; 2,211,816; 2,267,196; 2,369,020; 2,383,741; 2,503,244; 2,504,080; 2,510,824; 2,816,933; 2,828,279; 2,833,001; 2,846,467; 2,862,613; 2,912,209; 3,098,435; or 3,794,786;
wherein the reference genome is the Abacus Cannabis reference genome version Csat_AbacusV2, NCBI assembly accession GCA_025232715.1.

5. The method of claim 4 wherein the nucleotide position comprises on chromosome 8:
(a) a A/A or G/A genotype at position 19681;
(b) a A/A or T/A genotype at position 44981;
(c) a C/C or T/C genotype at position 51589;
(d) a G/G or A/G genotype at position 87904;
(e) a G/G or G/A genotype at position 159096;
(f) a G/G or G/C genotype at position 199126;
(g) a A/A or G/A genotype at position 371700;
(h) a G/G or G/T genotype at position 378368;
(i) a G/G or G/A genotype at position 385390;
(j) a G/G or A/G genotype at position 447673;
(k) a T/T or T/C genotype at position 555854;
(l) a T/T or T/C genotype at position 564634;
(m) a G/G or G/A genotype at position 568544;
(n) a T/T or T/G genotype at position 720538;
(o) a A/A or A/T genotype at position 936746;
(p) a G/G or G/A genotype at position 1042634;
(q) a C/C or T/C genotype at position 1088206;
(r) a C/C or C/T genotype at position 1147746;
(s) a C/C or T/C genotype at position 1191275;
(t) a G/G or G/A genotype at position 1200057;
(u) a T/T or C/T genotype at position 1211168;
(v) a T/T or T/C genotype at position 1216900;
(w) a G/G or A/G genotype at position 1218786;
(x) a T/T or G/T genotype at position 1277500;
(y) a T/C genotype at position 1305959;
(z) a A/A or A/G genotype at position 1397056;
(aa) a A/A or A/G genotype at position 1496730;
(ab) a T/T or T/C genotype at position 1515643;
(ac) a A/A or A/T genotype at position 1526023;
(ad) a G/G or A/G genotype at position 1577268;
(ae) a T/T or G/T genotype at position 1582128;
(af) a C/C or G/C genotype at position 1617208;
(ag) a C/C or A/C genotype at position 1659209;
(ah) a A/A or A/G genotype at position 1690294;
(ai) a G/G or G/A genotype at position 1725273;
(aj) a C/C or C/G genotype at position 1729625;
(ak) a G/G or T/G genotype at position 1735848;
(al) a A/A or T/A genotype at position 1816071;
(am) a C/C or A/C genotype at position 1877889;
(an) a C/C or C/A genotype at position 1933751;
(ao) a T/T or C/T genotype at position 1993648;
(ap) a A/A or C/A genotype at position 2002233;
(aq) a A/A or A/G genotype at position 2020279;
(ar) a C/C or C/A genotype at position 2253641;
(as) a C/C or C/A genotype at position 2279386;
(at) a G/G or A/G genotype at position 2364298;
(an) a G/G or A/G genotype at position 2404096;
(av) a C/C or G/C genotype at position 63750;
(aw) a C/C or T/C genotype at position 95593;
(ax) a T/T or C/T genotype at position 100604;
(ay) a A/A or T/A genotype at position 109674;
(az) a A/A or A/C genotype at position 146257;
(ba) a A/A or A/G genotype at position 241017;
(bb) a G/G or G/A genotype at position 321930;
(be) a T/T or T/A genotype at position 334676;
(bd) a A/A or A/T genotype at position 351874;
(be) a C/C or C/A genotype at position 395213;
(bf) a G/G or A/G genotype at position 413345;
(bg) a T/T or T/C genotype at position 479822;
(bh) a T/T or T/A genotype at position 491066;
(bi) a T/T or C/T genotype at position 498442;
(bj) a A/A or G/A genotype at position 512584;
(bk) a A/A or A/C genotype at position 531560;
(bl) a A/A or A/G genotype at position 537879;
(bm) a A/A or G/A genotype at position 606518;
(bn) a A/A or A/G genotype at position 639258;
(bo) a A/A or G/A genotype at position 673171;
(bp) a C/C or C/T genotype at position 690591;
(bq) a A/A or A/C genotype at position 703062;
(br) a C/C or A/C genotype at position 709849;
(bs) a A/A or G/A genotype at position 714581;
(bt) a G/G or G/A genotype at position 784607;
(bu) a C/C or C/T genotype at position 805642;
(by) a T/T or T/C genotype at position 827002;
(bw) a G/G or G/A genotype at position 859843;
(bx) a A/A or A/G genotype at position 935803;
(by) a A/A or T/A genotype at position 957083;
(bz) a C/C or C/T genotype at position 963275;
(ca) a T/T or T/A genotype at position 968829;
(cb) a C/C or C/A genotype at position 972965;
(cc) a A/A or A/G genotype at position 978102;
(cd) a T/T or T/A genotype at position 1008257;
(ce) a C/C or C/T genotype at position 1048013;
(cf) a G/G or G/T genotype at position 1076208;
(cg) a A/A or A/G genotype at position 1078159;
(ch) a A/A or G/A genotype at position 1108104;
(ci) a A/A or A/T genotype at position 1134931;
(cj) a G/G or A/G genotype at position 1154438;
(ck) a G/G or C/G genotype at position 1162631;
(cl) a G/G or G/A genotype at position 1193261;
(cm) a C/C or T/C genotype at position 1228323;
(en) a T/T or C/T genotype at position 1273758;
(co) a A/A or A/C genotype at position 1281517;
(cp) a A/A or G/A genotype at position 1319381;
(cq) a G/G or A/G genotype at position 1355612;
(cr) a G/G or A/G genotype at position 1376341;
(cs) a C/C or C/T genotype at position 1511014;
(ct) a G/G or G/T genotype at position 1557156;
(cu) a A/A or A/G genotype at position 1595751;
(cv) a A/A or A/G genotype at position 1600294;
(cw) a T/T or T/C genotype at position 1618178;
(cx) a G/G or G/A genotype at position 1622540;
(cy) a T/T or T/C genotype at position 1707047;
(cz) a G/G or T/G genotype at position 1753859;
(da) a G/G or A/G genotype at position 1783885;
(db) a G/G or A/G genotype at position 1790563;
(dc) a A/A or A/G genotype at position 1802247;
(dd) a A/A or C/A genotype at position 1810522;
(de) a C/C or C/A genotype at position 1813955;
(df) a C/C or C/G genotype at position 1835318;
(dg) a G/G or A/G genotype at position 1844801;
(dh) a G/G or T/G genotype at position 1872522;
(di) a T/T or C/T genotype at position 1909161;
(dj) a G/G or A/G genotype at position 1911974;
(dk) a A/A or A/G genotype at position 1944792;
(dl) a G/G or G/A genotype at position 1956392;
(dm) a C/C or C/T genotype at position 1971081;
(dn) a T/T or T/C genotype at position 1974016;
(do) a T/T or T/C genotype at position 1997626;
(dp) a A/A or T/A genotype at position 2061626;
(dq) a C/C or T/C genotype at position 2079392;
(dr) a G/G or T/G genotype at position 2079928;
(ds) a T/T or T/G genotype at position 2087331;
(dt) a C/C or T/C genotype at position 2099627;
(du) a T/T or A/T genotype at position 2101607;
(dv) a G/G or G/A genotype at position 2127039;
(dw) a A/A or G/A genotype at position 2160732;
(dx) a A/A or A/G genotype at position 2211816;
(dy) a T/T or C/T genotype at position 2267196;
(dz) a A/A or G/A genotype at position 2369020;
(ea) a C/C or T/C genotype at position 2383741;
(eb) a G/G or A/G genotype at position 2503244;
(ec) a A/A or A/G genotype at position 2504080;
(ed) a T/T or A/T genotype at position 2510824;
(ee) a T/T or C/T genotype at position 2816933;
(ef) a G/G or A/G genotype at position 2828279;
(eg) a A/A or G/A genotype at position 2833001;
(eh) a C/C or C/G genotype at position 2846467;
(ei) a T/T or T/A genotype at position 2862613;
(ej) a T/T or C/T genotype at position 2912209;
(ek) a T/T or C/T genotype at position 3098435; or
(el) a G/G or T/G genotype at position 3794786;
wherein the reference genome is the Abacus Cannabis reference genome version Csat_AbacusV2, NCBI assembly accession GCA_025232715.1.

6-8. (canceled)

9. The method of claim 1, wherein the increased daylight hours is a light period of 16 to 18 hours.

10. The method of claim 1, wherein the one or more markers comprise a polymorphism on chromosome 8 relative to a reference genome at nucleotide positions:
(i) 159,096; 109,674; 1,162,631; 241,017; 1,844,801; or 1,355,612;
(ii) 159,096; 241,017; 321,930; 334,676; 351,874; 479,822; 498,442; 531,560; 537,879; 606,518; 639,258; 673,171; 703,062; 709,849; 714,581; 784,607; 1,319,381; or 1,376,341; or
(iii) 159,096; 109,674; 19,681; 44,981; 87,904; 378,368: 479,822; 491,066; 568,544; 720,538; 968,829; 1,191,275: 1,200,057; 1,211,168: 1,273,758; 1,277,500; 1,305,959; 1,659,209; 1,690,294; 1,735,848; 1,816,071; 1,872,522; 1,993,648; 2,002,233; 2,020,279; 2,079,928; or 2,267,196; and
wherein the markers indicate flower initiation at increased daylight hours and increased daylight hours is a light period of 13 to 18 hours.

11. The method of claim 10, wherein the one or more markers comprise a marker provided in (i), and wherein the marker genotype is:
(a) a G/G or G/A genotype at position 159096;
(b) a A/A or T/A genotype at position 109674;
(c) a G/G or C/G genotype at position 1162631;
(d) a A/A or A/G genotype at position 241017;
(e) a G/G or A/G genotype at position 1844801; or
(f) a G/G or A/G genotype at position 1355612.

12. (canceled)

13. The method of claim 10, wherein the one or more markers comprise a marker provided in (ii), and wherein the marker genotype is:
(a) a G/G or G/A genotype at position 159096;
(b) a A/A or A/G genotype at position 241017;
(c) a G/G or G/A genotype at position 321930;
(d) a T/T or T/A genotype at position 334676;
(e) a A/A or A/T genotype at position 351874;
(f) a T/T or T/C genotype at position 479822;
(g) a T/T or C/T genotype at position 498442;
(h) a A/A or A/C genotype at position 531560;
(i) a A/A or A/G genotype at position 537879;
(i) a A/A or G/A genotype at position 606518;
(k) a A/A or A/G genotype at position 639258;
(l) a A/A or G/A genotype at position 673171;
(m) a A/A or A/C genotype at position 703062;
(n) a C/C or A/C genotype at position 709849;
(o) a A/A or G/A genotype at position 714581;
(p) a G/G or G/A genotype at position 784607;
(q) a A/A or G/A genotype at position 1319381; or
(r) a G/G or A/G genotype at position 1376341; and
wherein the markers further indicate flower initiation in a fewer number of days relative to a control.

14. The method of claim 10, wherein the one or more markers comprise a marker provided in (iii), and wherein the marker genotype is:
(a) a G/G or G/A genotype at position 159096;
(b) a A/A or T/A genotype at position 109674;
(c) a A/A or G/A genotype at position 19681;
(d) a A/A or T/A genotype at position 44981;
(e) a G/G or A/G genotype at position 87904;
(f) a G/G or G/T genotype at position 378368;
(g) a T/T or T/C genotype at position 479982;
(h) a T/T or T/A genotype at position 491068;
(i) a G/G or G/A genotype at position 5698544;
(j) a T/T or T/A genotype at position 720538;
(k) a T/T or T/A genotype at position 968842;
(l) a C/C or T/C genotype at position 1191275;
(m) a G/G or G/A genotype at position 1200057;
(n) a T/T or C/T genotype at position 1211168;
(o) a T/T or C/T genotype at position 1273758;
(p) a T/T or G/T genotype at position 1277500;
(q) a T/C or genotype at position 1305959;
(r) a C/C or A/C genotype at position 1659209;
(s) a A/A or A/G genotype at position 1690294;
(t) a G/G or T/G genotype at position 1735848;
(u) a A/A or T/A genotype at position 1816071;
(v) a G/G or T/G genotype at position 1872522;
(w) a T/T or C/T genotype at position 1993648;
(x) a A/A or C/A genotype at position 2002233;
(y) a A/A or A/G genotype at position 2020279;
(z) a G/G or T/G genotype at position 2079928; or
(aa) a T/T or C/T genotype at position 2267196.

15-17. (canceled)

18. The method of claim 1, wherein the detecting comprises using an oligonucleotide probe and/or primer.

19. The method of claim 1, further comprising crossing the progeny plants to produce one or more additional progeny plants, and detecting the one or more markers that indicate flower initiation at increased daylight hours in the one or more additional progeny plants.

20. The method of claim 1, wherein crossing comprises selfing, sibling crossing, outcrossing, or backcrossing.

21-22. (canceled)

23. A method of producing one or more Cannabis plants having modified flowering, comprising:
(i) obtaining a nucleic acid sample from a Cannabis plant or its germplasm;
(ii) detecting one or more nucleic acid polymorphisms associated with flowering at increased daylight hours or flowering in a fewer number of days in one or more of:
(a) EARLY FLOWERING 9 (ELF9);
(b) FLOWERING LOCUS T (FT);
(c) CYCLIC DOF FACTOR 2 (CDF2);
(d) ARGONAUTE 5 (AGO5);
(e) GAST1 PROTEIN HOMOLOG 4 (GASA4);
(f) CLP-SIMILAR PROTEIN 3 (CLPS3); and
(g) PISTILLATA (PI);
(iii) crossing the Cannabis plant comprising the one or more nucleic acid polymorphisms, and
(iv) obtaining progeny plants comprising the one or more nucleic acid polymorphisms, thereby producing one or more Cannabis plants having modified flowering.

24-35. (canceled)

36. A method of producing a genetically engineered Cannabis plant that initiates flowering at increased daylight hours, comprising:
introducing a genetic modification in ELF9 and FT, or
introducing a beneficial allele of ELF9 and FT.

37-44. (canceled)

45. A progeny plant produced by the method of claim 1.

46-47. (canceled)

48. A seed of the progeny plant of claim 45.

49. A protoplast or tissue culture of cells produced from the progeny plant of claim 45.

50. A plant generated from the tissue culture of claim 49.

51-52. (canceled)

53. A Cannabis product comprising the progeny plant of claim 45.

54. The Cannabis product of claim 53, wherein the product is a kief, hashish, bubble hash, an edible product, solvent reduced oil, sludge, e-juice, or tincture.
